SEQUENCE LISTING (9 sequences)

SEQ ID NO: 1 - DNA, 21 bp, Artificial Sequence
  Description of Artificial Sequence Synthetic oligonucleotide
cactggcaagcacgaagaaag

SEQ ID NO: 2 - DNA, 21 bp, Artificial Sequence
  Description of Artificial Sequence Synthetic oligonucleotide
caccggcaagcacgaggagcg

SEQ ID NO: 3 - protein, 205 aa, Homo sapiens
MTERRVPFSLLRGPSWDPFRDWYPHSRLFDQAFGLPRLPEEWSQWLGGSSWPGYVRPLPPAAIESPAVAAPAYSRALSRQLSSGVSEIRHTADRWRVSLDVNHFAPDELTVKTKDGVVEITGKHEERQDEHGYISRCFTRKYTLPPGVDPTQVSSSLSPEGTLTVEAPMPKLATQSNEITIPVTFESRAQLGGPEAAKSDETAAK

SEQ ID NO: 4 - protein, 240 aa, Homo sapiens
MFAEFFGGRNPFDTFFGQRNGEEGMDIDDPFSGFPMGMGGFTNVNFGRSRSAQEPARKKQDPPVTHDLRVSLEEIYSGCTKKMKISHKRLNPDGKSIRNEDKILTIEVKKGWKEGTKITFPKEGDQTSNNIPADIVFVLKDKPHNIFKRDGSDVIYPARISLREALCGCTVNVPTLDGRTIPVVFKDVIRPGMRRKVPGEGLPLPKTPEKRGDLIIEFEVIFPERIPQTSRTVLEQVLPI

SEQ ID NO: 5 - protein, 573 aa, Homo sapiens
MLRLPTVFRQMRPVSRVLAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 6 - protein, 840 aa, Homo sapiens
MSVVGIDLGFQSCYVAVARAGGIETIANEYSDRCTPACISFGPKNRSIGAAAKSQVISNAKNTVQGFKRFHGRAFSDPFVEAEKSNLAYDIVQLPTGLTGIKVTYMEEERNFTTEQVTAMLLSKLKETAESVLKKPVVDCVVSVPCFYTDAERRSVMDATQIAGLNCLRLMNETTAVALAYGIYKQDLPALEEKPRNVVFVDMGHSAYQVSVCAFNRGKLKVLATAFDTTLGGRKFDEVLVNHFCEEFGKKYKLDIKSKIRALLRLSQECEKLKKLMSANASDLPLSIECFMNDVDVSGTMNRGKFLEMCNDLLARVEPPLRSVLEQTKLKKEDIYAVEIVGGATRIPAVKEKISKFFGKELSTTLNADEAVTRGCALQCAILSPAFKVREFSITDVVPYPISLRWNSPAEEGSSDCEVFSKNHAAPFSKVLTFYRKEPFTLEAYYSSPQDLPYPDPAIAQFSVQKVTPQSDGSSSKVKVKVRVNVHGIFSVSSASLVEVHKSEENEEPMETDQNAKEEEKMQVDQEEPHVEEQQQQTPAENKAESEEMETSQAGSKDKKMDQPPQAKKAKVKTSTVDLPIENQLLWQIDREMLNLYIENEGKMIMQDKLEKERNDAKNAVEEYVYEMRDKLSGEYEKFVSEDDRNSFTLKLEDTENWLYEDGEDQPKQVYVDKLAELKNLGQPIKIRFQESEERPKLFEELGKQIQQYMKIISSFKNKEDQYDHLDAADMTKVEKSTNEAMEWMNNKLNLQNKQSLTMDPVVKSKEIEAKIKELTSTCSPIISKPKPKVEPPKEEQKNAEQNGPVDGQGDNPGPQAAEQGTDTAVPSDSDKKLPEMDID

SEQ ID NO: 7 - protein, 732 aa, Homo sapiens
MPEETQTQDQPMEEEEVETFAFQAEIAQLMSLIINTFYSNKEIFLRELISNSSDALDKIRYESLTDPSKLDSGKELHINLIPNKQDRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEDKEEEKEKEEKESEDKPEIEDVGSDEEEEKKDGDKKKKKKIKEKYIDQEELNKTKPIWTRNPDDITNEEYGEFYKSLTNDWEDHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYYITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTADDTSAAVTEEMPPLEGDDDTSRMEEVD

SEQ ID NO: 8 - protein, 2549 aa, Homo sapiens
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 9 - DNA, 21 bp, Unknown
  Description of Unknown Tsc2 target sequence
ggtgaagagagccgtatcaca

(19) United States
(12) Patent Application Publication
(10) Pub. No.: US 2023/0218577 A1
(43) Pub. Date: Jul. 13, 2023

(54) USE OF HEAT SHOCK PROTEIN INHIBITORS FOR THE TREATMENT OF NEURODEVELOPMENTAL DISORDERS

(71) Applicant: The Children's Medical Center Corporation, Boston, MA (US)

(72) Inventors: Alessia DI NARDO, Boston, MA (US); Mustafa SAHIN, Boston, MA (US)

(73) Assignee: The Children's Medical Center Corporation, Boston, MA (US)

(21) Appl. No.: 18/009,645

(22) PCT Filed: Jun. 8, 2021
(86) PCT No.: PCT/US2021/036402

§ 371 Date: Dec. 9, 2022

Related U.S. Application Data
(60) Provisional application No. 63/037,946, filed on Jun. 11, 2020.

(51) Int. Cl. A61K 31/4152; A61K 31/4422; A61K 31/553; A61K 31/277; A61K 31/5377; A61P 25/00; C12N 15/86

(52) U.S. Cl. CPC A61K 31/4152; A61K 31/4422; A61K 31/553; A61K 31/277; A61K 31/5377; A61P 25/00; C12N 15/86; C12N 2710/10041

(57) ABSTRACT

Provided herein are methods of treating neurodevelopmental disorders, including the treatment of Tuberous sclerosis complex (TSC), with pharmaceutical compositions containing heat shock protein (Hsp) inhibitors and/or mTOR inhibitors. Also provided herein are methods for inhibiting mechanistic target of rapamycin complex 1 (mTORC1) activity and/or increasing or normalizing ciliation.

### CROSS-REFERENCE TO RELATED APPLICATION

This application claims the benefit of the following U.S. Provisional Application No. 63/037,946, filed Jun. 11, 2020, the entire contents of which are incorporated herein by reference.

### BACKGROUND OF THE DISCLOSURE

Tuberous sclerosis complex (TSC) is a neurodevelopmental disorder with an incidence of 1 in 6,000 caused by mutations in either the TSC1 or TSC2 genes, which encode proteins that form the TSC1/2 protein complex. TSC is associated with benign tumors called hamartomas in multiple organs as well as central nervous system (CNS) manifestations including epilepsy, intellectual disability and autism spectrum disorder (ASD). The neurological symptoms of TSC have been correlated with brain lesions called cortical tubers, which are characterized by the presence of giant cells and dysmorphic neurons with immature features. The TSC1/2 complex is an inhibitory regulator of the mechanistic target of rapamycin complex 1 (mTORC1), which coordinates key neurodevelopmental processes. Disrupted mTORC1 signaling has been clearly implicated in several aspects of the CNS pathogenesis seen in TSC. However, the molecular mechanisms downstream of mTORC1 hyperactivation that contribute to the neuronal abnormalities remain unclear.

TSC is a multisystem genetic disorder with a broad range of clinical symptoms, making the identification of effective treatments particularly challenging. Despite the clear implications of elevated mTORC1 activity as the mechanistic basis of TSC, an important set of unanswered questions revolve around the identification of downstream signaling abnormalities due to disrupted mTORC1 signaling in specific cell types that are affected by the disorder. Notably, mTOR inhibitor-based therapies have thus far been unsuccessful in treating the neuropsychiatric features of TSC. Alternative pathways to restore other aspects of mTOR signaling may provide new drug targets and broaden the therapeutic landscape for this disease.

Therefore, there is a need for novel methods and compositions for treating patients with TSC and related neurodevelopmental disorders with mTOR pathway dysfunction.

### SUMMARY OF THE DISCLOSURE

As described below, the present disclosure features methods of treating neurodevelopmental disorders, mTORopathies, and neuronal ciliopathies, including the treatment of Tuberous sclerosis complex (TSC), with pharmaceutical compositions containing one or more heat shock protein (Hsp) inhibitors and/or mechanistic target of rapamycin (mTOR) inhibitors. Also provided herein are methods for inhibiting mechanistic target of rapamycin complex 1 (mTORC1) activity and/or increasing or normalizing ciliation.

In one aspect, the disclosure provides a method for increasing or normalizing ciliation in a cell, the method comprising contacting the cell with one or more mTOR inhibitors selected from the group consisting of MCI-186, Nicardipine-HCl, K252A, Tyrphostin 9, LY-294002, rapamycin and everolimus, thereby increasing or normalizing ciliation.

In another aspect, the disclosure provides a method for increasing or normalizing ciliation in a cell, the method comprising contacting the cell with one or more heat shock protein (Hsp) inhibitors selected from the group consisting of inhibitory nucleic acids, 17-Allylamino-geldanamycin (17-AGG), Geldanamycin (GA), CUDC-305, and NVP-HSP990, thereby increasing or normalizing ciliation.

In yet another aspect, the disclosure provides a method for increasing or normalizing ciliation in a cell, the method comprising contacting the cell with one or more heat shock protein (Hsp) inhibitors and with one or more mTOR inhibitors, thereby increasing or normalizing ciliation.

In one aspect, the disclosure provides a method for increasing or normalizing ciliation in a cell, the method comprising contacting the cell with one or more heat shock protein (Hsp) inhibitors selected from the group consisting of inhibitory nucleic acids, 17-Allylamino-geldanamycin (17-AGG), Geldanamycin (GA), CUDC-305, and NVP-HSP990 and with one or more mTOR inhibitors selected from the group consisting of MCI-186, Nicardipine-HCl, K252A, Tyrphostin 9, LY-294002, rapamycin, and everolimus, thereby increasing or normalizing ciliation.

In another aspect, the disclosure provides a method for reducing a ciliation defect in a cell, the method comprising contacting the cell with one or more mTOR inhibitors selected from the group consisting of MCI-186, Nicardipine-HCl, K252A, Tyrphostin 9, LY-294002, rapamycin and/or everolimus, thereby reducing a ciliation defect.

In yet another aspect, the disclosure provides a method for reducing a ciliation defect in a cell, the method comprising contacting the cell with one or more heat shock protein (Hsp) inhibitors selected from the group consisting of inhibitory nucleic acids, 17-Allylamino-geldanamycin (17-AGG), Geldanamycin (GA), CUDC-305, and NVP-HSP990, thereby reducing a ciliation defect.

In one aspect, the disclosure provides a method for reducing a ciliation defect in a cell, the method comprising contacting the cell with one or more heat shock protein (Hsp) inhibitors selected from the group consisting of inhibitory nucleic acids, 17-Allylamino-geldanamycin (17-AGG), Geldanamycin (GA), CUDC-305, and NVP-HSP990 and one or more mTOR inhibitors selected from the group consisting of MCI-186, Nicardipine-HCl, K252A, Tyrphostin 9, LY-294002, rapamycin, and everolimus, thereby reducing a ciliation defect.

In another aspect, the disclosure provides a method for reducing a ciliation defect in a cell, the method comprising contacting the cell with one or more heat shock protein (Hsp) inhibitors and with one or more mTOR inhibitors, thereby reducing a ciliation defect.

In some embodiments, ciliation is increased or normalized relative to a reference. In some embodiments, ciliation is increased or normalized at least about 5%, at least about 10%, at least about 20%, at least about 30%, at least about 40%, at least about 50%, at least about 60%, at least about 70%, at least about 80%, at least about 90%, or at least about 100% relative to a reference. In some embodiments, ciliation is increased relative to an untreated cell. In some embodiments, ciliation is normalized relative to a wild-type cell. In some embodiments, the one or more Hsp inhibitors is selected from the group consisting of inhibitory nucleic acids, 17-Allylamino-geldanamycin (17-AGG), Geldanamycin (GA), CUDC-305, and NVP-HSP990. In some embodiments, the one or more mTOR inhibitors is selected from the group consisting of MCI-186, Nicardipine-HCl, K252A, Tyrphostin 9, LY-294002, rapamycin, and everolimus. In some embodiments, the cell is a neuron. In some embodiments, the cell is in vivo or in vitro. In some embodiments, the Hsp inhibitor is an inhibitory nucleic acid selected from a siRNA, shRNA, or antisense RNA. In some embodiments, the inhibitory nucleic acid is a shRNA. In some embodiments, the shRNA decreases the gene expression of HSPB1. In some embodiments, the shRNA is at least 85%, at least 90%, at least 95%, at least 98%, at least 99%, or 100% identical to the following nucleic acid sequence: 5′-CAC TGG CAA GCA CGA AGA AAG-3′. In some embodiments, the shRNA is at least 85%, at least 90%, at least 95%, at least 98%, at least 99%, or 100% identical to the following nucleic acid sequence: 5′-CAC CGG CAA GCA CGA GGA GCG-3′.

In one aspect, the disclosure provides a pharmaceutical composition comprising one or more heat shock protein (Hsp) inhibitors and one or more mTOR inhibitors. In some embodiments, the one or more Hsp inhibitors is selected from the group consisting of inhibitory nucleic acids, 17-Allylamino-geldanamycin (17-AGG), Geldanamycin (GA), CUDC-305, and NVP-HSP990. In some embodiments, the one or more mTOR inhibitors is selected from the group consisting of MCI-186, Nicardipine-HCl, K252A, Tyrphostin 9, LY-294002, rapamycin, and everolimus. In some embodiments, the Hsp inhibitor is an inhibitory nucleic acid selected from a siRNA, shRNA, or antisense RNA. In some embodiments, the inhibitory nucleic acid is a shRNA. In some embodiments, the shRNA decreases the gene expression of HSPB1. In some embodiments, the shRNA is at least 85%, at least 90%, at least 95%, at least 98%, at least 99%, or 100% identical to the following nucleic acid sequence: 5′-CAC TGG CAA GCA CGA AGA AAG-3′. In some embodiments, the shRNA is at least 85%, at least 90%, at least 95%, at least 98%, at least 99%, or 100% identical to the following nucleic acid sequence: 5′-CAC CGG CAA GCA CGA GGA GCG-3′. In some embodiments, the inhibitory nucleic acid is a naked polynucleotide. In some embodiments, the pharmaceutical composition includes a vector encoding an inhibitory nucleic acid. In some embodiments, the vector is a viral vector. In some embodiments, the vector is an adeno-associated virus (AAV) vector. In some embodiments, the vector comprises a promoter that drives expression of the inhibitory nucleic acid. In some embodiments, the pharmaceutical composition further includes a pharmaceutically acceptable carrier, diluent, excipient, or vehicle.

In one aspect, the disclosure provides a method of treating a subject with a neurodevelopmental disorder, the method comprising administering to the subject an effective amount of any of the pharmaceutical compositions disclosed herein, thereby treating the neurodevelopmental disorder.

In another aspect, the disclosure provides a method of treating a subject with a neurodevelopmental disorder, the method comprising administering to the subject an effective amount of a pharmaceutical composition comprising one or more mTOR inhibitors selected from the group consisting of MCI-186, Nicardipine-HCl, K252A, Tyrphostin 9, LY-294002, rapamycin, and everolimus, thereby treating the neurodevelopmental disorder.

In yet another aspect, the disclosure provides a method of treating a subject with a neurodevelopmental disorder, the method comprising administering to the subject an effective amount of a pharmaceutical composition comprising one or more heat shock protein (Hsp) inhibitors selected from the group consisting of inhibitory nucleic acids, 17-Allylamino-geldanamycin (17-AGG), Geldanamycin (GA), CUDC-305, and NVP-HSP990, thereby treating the neurodevelopmental disorder.

In some embodiments, the neurodevelopmental disorder is caused by a mutation in a mechanistic target of rapamycin (mTOR) regulatory gene. In some embodiments, the mTOR regulatory gene is selected from the group consisting of TSC1, TSC2, AKT3, and DEPDC5. In some embodiments, the neurodevelopmental disorder is associated with a mutation in the TSC1 or TSC2 genes. In some embodiments, the neurodevelopmental disorder is associated with dysfunctional mechanistic target of rapamycin complex 1 (mTORC1) activity. In some embodiments, the mTORC1 activity is increased in a cell of the subject. In some embodiments, the cell is a neuron. In some embodiments, the neurodevelopmental disorder is associated with a decrease in neuronal cilia. In some embodiments, the neurodevelopmental disorder is Tuberous Sclerosis Complex (TSC), intellectual disability, brain malformations, cortical tubers, neural ciliopathy, epilepsy, neuropathy, autism, hemimegalencephaly, cortical dysplasia, focal cortical dysplasia, traumatic brain injury, brain tumours, and/or dementia, or a combination thereof.

In one aspect, the disclosure provides a method of treating a subject with a mTORopathy, the method comprising administering to the subject an effective amount of any of the pharmaceutical compositions as provided herein, thereby treating the mTORopathy.

In another aspect, the disclosure provides a method of treating a subject with a mTORopathy, the method comprising administering to a subject an effective amount of a pharmaceutical composition comprising one or more mTOR inhibitors selected from the group consisting of MCI-186, Nicardipine-HCl, K252A, Tyrphostin 9, and LY-294002, rapamycin, and everolimus, thereby treating the mTORopathy.

In yet another aspect, the disclosure provides a method of treating a subject with a mTORopathy, the method comprising administering to the subject an effective amount of a pharmaceutical composition comprising one or more heat shock protein (Hsp) inhibitors selected from the group consisting of inhibitory nucleic acids, 17-Allylamino-geldanamycin (17-AGG), Geldanamycin (GA), CUDC-305, and NVP-HSP990, thereby treating the mTORopathy.

In one aspect, the disclosure provides a method of treating a subject with Tuberous Sclerosis Complex (TSC), the method comprising administering to the subject an effective amount of any of the pharmaceutical compositions as provided herein, thereby treating TSC.

In another aspect, the disclosure provides a method of treating a subject with Tuberous Sclerosis Complex (TSC), the method comprising administering to a subject an effective amount of a pharmaceutical composition comprising one or more mTOR inhibitors selected from the group consisting of MCI-186, Nicardipine-HCl, K252A, Tyrphostin 9, LY-294002, rapamycin, and everolimus, thereby treating TSC.

In yet another aspect, the disclosure provides a method of treating a subject with Tuberous Sclerosis Complex (TSC), the method comprising administering to a subject an effective amount of a pharmaceutical composition comprising one or more heat shock protein (Hsp) inhibitors selected from the group consisting of inhibitory nucleic acids, 17-Allylamino-geldanamycin (17-AGG), Geldanamycin (GA), CUDC-305, and NVP-HSP990, thereby treating TSC.

In one aspect, the disclosure provides a method of treating a subject with neuronal ciliopathy, the method comprising administering to the subject an effective amount of any of the pharmaceutical compositions as provided herein, thereby treating the neuronal ciliopathy.

In another aspect, the disclosure provides a method for treating a subject with neuronal ciliopathy, the method comprising administering to a subject an effective amount of a pharmaceutical composition comprising one or more mTOR inhibitors selected from the group consisting of MCI-186, Nicardipine-HCl, K252A, Tyrphostin 9, and LY-294002, rapamycin, and everolimus, thereby treating the neuronal ciliopathy.

In yet another aspect, the disclosure provides a method for treating a subject with neuronal ciliopathy, the method comprising administering to a subject an effective amount of a pharmaceutical composition comprising one or more heat shock protein (Hsp) inhibitors selected from the group consisting of inhibitory nucleic acids, 17-Allylamino-geldanamycin (17-AGG), Geldanamycin (GA), CUDC-305, and NVP-HSP990, thereby treating the neuronal ciliopathy.

In some embodiments, the pharmaceutical composition comprising one or more mTOR inhibitors is administered simultaneously or sequentially with an effective amount of a pharmaceutical composition comprising one or more Hsp inhibitors. In some embodiments, the one or more Hsp inhibitors is selected from the group consisting of inhibitory nucleic acids, 17-Allylamino-geldanamycin (17-AGG), Geldanamycin (GA), CUDC-305, and NVP-HSP990. In some embodiments, the pharmaceutical composition comprising one or more Hsp inhibitors is administered simultaneously or sequentially with an effective amount of a pharmaceutical composition comprising one or more mTOR inhibitors. In some embodiments, the one or more mTOR inhibitors is selected from the group consisting of MCI-186, Nicardipine-HCl, K252A, Tyrphostin 9, LY-294002, rapamycin, and everolimus. In some embodiments, the Hsp inhibitor is an inhibitory nucleic acid selected from a siRNA, shRNA, or antisense RNA. In some embodiments, the inhibitory nucleic acid is a shRNA. In some embodiments, the shRNA decreases the gene expression of HSPB1. In some embodiments, the shRNA is at least 85%, at least 90%, at least 95%, at least 98%, at least 99%, or 100% identical to the following nucleic acid sequence: 5′-CAC TGG CAA GCA CGA AGA AAG-3′. In some embodiments, the shRNA is at least 85%, at least 90%, at least 95%, at least 98%, at least 99%, or 100% identical to the following nucleic acid sequence: 5′-CAC CGG CAA GCA CGA GGA GCG-3′. In some embodiments, the inhibitory nucleic acid is administered as a naked polynucleotide. In some embodiments, the pharmaceutical composition administered to the subject comprises a vector encoding the inhibitory nucleic acid. In some embodiments, the vector is a viral vector. In some embodiments, the vector is an adeno-associated virus (AAV) vector. In some embodiments, the vector comprises a promoter that drives expression of the inhibitory nucleic acid.

In some embodiments, any of the methods as provided herein is performed in vivo or in vitro. In some embodiments, the subject is a mammal or a human. In some embodiments, the subject is a postnatal subject. In some embodiments, administration is systemic or oral. In some embodiments, the pharmaceutical composition further comprises a pharmaceutically acceptable carrier, diluent, excipient, or vehicle. In some embodiments, the step of administering comprises one or more doses.

In one aspect, the disclosure provides a kit for the treatment of a subject with a neurodevelopmental disorder, the kit comprising any of the pharmaceutical compositions as provided herein, for administration to the subject.

In another aspect, the disclosure provides a kit for the treatment of a subject with a neurodevelopmental disorder, the kit comprising one or more mTOR inhibitors selected from the group consisting of MCI-186, Nicardipine-HCl, K252A, Tyrphostin 9, LY-294002, rapamycin, and everolimus, for administration to the subject.

In yet another aspect, the disclosure provides a kit for the treatment of a subject with a neurodevelopmental disorder, the kit comprising one or more heat shock protein (Hsp) inhibitors selected from the group consisting of inhibitory nucleic acids, 17-Allylamino-geldanamycin (17-AGG), Geldanamycin (GA), CUDC-305, and NVP-HSP990, for administration to the subject.

In one aspect, the disclosure provides a kit for the treatment of a subject with a neurodevelopmental disorder, the kit comprising one or more heat shock protein (Hsp) inhibitors and one or more mTOR inhibitors, for administration to the subject. In some embodiments, the neurodevelopmental disorder is Tuberous Sclerosis Complex (TSC). In some embodiments, the kit further includes instructions for treating the subject.

Compositions and articles defined by the disclosure were isolated or otherwise manufactured in connection with the examples provided below. Other features and advantages of the disclosure will be apparent from the detailed description, and from the claims.

### Definitions

Unless defined otherwise, all technical and scientific terms used herein have the meaning commonly understood by a person skilled in the art to which this disclosure pertains or relates. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); Benjamin Lewin, Genes V, published by Oxford University Press, 1994 (ISBN 0-19-854287-9); Kendrew et al. (eds.); The Encyclopedia of Molecular Biology, published by Blackwell Science Ltd., 1994 (ISBN 0-632-02182-9); Molecular Biology and Biotechnology: a Comprehensive Desk Reference, Robert A. Meyers (ed.), published by VCH Publishers, Inc., 1995 (ISBN 1-56081-569-8); and Hale & Marham, The Harper Collins Dictionary of Biology (1991). As used herein, the following terms have the meanings ascribed to them below, unless specified otherwise.

By “administer” or “administration” is meant giving, supplying, or dispensing a composition, agent, therapeutic and the like to a subject, or applying or bringing the composition and the like into contact with the subject. Administering or administration may be accomplished by any of a number of routes, such as, for example, without limitation, topical, oral, subcutaneous, intramuscular, intraperitoneal, intravenous (IV), injection, intrathecal, intramuscular, dermal, intradermal, intracranial, inhalation, rectal, intravaginal, or intraocular.

By “agent” is meant any small molecule chemical compound, antibody, nucleic acid molecule, peptide, polypeptide, or fragments thereof.

By “alteration” is meant a change (increase or decrease) in the expression levels, structure, or activity of a gene or polypeptide as detected by standard art known methods such as those described herein. As used herein, an alteration includes a 5% change in expression levels, a 10% change in expression levels, a 25% change, a 40% change, and a 50% or greater change in expression levels. Such alteration may be relative to a reference.

By “ameliorate” is meant decrease, reduce, delay diminish, suppress, attenuate, arrest, or stabilize the development or progression of a disease or pathological condition. Tuberous sclerosis is an exemplary disease or pathological condition.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “cilia” or “primary cilia” is meant evolutionarily conserved membrane extensions of the cell surface made of microtubules that extend from a centriole-derived structure called the basal body. Cilia are described, for example, by Lee and Gleeson, Cilia in the nervous system: linking cilia function and neurodevelopmental disorders; Current opinion in neurology 24, 98-105 (2011). Cilia coordinate extracellular ligand-based signaling, and play a critical role in tissue homeostasis (Gerdes et al., The vertebrate primary cilium in development, homeostasis, and disease. Cell 137, 32-45 (2009)).

By “ciliation” is meant the growth and development of cilia.

A “ciliopathy” or “ciliopathies” refer to genetic disorders caused by mutations in genes that function in cilia assembly and/or function.

In this disclosure, “comprises,” “comprising,” “containing” and “having” and the like can have the meaning ascribed to them in U.S. Patent law and can mean “includes,” “including,” and the like; “consisting essentially of or” “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of an analyte, compound, agent, or substance to be detected.

By “detectable label” is meant a composition that, when linked to a molecule of interest, renders the latter detectable, e.g., via spectroscopic, photochemical, biochemical, immunochemical, or chemical means. Nonlimiting examples of useful detectable labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition, disorder, or pathology that damages or interferes with the normal function of a cell, tissue, or organ. In some embodiments, the disease is a neurodevelopmental disorder, neuronal ciliopathy, and/or mTORopathy. In some embodiments, non-limiting examples of diseases include tuberous sclerosis complex (TSC), intellectual disability, brain malformations, cortical tubers, neural ciliopathy, epilepsy, neuropathy, autism, hemimegalencephaly, cortical dysplasia, focal cortical dysplasia, traumatic brain injury, brain tumours, and dementia, or a combination thereof. In some embodiments, the disease is tuberous sclerosis complex (TSC).

By “effective amount” is meant the quantity of an active agent, composition, compound, or biologic sufficient to achieve a desired effect in a subject being treated with that active agent, composition, compound, or biologic. In the context of the present disclosure, an effective amount of an agent, composition, compound, or biologic is an amount sufficient to prevent, ameliorate, reduce, improve, abrogate, diminish, eliminate, delay and/or treat a disease, the symptoms and/or effects of a disease, condition, or pathology relative to an untreated subject without causing a substantial cytotoxic effect in the treated subject.

In some embodiments, an effective amount of a pharmaceutical composition is the amount required to inhibit mechanistic target of rapamycin complex 1 (mTORC1) activity in a subject. In some embodiments, an effective amount of a pharmaceutical composition is the amount required to decrease in mechanistic target of rapamycin complex 1 (mTORC1) activity in a subject relative to a reference, e.g., untreated subject. In some embodiments, an effective amount of a pharmaceutical composition is the amount required to increase ciliation in a cell of a subject. In some embodiments, an effective amount of a pharmaceutical composition is the amount required to increase ciliation in a cell of a subject relative to a reference, e.g., untreated subject. In some embodiments, ciliation in a cell is increased at least about 5%, at least about 10%, at least about 20%, at least about 30%, at least about 40%, at least about 50%, at least about 60%, at least about 70%, at least about 80%, at least about 90%, or at least about 100% relative to a reference, e.g., untreated subject. In some embodiments, an effective amount of a pharmaceutical composition is the amount required to increase ciliation in a neuron of a subject.

In some embodiments, an effective amount of a pharmaceutical composition is the amount required to increase ciliation in a neuron of a subject relative to a reference, e.g., untreated subject. In some embodiments, ciliation in a neuron is increased at least about 5%, at least about 10%, at least about 20%, at least about 30%, at least about 40%, at least about 50%, at least about 60%, at least about 70%, at least about 80%, at least about 90%, or at least about 100% relative to a reference, e.g., untreated subject.

The effective amount of a composition as used to practice the methods of therapeutic treatment of a disease, condition, or pathology, varies depending upon the manner of administration, the age, body weight, and general health of the subject. Ultimately, the attending physician or veterinarian will decide the appropriate amount and dosage regimen. Such amount is referred to as an “effective” amount.

The disclosure herein provides a number of targets that are useful for the development of highly specific drugs to treat a disease or disorder characterized by the methods delineated herein. In addition, the methods of the disclosure provide a facile means to identify therapies that are safe for use in subjects. In addition, the methods of the disclosure provide a route for analyzing virtually any number of compounds for effects on a disease described herein with high-volume throughput, high sensitivity, and low complexity.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

By “heat shock protein” or “Hsp” is meant a polypeptide or fragment thereof, which is produced by cells in response to exposure a heat shock and having at least about 85% amino acid sequence identity to one of the following reference sequences: NP_001300893.1, NP_002147.2, NP_002145.3, NP_005339.3. In some embodiments, Hsps are produced in response to other forms of stress in addition to heat, such as, cold, UV light and during wound healing or tissue remodeling. Hsps are known in the art and described, for example, by Garrido et al., Heat shock proteins 27 and 70: anti-apoptotic proteins with tumorigenic properties; Cell Cycle 5, 2592-2601 (2006), which is incorporated by reference in its entirety. Hsps are divided into six families based on their size, and these proteins function in multi-component complexes that are closely inter-related (Chatterjee and Burns, Targeting Heat Shock Proteins in Cancer: A Promising Therapeutic Approach; Int J Mol Sci 18 (2017)). Many Hsp members perform chaperone functions by stabilizing new proteins to ensure correct folding or by helping to refold proteins that were damaged by the cell stress. Hsps are found in virtually all living organisms, from bacteria to humans. In some embodiments, the Hsps described herein are from humans.

Heat-shock proteins are named according to their molecular weight. In some embodiments, the heat shock proteins include heat shock protein 27 (Hsp27), heat shock protein 40 (Hsp40), heat shock protein 60 (Hsp60), heat shock protein 70 (Hsp70), and/or heat shock protein 90 (Hsp90). Hsp40, Hsp60, Hsp70 and Hsp90 refer to families of heat shock proteins on the order of 40, 60, 70 and 90 kilodaltons in size, respectively.

In some embodiments, the heat shock protein is Hsp27. In some embodiments, Hsp27 is a polypeptide or fragment thereof having an amino acid sequence that is at least 85%, at least 90%, at least 95%, at least 98%, at least 99%, or 100% identical to a Hsp27 amino acid sequence associated with NCBI Reference Sequence: NP_001531.1. In some embodiments, Hsp27 is at least 85%, at least 90%, at least 95%, at least 98%, at least 99%, or 100% identical to the Hsp27 in Homo sapiens. An exemplary Hsp27 full-length amino acid sequence from Homo sapiens is provided below:

```
    1 MTERRVPFSL LRGPSWDPFR DWYPHSRLFD
        QAFGLPRLPE EWSQWLGGSS WPGYVRPLPP
  61 AAIESPAVAA PAYSRALSRQ LSSGVSEIRH
        TADRWRVSLD VNHFAPDELT VKTKDGVVEI
121 TGKHEERQDE HGYISRCFTR KYTLPPGVDP
        TQVSSSLSPE GTLTVEAPMP KLATQSNEIT
181 IPVTFESRAQ LGGPEAAKSD ETAAK
```

In some embodiments, the heat shock protein is Hsp40. In some embodiments, Hsp40 is a polypeptide or fragment thereof having an amino acid sequence that is at least 85%, at least 90%, at least 95%, at least 98%, at least 99%, or 100% identical to a Hsp40 amino acid sequence associated with NCBI Reference Sequence: NP_001300893.1. In some embodiments, Hsp40 is at least 85%, at least 90%, at least 95%, at least 98%, at least 99%, or 100% identical to the Hsp40 in Homo sapiens. An exemplary Hsp40 full-length amino acid sequence from Homo sapiens is provided below:

```
    1 MFAEFFGGRN PFDTFFGQRN GEEGMDIDDP
        FSGFPMGMGG FTNVNFGRSR SAQEPARKKQ
  61 DPPVTHDLRV SLEEIYSGCT KKMKISHKRL
        NPDGKSIRNE DKILTIEVKK GWKEGTKITF
121 PKEGDQTSNN IPADIVFVLK DKPHNIFKRD
        GSDVIYPARI SLREALCGCT VNVPTLDGRT
181 IPVVFKDVIR PGMRRKVPGE GLPLPKTPEK
        RGDLIIEFEV IFPERIPQTS RTVLEQVLPI
```

In some embodiments, the heat shock protein is Hsp60. In some embodiments, Hsp60 is a polypeptide or fragment thereof having an amino acid sequence that is at least 85%, at least 90%, at least 95%, at least 98%, at least 99%, or 100% identical to a Hsp60 amino acid sequence associated with NCBI Reference Sequence: NP_002147.2. In some embodiments, Hsp60 is at least 85%, at least 90%, at least 95%, at least 98%, at least 99%, or 100% identical to the Hsp60 in Homo sapiens. An exemplary Hsp60 full-length amino acid sequence from Homo sapiens is provided below:

```
    1 MLRLPTVFRQ MRPVSRVLAP HLTRAYAKDV
        KFGADARALM LQGVDLLADA VAVTMGPKGR
  61 TVIIEQSWGS PKVTKDGVTV AKSIDLKDKY
        KNIGAKLVQD VANNTNEEAG DGTTTATVLA
121 RSIAKEGFEK ISKGANPVEI RRGVMLAVDA
        VIAELKKQSK PVTTPEEIAQ VATISANGDK
181 EIGNIISDAM KKVGRKGVIT VKDGKTLNDE
        LEIIEGMKFD RGYISPYFIN TSKGQKCEFQ
241 DAYVLLSEKK ISSIQSIVPA LEIANAHRKP
        LVIIAEDVDG EALSTLVLNR LKVGLQVVAV
301 KAPGFGDNRK NQLKDMAIAT GGAVFGEEGL
        TLNLEDVQPH DLGKVGEVIV TKDDAMLLKG
361 KGDKAQIEKR IQEIIEQLDV TTSEYEKEKL
        NERLAKLSDG VAVLKVGGTS DVEVNEKKDR
421 VTDALNATRA AVEEGIVLGG GCALLRCIPA
        LDSLTPANED QKIGIEIIKR TLKIPAMTIA
481 KNAGVEGSLI VEKIMQSSSE VGYDAMAGDF
        VNMVEKGIID PTKVVRTALL DAAGVASLLT
541 TAEVVVTEIP KEEKDPGMGA MGGMGGGMGG
        GMF
```

In some embodiments, the heat shock protein is Hsp70. In some embodiments, Hsp70 is a polypeptide or fragment thereof having an amino acid sequence that is at least 85%, at least 90%, at least 95%, at least 98%, at least 99%, or 100% identical to a Hsp70 amino acid sequence associated with NCBI Reference Sequence: NP_002145.3. In some embodiments, Hsp70 is at least 85%, at least 90%, at least 95%, at least 98%, at least 99%, or 100% identical to the Hsp70 in Homo sapiens. An exemplary Hsp70 full-length amino acid sequence from Homo sapiens is provided below:

```
    1 MSVVGIDLGF QSCYVAVARA GGIETIANEY
        SDRCTPACIS FGPKNRSIGA AAKSQVISNA
  61 KNTVQGFKRF HGRAFSDPFV EAEKSNLAYD
        IVQLPTGLTG IKVTYMEEER NFTTEQVTAM
121 LLSKLKETAE SVLKKPVVDC VVSVPCFYTD
        AERRSVMDAT QIAGLNCLRL MNETTAVALA
181 YGIYKQDLPA LEEKPRNVVF VDMGHSAYQV
        SVCAFNRGKL KVLATAFDTT LGGRKFDEVL
241 VNHFCEEFGK KYKLDIKSKI RALLRLSQEC
        EKLKKLMSAN ASDLPLSIEC FMNDVDVSGT
301 MNRGKFLEMC NDLLARVEPP LRSVLEQTKL
        KKEDIYAVEI VGGATRIPAV KEKISKFFGK
361 ELSTTLNADE AVTRGCALQC AILSPAFKVR
        EFSITDVVPY PISLRWNSPA EEGSSDCEVF
421 SKNHAAPFSK VLTFYRKEPF TLEAYYSSPQ
        DLPYPDPAIA QFSVQKVTPQ SDGSSSKVKV
481 KVRVNVHGIF SVSSASLVEV HKSEENEEPM
        ETDQNAKEEE KMQVDQEEPH VEEQQQQTPA
541 ENKAESEEME TSQAGSKDKK MDQPPQAKKA
        KVKTSTVDLP IENQLLWQID REMLNLYIEN
601 EGKMIMQDKL EKERNDAKNA VEEYVYEMRD
        KLSGEYEKFV SEDDRNSFTL KLEDTENWLY
661 EDGEDQPKQV YVDKLAELKN LGQPIKIRFQ
        ESEERPKLFE ELGKQIQQYM KIISSFKNKE
721 DQYDHLDAAD MTKVEKSTNE AMEWMNNKLN
        LQNKQSLTMD PVVKSKEIEA KIKELTSTCS
781 PIISKPKPKV EPPKEEQKNA EQNGPVDGQG
        DNPGPQAAEQ GTDTAVPSDS DKKLPEMDID
```

In some embodiments, the heat shock protein is Hsp90. In some embodiments, Hsp90 is a polypeptide or fragment thereof having an amino acid sequence that is at least 85%, at least 90%, at least 95%, at least 98%, at least 99%, or 100% identical to a Hsp90 amino acid sequence associated with NCBI Reference Sequence: NP_005339.3. In some embodiments, Hsp90 is at least 85%, at least 90%, at least 95%, at least 98%, at least 99%, or 100% identical to the Hsp90 in Homo sapiens. An exemplary Hsp90 full-length amino acid sequence from Homo sapiens is provided below:

```
    1 MPEETQTQDQ PMEEEEVETF AFQAEIAQLM
        SLIINTFYSN KEIFLRELIS NSSDALDKIR
  61 YESLTDPSKL DSGKELHINL IPNKQDRTLT
        IVDTGIGMTK ADLINNLGTI AKSGTKAFME
121 ALQAGADISM IGQFGVGFYS AYLVAEKVTV
        ITKHNDDEQY AWESSAGGSF TVRTDTGEPM
181 GRGTKVILHL KEDQTEYLEE RRIKEIVKKH
        SQFIGYPITL FVEKERDKEV SDDEAEEKED
241 KEEEKEKEEK ESEDKPEIED VGSDEEEEKK
        DGDKKKKKKI KEKYIDQEEL NKTKPIWTRN
301 PDDITNEEYG EFYKSLTNDW EDHLAVKHFS
        VEGQLEFRAL LFVPRRAPFD LFENRKKKNN
361 IKLYVRRVFI MDNCEELIPE YLNFIRGVVD
        SEDLPLNISR EMLQQSKILK VIRKNLVKKC
421 LELFTELAED KENYKKFYEQ FSKNIKLGIH
        EDSQNRKKLS ELLRYYTSAS GDEMVSLKDY
481 CTRMKENQKH IYYITGETKD QVANSAFVER
        LRKHGLEVIY MIEPIDEYCV QQLKEFEGKT
541 LVSVTKEGLE LPEDEEEKKK QEEKKTKFEN
        LCKIMKDILE KKVEKVVVSN RLVTSPCCIV
601 TSTYGWTANM ERIMKAQALR DNSTMGYMAA
        KKHLEINPDH SIIETLRQKA EADKNDKSVK
661 DLVILLYETA LLSSGFSLED PQTHANRIYR
        MIKLGLGIDE DDPTADDTSA AVTEEMPPLE
721 GDDDTSRMEE VD
```

By “heat shock protein (Hsp) inhibitor” is meant an agent, compound, or substance that inhibits the activity of at least one heat shock protein. In some embodiments, the heat shock protein (Hsp) inhibitors inhibit the activity of one or more heat shock proteins selected from heat shock protein 27 (Hsp27), heat shock protein 40 (Hsp40), heat shock protein 60 (Hsp60), heat shock protein 70 (Hsp70), and/or heat shock protein 90 (Hsp90). In some embodiments, an Hsp inhibitor is a small molecule. In some embodiments, an Hsp inhibitor is an inhibitory nucleic acid molecule (e.g., siRNA, shRNA, or antisense RNA) that decreases gene expression of at least one heat shock protein (e.g., Hsp27, Hsp40, Hsp60, Hsp70, Hsp90). In some embodiments, an Hsp inhibitor is a small interfering RNA (siRNA) that decreases gene expression of at least one heat shock protein (e.g., Hsp27, Hsp40, Hsp60, Hsp70, Hsp90). In some embodiments, an Hsp inhibitor is a short-hairpin RNA (shRNA) that decreases gene expression of at least one heat shock protein (e.g., Hsp27, Hsp40, Hsp60, Hsp70, Hsp90).

In some embodiments, the Hsp inhibitor is a Hsp27 inhibitor, and analogs thereof. In some embodiments, the Hsp27 inhibitor is a shRNA that decreases the gene expression of HSPB1, the gene that encodes Hsp27. In some embodiments, the shRNA targets the gene expression of rat HSPB1 for silencing. The nucleic acid sequence of an exemplary shRNA that targets gene expression of rat HSPB1 is as follows:

5′-CAC TGG CAA GCA CGA AGA AAG-3′

In some embodiments, the shRNA targets the gene expression of human HSPB1 for silencing. The nucleic acid sequence of an exemplary shRNA that targets gene expression of human HSPB1 is as follows:

5′-CAC CGG CAA GCA CGA GGA GCG-3′

In some embodiments, the Hsp inhibitor is a Hsp40 inhibitor, and analogs thereof. In some embodiments, the Hsp inhibitor is a Hsp60 inhibitor, and analogs thereof. In some embodiments, the Hsp inhibitor is a Hsp70 inhibitor, and analogs thereof. In some embodiments, the Hsp inhibitor is a Hsp90 inhibitor, and analogs thereof. Non-limiting examples of Hsp90 inhibitors include 17-Allylamino-geldanamycin (17-AGG, Enzo Life Sciences), Geldanamycin (GA, Enzo Life Sciences), CUDC-305 (Abmole), and NVP-HSP990 (Abmole), and analogs thereof. In some embodiments, non-limiting examples of Hsp90 inhibitors are also described in PCT/US2013/036783, the entire contents of which are incorporated herein by reference). Additional suitable HSP inhibitors will be apparent to those of skill in the art based on this disclosure.

In some embodiments, the Hsp inhibitor is 17-Allylamino-geldanamycin (17-AGG) (IUPAC Name: 3 S,5S,6R,7S,8E,10R,11S,12E,14E)-21-(allylamino)-6-hydroxy-5,11-dimethoxy-3,7,9,15-tetramethyl-16,20,22-trioxo-17-azabicyclo[16.3.1]docosa-8,12,14,18,21-pentaen-10-yl] carbamate), which has the chemical formula C₃₁H₄₃N₃O₈. 17-AGG inhibits the function of Hsp90 (Heat Shock Protein 90). In some embodiments, 17-AGG has the following chemical structure:

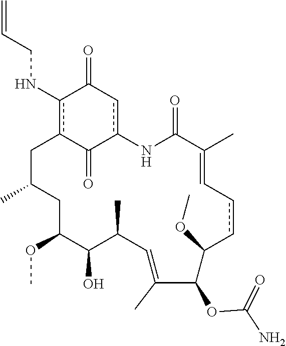

In some embodiments, the Hsp inhibitor is Geldanamycin (GA) (IUPAC Name: 4E,6Z,8S,9S,10E,12S,13R,14S,16R)-13-hydroxy-8,14,19-trimethoxy-4,10,12,16-tetramethyl-3,20,22-trioxo-2-azabicyclo[16.3.1]docosa-1(21),4,6,10,18-pentaen-9-yl carbamate), which has the chemical formula C₂₉H₄₀N₂O₉. GA is a 1,4-benzoquinone ansamycin antitumor antibiotic that inhibits the function of Hsp90 (Heat Shock Protein 90) by binding to the unusual ADP/ATP-binding pocket of the protein. In some embodiments, GA has the following chemical structure:

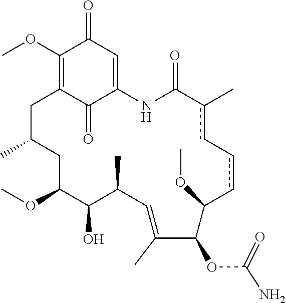

In some embodiments, the Hsp inhibitor is CUDC-305 (IUPAC Name: 4-amino-2-[[6-(dimethylamino)-1,3-benzodioxol-5-yl]thio]-N-(2,2-dimethylpropyl)-1H-imidazo[4,5-c]pyridine-1-ethanamine), which has the chemical formula C₂₂H₃₀N₆O₂S. CUDC-305 inhibits the function of Hsp90 (Heat Shock Protein 90). In some embodiments, CUDC-305 has the following chemical structure:

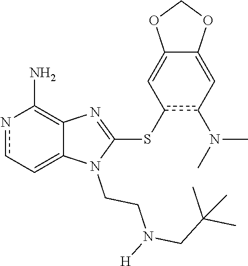

In some embodiments, the Hsp inhibitor is NVP-HSP990 (IUPAC Name: (R)-2-amino-7-((R)-4-fluoro-2-(6-methoxypyridin-2-yl)phenyl)-4-methyl-7,8-dihydropyrido[4,3-d]pyrimidin-5(6H)-one), which has the chemical formula C₂₀H₁₈FN₅O₂. NVP-HSP990 inhibits the function of Hsp90 (Heat Shock Protein 90). In some embodiments, NVP-HSP990 has the following chemical structure:

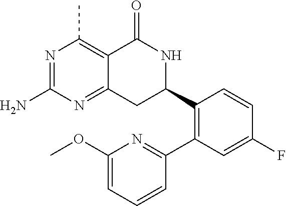

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein. In some embodiments, the inhibitory nucleic acid decreases gene expression of at least one heat shock protein (Hsp) (e.g., Hsp27, Hsp40, Hsp60, Hsp70, Hsp90). In some embodiments, the inhibitory nucleic acid is a small interfering RNA (siRNA). In some embodiments, the inhibitory nucleic acid is a short-hairpin RNA (shRNA). In some embodiments, the shRNA decreases the gene expression of HSPB1, the gene that encodes Hsp27. Exemplary shRNA nucleic acid sequences are provided below:

5′-CAC TGG CAA GCA CGA AGA AAG-3′
5′CAC CGG CAA GCA CGA GGA GCG-3′

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid, protein, or peptide is purified if it is substantially free of cellular material, debris, non-relevant viral material, or culture medium when produced by recombinant DNA techniques, or of chemical precursors or other chemicals when chemically synthesized. Purity and homogeneity are typically determined using standard purification methods and analytical chemistry techniques, for example, polyacrylamide gel electrophoresis or high-performance liquid chromatography.

The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified. The term “isolated” also embraces recombinant nucleic acids or proteins, as well as chemically synthesized nucleic acids or peptides.

By “isolated polynucleotide” is meant a nucleic acid (e.g., a DNA molecule) that is free of the genes which flank the gene, in the naturally-occurring genome of the organism from which the nucleic acid molecule of the disclosure is derived. The term includes, for example, a recombinant DNA that is incorporated into a vector; into an autonomously replicating plasmid or virus; into the genomic DNA of a prokaryote or eukaryote; or that exists as a separate molecule independent of other sequences (for example, a cDNA or a genomic or cDNA fragment produced by PCR or restriction endonuclease digestion). In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 40%, by weight, at least 50%, by weight, at least 60%, by weight, free from the proteins and naturally-occurring organic molecules with which it is naturally associated. In one embodiment, an isolated polypeptide preparation is at least 75%, at least 90%, and or at least 99%, by weight, free from the proteins and naturally-occurring organic molecules with which it is naturally associated. An isolated polypeptide may be obtained, for example, by extraction from a natural source; by expression of a recombinant nucleic acid encoding such a polypeptide; or by chemically synthesizing the protein. Purity can be measured by any standard, appropriate method, for example, column chromatography, polyacrylamide gel electrophoresis, or by HPLC analysis.

By “marker” is meant any protein or polynucleotide having an alteration in expression level or activity that is associated with a disease, condition, pathology, or disorder.

By “mechanistic target of rapamycin (mTOR)” is meant a serine/threonine protein kinase that is a member of the phosphatidylinositol 3-kinase-related kinase family of protein kinases and is encoded by the MTOR gene. mTOR links with other proteins and serves as a core component of two distinct protein complexes, mTOR complex 1 (mTORC1) and mTOR complex 2 (mTORC2), which regulate different cellular processes, including cell growth, cell proliferation, cell motility, cell survival, protein synthesis, autophagy, and transcription.

In some embodiments, mTOR is a polypeptide or fragment thereof having an amino acid sequence that is at least 85%, at least 90%, at least 95%, at least 98%, at least 99%, or 100% identical to a mTOR amino acid sequence associated with NCBI Reference Sequence: NP_004949.1. In some embodiments, mTOR is at least 85%, at least 90%, at least 95%, at least 98%, at least 99%, or 100% identical to the mTOR in Homo sapiens. An exemplary mTOR full-length amino acid sequence from Homo sapiens is provided below:

```
      1 MLGTGPAAAT TAATTSSNVS VLQQFASGLK
          SRNEETRAKA AKELQHYVTM ELREMSQEES
    61 TRFYDQLNHH IFELVSSSDA NERKGGILAI
          ASLIGVEGGN ATRIGRFANY LRNLLPSNDP
  121 VVMEMASKAI GRLAMAGDTF TAEYVEFEVK
          RALEWLGADR NEGRRHAAVL VLRELAISVP
  181 TFFFQQVQPF FDNIFVAVWD PKQAIREGAV
          AALRACLILT TQREPKEMQK PQWYRHTFEE
  241 AEKGFDETLA KEKGMNRDDR IHGALLILNE
          LVRISSMEGE RLREEMEEIT QQQLVHDKYC
  301 KDLMGFGTKP RHITPFTSFQ AVQPQQSNAL
          VGLLGYSSHQ GLMGFGTSPS PAKSTLVESR
  361 CCRDLMEEKF DQVCQWVLKC RNSKNSLIQM
          TILNLLPRLA AFRPSAFTDT QYLQDTMNHV
  421 LSCVKKEKER TAAFQALGLL SVAVRSEFKV
          YLPRVLDIIR AALPPKDFAH KRQKAMQVDA
  481 TVFTCISMLA RAMGPGIQQD IKELLEPMLA
          VGLSPALTAV LYDLSRQIPQ LKKDIQDGLL
  541 KMLSLVLMHK PLRHPGMPKG LAHQLASPGL
          TTLPEASDVG SITLALRTLG SFEFEGHSLT
  601 QFVRHCADHF LNSEHKEIRM EAARTCSRLL
          TPSIHLISGH AHVVSQTAVQ VVADVLSKLL
  661 VVGITDPDPD IRYCVLASLD ERFDAHLAQA
          ENLQALFVAL NDQVFEIREL AICTVGRLSS
  721 MNPAFVMPFL RKMLIQILTE LEHSGIGRIK
          EQSARMLGHL VSNAPRLIRP YMEPILKALI
  781 LKLKDPDPDP NPGVINNVLA TIGELAQVSG
          LEMRKWVDEL FIIIMDMLQD SSLLAKRQVA
  841 LWTLGQLVAS TGYVVEPYRK YPTLLEVLLN
          FLKTEQNQGT RREAIRVLGL LGALDPYKHK
  901 VNIGMIDQSR DASAVSLSES KSSQDSSDYS
          TSEMLVNMGN LPLDEFYPAV SMVALMRIFR
  961 DQSLSHHHTM VVQAITFIFK SLGLKCVQFL
          PQVMPTFLNV IRVCDGAIRE FLFQQLGMLV
1021 SFVKSHIRPY MDEIVTLMRE FWVMNTSIQS
          TIILLIEQIV VALGGEFKLY LPQLIPHMLR
1081 VFMHDNSPGR IVSIKLLAAI QLFGANLDDY
          LHLLLPPIVK LFDAPEAPLP SRKAALETVD
1141 RLTESLDFTD YASRIIHPIV RTLDQSPELR
          STAMDTLSSL VFQLGKKYQI FIPMVNKVLV
1201 RHRINHQRYD VLICRIVKGY TLADEEEDPL
          IYQHRMLRSG QGDALASGPV ETGPMKKLHV
1261 STINLQKAWG AARRVSKDDW LEWLRRLSLE
          LLKDSSSPSL RSCWALAQAY NPMARDLFNA
1321 AFVSCWSELN EDQQDELIRS IELALTSQDI
          AEVTQTLLNL AEFMEHSDKG PLPLRDDNGI
1381 VLLGERAAKC RAYAKALHYK ELEFQKGPTP
          AILESLISIN NKLQQPEAAA GVLEYAMKHF
1441 GELEIQATWY EKLHEWEDAL VAYDKKMDTN
          KDDPELMLGR MRCLEALGEW GQLHQQCCEK
1501 WTLVNDETQA KMARMAAAAA WGLGQWDSME
          EYTCMIPRDT HDGAFYRAVL ALHQDLFSLA
1561 QQCIDKARDL LDAELTAMAG ESYSRAYGAM
          VSCHMLSELE EVIQYKLVPE RREIIRQIWW
1621 ERLQGCQRIV EDWQKILMVR SLVVSPHEDM
          RTWLKYASLC GKSGRLALAH KTLVLLLGVD
1681 PSRQLDHPLP TVHPQVTYAY MKNMWKSARK
          IDAFQHMQHF VQTMQQQAQH AIATEDQQHK
1741 QELHKLMARC FLKLGEWQLN LQGINESTIP
          KVLQYYSAAT EHDRSWYKAW HAWAVMNFEA
1801 VLHYKHQNQA RDEKKKLRHA SGANITNATT
          AATTAATATT TASTEGSNSE SEAESTENSP
1861 TPSPLQKKVT EDLSKTLLMY TVPAVQGFFR
          SISLSRGNNL QDTLRVLTLW FDYGHWPDVN
1921 EALVEGVKAI QIDTWLQVIP QLIARIDTPR
          PLVGRLIHQL LTDIGRYHPQ ALIYPLTVAS
1981 KSTTTARHNA ANKILKNMCE HSNTLVQQAM
          MVSEELIRVA ILWHEMWHEG LEEASRLYFG
2041 ERNVKGMFEV LEPLHAMMER GPQTLKETSF
          NQAYGRDLME AQEWCRKYMK SGNVKDLTQA
2101 WDLYYHVFRR ISKQLPQLTS LELQYVSPKL
          LMCRDLELAV PGTYDPNQPI IRIQSIAPSL
2161 QVITSKQRPR KLTLMGSNGH EFVFLLKGHE
          DLRQDERVMQ LFGLVNTLLA NDPTSLRKNL
2221 SIQRYAVIPL STNSGLIGWV PHCDTLHALI
          RDYREKKKIL LNIEHRIMLR MAPDYDHLTL
2281 MQKVEVFEHA VNNTAGDDLA KLLWLKSPSS
          EVWFDRRTNY TRSLAVMSMV GYILGLGDRH
2341 PSNLMLDRLS GKILHIDFGD CFEVAMTREK
          FPEKIPFRLT RMLTNAMEVT GLDGNYRITC
2401 HTVMEVLREH KDSVMAVLEA FVYDPLLNWR
          LMDTNTKGNK RSRTRTDSYS AGQSVEILDG
2461 VELGEPAHKK TGTTVPESIH SFIGDGLVKP
          EALNKKAIQI INRVRDKLTG RDFSHDDTLD
2521 VPTQVELLIK QATSHENLCQ CYIGWCPFW
```

By “mechanistic target of rapamycin complex 1 (mTORC1)” is meant a protein complex composed of mTOR, regulatory-associated protein of mTOR (Raptor), mammalian lethal with SEC13 protein 8 (MLST8), Proline-rich AKT1 substrate 1 (PRAS40) and DEP domain-containing mTOR-interacting protein (DEPTOR). mTORC1 functions as a nutrient/energy/redox sensor and controls protein synthesis. The TSC1/2 complex is an inhibitory regulator of the mechanistic target of rapamycin complex 1 (mTORC1), which coordinates key neurodevelopmental processes. Increased mTOR signaling due to loss of either TSC1/2 results in profound changes in neuronal architecture and differentiation. An overview of mTOR signaling is provided by Lipton and Sahin, The neurology of mTOR; Neuron 84, 275-291 (2014), the entire contents of which is incorporated herein by reference.

As used herein the “mechanistic target of rapamycin (mTOR) pathway” refers to a signaling pathway that acts as a molecular systems integrator to support organismal and cellular interactions with the environment. The mTOR pathway regulates homeostasis by directly influencing protein synthesis, transcription, autophagy, metabolism, and organelle biogenesis and maintenance, and is implicated in the entire hierarchy of brain function including the proliferation of neural stem cells, the assembly and maintenance of circuits, experience-dependent plasticity, and regulation of complex behaviors like feeding, sleep, and circadian rhythms. Dysfunction of the mTOR pathway is implicated in neurodevelopmental disorders.

By “mechanistic target of rapamycin (mTOR) inhibitor” is meant an agent, compound, or substance that inhibits activity of the mTOR pathway. In some embodiments, the methods herein include administration of inhibitors of the mTOR pathway. In some embodiments, mTOR inhibitors inhibit S6 phosphorylation. In some embodiments, inhibitors of the mTOR pathway include, but are not limited to rapamycin, everolimus, Geldanamycin (GA), 17-Allylamino-geldanamycin (17-AGG), MCI-186, Nicardipine-HCl, K252A, Tyrphostin 9 and LY-294002. Rapamycin (also known as sirolimus) (IUPAC Name: 1R,9S,12S,15R,16E,18R,19R,21R,23 S,24E,26E,28E,30S,32S,35R)-1,18-dihydroxy-12-{(2R)-1-[(1S,3R,4R)-4-hydroxy-3-methoxycyclohexyl]-2-propanyl}-19,30-dimethoxy-15,17,21,23,29,35-hexamethyl-11,36-dioxa-4-azatricyclo[30.3.1.0˜4,9˜]hexatriaconta-16,24,26,28-tetraene-2,3,10,14,20-pentone) is a macrolide compound with the chemical formula C₅₁H₇₉NO₁₃. Rapamycin inhibits the mTOR pathway by directly binding to mTOR Complex 1 (mTORC1). In some embodiments, rapamycin has the following chemical structure:

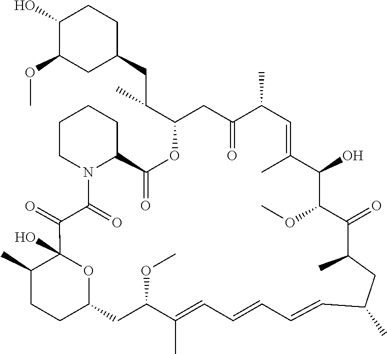

Everolimus (IUPAC Name: Dihydroxy-12-[(2R)-1-[(1 S,3R,4R)-4-(2-hydroxyethoxy) methoxycyclohexyl]propan-2-yl]-19,30-dimethoxy-15,17,21,23,29,35-hexamethyl-11,36-dioxa-4-azatricyclo[30.3.1.0 hexatriaconta-16,24,26,28-tetraene-2,3,10,14,20-pentone) has the chemical formula C₅₃H₈₃NO₁₄ and is the 40-O-(2-hydroxyethyl) derivative of sirolimus and works similarly as an inhibitor of mTOR. In some embodiments, everolimus has the following chemical structure:

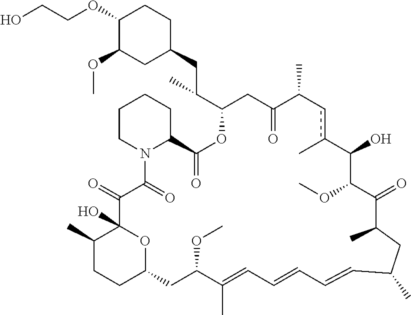

MCI-186 (also known as edaravone) (IUPAC Name: 5-methyl-2-phenyl-4H-pyrazol-3-one) has the chemical formula C₁₀H₁₀N₂O and functions as an anti-oxidant. In some embodiments, MCI-186 has the following chemical structure:

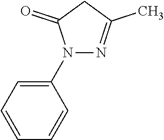

Nicardipine-HCl (IUPAC Name: 5-O-[2-[benzyl(methyl)amino]ethyl] 3-O-methyl 2,6-dimethyl-4-(3-nitrophenyl)-1,4-dihydropyridine-3,5-dicarboxylate; hydrochloride) has the chemical formula C₂₆H₃₀ClN₃O₆ and functions as a calcium channel blocker. In some embodiments, Nicardipine-HCl has the following chemical structure:

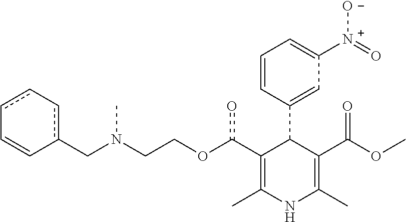

K252A (IUPAC Name: 9S-(9α,10β,12α))-2,3,9,10,11,12-hexahydro-10-hydroxy-10-(methoxycarbonyl)-9-methyl-9,12-epoxy-1H-diindolo[1,2,3-fg:3′,2′,1′-kl]pyrrolo[3,4-i][1,6]benzodiazocin-1-one) has the chemical formula C₂₇H₂₁N₃O₅ and functions as a kinase inhibitor. In some embodiments, K252A has the following chemical structure:

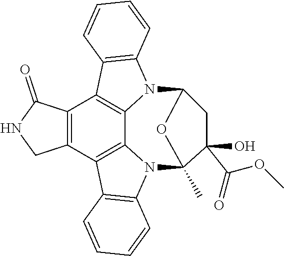 [SMILES: COC(=O)[C@@]1(O)C[C@H]2O[C@]1(C)n1c3ccccc3c3c4c(c5c6ccccc6n2c5c31)C(=O)NC4]

Tyrphostin 9 (also known as SF-6847 or Malonoben) (IUPAC Name: [4-Hydroxy-3,5-bis(2-methyl-2-propanyl)benzylidene]malononitrile) has the chemical formula C₁₈H₂₂N₂O and functions as an inhibitor of the PDGF receptor tyrosine kinase. In some embodiments, Tyrphostin 9 has the following chemical structure:

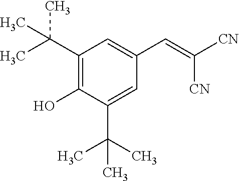

LY-294002 (IUPAC Name: 2-Morpholin-4-yl-8-phenylchromen-4-one) has the chemical formula C₁₉H₁₇NO₃ and functions as an inhibitor of phosphoinositide 3-kinases (PI3Ks). In some embodiments, LY-294002 has the following chemical structure:

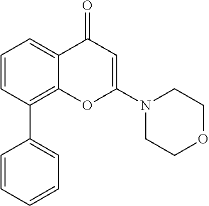 [SMILES: O=c1cc(N2CCOCC2)oc2c(-c3ccccc3)cccc12]

By “mechanistic target of rapamycin (mTOR) regulatory genes” is meant genes involved in regulating the mTOR pathway and/or mTORC1. Non-limiting examples of mTOR regulatory genes include Tuberous sclerosis 1 (TSC1), Tuberous sclerosis 2 (TSC2), AKT3, and DEP domain-containing 5 (DEPDC5).

The Tuberous sclerosis 1 (TSC1) gene encodes a protein that functions as a co-chaperone which inhibits the ATPase activity of the chaperone Hsp90 and decelerates its chaperone cycle. TSC1, TSC2 and TBC1D7 is a multi-protein complex also known as the TSC complex. This complex negatively regulates mTORC1 signaling by functioning as a GTPase-activating protein (GAP) for the small GTPase Rheb, an activator of mTORC1. The TSC1 gene is located on chromosome 9 in Homo sapiens. In some embodiments, TSC1 has a nucleotide sequence that is at least 85%, at least 90%, at least 95%, at least 98%, at least 99%, or 100% identical to a TSC1 nucleotide sequence associated with NCBI Reference Sequence: NG_012386.1. In some embodiments, TSC1 is at least 85%, at least 90%, at least 95%, at least 98%, at least 99%, or 100% identical to the TSC1 in Homo sapiens.

The Tuberous sclerosis 2 (TSC2) gene encodes a protein that functions as a tumor suppressor and is able to stimulate specific GTPases. The TSC2 gene is located on chromosome 16 in Homo sapiens In some embodiments, TSC2 has a nucleotide sequence that is at least 85%, at least 90%, at least 95%, at least 98%, at least 99%, or 100% identical to a TSC2 nucleotide sequence associated with NCBI Reference Sequence: NG_005895.1. In some embodiments, TSC2 is at least 85%, at least 90%, at least 95%, at least 98%, at least 99%, or 100% identical to the TSC2 in Homo sapiens. Mutations in either TSC1 or TSC2 cause the neurodevelopmental disorder Tuberous sclerosis complex (TSC).

The AKT3 gene encodes a RAC-gamma serine/threonine-protein kinase that functions as a regulator of cell signaling. The AKT3 gene is located on chromosome 1 in Homo sapiens In some embodiments, AKT3 has a nucleotide sequence that is at least 85%, at least 90%, at least 95%, at least 98%, at least 99%, or 100% identical to a AKT3 nucleotide sequence associated with NCBI Reference Sequence: NG_029764.2. In some embodiments, AKT3 is at least 85%, at least 90%, at least 95%, at least 98%, at least 99%, or 100% identical to the AKT3 in Homo sapiens.

The DEP domain-containing 5 (DEPDC5) gene encodes a protein involved in intracellular signal transduction. The DEPDC5 gene is located on chromosome 22 in Homo sapiens In some embodiments, DEPDC5 has a nucleotide sequence that is at least 85%, at least 90%, at least 95%, at least 98%, at least 99%, or 100% identical to a DEPDC5 nucleotide sequence associated with NCBI Reference Sequence: NG_034067.1. In some embodiments, DEPDC5 is at least 85%, at least 90%, at least 95%, at least 98%, at least 99%, or 100% identical to the DEPDC5 in Homo sapiens.

By “mTORopathy” is meant a disease or disorder caused by mutations in mTOR regulatory genes, from a disruption of the mTOR pathway, or from dysfunctional mTORC1 activity. In some embodiments, the mTORopathy is caused by a mutation in one or more mTOR regulatory genes, including but not limited to of TSC1, TSC2, AKT3, and DEPDC5. In some embodiments, the mTORopathy is caused by a mutation in TSC1 or TSC2. In some embodiments, the mTORopathy is caused by an increase in mTORC1 activity. In some embodiments, non-limiting examples of a mTORopathy include tuberous sclerosis complex (TSC), neural ciliopathy, epilepsy, neuropathy, autism, hemimegalencephaly, cortical dysplasia, focal cortical dysplasia, traumatic brain injury, brain tumours, and dementia, or a combination thereof. In some embodiments, the mTORopathy is tuberous sclerosis complex (TSC), and symptoms or complications thereof.

By “neurodevelopmental disorder” is meant a disease or disorder that impairs the growth and development of the brain and/or central nervous system. Neurodevelopmental disorders include, but are not limited to mTORopathies or neuronal ciliopathies. In some embodiments, non-limiting examples of neurodevelopmental disorders include tuberous sclerosis complex (TSC), intellectual disability, brain malformations, cortical tubers, neural ciliopathy, epilepsy, neuropathy, autism, hemimegalencephaly, cortical dysplasia, focal cortical dysplasia, traumatic brain injury, brain tumours, and dementia, or a combination thereof. In some embodiments, the neurodevelopmental disorder is tuberous sclerosis complex (TSC), and symptoms or complications thereof.

By “neuronal cilia” is meant cilia that project from the surface of neurons.

As used herein, a “neuronal ciliopathy” refers to a ciliopathy of the central nervous system (CNS). Neuronal ciliopathies are associated with severe neurodevelopmental outcomes including brain malformations, autism spectrum disorder (ASD), and intellectual disability (Bettencourt-Dias et al., Centrosomes and cilia in human disease; Trends Genet 27, 307-315 (2011); Guemez-Gamboa et al., Primary cilia in the developing and mature brain; Neuron 82, 511-521 (2014)). In some embodiments, the neuronal ciliopathy is a focal malformation of cortical developments (FMCDs) caused by somatic mutations in mechanistic target of rapamycin (mTOR). In some embodiments, the neuronal ciliopathy is tuberous sclerosis complex (TSC), and symptoms or complications thereof.

By “normalize,” “normalizing” or “normalization” is meant to bring or return to a normal or standard condition or state.

As used herein, “obtaining” as in “obtaining an agent” includes synthesizing, isolating, purchasing, or otherwise acquiring the agent.

The term “pharmaceutically acceptable vehicle” refers to conventional carriers (vehicles) and excipients that are physiologically and pharmaceutically acceptable for use, particularly in mammalian, e.g., human, subjects. Such pharmaceutically acceptable vehicles are known to the skilled practitioner in the pertinent art and can be readily found in Remington's Pharmaceutical Sciences, by E. W. Martin, Mack Publishing Co., Easton, Pa., 15th Edition (1975) and its updated editions, which describes compositions and formulations suitable for pharmaceutical delivery of one or more therapeutic or immunogenic compositions, such as one or more vaccines, and additional pharmaceutical agents. In general, the nature of a pharmaceutically acceptable carrier depends on the particular mode of administration being employed. For instance, parenteral formulations usually comprise injectable fluids/liquids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers may include, for example, pharmaceutical grades of mannitol, lactose, starch, or magnesium stearate, which typically stabilize and/or increase the half-life of a composition or drug. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

The terms “protein,” “peptide,” “polypeptide,” and their grammatical equivalents are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three (3) amino acids long. A protein, peptide, or polypeptide can refer to an individual protein or a collection of proteins. One or more of the amino acids in a protein, peptide, or polypeptide can be modified, such as glycoproteins, for example, by the addition of a chemical entity such as a carbohydrate group, a hydroxyl group, a phosphate group, a farnesyl group, an isofarnesyl group, a fatty acid group, a linker for conjugation, functionalization, or other modifications, etc. A protein, peptide, or polypeptide can also be a single molecule or can be a multi-molecular complex. A protein, peptide, or polypeptide can be just a fragment of a naturally occurring protein or peptide. A protein, peptide, or polypeptide can be naturally occurring, recombinant, or synthetic, or any combination thereof.

In some embodiments, a protein comprises a proteinaceous part, e.g., an amino acid sequence constituting a nucleic acid binding domain, and an organic compound, e.g., a compound that can act as a nucleic acid cleavage agent. In some embodiments, a protein is in a complex with, or is in association with, a nucleic acid, e.g., RNA or DNA. Any of the proteins provided herein can be produced by any method known in the art. For example, the proteins provided herein can be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

Conservative amino acid substitutions are those substitutions that, when made, least interfere with the properties of the original protein, that is, the structure and especially the function of the protein is conserved and is not significantly changed by such substitutions. Examples of conservative amino acid substitutions are known in the art, e.g., as set forth in, for example, U.S. Publication No. 2015/0030628. Conservative substitutions generally maintain (a) the structure of the polypeptide backbone in the area of the substitution, for example, as a sheet or helical conformation; (b) the charge or hydrophobicity of the molecule at the target site; and/or (c) the bulk of the side chain

The substitutions that are generally expected to produce the greatest changes in protein properties are non-conservative, for instance, changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

By “promoter” is meant an array of nucleic acid control sequences, which direct transcription of a nucleic acid. A promoter includes necessary nucleic acid sequences near the start site of transcription. A promoter also optionally includes distal enhancer or repressor sequence elements.

As will be appreciated by the skilled practitioner in the art, the term “purified” does not require absolute purity; rather, it is intended as a relative term. Thus, for example, a purified peptide, protein, or other active compound is one that is isolated in whole or in part from naturally associated proteins and other contaminants. In certain embodiments, the term “substantially purified” refers to a peptide, protein, or other active compound that has been isolated from a cell, cell culture medium, or other crude preparation and subjected to routine methods, such as fractionation, chromatography, or electrophoresis, to remove various components of the initial preparation, such as proteins, cellular debris, and other components.

A “recombinant” nucleic acid or protein is one that has a sequence that is not naturally occurring or that has a sequence that is made by an artificial combination of two otherwise separated segments of sequence. Such an artificial combination is often accomplished by chemical synthesis or by the artificial manipulation of isolated segments of nucleic acids, for example, by genetic engineering techniques. A “non-naturally occurring” nucleic acid or protein is one that may be made via recombinant technology, artificial manipulation, or genetic or molecular biological engineering procedures and techniques, such as those commonly practiced in the art.

By “reduces” is meant a negative alteration of at least 5%, 10%, 25%, 30%, 40%, 50%, 75%, 80%, 85%, 90%, 95%, 98%, or 100%.

By “reference” is meant a standard or control condition.

By “small hairpin RNA” or “shRNA” is meant an RNA sequence having a stem-loop structure. A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). The term “hairpin” is also used herein to refer to stem-loop structures. Such structures are well known in the art and the term is used consistently with its known meaning in the art. As is known in the art, the secondary structure does not require exact base-pairing. Thus, the stem can include one or more base mismatches or bulges. Alternatively, the base-pairing can be exact, i.e. not include any mismatches. The multiple stem-loop structures can be linked to one another through a linker, such as, for example, a nucleic acid linker, a miRNA flanking sequence, other molecule, or some combination thereof. While there may be some variation in range, a conventional stem-loop shRNA can comprise a stem ranging from 19 to 29 bp, and a loop ranging from 4 to 30 bp. “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. In some embodiments, the precursor miRNA molecule can include more than one stem-loop structure. MicroRNAs are endogenously encoded RNA molecules that are about 22-nucleotides long and generally expressed in a highly tissue- or developmental-stage-specific fashion and that post-transcriptionally regulate target genes. More than 200 distinct miRNAs have been identified in plants and animals. These small regulatory RNAs are believed to serve important biological functions by two prevailing modes of action: (1) by repressing the translation of target mRNAs, and (2) through RNA interference (RNAi), that is, cleavage and degradation of mRNAs. In the latter case, miRNAs function analogously to small interfering RNAs (siRNAs). Thus, one can design and express artificial miRNAs based on the features of existing miRNA genes.

In some embodiments, the shRNA decreases gene expression of at least one heat shock protein (e.g., Hsp27, Hsp40, Hsp60, Hsp70, Hsp90). In some embodiments, the shRNA decreases the gene expression of HSPB1, the gene that encodes Hsp27. Exemplary shRNA nucleic acid sequences are provided below:

5′-CAC TGG CAA GCA CGA AGA AAG-3′
5′CAC CGG CAA GCA CGA GGA GCG-3′

By “small interfering RNA” or “siRNA” is meant a double stranded RNA (dsRNA). Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. In some embodiments, siRNAs are introduced into the brain. Such siRNAs are used to downregulate mRNA levels or promoter activity. In some embodiments, siRNAs are used to downregulate the activity of at least one heat shock protein (e.g., Hsp27, Hsp40, Hsp60, Hsp70, Hsp90).

By “specifically binds” is meant a compound or antibody that recognizes and binds a polypeptide, but which does not substantially recognize and bind other molecules in a sample, for example, a biological sample, which naturally includes the polypeptide.

Nucleic acid molecules useful in the methods described herein include any nucleic acid molecule that encodes a polypeptide as described, or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In one embodiment, such a sequence is at least 60%, or at least 80% or 85%, or at least or equal to 90%, 95%, 98% or even 99% identical at the amino acid level or nucleic acid to the sequence used for comparison.

“Sequence identity” refers to the similarity between amino acid or nucleic acid sequences that is expressed in terms of the similarity between the sequences. Sequence identity is frequently measured in terms of percentage identity (or similarity or homology); the higher the percentage, the more similar the sequences are. Homologs or variants of a given gene or protein will possess a relatively high degree of sequence identity when aligned using standard methods. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence. In addition, other programs and alignment algorithms are described in, for example, Smith and Waterman, 1981, Adv. Appl. Math. 2:482; Needleman and Wunsch, 1970, J. Mol. Biol. 48:443; Pearson and Lipman, 1988, Proc. Natl. Acad. Sci. U.S.A. 85:2444; Higgins and Sharp, 1988, Gene 73:237-244; Higgins and Sharp, 1989, CABIOS 5:151-153; Corpet et al., 1988, Nucleic Acids Research 16:10881-10890; Pearson and Lipman, 1988, Proc. Natl. Acad. Sci. U.S.A. 85:2444; and Altschul et al., 1994, Nature Genet. 6:119-129. The NCBI Basic Local Alignment Search Tool (BLAST™) (Altschul et al. 1990, J. Mol. Biol. 215:403-410) is readily available from several sources, including the National Center for Biotechnology Information (NCBI, Bethesda, Md.) and on the Internet, for use in connection with the sequence analysis programs blastp, blastn, blastx, tblastn and tblastx.

By “subject” is meant an animal, e.g., a mammal, including, but not limited to, a human, a non-human primate, or a non-human mammal, such as a bovine, equine, canine, ovine, or feline mammal, or a sheep, goat, llama, camel, or a rodent (rat, mouse), gerbil, or hamster. In particular aspects as described herein, the subject is a human subject, such as a patient.

Ranges provided herein are understood to be shorthand for all of the values within the range, inclusive of the first and last stated values. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or greater, consecutively, such as to 100 or greater.

As used herein, the terms “treat,” “treating,” “treatment,” and the like refer to reducing, diminishing, decreasing, delaying, abrogating, ameliorating, or eliminating, a disease, condition, disorder, or pathology, and/or symptoms associated therewith. While not intending to be limiting, “treating” typically relates to a therapeutic intervention that occurs after a disease, condition, disorder, or pathology, and/or symptoms associated therewith, have begun to develop to reduce the severity of the disease, etc., and the associated signs and symptoms. It will be appreciated that, although not precluded, treating a disorder or condition does not require that the disease, condition, disorder, pathology, or the symptoms associated therewith, be completely eliminated.

As used herein, the terms “prevent,” “preventing,” “prevention,” “prophylactic treatment” and the like, refer to inhibiting or blocking a disease state, or the full development of a disease in a subject, or reducing the probability of developing a disease, disorder or condition in a subject, who does not have, but is at risk of developing, or is susceptible to developing, a disease, disorder, or condition.

As referred to herein, a “transformed” or “transfected” cell is a cell into which a nucleic acid molecule or polynucleotide sequence has been introduced by molecular biology techniques. As used herein, the term “transfection” encompasses all techniques by which a nucleic acid molecule or polynucleotide may be introduced into such a cell, including transfection with viral vectors, transformation with plasmid vectors, and introduction of naked nucleic acid (DNA or RNA) by electroporation, lipofection, and particle gun acceleration.

By “tuberous sclerosis complex (TSC)” is meant a rare multisystem autosomal dominant genetic disease that causes the formation of hamartia (malformed tissue such as the cortical tubers), hamartomas (benign growths such as facial angiofibroma and subependymal nodules), and very rarely, cancerous hamartoblastomas, in the brain and on other vital organs such as the kidneys, heart, liver, eyes, lungs and skin. The effect of TSC on the brain leads to neurological symptoms such as seizures, intellectual disability, developmental delay, and behavioral problems. TSC is caused by a mutation of either of two genes, TSC1 and TSC2, which encode the proteins hamartin and tuberin, respectively.

As used herein, a “vector” refers to a nucleic acid (polynucleotide) molecule into which foreign nucleic acid can be inserted without disrupting the ability of the vector to replicate in and/or integrate into a host cell. A vector can include nucleic acid sequences that permit it to replicate in a host cell, such as an origin of replication. An insertional vector is capable of inserting itself into a host nucleic acid. A vector can also include one or more selectable marker genes and other genetic elements. An expression vector is a vector that contains the necessary regulatory sequences to allow transcription and translation of inserted gene or genes in a host cell. In some embodiments, the vector is a viral vector. Exemplary viral vectors include, but are not limited to, retroviral, including lentiviral, adenoviral, baculoviral and avian viral vectors, and including such vectors allowing for stable, single-copy genomic integrations. In some embodiments, the vector is an adeno-associated virus (AAV) vector.

Unless specifically stated or obvious from context, as used herein, the term “or” is understood to be inclusive. Unless specifically stated or obvious from context, as used herein, the terms “a,” “an,” and “the” are understood to be singular or plural. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description.

Unless specifically stated or obvious from context, as used herein, the term “about” is understood as within a range of normal tolerance in the art, for example within two (2) standard deviations (SD) of the mean. About may be understood as being within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of some embodiments for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1E depict that brains of TSC patients and CNS-knockout mouse models have reduced ciliated neurons that is restored by rapamycin in vivo. Data are mean±SEM. Scale bar is 10 μm. Data are averages±SEM. FIG. 1A depicts a heat map of differential cilia gene expression in healthy control brains (Non-TSC, n=8) and TSC patient brains (TSC/tuber, n=16). (p<0.005). FIG. 1B depicts representative confocal images of epileptic brain specimens from Non-TSC (a,d) and TSC (b-c, e-f), patients stained with SMI311 (giant cell marker), ACIII (cilia marker) and Hoechst (nuclei marker). White arrows indicate cilia. FIG. 1C is a graph depicting the quantification of ciliation in the human tissues (Non-TSC n=6; TSC n=6; Kruskal-Wallis test, with Dunn's multiple comparison test, *p<0.05, ns=non-significant). Data represent mean±SEM. FIG. 1D depicts representative confocal images of hippocampi from vehicle-treated Tsc2 control (n=5) and Tsc2 mutant (n=6) and from rapamycin (Rapa)-treated Tsc2 control (n=5) and Tsc2 mutant (n=8) mice. Sections were stained with ACIII (a-d, e-h) and with the neuronal marker, NeuN (a-d). Rapamycin was given by intra-peritoneal injection beginning at P7 until P56 every other day at 3 mg/kg/day. FIG. 1E is a graph depicting the quantification of the percentage of ciliated neurons (400 neurons/mouse, One-way ANOVA with Tukey's post hoc test, *p<0.05, ns, non-significant).

FIGS. 2A-2E depict disinhibition of mTORC1 activity due to Tsc2-knockdown in neurons leads to reduced ciliation. FIG. 2A is a schematic depicting a time course experiment to monitor ACIII and pS6 at DIV2, 5, 7, 13, 20 in hippocampal neurons using the image-based high content assays for cilia (cilia^{HCA}) and mTORC1 (mTORC1^{HCA}) with the Arrayscan XTI. FIG. 2B depicts raw images of Cilia^{HCA} (a and e) and spot detector algorithm identification of ctrlsh and Tsc2-sh neurons stained with Hoechst (nuclei in b and j), GFP (LV-infected in neurons in c and g), and ACIII (cilia in d and h) at DIV13. Arrows indicate cilia. Scale bar is 10 μm. FIG. 2C depicts raw images of mTORC1^{HCA} (i and n) and spot detector algorithm identification of ctrl-sh and Tsc2-sh neurons stained with Hoechst (nuclei j and o), GFP (LV-infected neurons k and p) and pS6 (mTORC1 activity m and q) at DIV13. Scale bar is 100 μm. FIG. 2D is a graph depicting the quantification of ciliation. Data represent GFP⁺ ACIII⁺ neurons [n=5-18 biological replica/experiment, n=1-6 experiment/condition] as average percentage of the total number of GFP⁺ cells. Bars represent mean±SEM (unpaired t-test **p<0.005, ****p<0.0001). FIG. 2E is a graph depicting the quantification of mTORC1 activity. Data represent GFP⁺pS6⁺ neurons [n=7-8 biological replica/experiment, n=1-4 experiment/condition] as average percentage of the total number of GFP⁺ cells. Bars represent mean±SEM (unpaired t-test **p<0.005, ****p<0.0001).

FIGS. 3A-3L depict using a phenotypic screen with Tsc2-knockdown neurons to identify Hsp90 as drug target for mTORC1 through regulation of PI3K/Akt signaling components. FIG. 3A is a schematic depicting mTORC1 screen workflow. Tsc2-sh neurons were treated with screen compounds for 24 hrs between DIV19-DIV20. FIG. 3B is a graph depicting Z-score correlation between compound Replica 1 (R1) and Replica 2 (R2). Percent of GFP⁺ pS6⁺ neurons was converted to Z-score. Z-score of −1.8 (p<0.05) was marked by lines. Hits were considered compounds with a Z-score <−1.8 in the dotted box. Compounds used for follow-up experiments were rapamycin, geldanamycin (GA) and 17-allylamino-geldanamycin (17-AGG), all other compounds were labeled. FIG. 3C depicts a zoom of hits in the dotted box in FIG. 3B. FIG. 3D depicts a list of hits, Z-scores and compound concentrations in the library. FIG. 3E is a graph depicting the validation of GA by dose-response using the mTORC1^{HCA}. Tsc2-sh neurons were treated with vehicle and with a dose-curve of GA [IC50=65 nM] for 24 hrs between DIV19-20. Data are GFP⁺ pS6⁺ neurons expressed as average percentage of vehicle-treated Tsc2-sh neurons (n=7-16 biological replica/condition, One-way ANOVA with Dunnett's multiple comparison test **p<0.01, ***p<0.005, ****p<0.0001). Error bars indicate ±SEM. FIG. 3F is a graph depicting the validation of 17-AGG by dose-response using the mTORC1^{HCA}. Tsc2-sh neurons were treated with vehicle and with a dose-curve of 17-AGG [IC50=346 nM] for 24 hrs between DIV19-20. Data are GFP⁺ pS6⁺ neurons expressed as average percentage of vehicle-treated Tsc2-sh neurons (n=7-16 biological replica/condition, One-way ANOVA with Dunnett's multiple comparison test **p<0.01, ***p<0.005, ****p<0.0001). Error bars indicate ±SEM. FIG. 3G depicts a Western blot of protein lysates from ctrl-sh and Tsc2-sh treated with vehicle or with 17-AGG dose-curve. FIG. 3H is a graph depicting the quantification of pS6 expressed as average fold changes of vehicle-treated ctrl-sh neurons. Western blot data were normalized using GAPDH as loading control. Data quantification is relative to vehicle-treated Tsc2-sh neurons (n=3-4 experiment/condition, One-way ANOVA with Dunnett's multiple comparison test, *p<0.05, **p<0.01, ***p<0.005, ****p<0.0001). Error bars indicate ±SEM. FIG. 3I is a graph depicting the quantification of p-IGF-IRβ expressed as average fold changes of vehicle-treated ctrl-sh neurons. Data quantification is relative to vehicle-treated Tsc2-sh neurons (n=3-4 experiment/condition, One-way ANOVA with Dunnett's multiple comparison test, *p<0.05, **p<0.01, ***p<0.005, ****p<0.0001). Error bars indicate ±SEM. FIG. 3J is a graph depicting the quantification of total IGF-IRβ expressed as average fold changes of vehicle-treated ctrl-sh neurons. Data quantification is relative to vehicle-treated Tsc2-sh neurons (n=3-4 experiment/condition, One-way ANOVA with Dunnett's multiple comparison test, *p<0.05, **p<0.01, ***p<0.005, ****p<0.0001). Error bars indicate ±SEM. FIG. 3K is a Western blot depicting Hsp90 inhibition results in IGF-IRβ degradation through the proteasome. Wild-type cortical neurons were treated with vehicle and with 4 μM 17-AGG alone or in the presence of 100 nM bortezomib (BTZ, 24 hrs at DIV20). FIG. 3L is a graph depicting the quantification of the Western blot in FIG. 3K (n=5, One-way ANOVA with Tukey's multiple comparison test *p<0.05, **p<0.005). Western blot data were normalized using GAPDH as loading control. Data are mean±SEM.

FIGS. 4A-4K depict that reduced ciliation in the Tsc2-sh neurons is prevented by mTORC1 and Hsp90 inhibition during an age- and time-sensitive window. FIG. 4A depicts an experimental scheme of rapamycin time-course experiment using the cilia^{HCA} with endpoint at DIV13. FIG. 4B depicts an experimental scheme of rapamycin time-course experiment using the cilia^{HCA} with endpoint at DIV20. FIG. 4C is a graph depicting the quantification of cilia in ctrl-sh and in Tsc2-sh neurons vehicle-treated or rapamycin-treated (20 nM) with assay endpoint at DIV20 using the cilia^{HCA}. Data were quantified relative to each respective vehicle-treated group (n=8-18 biological replica/condition, Two-way ANOVA with Dunnett's multiple-comparison test, *p<0.05, **p<0.01, ns=non-significant) and are represented as mean±SEM. FIG. 4D is a graph depicting the quantification of cilia in ctrl-sh and in Tsc2-sh neurons vehicle-treated or rapamycin-treated (20 nM) with assay endpoint at DIV20 using the cilia^{HCA} Data were quantified relative to each respective vehicle-treated group (n=8-18 biological replica/condition, Two-way ANOVA with Dunnett's multiple-comparison test, *p<0.05, **p<0.01, ns=non-significant) and are represented as mean±SEM. FIG. 4E is a graph depicting the effect of 17-AGG dose-curve between DIV9-13 on ciliation using the cilia^{HCA}. Data represent GFP⁺ ACIII⁺ in the Tsc2-sh neurons as percent of vehicle-treated control neurons. Quantification was done relative to vehicle-treated Tsc2-sh neurons [n=8-16 biological replica/condition, One-way ANOVA with Dunnett's multiple-comparison test, *p<0.05, ****p<0.0001]. Data are mean±SEM. FIG. 4F is a graph depicting the effect of 17-AGG dose-curve between DIV9-13 on pS6 using the mTORC1^{HCA}. Data represent GFP⁺ pS6⁺ vin the Tsc2-sh neurons as percent of vehicle-treated Tsc2-sh neurons. Quantification was done relative to vehicle-treated Tsc2-sh neurons [n=15-64 biological replica/condition, One-way ANOVA with Dunnett's multiple-comparison test, *p<0.05, ****p<0.0001]. Data are mean±SEM. FIG. 4G depicts representative images of cilia in vehicle-treated ctrl-sh and in Tsc2-sh neurons vehicle-treated or treated with 50 nM 17-AGG between DIV9-13. Neurons and cilia were identified by GFP and ACIII staining. Scale bar is 25 μm. FIG. 4H depicts a Western blot of protein lysates from ctrl-sh and Tsc2-sh neurons treated with vehicle or with 17-AGG dose-curve between DIV9-13. FIG. 4I is a graph depicting the quantification of pS6. Western blot data were normalized using GAPDH as loading control. Data are average fold changes of vehicle-treated ctrl-sh neurons. Quantification is relative to vehicle-treated Tsc2-sh neurons (n=4 experiment/condition, One-way ANOVA with Dunnett's multiple-comparison test, *p<0.05, **p<0.01, ***p<0.005, ****p<0.0001). Error bars indicate ±SEM. FIG. 4J is a graph depicting the quantification of total IGF-IRβ. Western blot data were normalized using GAPDH as loading control. Data are average fold changes of vehicle-treated ctrl-sh neurons. Quantification is relative to vehicle-treated Tsc2-sh neurons (n=4 experiment/condition, One-way ANOVA with Dunnett's multiple-comparison test, *p<0.05, **p<0.01, ***p<0.005, ****p<0.0001). Error bars indicate ±SEM. FIG. 4K is a graph depicting the quantification of p-IGF-IRβ. Western blot data were normalized using GAPDH as loading control. Data are average fold changes of vehicle-treated ctrl-sh neurons. Quantification is relative to vehicle-treated Tsc2-sh neurons (n=4 experiment/condition, One-way ANOVA with Dunnett's multiple-comparison test, *p<0.05, **p<0.01, ***p<0.005, ****p<0.0001). Error bars indicate ±SEM.

FIGS. 5A-5O depict Hsp27 upregulation due to neuronal Tsc1/2 loss is reduced by 17-AGG and by rapamycin in the dose range and within the critical window that restores ciliation. FIG. 5A depicts a Western blot of protein lysates (Hsp27, Hsp40, Hsp60, Hsp70, Hsp90) from control and Tsc2-sh neurons treated with vehicle or with a four-day dose curve of 17-AGG with endpoint at DIV13. FIG. 5B is a graph depicting the quantification of Hsp27 from control and Tsc2-sh neurons treated with vehicle or with a four-day dose curve of 17-AGG with endpoint at DIV13. Hsp27 is expressed as average fold change of vehicle-treated ctrl-sh neurons. Western blot data were normalized using GAPDH as loading control. Data quantification was done relative to vehicle-treated Tsc2-sh neurons (n=3-4 experiment/condition, One-way ANOVA with Dunnett's multiple-comparison test, *p<0.05, **p<0.05, ***p<0.005, ****p<0.0001). Error bars indicate ±SEM. FIG. 5C is a graph depicting the quantification of Hsp40 from control and Tsc2-sh neurons treated with vehicle or with a four-day dose curve of 17-AGG with endpoint at DIV13. Hsp40 is expressed as average fold change of vehicle-treated ctrl-sh neurons. Western blot data were normalized using GAPDH as loading control. Data quantification was done relative to vehicle-treated Tsc2-sh neurons (n=3-4 experiment/condition, One-way ANOVA with Dunnett's multiple-comparison test, *p<0.05, **p<0.05, ***p<0.005, ****p<0.0001). Error bars indicate ±SEM. FIG. 5D is a graph depicting the quantification of Hsp60 from control and Tsc2-sh neurons treated with vehicle or with a four-day dose curve of 17-AGG with endpoint at DIV13. Hsp60 is expressed as average fold change of vehicle-treated ctrl-sh neurons. Western blot data were normalized using GAPDH as loading control. Data quantification was done relative to vehicle-treated Tsc2-sh neurons (n=3-4 experiment/condition, One-way ANOVA with Dunnett's multiple-comparison test, *p<0.05, **p<0.05, ***p<0.005, ****p<0.0001). Error bars indicate ±SEM. FIG. 5E is a graph depicting the quantification of Hsp70 from control and Tsc2-sh neurons treated with vehicle or with a four-day dose curve of 17-AGG with endpoint at DIV13. Hsp70 is expressed as average fold change of vehicle-treated ctrl-sh neurons. Western blot data were normalized using GAPDH as loading control. Data quantification was done relative to vehicle-treated Tsc2-sh neurons (n=3-4 experiment/condition, One-way ANOVA with Dunnett's multiple-comparison test, *p<0.05, **p<0.05, ***p<0.005, ****p<0.0001). Error bars indicate±SEM. FIG. 5F is a graph depicting the quantification of Hsp90 from control and Tsc2-sh neurons treated with vehicle or with a four-day dose curve of 17-AGG with endpoint at DIV13. Hsp90 is expressed as average fold change of vehicle-treated ctrl-sh neurons. Western blot data were normalized using GAPDH as loading control. Data quantification was done relative to vehicle-treated Tsc2-sh neurons (n=3-4 experiment/condition, One-way ANOVA with Dunnett's multiple-comparison test, *p<0.05, **p<0.05, ***p<0.005, ****p<0.0001). Error bars indicate ±SEM. FIG. 5G depicts a Western blot of protein lysates (Hsp27, Hsp40, Hsp60, Hsp70, Hsp90) from control and Tsc2-sh neurons treated with vehicle or with a four-day dose curve of 17-AGG with endpoint at DIV20. FIG. 5H is a graph depicting the quantification of Hsp27 from control and Tsc2-sh neurons treated with vehicle or with a four-day dose curve of 17-AGG with endpoint at DIV20. Hsp27 is expressed as average fold changes of vehicle-treated ctrl-sh neurons. Western blot data were normalized using GAPDH as loading control. Data quantification was done relative to vehicle-treated Tsc2-sh neurons (n=3-4 experiment/condition, One-way ANOVA with Dunnett's multiple-comparison test, *p<0.05, **p<0.05, ***p<0.005, ****p<0.0001). Error bars indicate ±SEM. FIG. 5I is a graph depicting the quantification of Hsp40 from control and Tsc2-sh neurons treated with vehicle or with a four-day dose curve of 17-AGG with endpoint at DIV20. Hsp40 is expressed as average fold changes of vehicle-treated ctrl-sh neurons. Western blot data were normalized using GAPDH as loading control. Data quantification was done relative to vehicle-treated Tsc2-sh neurons (n=3-4 experiment/condition, One-way ANOVA with Dunnett's multiple-comparison test, *p<0.05, **p<0.05, ***p<0.005, ****p<0.0001). Error bars indicate ±SEM. FIG. 5J is a graph depicting the quantification of Hsp60 from control and Tsc2-sh neurons treated with vehicle or with a four-day dose curve of 17-AGG with endpoint at DIV20. Hsp60 is expressed as average fold changes of vehicle-treated ctrl-sh neurons. Western blot data were normalized using GAPDH as loading control. Data quantification was done relative to vehicle-treated Tsc2-sh neurons (n=3-4 experiment/condition, One-way ANOVA with Dunnett's multiple-comparison test, *p<0.05, **p<0.05, ***p<0.005, ****p<0.0001). Error bars indicate ±SEM. FIG. 5K is a graph depicting the quantification of Hsp70 from control and Tsc2-sh neurons treated with vehicle or with a four-day dose curve of 17-AGG with endpoint at DIV20. Hsp70 is expressed as average fold changes of vehicle-treated ctrl-sh neurons. Western blot data were normalized using GAPDH as loading control. Data quantification was done relative to vehicle-treated Tsc2-sh neurons (n=3-4 experiment/condition, One-way ANOVA with Dunnett's multiple-comparison test, *p<0.05, **p<0.05, ***p<0.005, ****p<0.0001). Error bars indicate ±SEM. FIG. 5L is a graph depicting the quantification of Hsp90 from control and Tsc2-sh neurons treated with vehicle or with a four-day dose curve of 17-AGG with endpoint at DIV20. Hsp90 is expressed as average fold changes of vehicle-treated ctrl-sh neurons. Western blot data were normalized using GAPDH as loading control. Data quantification was done relative to vehicle-treated Tsc2-sh neurons (n=3-4 experiment/condition, One-way ANOVA with Dunnett's multiple-comparison test, *p<0.05, **p<0.05, ***p<0.005, ****p<0.0001). Error bars indicate ±SEM. FIG. 5M depicts a Western blot of protein lysates (Hsp27, pS6 (S240/244), S6) from control and Tsc2-sh neurons treated with vehicle or with 20 nM rapamycin (Rapa) for one day or four days with endpoint at DIV13. FIG. 5N is a graph depicting the quantification of Hsp27 (n=3). Western blot data normalized using GAPDH as loading control. Data are expressed as average fold changes of vehicle-treated ctrl-sh neurons (one-way ANOVA with Dunnett's multiple-comparison test, *p<0.05, **p<0.005, ****p<0.0001). Error bars indicate ±SEM. FIG. 5O is a graph depicting the quantification of pS6 (n=7). Western blot data normalized using GAPDH as loading control. Data are expressed as average fold changes of vehicle-treated ctrl-sh neurons (one-way ANOVA with Dunnett's multiple-comparison test, *p<0.05, **p<0.005, ****p<0.0001). Error bars indicate ±SEM.

FIGS. 6A-6K depict that 17-AGG improved ciliation downstream of mTORC1 activation through downregulation of HspB1 gene expression in Tsc2 knockdown neurons. FIG. 6A is a graph depicting HspB1 mRNA levels in vehicle-treated ctrl-sh and vehicle- or 50 nM 17-AGG-treated Tsc2-sh neurons (four-days, DIV13 endpoint). Data were normalized to GAPDH, and are averages±SEM. (One-way ANOVA with Tukey's multiple-comparison test, n=4 ****p<0.0005). FIG. 6B depicts a Western blot of protein lysates (Hsp27, pS6 (S240/244), S6) from control and Tsc2-sh neurons treated with vehicle or with 50 nM 17-AGG (four-days, DIV13 endpoint). FIG. 6C is a graph depicting the quantification of Hsp27 (n=3) from control and Tsc2-sh neurons treated with vehicle or with 50 nM 17-AGG (four-days, DIV13 endpoint). Western blot data were normalized using GAPDH as loading control. Data are average fold changes of ctrl-sh neurons±SEM (One-way ANOVA with Dunnett's multiple-comparison test, *p<0.05, **p<0.01). FIG. 6D is a graph depicting the quantification of pS6 (240/244)/S6 (n=4) from control and Tsc2-sh neurons treated with vehicle or with 50 nM 17-AGG (four-days, DIV13 endpoint). Western blot data were normalized using GAPDH as loading control. Data are average fold changes of ctrl-sh neurons±SEM (One-way ANOVA with Dunnett's multiple-comparison test, *p<0.05, **p<0.01). FIG. 6E is a graph depicting HSPB1 expression in Non-TSC (n=8) and TSC/Tuber (n=16) brains (unpaired t-test p**<0.005). Data are averages±SEM. FIG. 6F depicts a Western blot of protein lysates (Hsp27, pS6 (S240/244), S6) from control and Tsc2-sh neurons transduced with RFP-tagged scramble control shRNA (RFP-C) or with RFP-tagged hsp27 shRNA (RFP-hsp27sh). FIG. 6G is a graph depicting the quantification of Hsp27 from control and Tsc2-sh neurons treated with RFP-Hsp27sh or RFP-C from FIG. 6F. Western blot data were normalized using GAPDH as loading control. Data are average fold changes of ctrl-sh neurons±SEM (n=4, One-way ANOVA with Tukey's multiple-comparison test, ****p<0.005). FIG. 6H is a graph depicting the quantification of pS6 (240/244)/S6 from control and Tsc2-sh neurons treated with RFP-Hsp27sh or RFP-C. Western blot data were normalized using GAPDH as loading control. Data are average fold changes of ctrl-sh neurons±SEM (n=4, One-way ANOVA with Tukey's multiple-comparison test, ****p<0.005). FIG. 6I depicts the effect of Hsp27-knockdown on ciliation using the cilia^{HCA} assay. Representative images of ctrl-sh and Tsc2-sh neurons transduced with RFP-C or with RFP-hsp27sh are shown. Raw images (a, c, e) and spot detector algorithm identification (b, d, f) of ctrl-sh and Tsc2-sh neurons were stained with Hoechst (nuclei), GFP (ctrl-sh or Tsc2-sh infected neurons), ACIII (cilia) and RFP (RFP-C or RFP-hsp27sh transduced neurons). Arrows indicate cilia. Scale bar is 25μm. FIG. 6J is a graph depicting the quantification of ciliation. Data represent GFP⁺RFP⁺ neurons with cilia (ACIII⁺) as a percentage ciliated GFP⁺RFP⁺ controls±SEM (n=6-30 biological replica/experiment, n=2 experiment/condition, One-way ANOVA with Tukey's multiple comparison test *p<0.05). FIG. 6K depicts a schematic model for 17-AGG multi-faceted effects in Tsc1/2-deficient neurons. Panel a depicts that under Tsc1/2 loss, increased mTORC1 activity results in aberrant Hsp27 expression and reduced ciliated neurons. Panel b depicts that 17-AGG^{pS6} inhibits mTORC1 (IC50=1.8 μM) through Hsp90-dependent regulation of PI3K/Akt signaling components with no significant change in Hsp27. Panel c depicts that 17-AGG^{cilia} reverses reduced ciliation at 100-fold greater potency (EC50=15 nM) independently from mTORC1 through suppression of hspB1/Hsp27.

FIGS. 7A-7C depict that Tsc1 mutant mice have reduced ciliation in the cortex and in the dentate gyrus. FIG. 7A depicts representative confocal images from the cortex (a-b) and the dentate gyrus (c-d) of vehicle-treated Tsc1 control (n=3), vehicle-treated Tsc1 mutant (n=5), and rapamycin-treated Tsc1 mutant (n=2), stained with the neuronal marker, NeuN (a and c) and with the cilia marker ACIII (a-d). White arrows indicate cilia. FIG. 7B is a graph depicting the quantification of the percentage of ciliated neurons in the cortex (700 neurons/mouse, One-way ANOVA with Tukey's post hoc test, *p<0.05). Bars represent mean±SEM. Scale bar is 10 μm. FIG. 7C is a graph depicting the quantification of the percentage of ciliated neurons in the dentate gyms (700 neurons/mouse, One-way ANOVA with Tukey's post hoc test, *p<0.05). Bars represent mean±SEM. Scale bar is 10 μm.

FIGS. 8A-8J depict that Tsc2 gene knockdown in hippocampal neurons results in reduced ciliation by high content imaging and manual confocal microscopy. FIG. 8A depicts a representative Western blot of protein lysates (Tsc2, pS6 (S240/244), S6) from ctrl-sh and Tsc2-sh neurons to monitor Tsc2 and S6 phosphorylation in a time course experiment at DIV5, 7, 13, 20. FIG. 8B is a graph depicting the quantification of pS6 (n=4). Western blot data were normalized using GAPDH as loading control. Data are expressed as fold changes of vehicle-treated ctrl-sh neurons (Mann-Whitney Test, *p<0.05). Error bars indicate ±SEM. FIG. 8C is a schematic representation of focal planes and object identification for cilia^{HCA} for image-based high-content assays for cilia using the Arrayscan XTi. FIG. 8D depicts raw images of hippocampal neurons transduced with GFP-tagged lentivirus. Hoechst staining indicates nuclei, GFP staining indicates infected neurons, and ACIII staining indicates cilia. Image acquisition for the cilia^{HCA} was performed with a 40× (scale bar is 25 μm) objective. White masks show the region of interest (ROI) for object identification. FIG. 8E is a schematic representation of focal planes and object identification for mTORC1^{HCA} for image-based high-content assays for mTORC1 using the Arrayscan XTi. FIG. 8F depicts raw images of hippocampal neurons transduced with GFP-tagged lentivirus. Hoechst staining indicates nuclei, GFP staining indicates infected neurons, and pS6 staining indicates mTORC1 activity. Image acquisition for the mTORC1^{HCA} was performed with a 10× (scale bar is 100 μm) objective. White masks show the region of interest (ROI) for object identification. FIG. 8G depicts representative images of cilia in ctrl-sh and Tsc2-sh neurons using confocal microscopy. Neurons at DIV13 were stained with GFP (a and c), ACIII (a-d), centrin (a-d) and DAPI (a-d). Images on the left (a and c) include GFP staining to show the LV-transduced neurons and a white box indicating the regions enlarged for cilia and centrin staining magnified in the zoom (in b and d). Scale bar is 10 μm. FIG. 8H is a graph depicting quantification of the percentage of GFP⁺ neurons with cilia. Bars represent mean±SEM (n=3, 88-100 GFP neurons/exp; unpaired t-test, **p<0.01). FIG. 8I is a graph depicting cilia length. Bars represent mean±SEM (unpaired t-test, ns=non-significant). FIG. 8J is a graph depicting ACIII intensity. Bars represent mean±SEM (unpaired t-test, ns=non-significant).

FIGS. 9A-9M depict the robustness of mTORC1^{HCA} and the effect of the hits identified in the screen on the mTORC1 pathway and on ciliation between DIV19-20. FIG. 9A is a graph depicting mTORC1^{HCA} quality control by Z-scores calculated as the absolute deviation of GFP⁺ pS6⁺ neurons between positive (ctrl-sh neurons) and negative (Tsc2-sh) controls at DIV20 (n=8 wells/condition, Z′=0.18). FIG. 9B depicts a Western blot of protein lysates (p-Akt (S473), Akt, p-PRAS40 (Thr246), PRAS40, Raptor) from ctrl-sh and Tsc2-sh treated with vehicle or with 17-AGG dose curve for 24 hrs between DIV19-20. FIG. 9C is a graph depicting the quantification of p-Akt (Ser473). Western blot data were normalized using GAPDH as loading control. Data are fold changes of ctrl-sh neurons and are quantified relative to vehicle-treated Tsc2-sh neurons (n=4 experiment/condition, Kruskal-Wallis test followed by the Dunn's multiple comparison test, *p<0.05, **p<0.01). FIG. 9D is a graph depicting the quantification of Akt. Western blot data were normalized using GAPDH as loading control. Data are fold changes of ctrl-sh neurons and are quantified relative to vehicle-treated Tsc2-sh neurons (n=4 experiment/condition, One-way ANOVA with Dunnett's multiple comparison test, **p<0.01, ns=not significant). FIG. 9E is a graph depicting the quantification of p-PRAS40 (Thr246). Western blot data were normalized using GAPDH as loading control. Data are fold changes of ctrl-sh neurons and are quantified relative to vehicle-treated Tsc2-sh neurons (n=4 experiment/condition, Kruskal-Wallis test followed by the Dunn's multiple comparison test, *p<0.05, **p<0.01). FIG. 9F is a graph depicting the quantification of Raptor. Western blot data were normalized using GAPDH as loading control. Data are fold changes of ctrl-sh neurons and are quantified relative to vehicle-treated Tsc2-sh neurons (n=4 experiment/condition, One-way ANOVA with Dunnett's multiple comparison test, **p<0.01, ns=not significant). FIG. 9G depicts a Western blot of protein lysates (pS6 (S240/244), S6, IGF-IRβ) from ctrl-sh treated with vehicle or with 17-AGG dose curve. FIG. 9H is a graph depicting the quantification of pS6. Western blot data were normalized using GAPDH as loading control. Data are fold changes of vehicle-treated ctrl-sh neurons (n=3-4 experiment/condition, one-way ANOVA with Dunnett's multiple comparison test, *p<0.05, **p<0.01, ***p<0.001). FIG. 9I is a graph depicting the quantification of IGF-IRβ. Western blot data were normalized using GAPDH as loading control. Data are fold changes of vehicle-treated ctrl-sh neurons (n=3-4 experiment/condition, one-way ANOVA with Dunnett's multiple comparison test, *p<0.05, **p<0.01, ***p<0.001). FIG. 9J depicts a Western blot of protein lysates (pS6 (S240/244), S6, IGF-IRβ) from ctrl-sh and Tsc2-sh treated with vehicle or with GA dose curve. FIG. 9K is a graph depicting the quantification of pS6 (n=3). Western blot data were normalized using GAPDH as loading control. Data are fold changes of ctrl-sh neurons and are quantified relative to vehicle-treated Tsc2-sh neurons (One-way ANOVA with Dunnett's multiple comparison test, *p<0.05, ****p<0.0001). Error bars indicate ±SEM. FIG. 9L is a graph depicting the quantification of IGF-IRβ (n=4). Western blot data were normalized using GAPDH as loading control. Data are fold changes of ctrl-sh neurons and are quantified relative to vehicle-treated Tsc2-sh neurons (One-way ANOVA with Dunnett's multiple comparison test, *p<0.05, ****p<0.0001). Error bars indicate ±SEM. FIG. 9M is a graph depicting the quantification of ciliation using the cilia^{HCA} in ctrl-sh and in Tsc2-sh neurons treated with vehicle, 8.5 μM 17-AGG, 9 μM geldanamycin (GA) and 5.5 μM rapamycin for 24 hrs at DIV20 (n=2-4 wells/condition, one-way ANOVA with Sidak's multiple comparison test, ****p<0.0005).

FIGS. 10A-10J depict the effect of Hsp90 inhibition with 17-AGG on PI3K/Akt signaling in the Tsc2-knockdown neuronal cultures at DIV13. FIG. 10A depicts a Western blot of protein lysates (p-Akt (S473), Akt, p-PRAS (Thr246), PRAS, Raptor) from ctrl-sh and Tsc2-sh treated with vehicle or with 17-AGG dose curve. FIG. 10B is a graph depicting the quantification of p-Akt at 5473. Western blot data were normalized using GAPDH as loading control. Data are fold changes of ctrl-sh neurons and are quantified relative to vehicle-treated Tsc2-sh neurons (n=4 experiment/condition, Kruskal-Wallis test followed by Dunn's multiple comparison test, **p<0.01, ns=not significant). Error bars indicate ±SEM. FIG. 10C is a graph depicting the quantification of Akt. Western blot data were normalized using GAPDH as loading control. Data are fold changes of ctrl-sh neurons and are quantified relative to vehicle-treated Tsc2-sh neurons (n=4 experiment/condition, One-way ANOVA with Dunnett's multiple comparison test, ****p<0.0001, ns=not significant). Error bars indicate ±SEM. FIG. 10D is a graph depicting the quantification of p-PRAS at Thr246. Western blot data were normalized using GAPDH as loading control. Data are fold changes of ctrl-sh neurons and are quantified relative to vehicle-treated Tsc2-sh neurons (n=4 experiment/condition, Kruskal-Wallis test followed by Dunn's multiple comparison test, **p<0.01, ns=not significant). Error bars indicate ±SEM. FIG. 10E is a graph depicting the quantification of Raptor. Western blot data were normalized using GAPDH as loading control. Data are fold changes of ctrl-sh neurons and are quantified relative to vehicle-treated Tsc2-sh neurons (n=4 experiment/condition, One-way ANOVA with Dunnett's multiple comparison test, ****p<0.0001, ns=not significant). Error bars indicate ±SEM. FIG. 10F is a graph depicting the quantification of ciliation using the cilia^{HCA} in Tsc2-sh neurons treated with a four-day 17-AGG dose curve between DIV16-20 with assay endpoint at DIV20. Data quantification was done relative to vehicle-treated Tsc2-sh neurons (n=8-16 biological replica/condition, One-way ANOVA with Dunnett's multiple comparison test, ns=not significant). FIG. 10G is a graph depicting the quantification of ciliation using the cilia^{HCA} in ctrl-sh neurons treated with a four-day 17-AGG dose curve between DIV9-13 with assay endpoint at DIV13. Data were quantified relative to vehicle-treated ctrl-sh neurons (n=16-32 biological replica/condition, one-way ANOVA with Dunnett's multiple-comparison test, ****p<0.0001) and are expressed as average±SEM. FIG. 10H is depicts a Western blot of protein lysates (pS6 (S240/244), S6, IGF-IRβ) from ctrl-sh treated with vehicle or with 17-AGG dose curve. FIG. 10I is a graph depicting the quantification of pS6. Western blot data were normalized using GAPDH as loading control. Data are fold changes of vehicle-treated ctrl-sh neurons (n=4-8 experiment/condition, one-way ANOVA with Dunnett's multiple comparison test, ***p<0.001, ****p<0.0001). Error bars indicate ±SEM. FIG. 10J is a graph depicting the quantification of IGF-IRβ. Western blot data were normalized using GAPDH as loading control. Data are fold changes of vehicle-treated ctrl-sh neurons (n=4-8 experiment/condition, one-way ANOVA with Dunnett's multiple comparison test, ***p<0.001, ****p<0.0001). Error bars indicate ±SEM.

FIGS. 11A and 11B depict the effect of Hsp90 inhibitor CUDC-305 on ciliation in Tsc2-sh neurons. FIG. 11A is a graph depicting the toxicity of CUDC-305 after treating Tsc2-sh neurons with the indicated concentrations of CUDC-305 (0 is a vehicle control) for four days starting at DIV 9. Neurons were stained at DIV 13 and the number of nuclei per field were counted. FIG. 11B is a graph depicting the percentage of ciliated GFP-positive neurons. Tsc2-sh neurons were treated with CUDC-305 as in FIG. 11A and stained for GFP and cilia (ACIII).

FIGS. 12A and 12B depict the effect of Hsp90 inhibitor NPV-HSP-990 on ciliation in Tsc2-sh neurons. FIG. 12A is a graph depicting the toxicity of NPV-HSP-990 after treating Tsc2-sh neurons with the indicated concentrations of NPV-HSP-990 (0 is a vehicle control) for four days starting at DIV 9. Neurons were stained at DIV 13 and the number of nuclei per field were counted. FIG. 12B is a graph depicting the percentage of ciliated GFP-positive neurons. Tsc2-sh neurons were treated with NPV-HSP-990 as in FIG. 12A and stained for GFP and cilia (ACIII).

FIGS. 13A-13D depict the effect of HSP90 inhibitors on Hsp27 expression in Tsc2-sh neurons. FIG. 13A is a Western blot depicting control or Tsc2-sh neurons treated with increasing doses of CUDC-305 for four days from DIV 9-13 to determine the effect on Hsp27 expression. FIG. 13B is a graph depicting the quantification of the Western blot data in FIG. 13A. FIG. 13C is a Western blot depicting control or Tsc2-sh neurons treated with increasing doses of NPV-HSP-990 for four days from DIV 9-13 to determine the effect on Hsp27 expression. FIG. 13D is a graph depicting the quantification of the Western blot data in FIG.

FIGS. 14A and 14B depict ciliation of human neurons with mutations in TSC2. FIG. 14A are images depicting iPSC-derived neurons of TSC2+/+, TSC2+/−, and TSC2−/− genotypes stained with Dapi, GFP, and ACIII at 19 days of differentiation. FIG. 14A are images depicting iPSC-derived neurons of TSC2+/+, TSC2+/−, and TSC2−/− genotypes stained with Dapi, GFP, and Arl13b at 19 days of differentiation.

### DETAILED DESCRIPTION OF THE DISCLOSURE

Provided herein are methods of treating neurodevelopmental disorders, including the treatment of Tuberous sclerosis complex (TSC), with pharmaceutical compositions containing heat shock protein (Hsp) inhibitors. Also provided herein are methods for inhibiting mechanistic target of rapamycin complex 1 (mTORC1) activity and/or increasing or normalizing ciliation.

As reported in detail below, the invention is based, at least in part, on the discovery that inhibition of Heat shock protein 90 (Hsp90) is useful for the treatment of Tuberous Sclerosis Complex (TSC) and other diseases associated with ciliary deficits.

### Neurodevelopmental Disorders

The present disclosure features methods that are useful for the treatment of neurodevelopmental disorders (e.g., Tuberous Sclerosis Complex (TSC)), including mTORopathies and/or neuronal ciliopathies. In particular, the disclosure features methods that are useful for the treatment of Tuberous Sclerosis Complex (TSC). The present disclosure provides methods of treating disease and/or disorders or symptoms thereof which comprise administering a therapeutically effective amount of a pharmaceutical composition comprising a compound of the formulae herein to a subject (e.g., a mammal such as a human). Thus, one embodiment is a method of treating a subject suffering from or susceptible to a neurodevelopmental disease or disorder (e.g., Tuberous Sclerosis Complex (TSC)) or symptom thereof.

The therapeutic methods of the disclosure in general comprise administration of an effective amount of the compounds herein, such as a pharmaceutical composition herein, to a subject (e.g., animal, human) in need thereof, including a mammal, particularly a human, to treat the disease or disorder (e.g., neurodevelopmental disorder) or symptom thereof, under conditions such that the disease or disorder is treated to produce such effect. Such treatment will be suitably administered to subjects, particularly humans, suffering from, having, susceptible to, or at risk for a disease, disorder, or symptom thereof. Determination of those subjects in need of such treatment can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which the mechanistic target of rapamycin (mTOR) pathway may be implicated.

In some embodiments, the present disclosure features methods of treating a neurodevelopmental disorder (e.g., Tuberous Sclerosis Complex (TSC)) and symptoms thereof. Neurodevelopmental disorders impair the growth and development of the brain and/or central nervous system. In some embodiments, the neurodevelopmental disorders is an mTORopathy and/or neuronal ciliopathy. In some embodiments, non-limiting examples of neurodevelopmental disorders include tuberous sclerosis complex (TSC), intellectual disability, brain malformations, cortical tubers, neural ciliopathy, epilepsy, neuropathy, autism, hemimegalencephaly, cortical dysplasia, focal cortical dysplasia, traumatic brain injury, brain tumours, and dementia, or a combination thereof.

In some embodiments, the neurodevelopmental disorder is an mTORopathy. In some embodiments, the mTORopathy is a disease or disorder caused by mutations in mTOR regulatory genes, from a disruption of the mTOR pathway, or from dysfunctional mTORC1 activity. In some embodiments, the mTORopathy is caused by a mutation in TSC1 or TSC2. In some embodiments, the mTORopathy is caused by an increase in mTORC1 activity. In some embodiments, non-limiting examples of a mTORopathy include tuberous sclerosis complex (TSC), neural ciliopathy, epilepsy, neuropathy, autism, hemimegalencephaly, cortical dysplasia, focal cortical dysplasia, traumatic brain injury, brain tumours, and dementia, or a combination thereof. In some embodiments, the mTORopathy is tuberous sclerosis complex (TSC).

In some embodiments, the neurodevelopmental disorder or mTORopathy is caused by mutations in one or more mechanistic target of rapamycin (mTOR) regulatory genes. mTOR) regulatory genes are involved in regulating the mTOR pathway and/or mTORC1. Non-limiting examples of mTOR regulatory genes include Tuberous sclerosis 1 (TSC1), Tuberous sclerosis 2 (TSC2), AKT3, and DEP domain-containing 5 (DEPDC5). In some embodiments, the neurodevelopmental disorder is caused by mutations in the TSC1 or TSC2 genes, which encode the proteins hamartin and tuberin, respectively.

In some embodiments, the neurodevelopmental disorder or mTORopathy is caused by mutations in TSC1. TSC1 encodes the hamartin protein that functions as a co-chaperone which inhibits the ATPase activity of the chaperone Hsp90 and decelerates its chaperone cycle. TSC1, TSC2 and TBC1D7 is a multi-protein complex also known as the TSC complex. This complex negatively regulates mTORC1 signaling by functioning as a GTPase-activating protein (GAP) for the small GTPase Rheb, an activator of mTORC1. The TSC1 gene is located on chromosome 9 in Homo sapiens. In some embodiments, TSC1 has a nucleotide sequence that is at least 85%, at least 90%, at least 95%, at least 98%, at least 99%, or 100% identical to a TSC1 nucleotide sequence associated with NCBI Reference Sequence: NG_012386.1. In some embodiments, TSC1 is at least 85%, at least 90%, at least 95%, at least 98%, at least 99%, or 100% identical to the TSC1 in Homo sapiens.

In some embodiments, the neurodevelopmental disorder or mTORopathy is caused by mutations in TSC2. The TSC2 gene encodes the tuberin protein that functions as a tumor suppressor and is able to stimulate specific GTPases. The TSC2 gene is located on chromosome 16 in Homo sapiens In some embodiments, TSC2 has a nucleotide sequence that is at least 85%, at least 90%, at least 95%, at least 98%, at least 99%, or 100% identical to a TSC2 nucleotide sequence associated with NCBI Reference Sequence: NG_005895.1. In some embodiments, TSC2 is at least 85%, at least 90%, at least 95%, at least 98%, at least 99%, or 100% identical to the TSC2 in Homo sapiens.

In some embodiments, the neurodevelopmental disorder or mTORopathy is caused by mutations in AKT3. The AKT3 gene encodes a RAC-gamma serine/threonine-protein kinase that functions as a regulator of cell signaling. The AKT3 gene is located on chromosome 1 in Homo sapiens In some embodiments, AKT3 has a nucleotide sequence that is at least 85%, at least 90%, at least 95%, at least 98%, at least 99%, or 100% identical to a AKT3 nucleotide sequence associated with NCBI Reference Sequence: NG_029764.2. In some embodiments, AKT3 is at least 85%, at least 90%, at least 95%, at least 98%, at least 99%, or 100% identical to the AKT3 in Homo sapiens.

In some embodiments, the neurodevelopmental disorder or mTORopathy is caused by mutations in DEPDC5. The DEP domain-containing 5 (DEPDC5) gene encodes a protein involved in intracellular signal transduction. The DEPDC5 gene is located on chromosome 22 in Homo sapiens In some embodiments, DEPDC5 has a nucleotide sequence that is at least 85%, at least 90%, at least 95%, at least 98%, at least 99%, or 100% identical to a DEPDC5 nucleotide sequence associated with NCBI Reference Sequence: NG_034067.1. In some embodiments, DEPDC5 is at least 85%, at least 90%, at least 95%, at least 98%, at least 99%, or 100% identical to the DEPDC5 in Homo sapiens.

Mutations in either TSC1 or TSC2 cause the neurodevelopmental disorder Tuberous sclerosis complex (TSC). In some embodiments, the neurodevelopmental disorder or mTORopathy is tuberous sclerosis complex (TSC). TSC is associated with benign tumors called hamartomas in multiple organs as well as central nervous system (CNS) manifestations including epilepsy, intellectual disability and autism spectrum disorder (ASD) (Tsai and Sahin, Mechanisms of neurocognitive dysfunction and therapeutic considerations in tuberous sclerosis complex; Curr. Opin. Neurol. 24, 106-113 (2011)). In some instances, TSC leads to the formation of hamartoblastomas. The neurological symptoms of TSC have been correlated with brain lesions called cortical tubers, which are characterized by the presence of giant cells and dysmorphic neurons with immature features (Curatolo et al., Neurological and neuropsychiatric aspects of tuberous sclerosis complex; Lancet. Neurol. 14, 733-745 (2015)).

In some embodiments, the neurodevelopmental disorder or mTORopathy is caused by dysfunctional mechanistic target of rapamycin complex 1 (mTORC1) activity. The mechanistic target of rapamycin (mTOR) pathway acts as a molecular systems integrator to support organismal and cellular interactions with the environment. The mTOR pathway regulates homeostasis by directly influencing protein synthesis, transcription, autophagy, metabolism, and organelle biogenesis and maintenance, and is implicated in the entire hierarchy of brain function including the proliferation of neural stem cells, the assembly and maintenance of circuits, experience-dependent plasticity, and regulation of complex behaviors like feeding, sleep, and circadian rhythms. Dysfunction of the mTOR pathway is implicated in neurodevelopmental disorders.

The mTOR signaling pathway is mediated through two large biochemical complexes mTOR complex 1 (mTORC1) and mTOR complex 2 (mTORC2). These complexes regulate different cellular processes, including cell growth, cell proliferation, cell motility, cell survival, protein synthesis, autophagy, and transcription. mTORC1 is a protein complex composed of mTOR, regulatory-associated protein of mTOR (Raptor), mammalian lethal with SEC13 protein 8 (MLST8), Proline-rich AKT1 substrate 1 (PRAS40) and DEP domain-containing mTOR-interacting protein (DEPTOR). mTOR is a serine/threonine protein kinase that is a member of the phosphatidylinositol 3-kinase-related kinase family of protein kinases and is encoded by the MTOR gene.

In some embodiments, mTOR is a polypeptide or fragment thereof having an amino acid sequence that is at least 85%, at least 90%, at least 95%, at least 98%, at least 99%, or 100% identical to a mTOR amino acid sequence associated with NCBI Reference Sequence: NP_004949.1. In some embodiments, mTOR is at least 85%, at least 90%, at least 95%, at least 98%, at least 99%, or 100% identical to the mTOR in Homo sapiens.

mTORC1 functions as a nutrient/energy/redox sensor and controls protein synthesis. The TSC1/2 complex is an inhibitory regulator of the mechanistic target of rapamycin complex 1 (mTORC1), which coordinates key neurodevelopmental processes. Increased mTOR signaling due to loss of either TSC1/2 results in profound changes in neuronal architecture and differentiation. An overview of mTOR signaling is provided by Lipton and Sahin, The neurology of mTOR; Neuron 84, 275-291 (2014), the entire contents of which is incorporated herein by reference. In some embodiments, the neurodevelopmental disorder is characterized by an increase in mTORC1 activity.

In some embodiments, the neurodevelopmental disorder is a neuronal ciliopathy. Neuronal ciliopathies are genetic disorders of the central nervous system (CNS) caused by mutations in genes that function in neuronal cilia assembly and/or function. Neuronal cilia are membrane extensions of the surface of neurons made of microtubules that extend from a centriole-derived structure called the basal body (Lee and Gleeson, Cilia in the nervous system: linking cilia function and neurodevelopmental disorders; Current opinion in neurology 24, 98-105 (2011)). Neuronal cilia coordinate extracellular ligand-based signaling, and play a critical role in brain development.

Neuronal ciliopathies are associated with severe neurodevelopmental outcomes including brain malformations, autism spectrum disorder (ASD), and intellectual disability (Bettencourt-Dias et al., Centrosomes and cilia in human disease; Trends Genet 27, 307-315 (2011); Guemez-Gamboa et al., Primary cilia in the developing and mature brain; Neuron 82, 511-521 (2014)). In some embodiments, the neuronal ciliopathy is a focal malformation of cortical developments (FMCDs) caused by somatic mutations in mechanistic target of rapamycin (mTOR). In some embodiments, the neurodevelopmental disorder or neuronal ciliopathy is associated with a decrease in neuronal cilia. In some embodiments, the neuronal ciliopathy is tuberous sclerosis complex (TSC), and symptoms or complications thereof.

### Tuberous Sclerosis Complex (TSC)

Tuberous Sclerosis Complex (TSC) is a neurogenetic disorder that leads to elevated mechanistic target of rapamycin complex 1 (mTORC1) activity. Cilia can be affected by mTORC1 signaling, and ciliary deficits are associated with neurodevelopmental disorders. The present disclosure provides that neuronal cilia are affected in TSC and that cortical tubers from the brains of TSC patients have fewer cilia. Using high-content image-based assays, it was discovered that mTORC1 activity is inversely correlated with ciliation in TSC1/2-deficient neurons. Through the use of a phenotypic screen for mTORC1 inhibitors with TSC1/2-deficient neurons, heat shock proteins were identified as suppressing mTORC1 through regulation of PI3K/Akt signaling. In particular, pharmacological inhibition of Heat shock protein 90 (Hsp90) rescued ciliation through downregulation of Heat shock protein 27 (Hsp27). The present disclosure provides the use of heat shock machinery as a druggable signaling node to inhibit mTORC1 activity and increase or normalize cilia due to loss of TSC1/2 and provides broadly applicable platforms for studying TSC-related neuronal dysfunction.

### Heat Shock Protein (Hsp) Inhibitors

The present disclosure features heat shock protein (Hsp) inhibitors (e.g., inhibitors of Hsp27, Hsp40, Hsp60, Hsp70, Hsp90) with the ability to inhibit mTORC1 activity and/or increase or normalize neuronal cilia so as to treat a disease or disorder (e.g., neurodevelopmental disorder) and its symptoms, either prophylactically or therapeutically, caused by mutations in mTOR regulatory genes (e.g., TSC1, TSC2, AKT3, DEPDC5), mutations in genes that play a role in cilia assembly and/or function, from a disruption of the mTOR pathway, and/or from dysfunctional mTORC1 activity following administration and delivery to a subject.

A heat shock protein (Hsp) as described herein is a polypeptide or fragment thereof, which is produced by cells in response to exposure to stressful conditions, such as heat shock, cold, UV light and during wound healing or tissue remodeling (see e.g., Garrido et al., Heat shock proteins 27 and 70: anti-apoptotic proteins with tumorigenic properties; Cell Cycle 5, 2592-2601 (2006)). Hsps are divided into six families based on their size, and these proteins function in multi-component complexes that are closely inter-related (Chatterjee and Burns, Targeting Heat Shock Proteins in Cancer: A Promising Therapeutic Approach; Int J Mol Sci 18 (2017)). Many Hsp members perform chaperone functions by stabilizing new proteins to ensure correct folding or by helping to refold proteins that were damaged by the cell stress. Hsps are found in virtually all living organisms, from bacteria to humans. In some embodiments, the Hsps described herein are from humans.

Heat-shock proteins are named according to their molecular weight. In some embodiments, the heat shock proteins include heat shock protein 27 (Hsp27), heat shock protein 40 (Hsp40), heat shock protein 60 (Hsp60), heat shock protein 70 (Hsp70), and/or heat shock protein 90 (Hsp90). Hsp40, Hsp60, Hsp70 and Hsp90 refer to families of heat shock proteins on the order of 40, 60, 70 and 90 kilodaltons in size, respectively.

In some embodiments, the heat shock protein is Hsp27. In some embodiments, Hsp27 is a polypeptide or fragment thereof having an amino acid sequence that is at least 85%, at least 90%, at least 95%, at least 98%, at least 99%, or 100% identical to a Hsp27 amino acid sequence associated with NCBI Reference Sequence: NP_001531.1. In some embodiments, Hsp27 is at least 85%, at least 90%, at least 95%, at least 98%, at least 99%, or 100% identical to the Hsp27 in Homo sapiens.

In some embodiments, the heat shock protein is Hsp40. In some embodiments, Hsp40 is a protein or fragment thereof having an amino acid sequence that is at least 85%, at least 90%, at least 95%, at least 98%, at least 99%, or 100% identical to a Hsp40 amino acid sequence associated with NCBI Reference Sequence: NP_001300893.1. In some embodiments, Hsp40 is at least 85%, at least 90%, at least 95%, at least 98%, at least 99%, or 100% identical to the Hsp40 in Homo sapiens.

In some embodiments, the heat shock protein is Hsp60. In some embodiments, Hsp60 is a protein or fragment thereof having an amino acid sequence that is at least 85%, at least 90%, at least 95%, at least 98%, at least 99%, or 100% identical to a Hsp60 amino acid sequence associated with NCBI Reference Sequence: NP_002147.2. In some embodiments, Hsp60 is at least 85%, at least 90%, at least 95%, at least 98%, at least 99%, or 100% identical to the Hsp60 in Homo sapiens.

In some embodiments, the heat shock protein is Hsp70. In some embodiments, Hsp70 is a protein or fragment thereof having an amino acid sequence that is at least 85%, at least 90%, at least 95%, at least 98%, at least 99%, or 100% identical to a Hsp70 amino acid sequence associated with NCBI Reference Sequence: NP_002145.3. In some embodiments, Hsp70 is at least 85%, at least 90%, at least 95%, at least 98%, at least 99%, or 100% identical to the Hsp70 in Homo sapiens.

In some embodiments, the heat shock protein is Hsp90. In some embodiments, Hsp90 is a protein or fragment thereof having an amino acid sequence that is at least 85%, at least 90%, at least 95%, at least 98%, at least 99%, or 100% identical to a Hsp90 amino acid sequence associated with NCBI Reference Sequence: NP_005339.3. In some embodiments, Hsp90 is at least 85%, at least 90%, at least 95%, at least 98%, at least 99%, or 100% identical to the Hsp90 in Homo sapiens.

The heat shock protein (Hsp) inhibitors of the present disclosure inhibit the activity of at least one heat shock protein (e.g., Hsp27, Hsp40, Hsp60, Hsp70, Hsp90). In some embodiments, the heat shock protein (Hsp) inhibitors inhibit the activity of one or more heat shock proteins selected from heat shock protein 27 (Hsp27), heat shock protein 40 (Hsp40), heat shock protein 60 (Hsp60), heat shock protein 70 (Hsp70), and/or heat shock protein 90 (Hsp90). In some embodiments, an Hsp inhibitor is a small molecule. In some embodiments, an Hsp inhibitor is an inhibitory nucleic acid (e.g., shRNA, siRNA). In some embodiments, an Hsp inhibitor is a small interfering RNA (siRNA) that decreases gene expression of at least one heat shock protein (e.g., Hsp27, Hsp40, Hsp60, Hsp70, Hsp90). In some embodiments, an Hsp inhibitor is a short-hairpin RNA (shRNA) that decreases gene expression of at least one heat shock protein (e.g., Hsp27, Hsp40, Hsp60, Hsp70, Hsp90).

In some embodiments, the Hsp inhibitor is a Hsp27 inhibitor, and analogs thereof. In some embodiments, the Hsp27 inhibitor is a shRNA that decreases the gene expression of HSPB1, the gene that encodes Hsp27. In some embodiments, the shRNA targets the gene expression of rat HSPB1 for silencing. In some embodiments, the shRNA targets the gene expression of human HSPB1 for silencing.

In some embodiments, a shRNA that targets gene expression of HSPB1 is at least 85%, at least 90%, at least 95%, at least 98%, at least 99%, or 100% identical to the following nucleic acid sequence:

5′-CAC TGG CAA GCA CGA AGA AAG-3′

In some embodiments, a shRNA that targets gene expression of HSPB1 is at least 85%, at least 90%, at least 95%, at least 98%, at least 99%, or 100% identical to the following nucleic acid sequence:

5′-CAC CGG CAA GCA CGA GGA GCG-3′

In some embodiments, the Hsp inhibitor is a Hsp40 inhibitor, and analogs thereof. In some embodiments, the Hsp inhibitor is a Hsp60 inhibitor, and analogs thereof. In some embodiments, the Hsp inhibitor is a Hsp70 inhibitor, and analogs thereof. In some embodiments, the Hsp inhibitor is a Hsp90 inhibitor, and analogs thereof. In some embodiments, the Hsp inhibitor inhibits both Hsp 27 and Hsp90, and analogs thereof. In some embodiments, non-limiting examples of Hsp90 inhibitors include 17-Allylamino-geldanamycin (17-AGG, Enzo Life Sciences), Geldanamycin (GA, Enzo Life Sciences), CUDC-305 (Abmole), and NVP-HSP990 (Abmole), and analogs thereof. In some embodiments, non-limiting examples of Hsp90 inhibitors are also described in PCT/US2013/036783, the entire contents of which are incorporated herein by reference). Additional suitable Hsp inhibitors will be apparent to those of skill in the art based on this disclosure.

In some embodiments, the Hsp inhibitor is 17-Allylamino-geldanamycin (17-AGG) (IUPAC Name: 3 S,5S,6R,7S,8E,10R,11S,12E,14E)-21-(allylamino)-6-hydroxy-5,11-dimethoxy-3,7,9,15-tetramethyl-16,20,22-trioxo-17-azabicyclo[16.3.1]docosa-8,12,14,18,21-pentaen-10-yl] carbamate), which has the chemical formula C₃₁H₄₃N₃O₈. 17-AGG inhibits the function of Hsp90 (Heat Shock Protein 90).

In some embodiments, the Hsp inhibitor is Geldanamycin (GA) (IUPAC Name: 4E,6Z,8S,9S,10E,12S,13R,14S,16R)-13-hydroxy-8,14,19-trimethoxy-4,10,12,16-tetramethyl-3,20,22-trioxo-2-azabicyclo[16.3.1]docosa-1(21),4,6,10,18-pentaen-9-yl carbamate), which has the chemical formula C₂₉H₄₀N₂O₉. GA is a 1,4-benzoquinone ansamycin antitumor antibiotic that inhibits the function of Hsp90 (Heat Shock Protein 90) by binding to the unusual ADP/ATP-binding pocket of the protein.

In some embodiments, the Hsp inhibitor is CUDC-305 (IUPAC Name: 4-amino-2-[[6-(dimethylamino)-1,3-benzodioxol-5-yl]thio]-N-(2,2-dimethylpropyl)-1H-imidazo[4,5-c]pyridine-1-ethanamine), which has the chemical formula C₂₂H₃₀N₆O₂S. CUDC-305 inhibits the function of Hsp90 (Heat Shock Protein 90).

In some embodiments, the Hsp inhibitor is NVP-HSP990 (IUPAC Name: (R)-2-amino-7-((R)-4-fluoro-2-(6-methoxypyridin-2-yl)phenyl)-4-methyl-7,8-dihydropyrido[4,3-d]pyrimidin-5(6H)-one), which has the chemical formula C₂₀H₁₈FN₅O₂. NVP-HSP990 inhibits the function of Hsp90 (Heat Shock Protein 90).

The heat shock protein (Hsp) inhibitors (e.g., inhibitors of Hsp27, Hsp40, Hsp60, Hsp70, Hsp90) of the disclosure may be incorporated into a pharmaceutical composition as provided herein for administration to a subject. In some embodiments, one or more Hsp inhibitors (e.g., inhibitors of Hsp27, Hsp40, Hsp60, Hsp70, Hsp90) are formulated for administration to a subject in need. In some embodiments, one or more Hsp inhibitors (e.g., inhibitors of Hsp27, Hsp40, Hsp60, Hsp70, Hsp90) are incorporated into a pharmaceutical composition for administration to a subject. In some embodiments, one or more Hsp inhibitors (e.g., inhibitors of Hsp27, Hsp40, Hsp60, Hsp70, Hsp90) are administered to a subject in need thereof to treat a disease or disorder (e.g., neurodevelopmental disorder) and symptoms thereof, caused by mutations in mTOR regulatory genes (e.g., TSC1, TSC2, AKT3, DEPDC5), mutations in genes that play a role in cilia assembly and/or function, from a disruption of the mTOR pathway, and/or from dysfunctional mTORC1 activity in the subject. The Hsp inhibitors (e.g., inhibitors of Hsp27, Hsp40, Hsp60, Hsp70, Hsp90) of the disclosure may be used in combination with the mTOR inhibitors (e.g., MCI-186, Nicardipine-HCl, K252A, Tyrphostin 9, LY-294002, rapamycin, everolimus) described herein. Pharmaceutical compositions as provided herein for administration to a subject may be formulated to include one or more Hsp inhibitors and/or mTOR inhibitors.

The therapeutic methods of the disclosure in general comprise administration of an effective amount of the Hsp inhibitors described herein, such as a pharmaceutical composition herein, to a subject (e.g., animal, human) in need thereof, including a mammal, particularly a human, to treat the disease or disorder or symptom thereof, under conditions such that the disease or disorder is treated to produce such effect. Such treatment will be suitably administered to subjects, particularly humans, suffering from, having, susceptible to, or at risk for a disease, disorder, or symptom thereof. Determination of those subjects in need of such treatment can be made by any objective or subjective determination by a diagnostic test or opinion of a subject or health care provider (e.g., genetic test, enzyme or protein marker, Marker (as defined herein), family history, and the like). The Hsp inhibitors herein may be also used in the treatment of any other disorders in which the mechanistic target of rapamycin (mTOR) pathway may be implicated.

mTOR Inhibitors

The present disclosure features mechanistic target of rapamycin (mTOR) inhibitors with the ability to inhibit mTORC1 activity and/or increase or normalize neuronal cilia so as to treat a disease or disorder (e.g., neurodevelopmental disorder) and its symptoms, either prophylactically or therapeutically, caused by mutations in mTOR regulatory genes (e.g., TSC1, TSC2, AKT3, DEPDC5), mutations in genes that play a role in cilia assembly and/or function, from a disruption of the mTOR pathway, and/or from dysfunctional mTORC1 activity following administration and delivery to a subject.

A mechanistic target of rapamycin (mTOR) inhibitor as described herein is meant an agent, compound, or substance that inhibits at least one activity of the mTOR pathway. In some embodiments, the methods herein include administration of inhibitors of the mTOR pathway to a subject in need. In some embodiments, mTOR inhibitors inhibit S6 phosphorylation. In some embodiments, mTOR inhibitors include, but are not limited to rapamycin, everolimus, Geldanamycin (GA), 17-Allylamino-geldanamycin (17-AGG), MCI-186, Nicardipine-HCl, K252A, Tyrphostin 9 and LY-294002. Additional suitable mTOR inhibitors will be apparent to those of skill in the art based on this disclosure.

In some embodiments, the mTOR inhibitor is rapamycin. Rapamycin (also known as sirolimus) (IUPAC Name: 1R,9S,12S,15R,16E,18R,19R,21R,23S,24E,26E,28E,30S,32S,35R)-1,18-dihydroxy-12-{(2R)-1-[(1S,3R,4R)-4-hydroxy-3-methoxycyclohexyl]-2-propanyl}-19,30-dimethoxy-15,17,21,23,29,35-hexamethyl-11,36-dioxa-4-azatricyclo[30.3.1.0˜4,9˜]hexatriaconta-16,24,26,28-tetraene-2,3,10,14,20-pentone) is a macrolide compound with the chemical formula C₅₁H₇₉NO₁₃. Rapamycin inhibits the mTOR pathway by directly binding to mTOR Complex 1 (mTORC1).

In some embodiments, the mTOR inhibitor is everolimus. Everolimus (IUPAC Name: Dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0 hexatriaconta-16,24,26,28-tetraene-2,3,10,14,20-pentone) has the chemical formula C₅₃H₈₃NO₁₄ and is the 40-O-(2-hydroxyethyl) derivative of sirolimus and works similarly as an inhibitor of mTOR.

In some embodiments, the mTOR inhibitor is MCI-186. MCI-186 (also known as edaravone) (IUPAC Name: 5-methyl-2-phenyl-4H-pyrazol-3-one) has the chemical formula C₁₀H₁₀N₂O and functions as an anti-oxidant.

In some embodiments, the mTOR inhibitor is Nicardipine-HCl. Nicardipine-HCl (IUPAC Name: 5-O-[2-[benzyl(methyl)amino]ethyl] 3-O-methyl 2,6-dimethyl-4-(3-nitrophenyl)-1,4-dihydropyridine-3,5-dicarboxylate; hydrochloride) has the chemical formula C₂₆H₃₀ClN₃O₆ and functions as a calcium channel blocker.

In some embodiments, the mTOR inhibitor is K252A. K252A (IUPAC Name: 9S-(9α,10β,12α))-2,3,9,10,11,12-hexahydro-10-hydroxy-10-(methoxycarbonyl)-9-methyl-9,12-epoxy-1H-diindolo[1,2,3-fg:3′,2′,1′-kl]pyrrolo[3,4-i][1,6]benzodiazocin-1-one) has the chemical formula C₂₇H₂₁N₃O₅ and functions as a kinase inhibitor.

In some embodiments, the mTOR inhibitor is Tyrphostin 9. Tyrphostin 9 (also known as SF-6847 or Malonoben) (IUPAC Name: [4-Hydroxy-3,5-bis(2-methyl-2-propanyl)benzylidene]malononitrile) has the chemical formula C₁₈H₂₂N₂O and functions as an inhibitor of the PDGF receptor tyrosine kinase.

In some embodiments, the mTOR inhibitor is LY-294002. LY-294002 (IUPAC Name: 2-Morpholin-4-yl-8-phenylchromen-4-one) has the chemical formula C₁₉H₁₇NO₃ and functions as an inhibitor of phosphoinositide 3-kinases (PI3Ks).

The mTOR inhibitors (e.g., MCI-186, Nicardipine-HCl, K252A, Tyrphostin 9, LY-294002, rapamycin, everolimus) of the disclosure may be incorporated into a pharmaceutical composition as provided herein for administration to a subject. In some embodiments, one or more mTOR inhibitors (e.g., MCI-186, Nicardipine-HCl, K252A, Tyrphostin 9, LY-294002, rapamycin, everolimus) are formulated for administration to a subject in need. In some embodiments, one or more mTOR inhibitors (e.g., MCI-186, Nicardipine-HCl, K252A, Tyrphostin 9, LY-294002, rapamycin, everolimus) are incorporated into a pharmaceutical composition for administration to a subject. In some embodiments, one or more mTOR inhibitors (e.g., MCI-186, Nicardipine-HCl, K252A, Tyrphostin 9, LY-294002, rapamycin, everolimus) are administered to a subject in need thereof to treat a disease or disorder (e.g., neurodevelopmental disorder) and symptoms thereof, caused by mutations in mTOR regulatory genes (e.g., TSC1, TSC2, AKT3, DEPDC5), mutations in genes that play a role in cilia assembly and/or function, from a disruption of the mTOR pathway, and/or from dysfunctional mTORC1 activity in the subject. The mTOR inhibitors (e.g., MCI-186, Nicardipine-HCl, K252A, Tyrphostin 9, LY-294002, rapamycin, everolimus) of the disclosure may be used in combination with the Hsp inhibitors (e.g., inhibitors of Hsp27, Hsp40, Hsp60, Hsp70, Hsp90) described herein. Pharmaceutical compositions as provided herein for administration to a subject may be formulated to include one or more Hsp inhibitors (e.g., inhibitors of Hsp27, Hsp40, Hsp60, Hsp70, Hsp90) and/or mTOR inhibitors (e.g., MCI-186, Nicardipine-HCl, K252A, Tyrphostin 9, LY-294002, rapamycin, everolimus).

The therapeutic methods of the disclosure in general comprise administration of an effective amount of the mTOR inhibitors (e.g., MCI-186, Nicardipine-HCl, K252A, Tyrphostin 9, LY-294002, rapamycin, everolimus) described herein, such as a pharmaceutical composition herein, to a subject (e.g., animal, human) in need thereof, including a mammal, particularly a human, to treat the disease or disorder or symptom thereof, under conditions such that the disease or disorder is treated to produce such effect. Such treatment will be suitably administered to subjects, particularly humans, suffering from, having, susceptible to, or at risk for a disease, disorder, or symptom thereof. Determination of those subjects in need of such treatment can be made by any objective or subjective determination by a diagnostic test or opinion of a subject or health care provider (e.g., genetic test, enzyme or protein marker, Marker (as defined herein), family history, and the like). The mTOR inhibitors (e.g., MCI-186, Nicardipine-HCl, K252A, Tyrphostin 9, LY-294002, rapamycin, everolimus) herein may be also used in the treatment of any other disorders in which the mechanistic target of rapamycin (mTOR) pathway may be implicated.

### Inhibitory Nucleic Acids

Inhibitory nucleic acid molecules are oligonucleotides that inhibit the expression or activity of a polypeptide that is overexpressed in neurodevelopmental disorders (e.g., tuberous sclerosis complex (TSC)). Such oligonucleotides include single and double stranded nucleic acid molecules (e.g., DNA, RNA, and analogs thereof) that bind a nucleic acid molecule that encodes a polypeptide (e.g., antisense molecules, siRNA, shRNA) that is overexpressed in neurodevelopmental disorders (e.g., tuberous sclerosis complex (TSC)) as well as nucleic acid molecules that bind directly to the polypeptide to modulate its biological activity. In some embodiments, the inhibitory nucleic acid molecule is a double-stranded RNA, siRNA, shRNA, or antisense RNA, or a fragment thereof.

An inhibitory nucleic acid molecule that “corresponds” to a gene (e.g. HSPB1) encoding a heat shock protein (Hsp) (e.g., Hsp27, Hsp40, Hsp60, Hsp70, Hsp90) comprises at least a fragment of the double-stranded gene, such that each strand of the double-stranded inhibitory nucleic acid molecule is capable of binding to the complementary strand of the target Hsp gene (e.g. HSPB1). The inhibitory nucleic acid molecule need not have perfect correspondence to the reference gene sequence. In one embodiment, an siRNA has at least about 85%, 90%, 95%, 96%, 97%, 98%, or even 99% sequence identity with the target nucleic acid. For example, a 19 base pair duplex having 1-2 base pair mismatch is considered useful in the methods of the invention. In other embodiments, the nucleobase sequence of the inhibitory nucleic acid molecule exhibits 1, 2, 3, 4, 5 or more mismatches.

In one embodiment, an inhibitory nucleic acid molecule is a double-stranded RNA used for RNA interference (RNAi)-mediated knock-down of heat shock protein gene expression (e.g., HSPB1). In one embodiment, expression of a heat shock protein gene (e.g., HSPB1) is reduced in a neuron. RNA interference (RNAi) is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). In RNAi, gene silencing is typically triggered post-transcriptionally by the presence of double-stranded RNA (dsRNA) in a cell. This dsRNA is processed intracellularly into shorter pieces called small interfering RNAs (siRNAs).

Small interfering RNAs (siRNAs), which are typically short twenty-one to twenty-five nucleotide double-stranded RNAs, are effective at down-regulating gene expression (Zamore et al., Cell 101: 25-33; Elbashir et al., Nature 411: 494-498, 2001, hereby incorporated by reference). The therapeutic effectiveness of an siRNA approach in mammals was demonstrated in vivo by McCaffrey et al. (Nature 418: 38-39.2002).

siRNAs may be designed to inactivate a specific target gene sequence. Such siRNAs, for example, could be administered directly to an affected tissue (e.g., brain), or administered systemically. The nucleic acid sequence of a gene can be used to design small interfering RNAs (siRNAs). The 21 to 25 nucleotide siRNAs may be used, for example, as therapeutics to treat neurodevelopmental disorders (e.g., tuberous sclerosis complex (TSC)).

The introduction of siRNAs into cells either by transfection of dsRNAs or through expression of shRNAs using a plasmid-based expression system is currently being used to create loss-of-function phenotypes in mammalian cells. As described herein, siRNAs that target heat shock protein (Hsp) genes (e.g. HSPB1) decrease heat shock protein (e.g., Hsp27, Hsp40, Hsp60, Hsp70, Hsp90) expression in cells (e.g., neurons). The inhibitory nucleic acid molecules provided by the invention are not limited to siRNAs, but include any nucleic acid molecule sufficient to decrease the expression of a heat shock protein (Hsp) (e.g., Hsp27, Hsp40, Hsp60, Hsp70, Hsp90) nucleic acid molecule or polypeptide. Each of the DNA sequences provided herein may be used, for example, in the discovery and development of therapeutic antisense nucleic acid molecule to decrease the expression of a heat shock protein (Hsp) (e.g., Hsp27, Hsp40, Hsp60, Hsp70, Hsp90).

In one embodiment, a double-stranded RNA (dsRNA) molecule is made that includes between eight and twenty-five (e.g., 8, 10, 12, 15, 16, 17, 18, 19, 20, 21, 22, 23, 24, 25) consecutive nucleobases. The dsRNA can be two complementary strands of RNA that have duplexed, or a single RNA strand that has self-duplexed (small hairpin RNA (shRNA)). Typically, dsRNAs are about 21 or 22 base pairs, but may be shorter or longer (up to about 29 nucleobases) if desired. Double stranded RNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

Small hairpin RNAs (shRNAs) comprise an RNA sequence having a stem-loop structure. Such structures are well known in the art and the term is used consistently with its known meaning in the art. As is known in the art, the secondary structure does not require exact base-pairing. Thus, the stem can include one or more base mismatches or bulges. Alternatively, the base-pairing can be exact, i.e. not include any mismatches. The multiple stem-loop structures can be linked to one another through a linker, such as, for example, a nucleic acid linker, a micro-RNA (miRNA) flanking sequence, other molecule, or some combination thereof. While there may be some variation in range, a conventional stem-loop shRNA can comprise a stem ranging from 19 to 29 bp, and a loop ranging from 4 to 30 bp.

MicroRNAs (miRNAs) are endogenously encoded RNA molecules that are about 22-nucleotides long and generally expressed in a highly tissue- or developmental-stage-specific fashion and that post-transcriptionally regulate target genes. In some embodiments, the precursor miRNA molecule can include more than one stem-loop structure. More than 200 distinct miRNAs have been identified in plants and animals. These small regulatory RNAs are believed to serve important biological functions by two prevailing modes of action: (1) by repressing the translation of target mRNAs, and (2) through RNA interference (RNAi), that is, cleavage and degradation of mRNAs. In the latter case, miRNAs function analogously to small interfering RNAs (siRNAs). Thus, one can design and express artificial miRNAs based on the features of existing miRNA genes.

shRNAs can be expressed from DNA vectors to provide sustained silencing and high yield delivery into almost any cell type. In some embodiments, the vector is a viral vector. Exemplary viral vectors include retroviral, including lentiviral, adenoviral, baculoviral and avian viral vectors, and including such vectors allowing for stable, single-copy genomic integrations. Retroviruses from which the retroviral plasmid vectors can be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus. In some embodiments, the vector is an adeno-associated viral (AAV) vector.

A retroviral plasmid vector can be employed to transduce packaging cell lines to form producer cell lines. Examples of packaging cells which can be transfected include, but are not limited to, the PE501, PA317, R-2, R-AM, PA12, T19-14x, VT-19-17-H₂, RCRE, RCRIP, GP+E-86, GP+envAm12, and DAN cell lines as described in Miller, Human Gene Therapy 1:5-14 (1990), which is incorporated herein by reference in its entirety. The vector can transduce the packaging cells through any means known in the art. A producer cell line generates infectious retroviral vector particles which include polynucleotide encoding a DNA replication protein. Such retroviral vector particles then can be employed, to transduce eukaryotic cells, either in vitro or in vivo. The transduced eukaryotic cells will express a DNA replication protein.

Essentially any method for introducing a nucleic acid construct into cells can be employed. Physical methods of introducing nucleic acids include injection of a solution containing the construct, bombardment by particles covered by the construct, soaking a cell, tissue sample or organism in a solution of the nucleic acid, or electroporation of cell membranes in the presence of the construct. A viral construct packaged into a viral particle can be used to accomplish both efficient introduction of an expression construct into the cell and transcription of the encoded shRNA. Other methods known in the art for introducing nucleic acids to cells can be used, such as lipid-mediated carrier transport, chemical mediated transport, such as calcium phosphate, and the like. Thus, the shRNA-encoding nucleic acid construct can be introduced along with components that perform one or more of the following activities: enhance RNA uptake by the cell, promote annealing of the duplex strands, stabilize the annealed strands, or otherwise increase inhibition of the target gene.

For expression within cells, DNA vectors, for example plasmid vectors comprising either an RNA polymerase II or RNA polymerase III promoter can be employed. Expression of endogenous miRNAs is controlled by RNA polymerase II (Pol II) promoters and in some cases, shRNAs are most efficiently driven by Pol II promoters, as compared to RNA polymerase III promoters (Dickins et al., 2005, Nat. Genet. 39: 914-921). In some embodiments, expression of the shRNA can be controlled by an inducible promoter or a conditional expression system, including, without limitation, RNA polymerase type II promoters. Examples of useful promoters in the context of the invention are tetracycline-inducible promoters (including TRE-tight), IPTG-inducible promoters, tetracycline transactivator systems, and reverse tetracycline transactivator (rtTA) systems. Constitutive promoters can also be used, as can cell- or tissue-specific promoters. Many promoters will be ubiquitous, such that they are expressed in all cell and tissue types. A certain embodiment uses tetracycline-responsive promoters, one of the most effective conditional gene expression systems in in vitro and in vivo studies. See International Patent Application PCT/US2003/030901 (Publication No. WO 2004-029219 A2) and Fewell et al., 2006, Drug Discovery Today 11: 975-982, for a description of inducible shRNA.

### Delivery of Inhibitory Nucleic Acids

Naked polynucleotides, or analogs thereof, are capable of entering mammalian cells (e.g., neurons) and inhibiting expression of a gene of interest (e.g., HSPB1). Nonetheless, it may be desirable to utilize a formulation that aids in the delivery of an inhibitory nucleic acid molecule to cells (e.g., neurons) (see, e.g., U.S. Pat. Nos. 5,656,611, 5,753,613, 5,785,992, 6,120,798, 6,221,959, 6,346,613, and 6,353,055, each of which is hereby incorporated by reference).

### Pharmaceutical Compositions

Compositions comprising Hsp inhibitors (e.g., inhibitors of Hsp27, Hsp40, Hsp60, Hsp70, Hsp90) and/or mTOR inhibitors (e.g., MCI-186, Nicardipine-HCl, K252A, Tyrphostin 9, LY-294002, rapamycin, everolimus), as described herein are provided. In some embodiments, pharmaceutical compositions of the present disclosure include one or more of a heat shock protein (Hsp) inhibitor (e.g., inhibitors of Hsp27, Hsp40, Hsp60, Hsp70, Hsp90), MCI-186, Nicardipine-HCl, K252A, Tyrphostin 9, LY-294002, rapamycin, and/or everolimus. In some embodiments, pharmaceutical compositions of the present disclosure include, but are not limited to, one or more heat shock protein inhibitors selected from an inhibitor of Heat shock protein 27, (Hsp27), Heat shock protein 90 (Hsp90), Heat shock protein 27 (Hsp27), Heat shock protein 70 (Hsp70), Heat shock protein 60 (Hsp60), Heat shock protein 40 (Hsp40), and analogs thereof. In some embodiments, the pharmaceutical composition includes an inhibitor of Hsp27, and analogs thereof. In some embodiments, the pharmaceutical composition includes an inhibitor of Hsp90, and analogs thereof. In some embodiments, the pharmaceutical composition includes an inhibitor of Hsp27 and Hsp90, and analogs thereof. In some embodiments, the mTOR inhibitors include MCI-186, Nicardipine-HCl, K252A, Tyrphostin 9, LY-294002, rapamycin, and/or everolimus, and analogs thereof. In some embodiments, the pharmaceutical composition includes MCI-186, and analogs thereof. In some embodiments, the pharmaceutical composition includes Nicardipine-HCl, and analogs thereof. In some embodiments, the pharmaceutical composition includes K252A, or analogs thereof. In some embodiments, the pharmaceutical composition includes Tyrphostin 9, or analogs thereof. In some embodiments, the pharmaceutical composition includes LY-294002, or analogs thereof. In some embodiments, the pharmaceutical composition includes rapamycin, or analogs thereof. In some embodiments, the pharmaceutical composition includes everolimus, or analogs thereof. In some embodiments, the pharmaceutical compositions herein further include a pharmaceutically acceptable carrier, diluent, excipient, or vehicle.

Compositions and preparations (e.g., physiologically or pharmaceutically acceptable compositions) containing Hsp inhibitors and/or mTOR inhibitors for administration include, without limitation, sterile aqueous or non-aqueous solutions, suspensions, and emulsions. Nonlimiting examples of non-aqueous solvents include propylene glycol, polyethylene glycol, vegetable oils, such as olive oil and canola oil, and injectable organic esters, such as ethyl oleate. Aqueous carriers include water, alcoholic/aqueous solutions, emulsions or suspensions, including saline and buffered media. Parenteral vehicles include, for example, sodium chloride solution, Ringer's dextrose, dextrose and sodium chloride, lactated Ringer's, or fixed oils. Intravenous vehicles include, for example, fluid and nutrient replenishers, electrolyte replenishers (such as those based on Ringer's dextrose), and the like. Preservatives and other additives may also be present in such compositions and preparations, such as, for example, antimicrobials, antioxidants, chelating agents, colorants, stabilizers, inert gases and the like.

Some of the compositions may potentially be administered as a pharmaceutically acceptable acid- or base-addition salt, formed by reaction with inorganic acids, such as hydrochloric acid, hydrobromic acid, perchloric acid, nitric acid, thiocyanic acid, sulfuric acid, and phosphoric acid, and organic acids, such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, tri-alkyl and aryl amines and substituted ethanolamines.

Provided herein are pharmaceutical compositions which include an effective amount of Hsp inhibitors (e.g., inhibitors of Hsp27, Hsp40, Hsp60, Hsp70, Hsp90) and/or mTOR inhibitors (e.g., MCI-186, Nicardipine-HCl, K252A, Tyrphostin 9, LY-294002, rapamycin, everolimus), alone, or in combination with a pharmaceutically acceptable carrier. Pharmaceutically acceptable carriers include, but are not limited to, saline, buffered saline, dextrose, water, glycerol, ethanol, and combinations thereof. The carrier and composition can be sterile, and the formulation suits the mode of administration. The composition can also contain minor amounts of wetting or emulsifying agents, or pH buffering agents. The composition can be a liquid or aqueous solution, suspension, emulsion, dispersion, tablet, pill, capsule, powder, or sustained release formulation. A liquid or aqueous composition can be lyophilized and reconstituted with a solution or buffer prior to use. The composition can be formulated as a suppository, with traditional binders and carriers such as triglycerides. Oral formulations can include standard carriers, such as pharmaceutical grades of mannitol, lactose, starch, magnesium stearate, sodium saccharine, cellulose, and magnesium carbonate. Any of the commonly known pharmaceutical carriers, such as sterile saline solution or sesame oil, can be used. The medium can also contain conventional pharmaceutical adjunct materials such as, for example, pharmaceutically acceptable salts to adjust the osmotic pressure, buffers, preservatives and the like. Other media that can be used in the compositions and administration methods as described are normal saline and sesame oil.

### Methods of Treatment, Methods of Use, Administration and Delivery

Methods of treating a disease, or symptoms thereof, are provided. The present disclosure features methods that are useful for the treatment of neurodevelopmental disorders (e.g., tuberous sclerosis complex (TSC)). The present disclosure also features methods that are useful for the treatment of mTORopathies and/or neuronal ciliopathies. In some embodiments, the present disclosure features methods that are useful for the treatment of disease caused by mutations in one or more mechanistic target of rapamycin (mTOR) regulatory genes (e.g., TSC1, TSC2, AKT3, DEPDC5). In some embodiments, the one or more mTOR regulatory genes are selected from the group consisting of TSC1, TSC2, AKT3, and DEPDC5. In some embodiments, the present disclosure features methods that are useful for the treatment of disease caused by mutations in the TSC1 or TSC2 genes (e.g., tuberous sclerosis complex (TSC)). In some embodiments, the present disclosure features methods that are useful for the treatment of disease caused by dysfunctional mechanistic target of rapamycin complex 1 (mTORC1) activity. In some embodiments, the mTORC1 activity is increased. In some embodiments, the present disclosure features methods that are useful for the treatment of disease associated with a decrease in neuronal cilia. Nonlimiting examples of diseases or disorders that can be treated using the methods provided herein include, but are not limited to Tuberous Sclerosis Complex (TSC), intellectual disability, brain malformations, cortical tubers, neural ciliopathy, epilepsy, neuropathy, autism, hemimegalencephaly, cortical dysplasia, focal cortical dysplasia, traumatic brain injury, brain tumours, and/or dementia, or a combination thereof. In particular, the disclosure features methods that are useful for the treatment of Tuberous Sclerosis Complex (TSC).

The present disclosure provides methods of treating disease and/or disorders or symptoms thereof which comprise administering a therapeutically effective amount of a pharmaceutical composition comprising a compound of the formulae herein to a subject (e.g., a mammal such as a human). The methods comprise administering an effective amount of one or more Hsp inhibitors (e.g., inhibitors of Hsp27, Hsp40, Hsp60, Hsp70, Hsp90) and/or mTOR inhibitors (e.g., MCI-186, Nicardipine-HCl, K252A, Tyrphostin 9, LY-294002, rapamycin, everolimus), as described herein, or a pharmaceutical composition comprising one or more Hsp inhibitors (e.g., inhibitors of Hsp27, Hsp40, Hsp60, Hsp70, Hsp90) and/or mTOR inhibitors (e.g., MCI-186, Nicardipine-HCl, K252A, Tyrphostin 9, LY-294002, rapamycin, everolimus), to a subject (e.g., a mammal), in particular, a human subject. The disclosure provides methods of treating a subject suffering from, or at risk of, or susceptible to disease, or a symptom thereof, or delaying the progression of a disease (e.g., neurodevelopmental disorder) caused by mutations in mTOR regulatory genes (e.g., TSC1, TSC2, AKT3, DEPDC5), mutations in genes that play a role in cilia assembly and/or function, from a disruption of the mTOR pathway, and/or from dysfunctional mTORC1 activity. In some embodiments, the method includes administering to the subject (e.g., a mammalian subject), an amount or an effective amount of an pharmaceutical composition comprising one or more Hsp inhibitors (e.g., inhibitors of Hsp27, Hsp40, Hsp60, Hsp70, Hsp90) and/or mTOR inhibitors (e.g., MCI-186, Nicardipine-HCl, K252A, Tyrphostin 9, LY-294002, rapamycin, everolimus), sufficient to treat the disease, delay the growth of, or treat the symptoms thereof, caused by mutations in mTOR regulatory genes (e.g., TSC1, TSC2, AKT3, DEPDC5), mutations in genes that play a role in cilia assembly and/or function, from a disruption of the mTOR pathway, and/or from dysfunctional mTORC1 activity under conditions in which the disease and/or the symptoms thereof are treated.

In some embodiments, the methods herein include administering to the subject (including a human subject identified as in need of such treatment) an effective amount of one or more Hsp inhibitors (e.g., inhibitors of Hsp27, Hsp40, Hsp60, Hsp70, Hsp90) and/or mTOR inhibitors (e.g., MCI-186, Nicardipine-HCl, K252A, Tyrphostin 9, LY-294002, rapamycin, everolimus), or a pharmaceutical composition thereof, as described herein to produce such effect. In some embodiments, the one or more heat shock protein inhibitors includes, but is not limited to, inhibitors of Heat shock protein 90 (Hsp90), Heat shock protein 27 (Hsp27), Heat shock protein 70 (Hsp70), Heat shock protein 60 (Hsp60), Heat shock protein 40 (Hsp40), and analogs thereof. In some embodiments, an Hsp inhibitor is a small molecule. In some embodiments, an Hsp inhibitor is a short-hairpin RNA (shRNA) that decreases gene expression of a heat shock protein. In some embodiments, the Hsp inhibitor is a Hsp27 inhibitor, and analogs thereof. In some embodiments, the Hsp27 inhibitor is a shRNA that decreases the gene expression of HSPB1, the gene that encodes Hsp27. In some embodiments, the Hsp inhibitor is an inhibitor of Hsp90, or an analog thereof. In some embodiments, the Hsp inhibitor is an inhibitor of Hsp27 and Hsp90, or an analog thereof. In some embodiments, the mTOR inhibitors include MCI-186, Nicardipine-HCl, K252A, Tyrphostin 9, LY-294002, rapamycin, and/or everolimus, or analogs thereof. In some embodiments, the mTOR inhibitor is MCI-186, or an analog thereof. In some embodiments, the mTOR inhibitor is Nicardipine-HCl, or an analog thereof. The treatment methods are suitably administered to subjects, particularly humans, suffering from, are susceptible to, or at risk of having a disease, or symptoms thereof, caused by mutations in mTOR regulatory genes (e.g., TSC1, TSC2, AKT3, DEPDC5), mutations in genes that play a role in cilia assembly and/or function, from a disruption of the mTOR pathway, and/or from dysfunctional mTORC1 activity, namely, neurodevelopmental disorders (e.g., mTORopathies, neuronal ciliopathies).

Identifying a subject in need of such treatment can be based on the judgment of the subject or of a health care professional and can be subjective (e.g., opinion) or objective (e.g., measurable by a test or diagnostic method). Briefly, the determination of those subjects who are in need of treatment or who are “at risk” or “susceptible” can be made by any objective or subjective determination by a diagnostic test (e.g., blood sample, biopsy, genetic test, enzyme or protein marker assay), marker analysis, family history, and the like, including an opinion of the subject or a health care provider. In some embodiments, the subject in need of treatment can be identified by, for example, measuring protein levels (e.g. mTORC1, TSC1, TSC2) or cilia length from cortical tubers collected from a patient (e.g., tissue sample) (see Example 2). The Hsp inhibitors and/or mTOR inhibitors as described herein, may also be used in the treatment of any other disorders in which disease caused by mTOR pathway dysfunction may be implicated. A subject undergoing treatment can be a non-human mammal, such as a veterinary subject, or a human subject (also referred to as a “patient”).

In another embodiment, a method of monitoring the progress of a disease (e.g., neurodevelopmental disorder) caused by mutations in mTOR regulatory genes (e.g., TSC1, TSC2, AKT3, DEPDC5), mutations in genes that play a role in cilia assembly and/or function, from a disruption of the mTOR pathway, and/or from dysfunctional mTORC1 activity, or monitoring treatment of the disease is provided. The method includes a diagnostic measurement (e.g., biopsy, CT scan, screening assay or detection assay) in a subject suffering from or susceptible to disease or symptoms thereof associated with neurodevelopmental disorders (e.g., mTORopathies (e.g., TSC), ciliopathies), in which the subject has been administered an amount (e.g., a therapeutic amount) of a pharmaceutical composition as described herein, sufficient to treat the disease or symptoms thereof. The diagnostic measurement in the method can be compared to samples from healthy, normal controls; in a pre-disease sample of the subject; or in other afflicted/diseased patients to establish the treated subject's disease status. For monitoring, a second diagnostic measurement may be obtained from the subject at a time point later than the determination of the first diagnostic measurement, and the two measurements can be compared to monitor the course of disease or the efficacy of the therapy/treatment. In certain embodiments, a pre-treatment measurement in the subject (e.g., in a sample or biopsy obtained from the subject or CT scan) is determined prior to beginning treatment as described; this measurement can then be compared to a measurement in the subject after the treatment commences and/or during the course of treatment to determine the efficacy of (monitor the efficacy of) the disease treatment.

The pharmaceutical compositions provided herein can be administered to a subject by any of the routes normally used for introducing a compound into a subject. Routes and methods of administration include, without limitation, intradermal, intramuscular, intraperitoneal, intrathecal, parenteral, such as intravenous (IV) or subcutaneous (SC), vaginal, rectal, intranasal, inhalation, intraocular, intracranial, or oral. Parenteral administration, such as subcutaneous, intravenous or intramuscular administration, is generally achieved by injection (immunization). Injectables can be prepared in conventional forms and formulations, either as liquid solutions or suspensions, solid forms (e.g., lyophilized forms) suitable for solution or suspension in liquid prior to injection, or as emulsions. Injection solutions and suspensions can be prepared from sterile powders, granules, and tablets. Administration can be systemic or local. In some embodiments, administration of Hsp inhibitors (e.g., inhibitors of Hsp27, Hsp40, Hsp60, Hsp70, Hsp90) and/or mTOR inhibitors (e.g., MCI-186, Nicardipine-HCl, K252A, Tyrphostin 9, LY-294002, rapamycin, everolimus), or pharmaceutical compositions thereof, is oral.

Ciliation refers to the growth and development of cilia. The present disclosure also provides methods for increasing or normalizing ciliation in a cell. In some embodiments, the methods provided herein increase or normalize ciliation in a cell by administering to a cell one or more heat shock protein (Hsp) inhibitors (e.g., inhibitors of Hsp27, Hsp40, Hsp60, Hsp70, Hsp90) and/or mTOR inhibitors (e.g., MCI-186, Nicardipine-HCl, K252A, Tyrphostin 9, LY-294002, rapamycin, everolimus). In some embodiments, the methods provided herein increase or normalize ciliation in a cell by administering to a cell one or more heat shock protein (Hsp) inhibitors of Hsp27, Hsp40, Hsp60, Hsp70, and Hsp90. In some embodiments, the methods provided herein increase or normalize ciliation in a cell by administering to a cell one or more Hsp27 inhibitors. In some embodiments, the Hsp27 inhibitor is an inhibitory nucleic acid (e.g., shRNA). In some embodiments, the methods provided herein increase or normalize ciliation in a cell by administering to a cell one or more Hsp90 inhibitors. In some embodiments, the Hsp90 inhibitor is selected from 17-Allylamino-geldanamycin (17-AGG), Geldanamycin (GA), CUDC-305, and/or NVP-HSP990. In some embodiments, the methods provided herein increase or normalize ciliation in a cell by administering to a cell one or more mTOR inhibitors. In some embodiments, the methods provided herein increase or normalize ciliation in a cell by administering to a cell one or more mTOR inhibitors selected from MCI-186, Nicardipine-HCl, K252A, Tyrphostin 9, LY-294002, rapamycin, and/or everolimus. Ciliation may be measured relative to a reference (e.g., untreated cell and/or a wild-type cell). In some embodiments, ciliation is measured relative to a wild-type cell. In some embodiments, ciliation is measured relative to an untreated cell. In some embodiments, the cell is a neuron. In some embodiments, the method for increasing or normalizing ciliation is performed in vivo. In some embodiments, the method for increasing or normalizing ciliation is performed in vitro.

In some embodiments, methods for increasing or normalizing ciliation in neurons are provided. In some embodiments, the methods provided herein increase or normalize ciliation in a neuron by administering to a neuron one or more heat shock protein (Hsp) inhibitors (e.g., inhibitors of Hsp27, Hsp40, Hsp60, Hsp70, Hsp90) and/or mTOR inhibitors (e.g., MCI-186, Nicardipine-HCl, K252A, Tyrphostin 9, LY-294002, rapamycin, everolimus). In some embodiments, the methods provided herein increase or normalize neuronal ciliation by administering to a neuron one or more heat shock protein (Hsp) inhibitors of Hsp27, Hsp40, Hsp60, Hsp70, and Hsp90. In some embodiments, the methods provided herein increase or normalize ciliation by administering to a neuron one or more Hsp27 inhibitors. In some embodiments, the Hsp27 inhibitor is an inhibitory nucleic acid (e.g., shRNA). In some embodiments, the methods provided herein increase or normalize neuronal ciliation by administering to a neuron one or more Hsp90 inhibitors. In some embodiments, the Hsp90 inhibitor is selected from 17-Allylamino-geldanamycin (17-AGG), Geldanamycin (GA), CUDC-305, and/or NVP-HSP990. In some embodiments, the methods provided herein increase or normalize neuronal ciliation by administering to a neuron one or more mTOR inhibitors. In some embodiments, the methods provided herein increase or normalize neuronal ciliation by administering to a neuron one or more mTOR inhibitors selected from MCI-186, Nicardipine-HCl, K252A, Tyrphostin 9, LY-294002, rapamycin, and/or everolimus. Neuronal ciliation may be measured relative to a reference (e.g., untreated neuron and/or wild-type neuron). In some embodiments, neuronal ciliation is measured relative to a wild-type neuron. In some embodiments, neuronal ciliation is measured relative to an untreated neuron. In some embodiments, the method for increasing or normalizing ciliation in neurons is performed in vivo. In some embodiments, the method for increasing or normalizing ciliation in neurons is performed in vitro.

The present disclosure also provides methods for decreasing a ciliation defect in a cell. In some embodiments, the methods provided herein decrease a ciliation defect in a cell by administering one or more heat shock protein (Hsp) inhibitors (e.g., inhibitors of Hsp27, Hsp40, Hsp60, Hsp70, Hsp90) and/or mTOR inhibitors (e.g., MCI-186, Nicardipine-HCl, K252A, Tyrphostin 9, LY-294002, rapamycin, everolimus). In some embodiments, the methods provided herein decrease a ciliation defect in a cell by administering to a cell one or more heat shock protein (Hsp) inhibitors of Hsp27, Hsp40, Hsp60, Hsp70, and Hsp90. In some embodiments, the methods provided herein decrease a ciliation defect in a cell by administering to a cell one or more Hsp27 inhibitors. In some embodiments, the Hsp27 inhibitor is an inhibitory nucleic acid (e.g., shRNA). In some embodiments, the methods provided herein decrease a ciliation defect in a cell by administering to a cell one or more Hsp90 inhibitors. In some embodiments, the Hsp90 inhibitor is selected from 17-Allylamino-geldanamycin (17-AGG), Geldanamycin (GA), CUDC-305, and/or NVP-HSP990. In some embodiments, the methods provided herein decrease a ciliation defect in a cell by administering to a cell one or more mTOR inhibitors. In some embodiments, the methods provided herein decrease a ciliation defect in a cell by administering to a cell one or more mTOR inhibitors selected from MCI-186, Nicardipine-HCl, K252A, Tyrphostin 9, LY-294002, rapamycin, and/or everolimus. A ciliation defect in a cell may be measured relative to a reference (e.g., untreated cell and/or a wild-type cell). In some embodiments, the ciliation defect is measured relative to a wild-type cell. In some embodiments, the ciliation defect is measured relative to an untreated cell. In some embodiments, the cell is a neuron. In some embodiments, the method for decreasing a ciliation defect is performed in vivo. In some embodiments, the method for decreasing a ciliation defect is performed in vitro.

Further provided are methods for inhibiting mechanistic target of rapamycin complex 1 (mTORC1) activity. The present disclosure provides methods use for inhibiting mechanistic target of rapamycin complex 1 (mTORC1) activity by administering to a cell one or more heat shock protein (Hsp) inhibitors (e.g., inhibitors of Hsp27, Hsp40, Hsp60, Hsp70, Hsp90) and/or mTOR inhibitors (e.g., MCI-186, Nicardipine-HCl, K252A, Tyrphostin 9, LY-294002, rapamycin, everolimus). In some embodiments, the methods provided herein inhibit of mTORC1 activity in a cell by administering to a cell one or more heat shock protein (Hsp) inhibitors of Hsp27, Hsp40, Hsp60, Hsp70, and Hsp90. In some embodiments, the methods provided herein inhibit of mTORC1 activity in a cell by administering to a cell one or more Hsp27 inhibitors. In some embodiments, the Hsp27 inhibitor is an inhibitory nucleic acid (e.g., shRNA). In some embodiments, the methods provided herein inhibit of mTORC1 activity in a cell by administering to a cell one or more Hsp90 inhibitors. In some embodiments, the Hsp90 inhibitor is selected from 17-Allylamino-geldanamycin (17-AGG), Geldanamycin (GA), CUDC-305, and/or NVP-HSP990. In some embodiments, the methods provided herein inhibit of mTORC1 activity in a cell by administering to a cell one or more mTOR inhibitors. In some embodiments, the methods provided herein inhibit of mTORC1 activity in a cell by administering to a cell one or more mTOR inhibitors selected from MCI-186, Nicardipine-HCl, K252A, Tyrphostin 9, LY-294002, rapamycin, and/or everolimus. mTORC1 activity may be measured relative to a reference (e.g., untreated cell and/or a wild-type cell). In some embodiments, mTORC1 activity is measured relative to a wild-type cell. In some embodiments, mTORC1 activity is measured relative to an untreated cell. In some embodiments, the cell is a neuron. In some embodiments, the method for inhibiting mTORC1 activity is performed in vivo. In some embodiments, the method for inhibiting mTORC1 activity is performed in vitro.

The one or more heat shock protein (Hsp) inhibitors (e.g., inhibitors of Hsp27, Hsp40, Hsp60, Hsp70, Hsp90) and/or mTOR inhibitors (e.g., MCI-186, Nicardipine-HCl, K252A, Tyrphostin 9, LY-294002, rapamycin, everolimus) or compositions thereof, can be administered as described herein in any suitable manner, such as with pharmaceutically acceptable carriers, diluents, or excipients as described supra. Pharmaceutically acceptable carriers are determined in part by the particular immunogen or composition being administered, as well as by the particular method used to administer the composition. Accordingly, pharmaceutical composition comprising one or more heat shock protein (Hsp) inhibitors (e.g., inhibitors of Hsp27, Hsp40, Hsp60, Hsp70, Hsp90) and/or mTOR inhibitors (e.g., MCI-186, Nicardipine-HCl, K252A, Tyrphostin 9, LY-294002, rapamycin, everolimus) can be prepared using a wide variety of suitable and physiologically and pharmaceutically acceptable formulations for use in the methods of the present disclosure.

Administration of the one or more heat shock protein (Hsp) inhibitors (e.g., inhibitors of Hsp27, Hsp40, Hsp60, Hsp70, Hsp90) and/or mTOR inhibitors (e.g., MCI-186, Nicardipine-HCl, K252A, Tyrphostin 9, LY-294002, rapamycin, everolimus) or pharmaceutical compositions thereof, can be accomplished by single or multiple doses. The dose administered to a subject should be sufficient to induce a beneficial therapeutic response in a subject over time, such as to inhibit, block, reduce, ameliorate, protect against, or prevent disease (e.g., neurodevelopmental disorders (e.g., tuberous sclerosis complex (TSC))) caused by mutations in mTOR regulatory genes (e.g., TSC1, TSC2, AKT3, DEPDC5), mutations in genes that play a role in cilia assembly and/or function, from a disruption of the mTOR pathway, and/or from dysfunctional mTORC1 activity. The dose required will vary from subject to subject depending on the species, age, weight and general condition of the subject, by the severity of the disease or disorder being treated, by the particular composition being used and by the mode of administration. An appropriate dose can be determined by a person skilled in the art, such as a clinician or medical practitioner, using only routine experimentation. One of skill in the art is capable of determining therapeutically effective amounts of the compositions provided herein, that provide a therapeutic effect or protection against diseases (e.g., neurodevelopmental disorders (e.g., tuberous sclerosis complex (TSC))) caused by mutations in mTOR regulatory genes (e.g., TSC1, TSC2, AKT3, DEPDC5), mutations in genes that play a role in cilia assembly and/or function, from a disruption of the mTOR pathway, and/or from dysfunctional mTORC1 activity suitable for administering to a subject in need of treatment or protection.

### Inhibitory Nucleic Acid Therapy

Polynucleotide therapy featuring a polynucleotide encoding a heat shock protein (Hsp) (e.g., Hsp27, Hsp40, Hsp60, Hsp70, Hsp90) inhibitory nucleic acid molecule (e.g., siRNA, shRNA, or antisense RNA) or an analog thereof is another therapeutic approach for treating a neurodevelopmental disorder (e.g., TSC) in a subject. Expression vectors encoding inhibitory nucleic acid molecules can be delivered to cells (e.g., neurons) of a subject having a neurodevelopmental disorder (e.g., TSC). The nucleic acid molecules must be delivered to the cells (e.g., neurons) of a subject in a form in which they can be taken up and are advantageously expressed so that therapeutically effective levels can be achieved.

Provided herein are inhibitory nucleic acid molecules (e.g., siRNA, shRNA, or antisense RNA) that target heat shock protein (Hsp) (e.g., Hsp27, Hsp40, Hsp60, Hsp70, Hsp90) gene expression. Such inhibitory nucleic acid molecules can be delivered to cells (e.g., neurons) of a subject having a neurodevelopmental disorder (e.g., TSC). Methods for delivery of the polynucleotides to the cell (e.g., neuron) according to the invention include using a delivery system such as liposomes, polymers, microspheres, gene therapy vectors, and naked DNA vectors.

Transducing viral (e.g., retroviral, adenoviral, lentiviral and adeno-associated viral (AAV)) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding a heat shock protein (Hsp) (e.g., Hsp27, Hsp40, Hsp60, Hsp70, Hsp90) inhibitory nucleic acid molecule, can be cloned into a retroviral vector and expression can be driven from its endogenous promoter, from the retroviral long terminal repeat, or from a promoter specific for a target cell type (e.g., neuron) of interest. In some embodiments, the target cell type of interest is a neuron.

Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). In some embodiments, a viral vector is used to administer a polynucleotide encoding inhibitory nucleic acid molecules that inhibit heat shock protein (Hsp) (e.g., Hsp27, Hsp40, Hsp60, Hsp70, Hsp90) expression. In some embodiments, the vector is an AAV vector.

Non-viral approaches can also be employed for the introduction of a heat shock protein (Hsp) (e.g., Hsp27, Hsp40, Hsp60, Hsp70, Hsp90) inhibitory nucleic acid molecule therapeutic to a cell (e.g., neuron) of a patient diagnosed as having a neurodevelopmental disorder (e.g., TSC). For example, a heat shock protein (Hsp) (e.g., Hsp27, Hsp40, Hsp60, Hsp70, Hsp90) inhibitory nucleic acid molecule can be introduced into a cell (e.g., neuron) by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). In one embodiment, the heat shock protein (Hsp) (e.g., Hsp27, Hsp40, Hsp60, Hsp70, Hsp90) inhibitory nucleic acid molecules are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell (e.g., neuron). Transplantation of polynucleotide encoding inhibitory nucleic acid molecules into the affected tissues of a patient can also be accomplished by transferring a polynucleotide encoding the inhibitory nucleic acid into a cultivatable cell type ex vivo (e.g., an autologous or heterologous primary cell or progeny thereof), after which the cell (or its descendants) are injected into a targeted tissue.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types (e.g., neurons) can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

In some embodiments, the inhibitory nucleic acid molecule is selectively expressed in a neuron. In some other embodiments, the inhibitory nucleic acid molecule is expressed in a neuron using a lentiviral vector. In still other embodiments, the inhibitory nucleic acid molecule is administered intrathecally. Selective targeting or expression of inhibitory nucleic acid molecules to a neuron is described in, for example, Nielsen et al., J Gene Med. 2009 July; 11(7):559-69. doi: 10.1002/jgm.1333.

For any particular subject, the specific dosage regimes should be adjusted over time according to the individual need and the professional judgment of the person administering or supervising the administration of the compositions.

### Combination Therapies

The one or more heat shock protein (Hsp) inhibitors (e.g., inhibitors of Hsp27, Hsp40, Hsp60, Hsp70, Hsp90) and/or mTOR inhibitors (e.g., MCI-186, Nicardipine-HCl, K252A, Tyrphostin 9, LY-294002, rapamycin, everolimus) or pharmaceutical compositions thereof can be administered alone or in combination with each other to treat a neurodevelopmental disorder (e.g., TSC) in a subject. The one or more heat shock protein (Hsp) inhibitors (e.g., inhibitors of Hsp27, Hsp40, Hsp60, Hsp70, Hsp90) and/or mTOR inhibitors (e.g., MCI-186, Nicardipine-HCl, K252A, Tyrphostin 9, LY-294002, rapamycin, everolimus) or pharmaceutical compositions thereof can also be administered alone or in combination with one or more other therapeutic agents to treat a neurodevelopmental disorder (e.g., TSC) in a subject. Non-limiting examples include combining one or more heat shock protein (Hsp) inhibitors (e.g., inhibitors of Hsp27, Hsp40, Hsp60, Hsp70, Hsp90) with one or more mTOR inhibitors (e.g., MCI-186, Nicardipine-HCl, K252A, Tyrphostin 9, LY-294002, rapamycin, everolimus). In some embodiments, one or more Hsp inhibitors selected from inhibitory nucleic acids, 17-Allylamino-geldanamycin (17-AGG), Geldanamycin (GA), CUDC-305, and NVP-HSP990 or an analog thereof is administered in combination with one or more mTOR inhibitors selected from MCI-186, Nicardipine-HCl, K252A, Tyrphostin 9, LY-294002, rapamycin, and everolimus. In some embodiments, the one or more Hsp inhibitor is an inhibitory nucleic acid selected from a siRNA, shRNA, or antisense RNA. In some embodiments, the inhibitory nucleic acid is a shRNA.

In some embodiments, one or more heat shock protein (Hsp) inhibitors (e.g., inhibitors of Hsp27, Hsp40, Hsp60, Hsp70, Hsp90) is administered simultaneously or sequentially with one or more mTOR inhibitors (e.g., MCI-186, Nicardipine-HCl, K252A, Tyrphostin 9, LY-294002, rapamycin, everolimus). In some embodiments, one or more heat shock protein (Hsp) inhibitors (e.g., inhibitors of Hsp27, Hsp40, Hsp60, Hsp70, Hsp90) are administered in combination with rapamycin and/or everolimus. In some embodiments, the heat shock protein inhibitor is selected from an inhibitor of Heat shock protein 90 (Hsp90), Heat shock protein 27 (Hsp27), Heat shock protein 70 (Hsp70), Heat shock protein 60 (Hsp60), Heat shock protein 40 (Hsp40), and analogs thereof. In some embodiments, an inhibitor of Hsp27 (e.g., inhibitory nucleic acid) or an analog thereof is administered in combination with rapamycin and/or everolimus. In some embodiments, an inhibitor of Hsp90 (e.g., 17-AGG, GA, CUDC-305, NVP-HSP990) or an analog thereof is administered in combination with rapamycin and/or everolimus. In some embodiments, an inhibitor of Hsp27 and Hsp90 or an analog thereof is administered in combination with rapamycin and/or everolimus. In some embodiments, an Hsp inhibitor selected from inhibitory nucleic acids, 17-Allylamino-geldanamycin (17-AGG), Geldanamycin (GA), CUDC-305, and NVP-HSP990 or an analog thereof is administered in combination with rapamycin and/or everolimus. In some embodiments, 17-AGG is administered in combination with rapamycin and/or everolimus. In some embodiments, an inhibitory nucleic acid that decreases the gene expression of at least one heat shock protein (Hsp) is administered in combination with rapamycin and/or everolimus. In some embodiments, the inhibitory nucleic acid is an shRNA. In some embodiments, an shRNA that decreases the gene expression of HSPB1 is administered in combination with rapamycin and/or everolimus.

While treatment methods may involve the administration of a one or more heat shock protein (Hsp) inhibitors (e.g., inhibitors of Hsp27, Hsp40, Hsp60, Hsp70, Hsp90) and/or mTOR inhibitors (e.g., MCI-186, Nicardipine-HCl, K252A, Tyrphostin 9, LY-294002, rapamycin, everolimus) as described herein, one skilled in the art will appreciate that the one or more heat shock protein (Hsp) inhibitors (e.g., inhibitors of Hsp27, Hsp40, Hsp60, Hsp70, Hsp90) and/or mTOR inhibitors (e.g., MCI-186, Nicardipine-HCl, K252A, Tyrphostin 9, LY-294002, rapamycin, everolimus), as a component of a pharmaceutically acceptable composition, can be administered to a subject in need thereof to treat a neurodevelopmental disorder in the subject.

### Kits

Various aspects of this disclosure provide kits comprising one or more heat shock protein (Hsp) inhibitors (e.g., inhibitors of Hsp27, Hsp40, Hsp60, Hsp70, Hsp90) and/or mTOR inhibitors (e.g., MCI-186, Nicardipine-HCl, K252A, Tyrphostin 9, LY-294002, rapamycin, everolimus) or pharmaceutical compositions thereof. In some embodiments, the kit includes one or more heat shock protein inhibitors selected from, but not limited to, inhibitors of Heat shock protein 90 (Hsp90), Heat shock protein 27 (Hsp27), Heat shock protein 70 (Hsp70), Heat shock protein 60 (Hsp60), Heat shock protein 40 (Hsp40), and analogs thereof. In some embodiments, the kit includes an inhibitor of Hsp27 (e.g., inhibitory nucleic acid), or an analog thereof. In some embodiments, the kit includes an inhibitor of Hsp90 (e.g., 17-AGG, GA, CUDC-305, NVP-HSP990), or an analog thereof. In some embodiments, the kit includes an inhibitor of Hsp27 and Hsp90, or an analog thereof. In some embodiments, kit includes one or more inhibitory nucleic acid, MCI-186, Nicardipine-HCl, K252A, Tyrphostin 9, LY-294002, rapamycin, and/or everolimus, or analogs thereof. In some embodiments, the kit includes MCI-186, or an analog thereof. In some embodiments, the kit includes Nicardipine-HCl, or an analog thereof. In certain embodiments, the kit is useful for the treatment of a subject having a neurodevelopmental disorder (e.g., mTORopathy, neuronal ciliopathy).

The kits of the present disclosure may also comprise instructions for performing one or more methods described herein and/or a description of one or more compositions or reagents described herein. Instructions and/or descriptions may be in printed form and may be included in a kit insert. A kit also may include a written description of an Internet location that provides such instructions or descriptions.

In yet another embodiment, the kit can comprise one or more containers with appropriate positive and negative controls or control samples, to be used as standard(s) for detection, calibration, or normalization. The kit can further comprise a second container comprising a pharmaceutically-acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

The kits of the present disclosure may also comprise one or more of the compositions or reagents described herein in any number of separate containers, packets, tubes (e.g., <0.2 ml, 0.2 ml, 0.6 ml, 1.5 ml, 5.0 ml, >5.0 ml), vials, microtiter plates (e.g., <96-well, 96-well, 384-well, 1536-well, >1536-well), ArrayTape, and the like, or the compositions or reagents described herein may be combined in various combinations in such containers. In yet other embodiments, the kit comprises a sterile container which contains the one or more compositions or reagents described herein; such containers can be boxes, ampules, bottles, vials, tubes, bags, pouches, blister-packs, or other suitable container form known in the art. Such containers can be made of plastic, glass, laminated paper, metal foil, or other materials suitable for holding nucleic acids.

The components may, for example, be dried (e.g., dry residue), lyophilized (e.g., dry cake) or in a stable buffer (e.g., chemically stabilized, thermally stabilized). Dry components may, for example, be prepared by lyophilization, vacuum and centrifugal assisted drying and/or ambient drying.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the disclosure. Useful techniques for particular embodiments will be discussed in the sections that follow.

The following examples are put forth to provide those of ordinary skill in the art with a complete disclosure and description of how to make and use the assay, screening, and therapeutic methods of the disclosure, and are not intended to limit the scope of what the inventors regard as their disclosure.

### EXAMPLES

The following examples are provided to illustrate certain particular features and/or embodiments. The examples should not be construed to limit the disclosure to the particular features or embodiments described.

### Example 1: A Phenotypic Screen with TSC-Deficient Neurons Exhibiting Reduced Cilia Reveal the Heat Shock Machinery as a Druggable Pathway for mTORC1 Dysfunction

Tuberous Sclerosis Complex (TSC) is a neurogenetic disorder that leads to elevated mechanistic target of rapamycin complex 1 (mTORC1) activity. Cilia can be affected by mTORC1 signaling, and ciliary deficits are associated with neurodevelopmental disorders. Primary cilia are evolutionarily conserved membrane extensions of the cell surface made of microtubules that extend from a centriole-derived structure called the basal body (Lee and Gleeson, Cilia in the nervous system: linking cilia function and neurodevelopmental disorders; Current opinion in neurology 24, 98-105 (2011)). Cilia are often referred to as sensory antenna since they coordinate extracellular ligand-based signaling, playing a critical role in tissue homeostasis (Gerdes et al., he vertebrate primary cilium in development, homeostasis, and disease. Cell 137, 32-45 (2009)). Mutations in genes that play a role in cilia assembly and/or function underlie a broad spectrum of genetic disorders called ciliopathies. In the CNS, ciliopathies are associated with severe neurodevelopmental outcomes including brain malformations, ASD, and intellectual disability (Bettencourt-Dias et al., Centrosomes and cilia in human disease; Trends Genet 27, 307-315 (2011); Guemez-Gamboa et al., Primary cilia in the developing and mature brain; Neuron 82, 511-521 (2014)). A recent study showed that patients with focal malformation of cortical developments (FMCDs) caused by somatic mutations in MTOR have a reduction in neuronal cilia (Park et al., Brain Somatic Mutations in MTOR Disrupt Neuronal Ciliogenesis, Leading to Focal Cortical Dyslamination; Neuron 99, 83-97 e87 (2018)). However, elevated mTORC1 activity caused by loss of TSC1 or TSC2 genes in mouse embryonic fibroblasts (MEFs) resulted in a rapamycin-insensitive increase of cilia and ciliary length (Hartman et al., The tuberous sclerosis proteins regulate formation of the primary cilium via a rapamycin-insensitive and polycystin 1-independent pathway; Hum Mol Genet 18, 151-163 (2009)). These and other studies indicate a connection between the mTORC1 and cilia with different outcomes dependent on the cellular type and on the etiology of disrupted mTORC1 signaling (DiBella et al., Zebrafish Tsc1 reveals functional interactions between the cilium and the TOR pathway; Hum Mol Genet 18, 595-606 (2009); Rosengren et al., TSC1 and TSC2 regulate cilia length and canonical Hedgehog signaling via different mechanisms; Cell Mol Life Sci 75, 2663-2680 (2018)).

To examine whether neuronal cilia are affected in TSC, the effect of disinhibition of mTORC1 on cilia was investigated using in vivo and in vitro models of TSC and specimens from patients. It was observed that neuronal ciliation was reduced in brains of TSC1 and TSC2 conditional knockout mice and in cortical tubers resected from TSC patients with refractory epilepsy. To investigate the mechanism by which disinhibition of mTORC1 affects neuronal cilia, a phenotypic screen was performed for mTORC1 inhibitors using TSC2 gene knockdown in hippocampal neurons. Inhibitors of the molecular chaperone the heat shock protein 90 (Hsp90), Geldanamycin (GA) and 17-Allylamino-geldanamycin (17-AGG) were identified as compounds that suppress mTORC1 through regulation of PI3K/Akt signaling components. Notably, 17-AGG improved ciliation at doses far below mTORC1 inhibition during a specific developmental window and further demonstrated that this effect was through reduced expression of HspB1 gene expression, which encodes the small heat shock protein 27 (Hsp27). Together, these data indicate that TSC displays features of a ciliopathy and identify the heat shock response as a regulator at different nodes within the mTORC1 signaling cascade.

### Example 2: Brains of TSC Patients and CNS-Knockout Mouse Models have Reduced Ciliated Neurons that is Restored by Rapamycin In Vivo

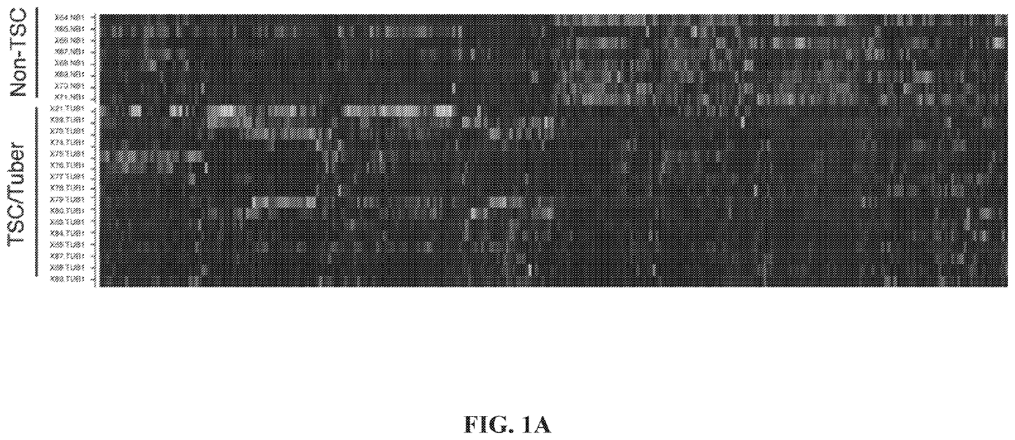

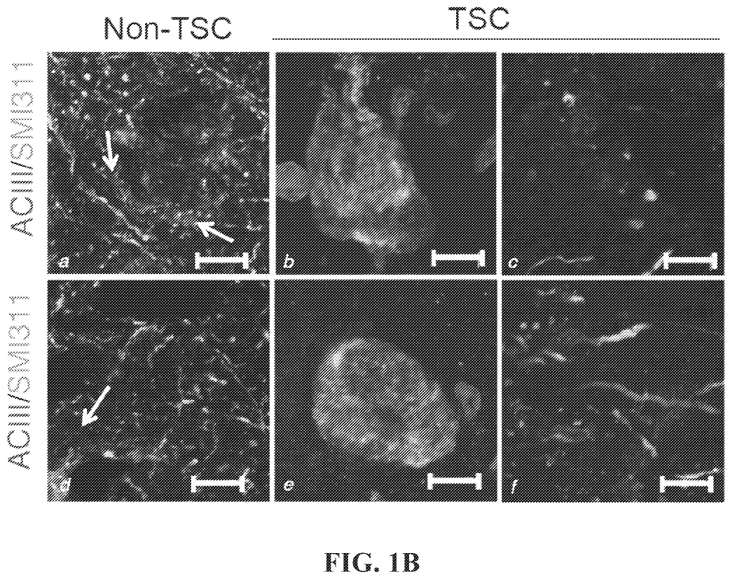

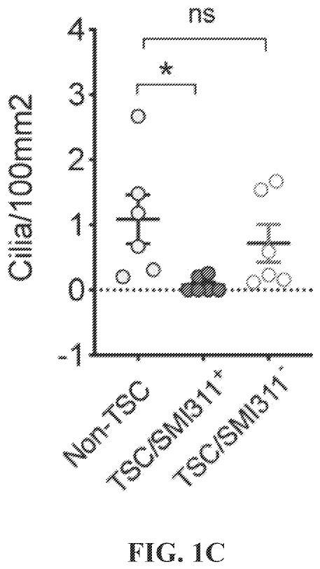

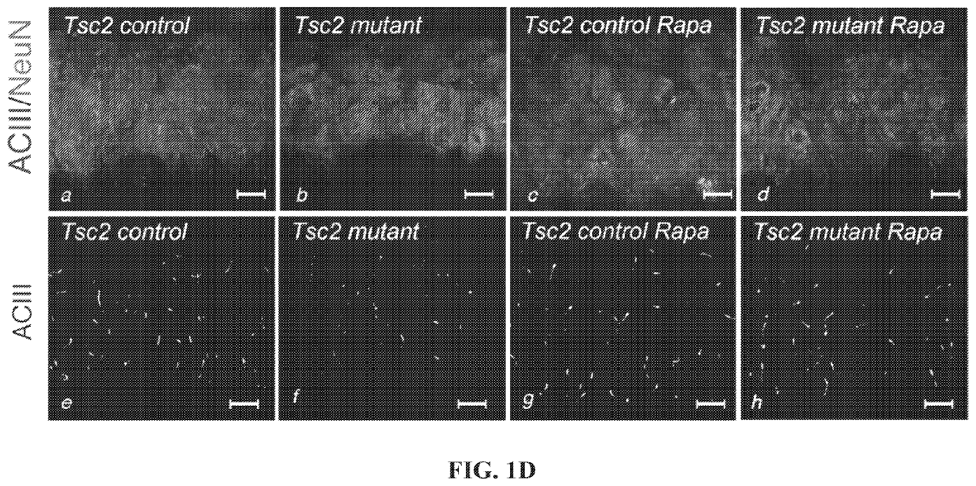

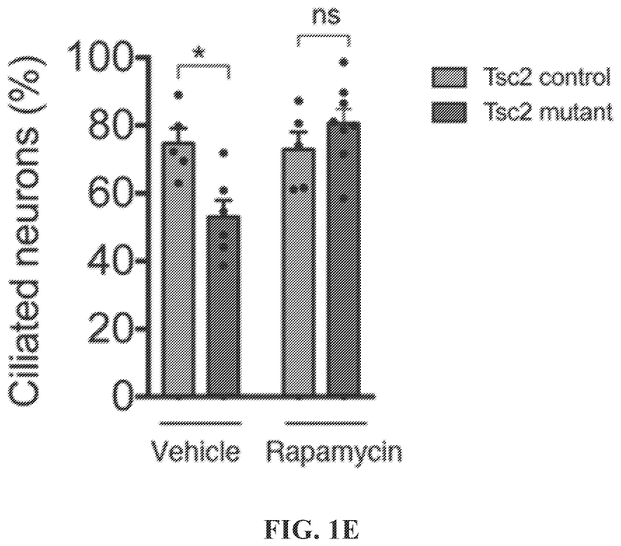

Altered cilia gene expression is a risk factor for neuropsychiatric disorders (Marley and von Zastrow, A simple cell-based assay reveals that diverse neuropsychiatric risk genes converge on primary cilia; PLoS One 7, e46647 (2012); Migliavacca et al., A Potential Contributory Role for Ciliary Dysfunction in the 16p11.2 600 kb BP4-BP5 Pathology; American journal of human genetics 96, 784-796 (2015)). To determine whether the ciliary gene signature might be altered in TSC patients, the expression of cilia genes from the Syscilia database in a comprehensive set of TSC-associated cortical tubers and healthy controls recently reported in a genomic study (Martin et al., The genomic landscape of tuberous sclerosis complex; Nat Commun 8, 15816 (2017)) was examined. It was discovered that genes associated with cilia were more likely to be differentially expressed compared to random genes (FIGS. 1A, Table 1). To investigate whether differential expression of cilia genes reflected changes in ciliation, cilia in brain specimens resected from patients with refractory epilepsy with or without TSC (Tables 2A and 2B) was examined. The ciliary membrane-bound adenylyl cyclase III (ACIII) (Bishop et al., Type III adenylyl cyclase localizes to primary cilia throughout the adult mouse brain; J Comp Neurol 505, 562-571 (2007)) was stained to identify cilia and co-labeled with the pan-neurofilament marker SMI311 (Talos et al., Altered inhibition in tuberous sclerosis and type IIb cortical dysplasia. Annals of neurology 71, 539-551 (2012)) to identify the giant cells present in the cortical tubers of TSC patients. Compared to the brains of the non-TSC cases, giant cells in the cortical tubers had a significant reduction in ciliation (FIGS. 1B-C).

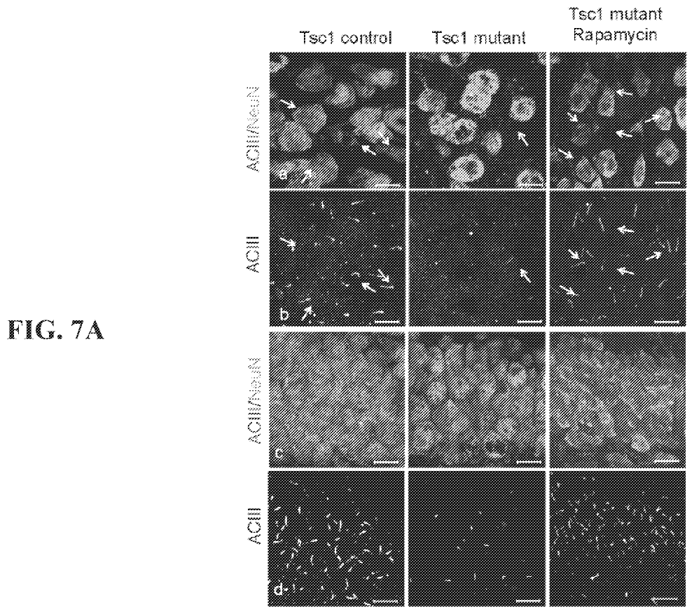

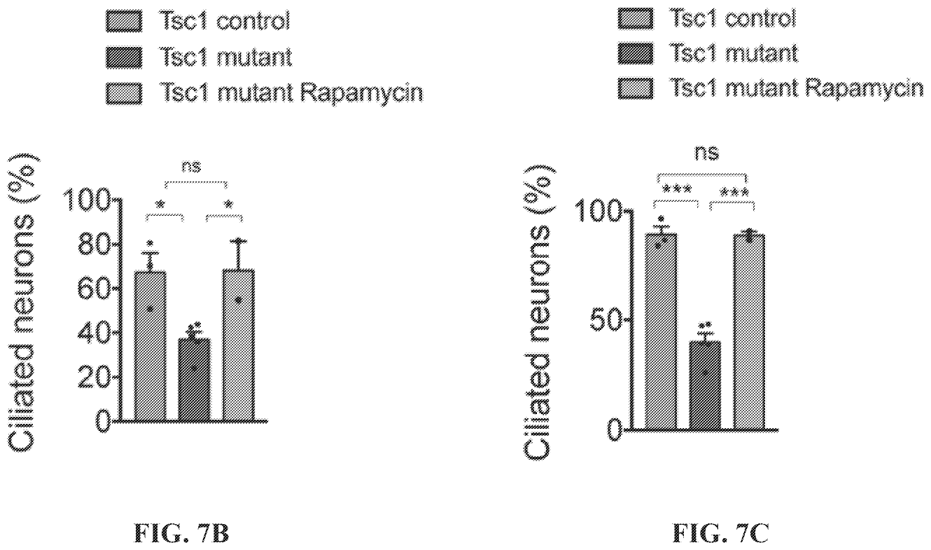

Neuronal cilia in TSC mouse models was examined with either a conditional deletion of TSC1 or TSC2 driven by the Synapsin-1 promoter, which results in loss of TSC1/2 proteins in post-mitotic neurons of the cortex and of the hippocampus. The TSC1/SynCre mice (Tsc1 mutant) have a shorter life span (median age postnatal day 35, P35), and they recapitulate many of the neurological manifestations of TSC, including seizures and presence of ectopic giant cells (Meikle et al., A mouse model of tuberous sclerosis: neuronal loss of Tsc1 causes dysplastic and ectopic neurons, reduced myelination, seizure activity, and limited survival; J Neurosci 27, 5546-5558 (2007)). The TSC2del3/SynCre mice (Tsc2 mutant) are globally heterozygous for the Tsc2 knockout allele throughout the body and also carry a hypomorphic Tsc2 (del3) allele that retains partial function in Synapsin-1 expressing post-mitotic neurons (Pollizzi et al., A hypomorphic allele of Tsc2 highlights the role of TSC1/TSC2 in signaling to AKT and models mild human TSC2 alleles; Hum Mol Genet 18, 2378-2387 (2009)). Due to partially retained TSC2 expression, these mice develop seizures around eight weeks, and they survive until about twelve weeks of age. Cilia were assayed in the Tsc2 mutant animals at eight weeks (P56) by staining with ACIII and co-labeling with NeuN to identify neurons. Consistent with the findings in TSC patient brains, Tsc2 mutant mice were found to have decreased cilia in pyramidal neurons of the CA1 region of the hippocampus compared to controls (FIGS. 1D-E). Similarly, ciliation was reduced in the cortex and hippocampus in the Tsc1 mutant mice at P21 (FIGS. 7A-C). Notably, these ciliation defects were prevented by mTORC1 inhibition with early rapamycin treatment starting at P7 in both TSC models (FIGS. 1D-E, FIGS. 7A-C).

TABLE 1
Differential cilia gene expression between brain from TSC patients and healthy
controls.

| | | | Fold change | P-value |
|---|---|---|---|---|
| Gene | Entrez | | TSC/Tuber; | TSC/Tuber; |
| Symbol | ID | | Non-TSC | Non-TSC |
| adcy3 | 109 | adenylate cyclase 3 | −0.222792625 | 0.39974185 |
| ahi1 | 54806 | Abelson helper integration site 1 | −0.008346937 | 0.967683871 |
| ak8 | 158067 | adenylate kinase 8 | 0.647238963 | 0.008043103 |
| alms1 | 7840 | Alstrom syndrome 1 | 0.310952063 | 0.045068053 |
| alms1 | 7840 | Alstrom syndrome 1 | 0.310952063 | 0.045068053 |
| arf4 | 378 | ADP-ribosylation factor 4 | −0.08855825 | 0.517478827 |
| arl13b | 200894 | ADP-ribosylation factor-like | 0.409963375 | 0.042599856 |
| | | 13B | | |
| arl3 | 403 | ADP-ribosylation factor-like 3 | −0.273946062 | 0.062968172 |
| atxn10 | 25814 | ataxin 10 | −0.064185187 | 0.718340115 |
| b9d1 | 27077 | B9 protein domain 1 | −0.101135125 | 0.684385955 |
| b9d1 | 27077 | B9 protein domain 1 | −0.101135125 | 0.684385955 |
| b9d2 | 80776 | B9 protein domain 2 | 0.291638919 | 0.153659453 |
| bbs1 | 582 | Bardet-Biedl syndrome 1 | −0.119310875 | 0.361132584 |
| bbs2 | 583 | Bardet-Biedl syndrome 2 | 0.416169 | 0.018289421 |
| bbs4 | 585 | Bardet-Biedl syndrome 4 | 0.063739563 | 0.536927699 |
| bbs5 | 129880 | Bardet-Biedl syndrome 5 | 0.280131063 | 0.370179682 |
| bbs7 | 55212 | Bardet-Biedl syndrome 7 | −0.905521063 | 2.01E−05 |
| c2cd3 | 26005 | C2 calcium-dependent domain | −0.382401 | 0.19203433 |
| | | containing 3 | | |
| cby1 | 25776 | chibby homolog 1 (Drosophila) | 0.732919188 | 1.80E−05 |
| cede114 | 93233 | coiled-coil domain containing | 0.930751261 | 0.129265996 |
| | | 114 | | |
| cdh23 | 64072 | cadherin-related 23 | 1.192914338 | 0.014900779 |
| cenpj | 55835 | centromere protein J | 0.00183825 | 0.992930658 |
| cep104 | 9731 | centrosomal protein 104 kDa | −0.18836775 | 0.287133922 |
| cep290 | 80184 | centrosomal protein 290 kDa | −0.002345125 | 0.98986613 |
| cep41 | 95681 | centrosomal protein 41 kDa | −0.483510062 | 0.016288067 |
| cep89 | 84902 | centrosomal protein 89 kDa | 0.356449 | 0.015038457 |
| cep97 | 79598 | centrosomal protein 97 kDa | −1.022965375 | 0.002379425 |
| cldn2 | 9075 | claudin 2 | −0.042784819 | 0.955807235 |
| cnga4 | 1262 | cyclic nucleotide gated channel | 0.988868583 | 0.045118783 |
| | | alpha 4 | | |
| cngb1 | 1258 | cyclic nucleotide gated channel | −1.380422419 | 0.09957032 |
| | | beta 1 | | |
| crb3 | 92359 | crumbs family member 3 | −1.158082063 | 0.003323016 |
| crocc | 9696 | ciliary rootlet coiled-coil, | 0.556776375 | 0.013378457 |
| | | rootletin | | |
| ctnnb1 | 1499 | catenin (cadherin-associated | 0.010081875 | 0.952316708 |
| | | protein), beta 1, 88 kDa | | |
| dcdc2 | 51473 | doublecortin domain containing | 0.194454325 | 0.70247791 |
| | | 2 | | |
| dnaaf1 | 123872 | dynein, axonemal, assembly | −0.55542055 | 0.395935218 |
| | | factor 1 | | |
| dnaaf2 | 55172 | dynein, axonemal, assembly | −0.312116 | 0.009711766 |
| | | factor 2 | | |
| dnaaf3 | 352909 | dynein, axonemal, assembly | 2.338232231 | 0.000203515 |
| | | factor 3 | | |
| dnaaf3 | 352909 | dynein, axonemal, assembly | 2.338232231 | 0.000203515 |
| | | factor 3 | | |
| dnah1 | 25981 | dynein, axonemal, heavy chain 1 | 0.475404812 | 0.067602773 |
| dnai1 | 27019 | dynein, axonemal, intermediate | −0.698164381 | 0.136843165 |
| | | chain 1 | | |
| dnai2 | 64446 | dynein, axonemal, intermediate | 0.536670062 | 0.481443024 |
| | | chain 2 | | |
| dnai1 | 83544 | dynein, axonemal, light chain 1 | −0.161576125 | 0.335906048 |
| dnali1 | 7802 | dynein, axonemal, light | 1.722177875 | 5.68E−06 |
| | | intermediate chain 1 | | |
| dpcd | 25911 | deleted in primary ciliary | −0.35181 | 0.068529127 |
| | | dyskinesia homolog (mouse) | | |
| drd2 | 1813 | dopamine receptor D2 | 1.054212731 | 0.09685067 |
| drd5 | 1816 | dopamine receptor D5 | −0.853615834 | 0.153637301 |
| dvl1 | 1855 | dishevelled segment polarity | −0.161890687 | 0.299715145 |
| | | protein 1 | | |
| dync2h1 | 79659 | dynein, cytoplasmic 2, heavy | 0.218604625 | 0.076885221 |
| | | chain 1 | | |
| dynlt1 | 6993 | dynein, light chain, Tctex-type 1 | 1.5002675 | 5.62E−07 |
| efhc1 | 114327 | EF-hand domain (C-terminal) | 0.1235765 | 0.449744059 |
| | | containing 1 | | |
| evc | 2121 | Ellis van Creveld syndrome | 1.070910755 | 0.021410494 |
| evc | 2121 | Ellis van Creveld syndrome | 1.070910755 | 0.021410494 |
| evc2 | 132884 | Ellis van Creveld syndrome 2 | 2.589255475 | 1.20E−06 |
| exoc3 | 11336 | exocyst complex component 3 | −0.349775187 | 0.013503554 |
| exoc4 | 60412 | exocyst complex component 4 | 0.21231825 | 0.133019011 |
| exoc5 | 10640 | exocyst complex component 5 | −0.2895285 | 0.013813096 |
| exoc6 | 54536 | exocyst complex component 6 | −0.406923062 | 0.091575907 |
| fam161a | 84140 | family with sequence similarity | −0.051328 | 0.812243085 |
| | | 161, member A | | |
| flna | 2316 | filamin A, alpha | 1.947186438 | 6.33E−05 |
| fopnl | 123811 | FGFR1OP N-terminal like | −0.194922 | 0.143333998 |
| fuz | 80199 | fuzzy planar cell polarity protein | 0.357392625 | 0.020784429 |
| gas8 | 2622 | growth arrest-specific 8 | −0.1304085 | 0.344917671 |
| gli1 | 2735 | GLI family zinc finger 1 | 0.174510836 | 0.734426492 |
| gli2 | 2736 | GLI family zinc finger 2 | 1.843002191 | 6.24E−05 |
| gli3 | 2737 | GLI family zinc finger 3 | 1.038155875 | 0.018550776 |
| glis2 | 84662 | GLIS family zinc finger 2 | 0.659983125 | 0.052075842 |
| gsk3b | 2932 | glycogen synthase kinase 3 beta | −1.191554375 | 2.13E−05 |
| hap1 | 9001 | huntingtin-associated protein 1 | 2.0835225 | 0.000184266 |
| hap1 | 9001 | huntingtin-associated protein 1 | 2.0835225 | 0.000184266 |
| hap1 | 9001 | huntingtin-associated protein 1 | 2.0835225 | 0.000184266 |
| hnf1b | 6928 | HNF1 homeoboxB | 0.433988437 | 0.398956181 |
| hnf1b | 6928 | HNF1 homeoboxB | 0.433988437 | 0.398956181 |
| hnf1b | 6928 | HNF1 homeoboxB | 0.433988437 | 0.398956181 |
| htt | 3064 | huntingtin | −0.450591813 | 0.017840941 |
| ift20 | 90410 | intraflagellar transport 20 | 0.509257438 | 0.003254735 |
| ift27 | 11020 | intraflagellar transport 27 | 0.223639938 | 0.077730432 |
| ift43 | 112752 | intraflagellar transport 43 | 0.58846575 | 0.001927062 |
| ift46 | 56912 | intraflagellar transport 46 | 0.188461563 | 0.134112574 |
| ift52 | 51098 | intraflagellar transport 52 | 0.1833125 | 0.10532074 |
| ift52 | 51098 | intraflagellar transport 52 | 0.1833125 | 0.10532074 |
| ift57 | 55081 | intraflagellar transport 57 | −0.361493812 | 0.034495792 |
| ift74 | 80173 | intraflagellar transport 74 | 0.55266625 | 0.00020071 |
| ift81 | 28981 | intraflagellar transport 81 | 0.25705525 | 0.118021046 |
| ift88 | 8100 | intraflagellar transport 88 | 0.547650188 | 0.010188254 |
| inpp5e | 56623 | inositol polyphosphate-5- | −0.1941205 | 0.256251411 |
| | | phosphatase, 72 kDa | | |
| invs | 27130 | inversin | −0.017952875 | 0.91164163 |
| iqcb1 | 9657 | IQ motif containing B1 | −0.457224812 | 0.001936797 |
| kifl9 | 124602 | kinesin family member 19 | 0.65829965 | 0.501779867 |
| kif3b | 9371 | kinesin family member 3B | −0.077626625 | 0.726893648 |
| kifc | 3797 | kinesin family member 3C | −0.8278265 | 0.016408914 |
| lca5 | 167691 | Leber congenital amaurosis 5 | 0.045049125 | 0.843723484 |
| lrrc6 | 23639 | leucine rich repeat containing 6 | −0.272316812 | 0.23835536 |
| Iztfl1 | 54585 | leucine zipper transcription | −0.680955812 | 0.001448264 |
| | | factor-like 1 | | |
| mak | 4117 | male germ cell-associated | 0.100361735 | 0.766262977 |
| | | kinase | | |
| mal | 4118 | mal, T-cell differentiation | −0.129506187 | 0.907993499 |
| | | protein | | |
| mapre1 | 22919 | microtubule-associated protein, | 0.955203063 | 3.05E−06 |
| | | RP/EB family, member 1 | | |
| mchr1 | 2847 | melanin-concentrating hormone | −0.548082383 | 0.411966973 |
| | | receptor 1 | | |
| mdm1 | 56890 | Mdml nuclear protein homolog | 0.545429563 | 0.001111682 |
| | | (mouse) | | |
| mkks | 8195 | McKusick-Kaufman syndrome | −0.535228375 | 0.005808141 |
| mks1 | 54903 | Meckel syndrome, type 1 | 0.360496313 | 0.01757115 |
| mlf1 | 4291 | myeloid leukemia factor 1 | 0.3733495 | 0.158776882 |
| mns1 | 55329 | meiosis-specific nuclear | 0.738988063 | 0.010710056 |
| | | structural 1 | | |
| myo7a | 4647 | myosin VIIA | 0.500963131 | 0.321100773 |
| nek1 | 4750 | NIMA-related kinase 1 | 0.328788437 | 0.030198217 |
| nek2 | 4751 | NIMA-related kinase 2 | −2.150074237 | 0.000994733 |
| nek8 | 284086 | NIMA-related kinase 8 | 0.910411003 | 0.001880214 |
| ngfr | 4804 | nerve growth factor receptor | 0.40201765 | 0.551550357 |
| nme7 | 29922 | NME/NM23 family member 7 | −0.0176325 | 0.940730777 |
| nme8 | 51314 | NME/NM23 family member 8 | 0.480194144 | 0.413915933 |
| nphp1 | 4867 | nephronophthisis 1 (juvenile) | 1.11667296 | 6.45E−06 |
| nphp1 | 4867 | nephronophthisis 1 (juvenile) | 1.11667296 | 6.45E−06 |
| nphp1 | 4867 | nephronophthisis 1 (juvenile) | 1.11667296 | 6.45E−06 |
| nphp3 | 27031 | nephronophthisis 3 (adolescent) | 1.198673313 | 6.72E−05 |
| nphp4 | 261734 | nephronophthisis 4 | −0.029097812 | 0.881303419 |
| nup214 | 8021 | nucleoporin 214 kDa | 0.131090938 | 0.329136501 |
| nup35 | 129401 | nucleoporin 35 kDa | 0.073909938 | 0.597294525 |
| nup37 | 79023 | nucleoporin 37 kDa | 1.20744425 | 1.55E−05 |
| nup93 | 9688 | nucleoporin 93 kDa | −0.250205563 | 0.153011215 |
| ocr1 | 4952 | oculocerebrorenal syndrome of | −0.478746687 | 0.013813838 |
| | | Lowe | | |
| odf2 | 4957 | outer dense fiber of sperm tails 2 | 0.23365825 | 0.051960803 |
| odf2 | 4957 | outer dense fiber of sperm tails 2 | 0.23365825 | 0.051960803 |
| ofd1 | 8481 | oral-facial-digital syndrome 1 | 1.10307625 | 0.000335398 |
| orc1 | 4998 | origin recognition complex, | −0.396539944 | 0.194040207 |
| | | subunit 1 | | |
| pafah1b1 | 5048 | platelet-activating factor | −0.668409125 | 0.008705083 |
| | | acetylhydrolase lb, regulatory | | |
| | | subunit 1 (45 kDa) | | |
| pard3 | 56288 | par-3 family cell polarity | 1.920193813 | 3.74E−07 |
| | | regulator | | |
| pard6a | 50855 | par-6 family cell polarity | −0.785137937 | 1.95E−05 |
| | | regulator alpha | | |
| pard6a | 50855 | par-6 family cell polarity | −0.785137937 | 1.95E−05 |
| | | regulator alpha | | |
| pcm1 | 5108 | pericentriolar material 1 | −0.191198312 | 0.217281987 |
| pde6d | 5147 | phosphodiesterase 6D, cGMP- | 0.293929063 | 0.053060946 |
| | | specific, rod, delta | | |
| pdzd7 | 79955 | PDZ domain containing 7 | −0.937288563 | 0.012087355 |
| pibf1 | 10464 | progesterone | −0.09440906 | 0.380215024 |
| | | immunomodulatory binding | | |
| | | factor 1 | | |
| pkd1 | 5310 | polycystic kidney disease 1 | −0.187093313 | 0.416372297 |
| | | (autosomal dominant) | | |
| pkd1 | 5310 | polycystic kidney disease 1 | −0.187093313 | 0.416372297 |
| | | (autosomal dominant) | | |
| pkd2 | 5311 | polycystic kidney disease 2 | 0.901317813 | 0.000423459 |
| | | (autosomal dominant) | | |
| plk1 | 5347 | polo-like kinase 1 | 0.678344748 | 0.02518388 |
| poc1a | 25886 | POC1 centriolar protein A | −0.179653956 | 0.492650521 |
| ptch1 | 5727 | patched 1 | −0.158752 | 0.693622012 |
| ptpdc1 | 138639 | protein tyrosine phosphatase | −0.86243575 | 0.023223622 |
| | | domain containing 1 | | |
| rab11a | 8766 | RAB11A, member RAS | −0.41064225 | 0.003856 |
| | | oncogene family | | |
| rab17 | 64284 | RAB17, member RAS oncogene | −2.087746813 | 0.000576014 |
| | | family | | |
| rab23 | 51715 | RAB23, member RAS oncogene | 0.490641688 | 0.088808493 |
| | | family | | |
| rab3ip | 117177 | RAB3A interacting protein | 0.140846813 | 0.524420306 |
| rab8a | 4218 | RAB8A, member RAS | 0.25576075 | 0.088900467 |
| | | oncogene family | | |
| ran | 5901 | RAN, member RAS oncogene | −0.2585665 | 0.187191371 |
| | | family | | |
| ranbp1 | 5902 | RAN binding protein 1 | −0.352029125 | 0.019601184 |
| rfx3 | 5991 | regulatory factor X, 3 | −0.564691156 | 0.07313726 |
| | | (influences HLA class II | | |
| | | expression) | | |
| rfx3 | 5991 | regulatory factor X, 3 | −0.564691156 | 0.07313726 |
| | | (influences HLA class II | | |
| | | expression) | | |
| rilpl1 | 353116 | Rab interacting lysosomal | 0.0456635 | 0.731582013 |
| | | protein-like 1 | | |
| rilpl2 | 196383 | Rab interacting lysosomal | 0.45641575 | 0.045217712 |
| | | protein-like 2 | | |
| rp2 | 6102 | retinitis pigmentosa 2 (X-linked | 0.702960188 | 0.000632734 |
| | | recessive) | | |
| rpgrip1l | 23322 | RPGRIP1-like | −0.588934188 | 0.019383986 |
| rsph1 | 89765 | radial spoke head 1 homolog | 0.719706625 | 0.081492286 |
| | | (Chlamydomonas) | | |
| rsph9 | 221421 | radial spoke head 9 homolog | 1.404178791 | 0.000539961 |
| | | (Chlamydomonas) | | |
| rttn | 25914 | rotatin | 0.8020281 | 0.009066656 |
| rttn | 25914 | rotatin | 0.8020281 | 0.009066656 |
| sass6 | 163786 | spindle assembly 6 homolog (C. | 0.358727975 | 0.109664413 |
| | | elegans) | | |
| scit1 | 132320 | sodium channel and clathrin | 0.456065 | 0.013599735 |
| | | linker 1 | | |
| sdccag8 | 10806 | serologically defined colon | 0.364890625 | 0.003133161 |
| | | cancer antigen 8 | | |
| shh | 6469 | sonic hedgehog | −0.81833149 | 0.058379038 |
| slc47a2 | 146802 | solute carrier family 47 | 3.659560848 | 2.95E−06 |
| | | (multidrug and toxin extrusion), | | |
| | | member 2 | | |
| smo | 6608 | smoothened, frizzled class | 1.798810355 | 3.00E−05 |
| | | receptor | | |
| snap25 | 6616 | synaptosomal-associated | −1.469181313 | 0.002541125 |
| | | protein, 25 kDa | | |
| snap25 | 6616 | synaptosomal-associated | −1.469181313 | 0.002541125 |
| | | protein, 25 kDa | | |
| snap25 | 6616 | synaptosomal-associated | −1.469181313 | 0.002541125 |
| | | protein, 25 kDa | | |
| snx10 | 29887 | sorting nexin 10 | −0.598017187 | 0.207725425 |
| spa17 | 53340 | sperm autoantigenic protein 17 | −0.014394687 | 0.957926284 |
| spata7 | 55812 | spermatogenesis associated 7 | −0.227978937 | 0.149234809 |
| spef2 | 79925 | sperm flagellar 2 | −0.22806 | 0.335475715 |
| sstr3 | 6753 | somatostatin receptor 3 | −1.3132037 | 0.033811502 |
| stk36 | 27148 | serine/threonine kinase 36 | 1.42793475 | 4.28E−05 |
| stk381 | 23012 | serine/threonine kinase 38 like | 0.009023938 | 0.970446143 |
| stx3 | 6809 | syntaxin 3 | −0.253552812 | 0.344032581 |
| sufu | 51684 | suppressor of fused homolog | 0.462835313 | 0.002907531 |
| | | (Drosophila) | | |
| tbc1d7 | 51256 | TBC1 domain family, member 7 | −0.314918937 | 0.364329681 |
| tctn1 | 79600 | tectonic family member 1 | 0.930323125 | 0.000293111 |
| tekt2 | 27285 | tektin 2 (testicular) | 1.834908931 | 0.002687057 |
| tekt4 | 150483 | tektin 4 | −0.236651866 | 0.685066319 |
| tmem138 | 51524 | transmembrane protein 138 | 0.7518405 | 0.000189422 |
| tmem216 | 51259 | transmembrane protein 216 | 1.050876538 | 2.45E−06 |
| tmem67 | 91147 | transmembrane protein 67 | 0.59243275 | 0.015728578 |
| tnpo1 | 3842 | transportin 1 | 0.094361313 | 0.476266224 |
| topors | 10210 | topoisomerase I binding, | 0.117762172 | 0.466634782 |
| | | arginine/serine-rich, E3 | | |
| | | ubiquitin protein ligase | | |
| tppp2 | 122664 | tubulin polymerization- | 1.062016838 | 0.053686611 |
| | | promoting protein family | | |
| | | member 2 | | |
| trafip1 | 26146 | TNF receptor-associated factor | −0.005247937 | 0.969005936 |
| | | 3 interacting protein 1 | | |
| trappe10 | 7109 | trafficking protein particle | −0.487046187 | 0.034400416 |
| | | complex 10 | | |
| trappc3 | 27095 | trafficking protein particle | 0.076370875 | 0.592557777 |
| | | complex 3 | | |
| trappc9 | 83696 | trafficking protein particle | −0.306270375 | 0.013781618 |
| | | complex 9 | | |
| ttbk2 | 146057 | tau tubulin kinase 2 | −1.682938438 | 6.01E−06 |
| ttc12 | 54970 | tetratricopeptide repeat domain | 0.538371736 | 0.069239192 |
| | | 12 | | |
| ttc21b | 79809 | tetratricopeptide repeat domain | −0.54081425 | 0.002822243 |
| | | 21B | | |
| ttc26 | 79989 | tetratricopeptide repeat domain | 0.245736592 | 0.254024727 |
| | | 26 | | |
| ttc29 | 83894 | tetratricopeptide repeat domain | −0.717591775 | 0.363818108 |
| | | 29 | | |
| ttc30a | 92104 | tetratricopeptide repeat domain | 0.423200625 | 0.055168496 |
| | | 30A | | |
| ttc8 | 123016 | tetratricopeptide repeat domain | −0.032124562 | 0.902072744 |
| | | 8 | | |
| ttk | 7272 | TTK protein kinase | 1.102875203 | 0.041679116 |
| ttll3 | 26140 | tubulin tyrosine ligase-like | 1.033588688 | 0.000779527 |
| | | family member 3 | | |
| ttll9 | 164395 | tubulin tyrosine ligase-like | 0.392160221 | 0.390716311 |
| | | family member 9 | | |
| tuba1a | 7846 | tubulin, alpha 1a | 0.40124175 | 0.111421523 |
| tuba4a | 7277 | tubulin, alpha 4a | −0.855843188 | 0.022910915 |
| tubb2a | 7280 | tubulin, beta 2A class IIa | 0.0773185 | 0.786864964 |
| tubb2b | 347733 | tubulin, beta 2B class IIb | 0.758292438 | 0.006158049 |
| tubb3 | 10381 | tubulin, beta 3 class III | −0.250513719 | 0.393584102 |
| tube1 | 51175 | tubulin, epsilon 1 | 0.418629375 | 0.064596271 |
| tubgcp2 | 10844 | tubulin, gamma complex | −0.420919125 | 0.027233016 |
| | | associated protein 2 | | |
| tubgcp3 | 10426 | tubulin, gamma complex | −0.101359447 | 0.345911348 |
| | | associated protein 3 | | |
| tubgcp5 | 114791 | tubulin, gamma complex | −0.562101313 | 0.004363719 |
| | | associated protein 5 | | |
| tubgcp6 | 85378 | tubulin, gamma complex | 0.079673625 | 0.719938444 |
| | | associated protein 6 | | |
| tulp1 | 7287 | tubby like protein 1 | −0.50249225 | 0.389388936 |
| ush1c | 10083 | Usher syndrome 1C (autosomal | 0.673252482 | 0.47438415 |
| | | recessive, severe) | | |
| ush2a | 7399 | Usher syndrome 2A (autosomal | 0.499538903 | 0.1465933 |
| | | recessive, mild) | | |
| vdac3 | 7419 | voltage-dependent anion | −0.47323775 | 0.002346675 |
| | | channel 3 | | |
| vhl | 7428 | von Hippel-Lindau tumor | 0.09062075 | 0.516656493 |
| | | suppressor, E3 ubiquitin protein | | |
| | | ligase | | |
| wdr19 | 57728 | WD repeat domain 19 | 0.41106625 | 5.83E−05 |
| wdr35 | 57539 | WD repeat domain 35 | 0.221804125 | 0.142336867 |
| wdr60 | 55112 | WD repeat domain 60 | −0.10830825 | 0.611074481 |
| wdr78 | 79819 | WD repeat domain 78 | 0.918277238 | 0.011522144 |

TABLE 2A
Clinical and molecular genetic information of individuals.

| Patient | Mutated | | | |
|---|---|---|---|---|
| ID | gene | Mutation type | Nucleotide changes | Protein change |
| E200 | TSC2 | Parents tested negative- | c.847A > T | p.Arg283X |
| | | likely de novo | | |
| E211 | TSC2 | Reported de novo | c.4952A > G | p.Asn1651Ser |
| E185 | TSC2 | Parents tested negative- | A to G at IVS18-2 | altered slicing |
| | | likely de novo | splice site | |
| E172 | TSC2 | Reported as only family | C.5227C > T | p. Arg1743Trp |
| | | member with the | | |
| | | mutation-likely de | | |
| | | novo | | |
| E351 | TSC1 | Parents tested negative- | c.737 + 3A > G | intronic sequence variant |
| | | likely de novo | | |
| E358 | TSC2 | Parents tested negative- | 1 bp deletion of G at | codon 1356, frameshift |
| | | likely de novo | nucleotide position | mutation |
| | | | 4067 | |
| E189 | N/A | N/A | N/A | N/A |
| E173 | N/A | N/A | N/A | N/A |
| E180 | N/A | N/A | N/A | N/A |
| E155 | N/A | N/A | N/A | N/A |
| E174 | N/A | N/A | N/A | N/A |
| E220 | N/A | N/A | N/A | N/A |

TABLE 2B
Clinical and molecular genetic information of individuals.

| | | Age at | | | |
|---|---|---|---|---|---|
| Patient | | surgery | | Brain | |
| ID | Ethnicity | (years) | Sex | Region | Diagnosis |
| E200 | White | 20 months | Female | Left | Intractable epilepsy with seizures coming |
| | | | | Temporal | mostly from the left temporal lobe. |
| | | | | Lobe | |
| E211 | Asian | 6 years 6 | Female | Left | Medically refractory epilepsy with seizures |
| | | months | | Occipital | originating from the occipital lobe. |
| | | | | Lobe | Multiple tubers. |
| E185 | White | 2 years | Male | Right Frontal | Medically refractory epilepsy with |
| | | | | Tuber | multiple epileptogenic frontal lobe tubers |
| | | | | | adjacent to the motor area. |
| E172 | White | 1 year, 10 | Male | Right | Medically refractory epilepsy. |
| | | months | | Parietal | Predominant seizure focus related to a |
| | | | | Occipital | right parietal tuber. Complex partial |
| | | | | Tuber | seizures. |
| E351 | White | 2 years, 2 | Female | Right Frontal | Medically refractory epilepsy. |
| | | months | | Lobe | Hypothyroidism |
| E358 | Black | 14 years | Male | Right Tuber | Medically refractory epilepsy. |
| | | and 4 | | | |
| | | months | | | |
| E189 | Black | 11 years, 7 | Female | Right | Medically refractory epilepsy with left |
| | | months | | hippocampus | hemiparesis, subsequent to neonatal injury. |
| E173 | White | 15 years, 4 | Female | Lateral | Medically refractory epilepsy status post |
| | | months | | temporal | resection of posterior temporal lesion with |
| | | | | Lobe | suspected onset of seizures in the anterior |
| | | | | | residual temporal lobe. |
| E180 | White | 17 years, 2 | Male | Left temporal | Medically refractory epilepsy with |
| | | months | | tip | extensive left hemisphere perinatal infarct. |
| E155 | Unknown | 16 years, 1 | Unknown | Left temporal | Medically refractory epilepsy with |
| | | month | | lobe | seizures of left temporal lobe origin. |
| E174 | White | 4 months | Female | Right | Medically refractory epilepsy with right- |
| | | | | temporal | sided hemimegalencephaly. Extensive |
| | | | | lobe | polymicrogyria, heterotopia, and abnormal |
| | | | | | white matter signal intensity. |
| E220 | White | 15 years 8 | Female | Lateral | Seizure |
| | | months | | temporal | |
| | | | | lobe | |

### Example 3: Disinhibition of mTORC1 Activity Due to Tsc2-Knockdown in Neurons Leads to Reduced Ciliation

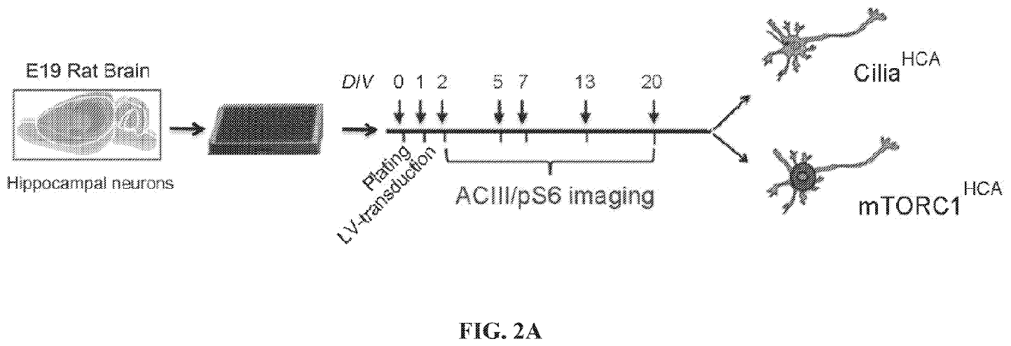

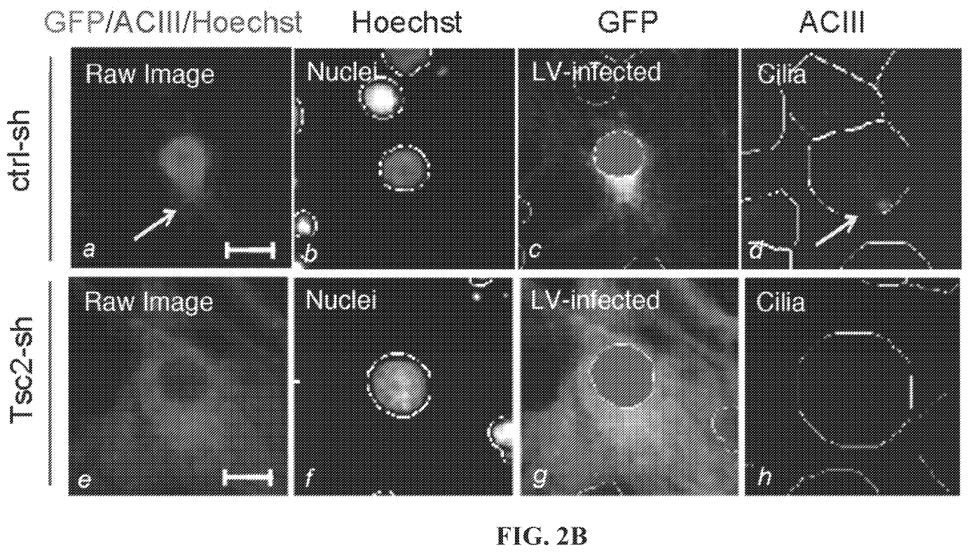

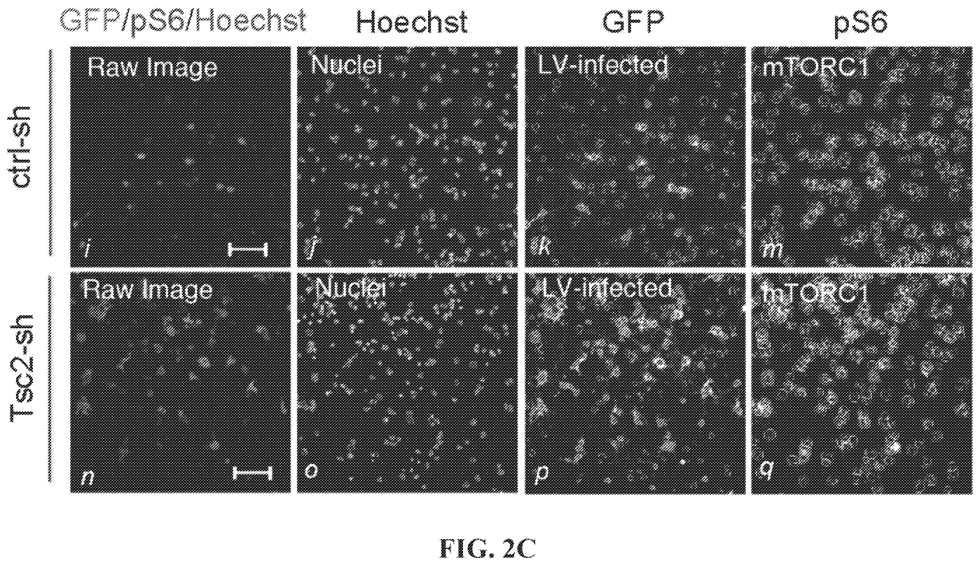

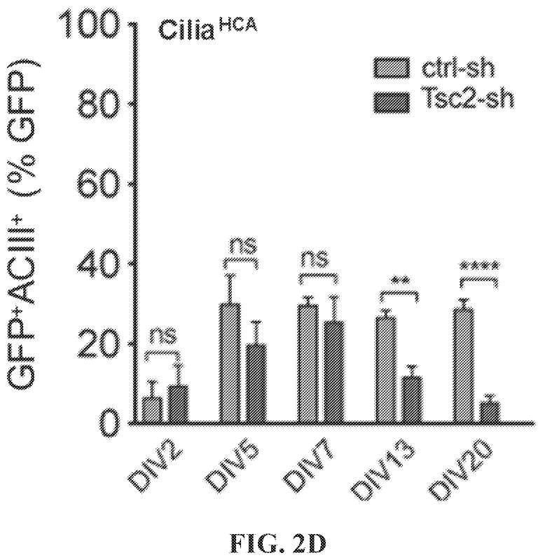

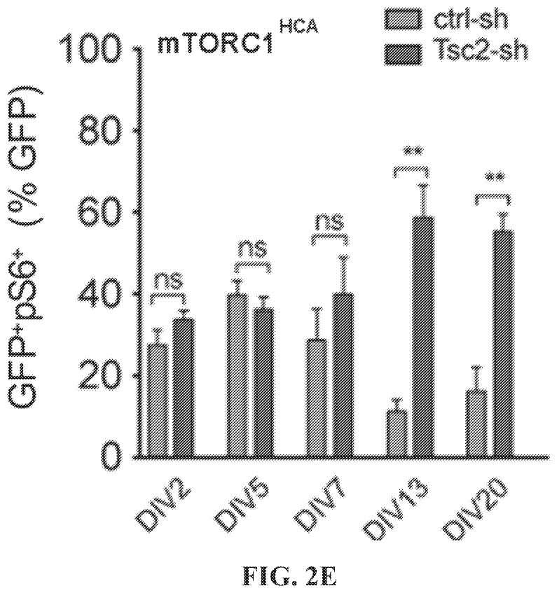

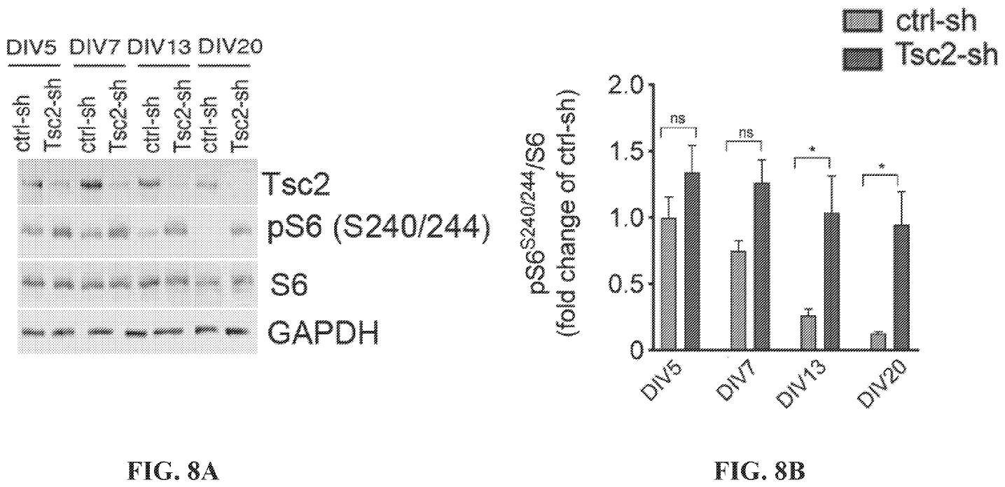

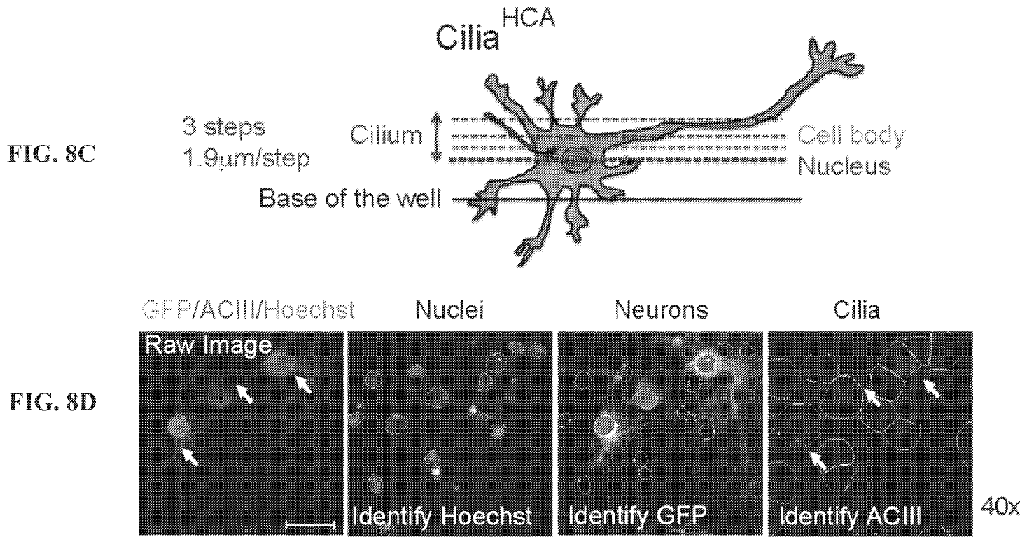

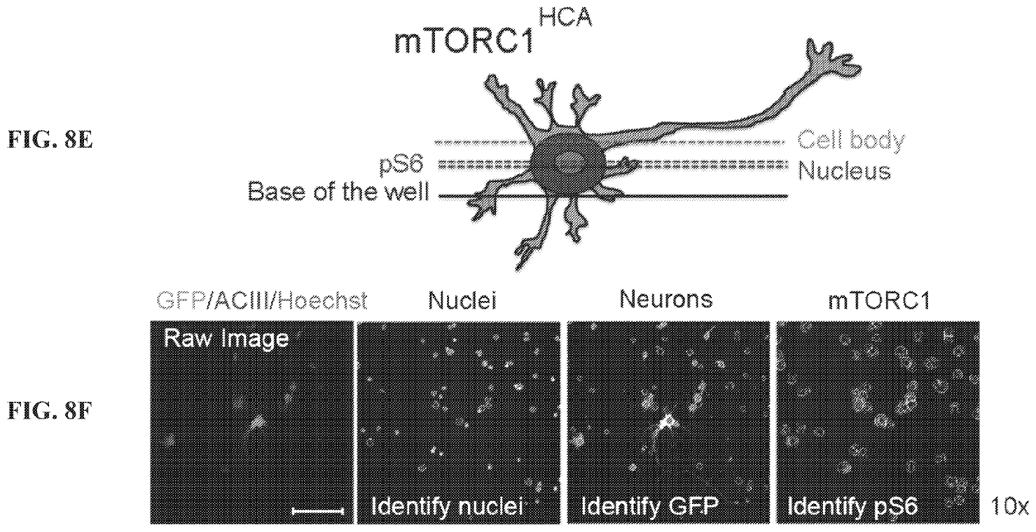

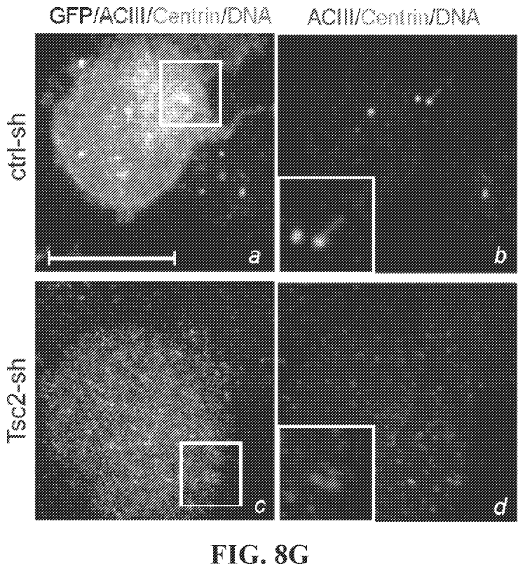

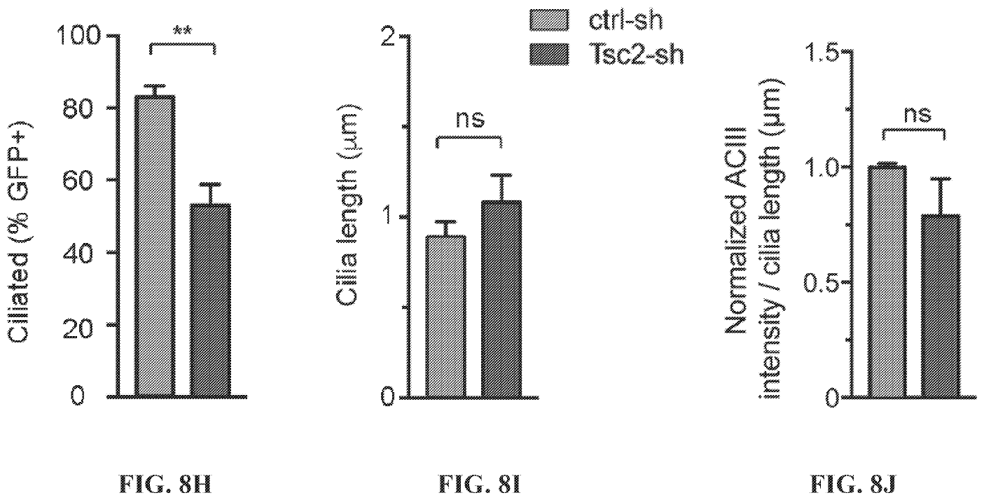

To investigate how mTORC1 dysregulation due to neuronal TSC loss affected ciliation, High Content image-based Assays (HCAs) were developed for unbiased quantification of cilia and mTORC1 activity (hereafter cilia^{HCA} and mTORC1^{HCA}) in primary neurons. As an in vitro model of TSC, rat hippocampal neurons transduced with lentiviral vectors (LV) expressing a short hairpin RNA (shRNA) were used directed against either the Tsc2 (Tsc2-sh) or the luciferase gene as a control (ctrl-sh) tagged with GFP. LV-mediated Tsc2 knockdown recapitulates several in vivo manifestations observed in mouse models of TSC (Di Nardo et al., Tuberous sclerosis complex activity is required to control neuronal stress responses in an mTOR-dependent manner; J Neurosci 29, 5926-5937 (2009); Di Nardo et al., Neuronal Tsc1/2 complex controls autophagy through AMPK-dependent regulation of ULK1; Hum Mol Genet 23, 3865-3874 (2014); Ebrahimi-Fakhari et al., Impaired Mitochondrial Dynamics and Mitophagy in Neuronal Models of Tuberous Sclerosis Complex; Cell Rep 17, 1053-1070 (2016); Ercan et al., Neuronal CTGF/CCN2 negatively regulates myelination in a mouse model of tuberous sclerosis complex; The Journal of experimental medicine 214, 681-697 (2017); Nie et al., The Stress-Induced Atf3-Gelsolin Cascade Underlies Dendritic Spine Deficits in Neuronal Models of Tuberous Sclerosis Complex; J Neurosci 35, 10762-10772 (2015); Nie et al., Tsc2-Rheb signaling regulates EphA-mediated axon guidance. Nat Neurosci 13, 163-172 (2010)). In vitro phenotypes include robust TSC2 protein downregulation and mTORC1 activation as shown by the time course of increased phosphorylation of ribosomal protein S6 at serine 240/244 (pS6) (FIGS. 8A-B). Both the cilia^{HCA} and mTORC1^{HCA} were optimized as imaged-based assays, where cilia and mTORC1 activity was monitored by immunofluorescent staining of ACIII to identify cilia and pS6 as a proxy of mTORC1 activation (FIGS. 8C-F). Neurons were transduced in culture at day in vitro (DIV) 1, and cilia and mTORC1 activity were assessed in a time course experiment at DIV2, 5, 7, 13, and 20 (FIG. 2A). LV-infected neurons with cilia or with phosphorylated S6 (GFP⁺ ACIII⁺ and GFP⁺pS6⁺ respectively) were identified by automated algorithms to detect subcellular structures and their co-localization specifically optimized for each assay. In control neurons, cilia development was observed between DIV2-5, which remained stable between DIV7 and DIV20 (FIGS. 2B, D). In contrast, the percentage of ciliated Tsc2-deficient neurons progressively decreased between DIV7-20 (FIGS. 2B, D). Notably, in the Tsc2-deficient neurons, the decrease in ciliation between DIV7 and DIV13 correlated with an increase in mTORC1 activation, unlike in control neurons where mTORC1 activity declined (FIGS. 2C, 2E, 8A, 8B). Reduced ciliation was validated by manual imaging and quantification of LV-infected neurons co-labeled with ACIII/centrin (FIGS. 8G-H). No changes in size or ACIII intensity were detected in the remaining cilia of the Tsc2-sh neurons (FIGS. 8I-J). These data show that mTORC1 activity is inversely correlated with cilia in neurons and suggest that mTORC1 disinhibition acts as a brake on ciliation.

### Example 4: A Phenotypic Screen in Tsc2-Knockdown Neurons Identifies Hsp90 as Drug Target for mTORC1 Through Regulation of PI3K/Akt Signaling Components

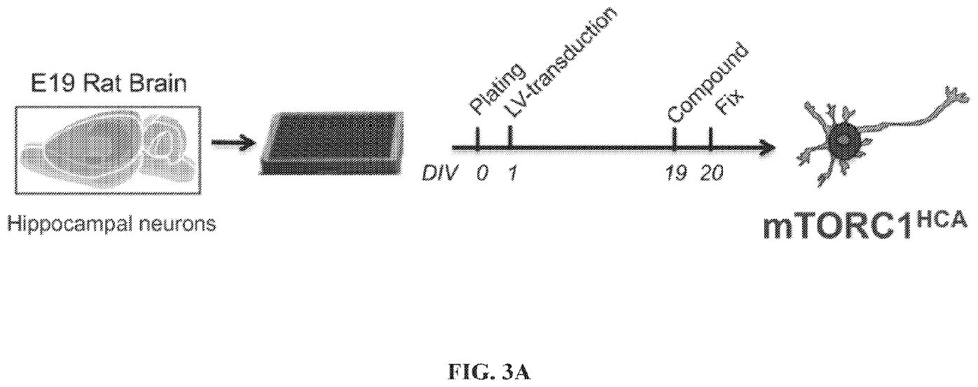

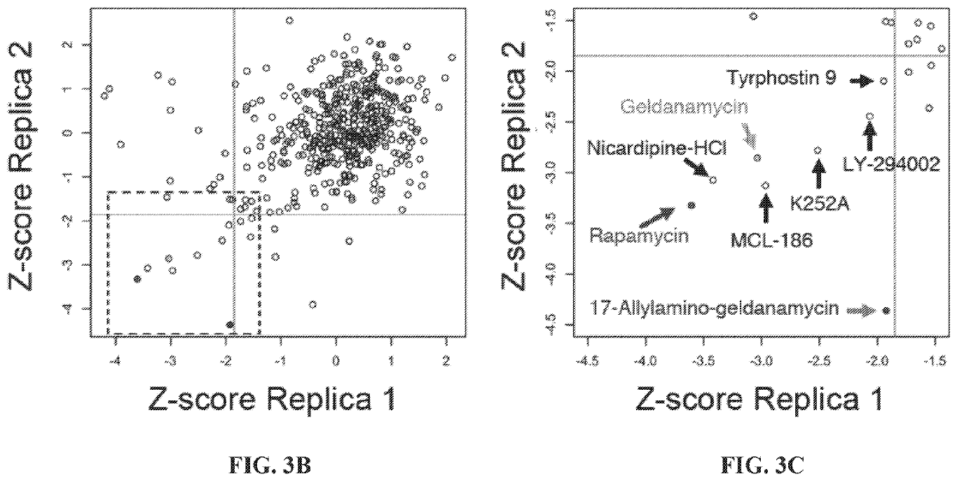

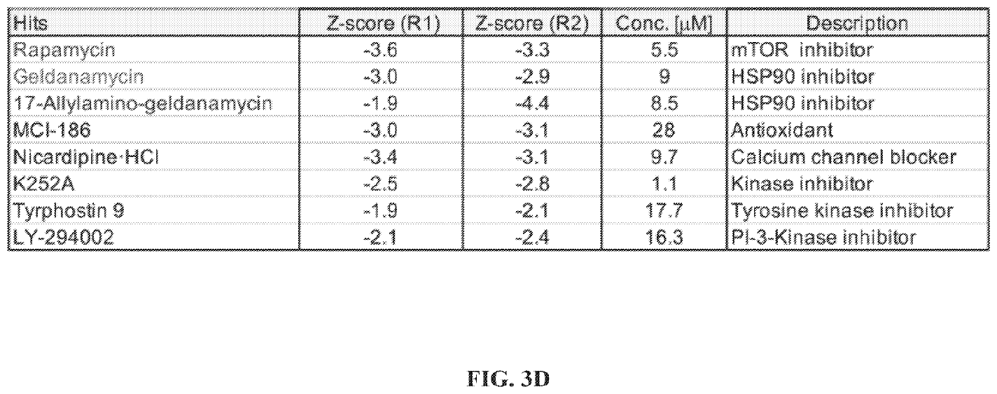

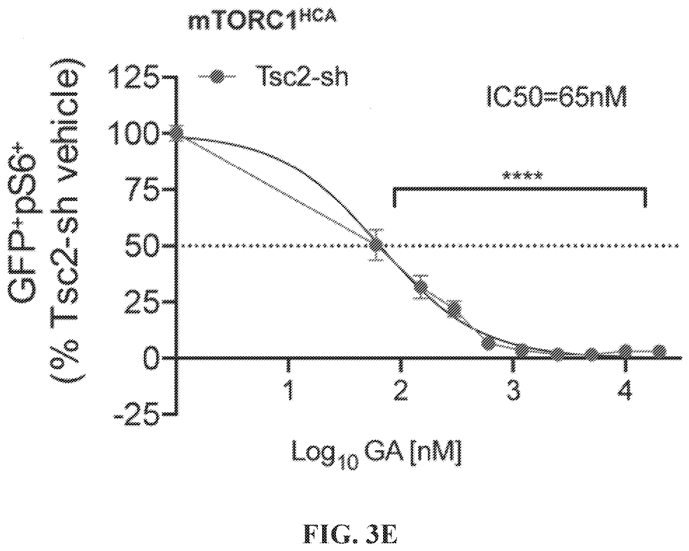

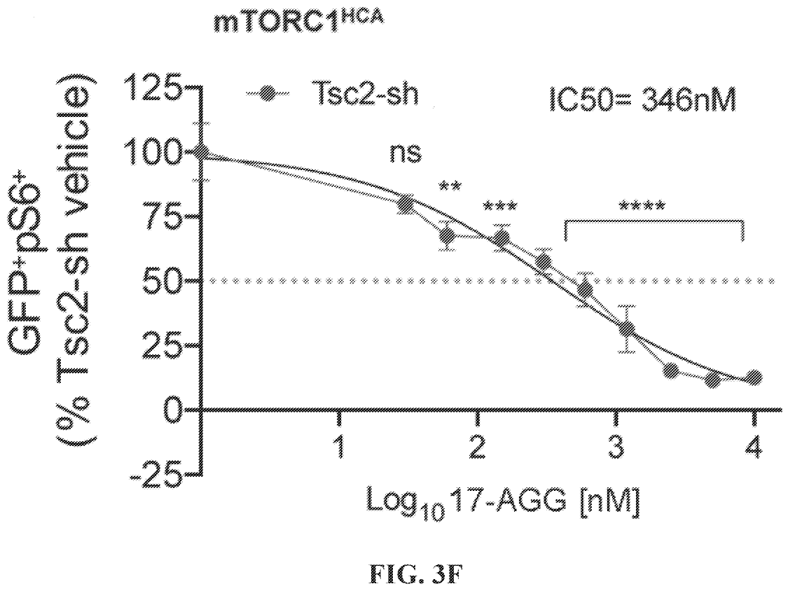

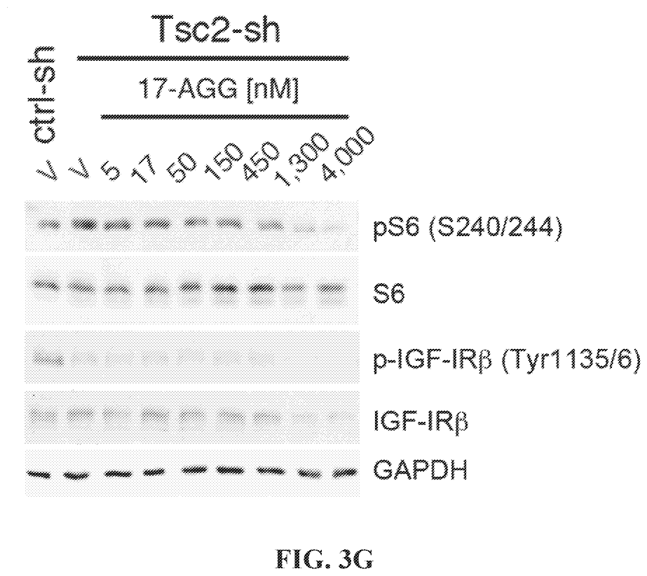

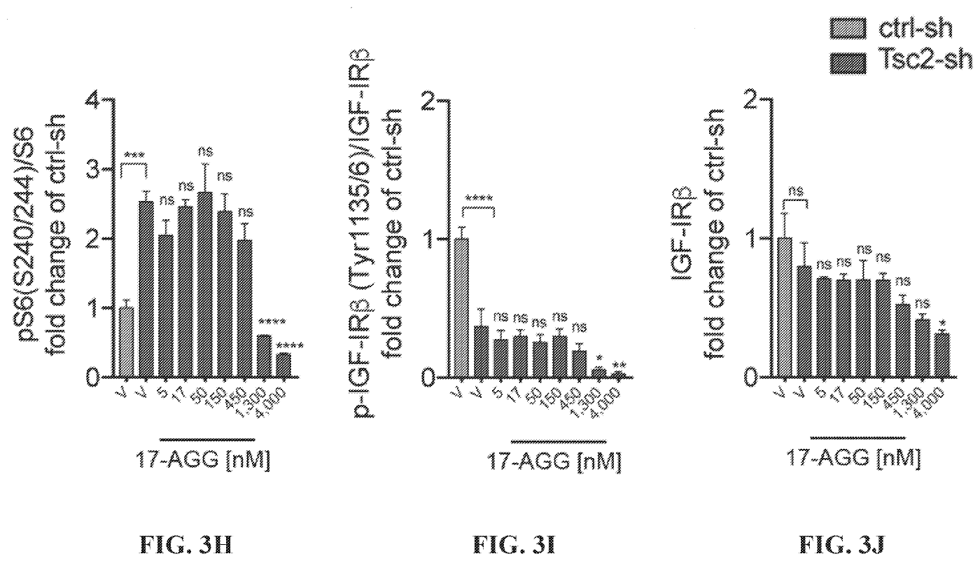

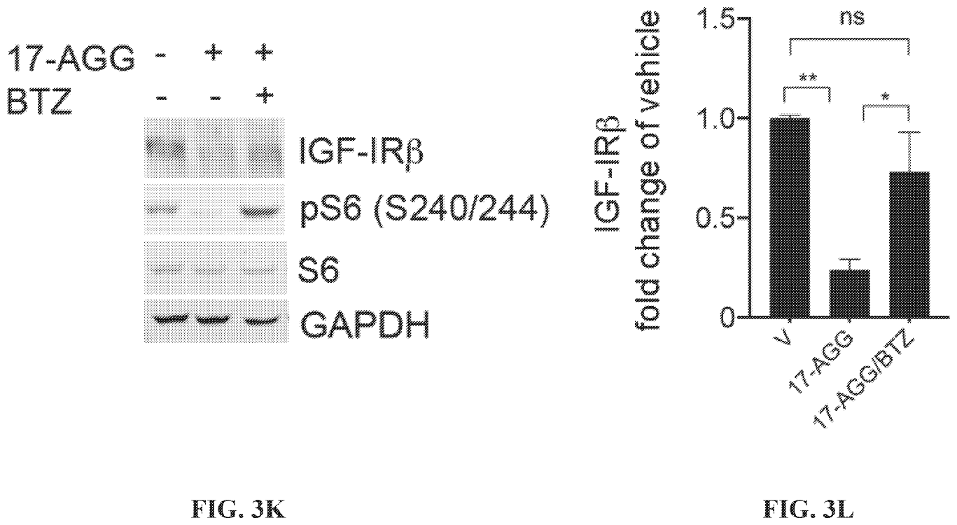

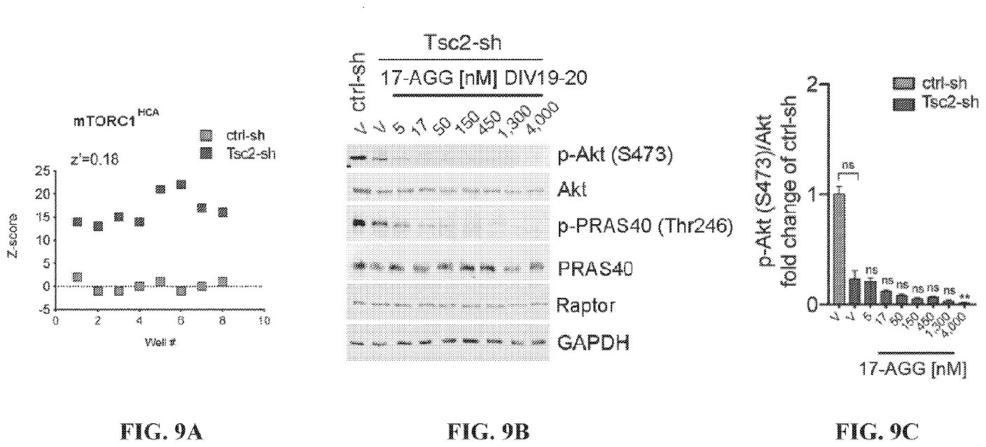

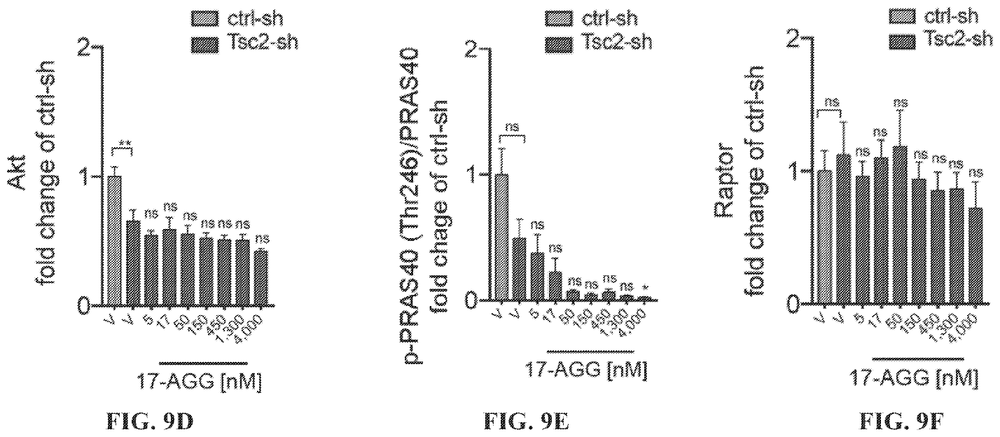

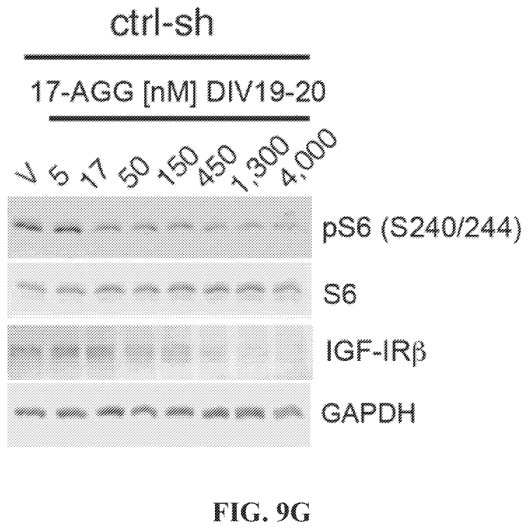

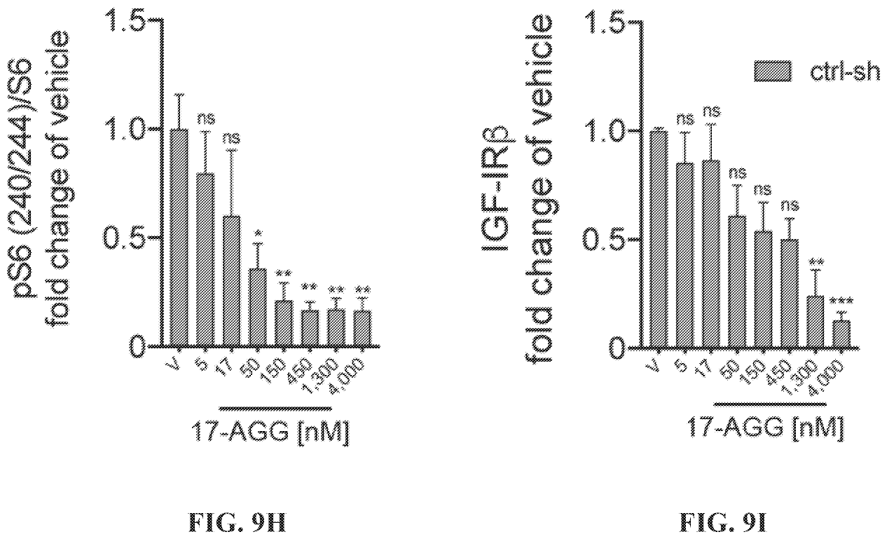

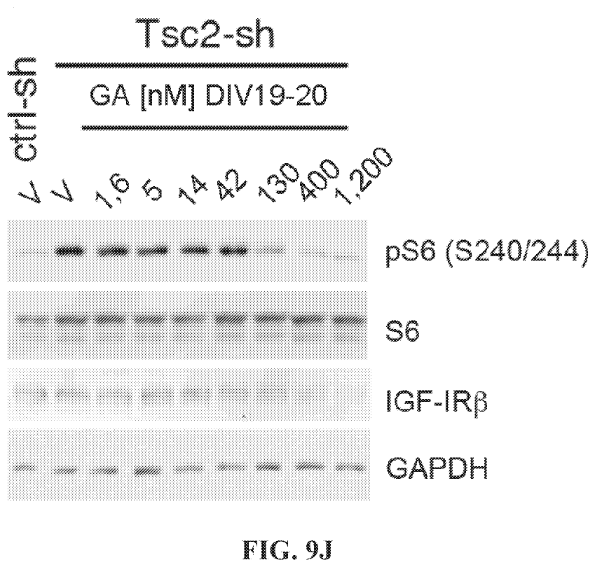

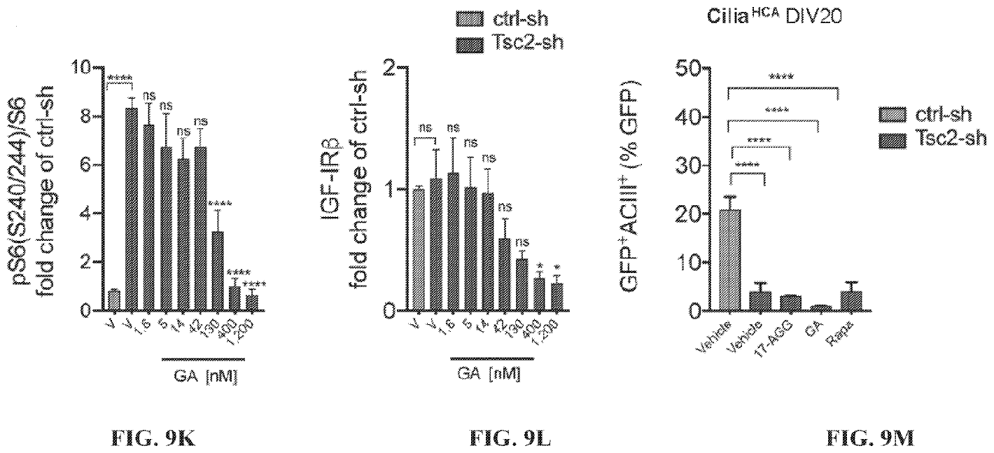

To explore potential mTORC1-dependent pathways involved in disrupted ciliation in Tsc2-deficient neurons, a high-content screen was performed to identify bioactive compounds that inhibit S6 phosphorylation using the mTORC1^{HCA}. Control and Tsc2-deficient neurons were transduced at DIV1, and the screen was performed at DIV20 since optimal assay robustness and reproducibility was found at that age in culture (Z prime=0.18, FIG. 3A, FIG. 9A). The screen was carried out in duplicate using the Biomol collection library, which included well-characterized bioactive compounds with known mechanism of action. The neurons were treated with each compound in duplicate, followed by fixation and immunofluorescent staining with GFP, pS6 and Hoechst. Staining for GFP was used to identify the LV-infected neurons, pS6 intensity was used to determine mTORC1 activity, and Hoechst was used to identify the nuclei. Plate normalization was done by converting the percent of GFP⁺pS6⁺ neurons of compound-treated Tsc2-knockdown wells into Z-scores (FIGS. 3B-C, Table 3). Rapamycin was the top hit in the screen, indicating robustness of the mTORC1^{HCA}. Other than rapamycin, positive hits included: two inhibitors of the heat shock protein 90 (Hsp90), Geldanamycin (GA) and 17-Allylamino-geldanamycin (17-AGG), the anti-oxidant MCI-186, the calcium channel blocker Nicardipine-HCl, and the kinase inhibitors K252A, Tyrphostin 9 and LY-294002 (FIG. 3D). A confirmatory 9-point dose-response experiments was performed for both GA and 17-AGG because these compounds hit the same molecular target. Using the mTORC1^{HCA}, GA was more potent (IC50^{pS6}=65 nM) than 17-AGG (IC50^{pS6}=346 nM) (FIGS. 3E-F). These IC50s were in the range of the average pharmacological potencies for GA and 17-AGG against Hsp90 (GA mean IC50=50 nM and 17-AGG mean IC50=220 nM) from a panel of human cancer cell lines (Kelland et al., DT-Diaphorase expression and tumor cell sensitivity to 17-allylamino, 17-demethoxygeldanamycin, an inhibitor of heat shock protein 90; J Natl Cancer Inst 91, 1940-1949 (1999)). Given that these compounds affect the same target and display rank order potencies that are well aligned with the literature, it suggests that Hsp90 is a potential regulator of mTORC1 activity in neurons.

Hsp90 is a molecular chaperone that protects its client proteins from degradation, and many of its substrates are oncogenic proteins (Neckers and Workman, Hsp90 molecular chaperone inhibitors: are we there yet?; Clin Cancer Res 18, 64-76 (2012)). Among these, insulin-growth factor-1 Receptor β (IGF-IRβ), Akt, and Raptor are components of the PI3K/mTOR pathway that have been identified as Hsp90 substrates in non-neuronal cells (Basso et al., Akt forms an intracellular complex with heat shock protein 90 (Hsp90) and Cdc37 and is destabilized by inhibitors of Hsp90 function; J Biol Chem 277, 39858-39866 (2002); Ohji et al., Suppression of the mTOR-raptor signaling pathway by the inhibitor of heat shock protein 90 geldanamycin; J Biochem 139, 129-135 (2006)). To test whether Hsp90 affects mTORC1 activity through regulation of IGF-IRβ, Akt, or Raptor in neurons, the expression of these potential client proteins were examined in Tsc2-knockdown neurons treated with vehicle or with a 7-point dose-response curve at a three-fold dilution of 17-AGG (assay endpoint DIV20). 17-AGG significantly reduced total IGF-IRβ protein level and pS6 phosphorylation at a dose of 4 μM, while there was no effect on Akt or Raptor levels (FIGS. 3G-I and FIGS. 9B, D, F). Similarly, 17-AGG reduced S6 phosphorylation and IGF-IRβ levels in ctrl-sh neurons, indicating that the interaction between Hsp90 and IGF-IRβ represents a general mechanism in neurons (FIGS. 9G-I). GA was then tested in the Tsc2-sh neurons, which resulted in a similar reduction of IGF-IRβ expression at dosing in the range of inhibition of S6 phosphorylation (FIGS. 9J-L). Therefore, these data demonstrate that Hsp90 inhibition can reduce the activity of mTORC1, as well as decrease the expression of upstream signaling components.

To investigate whether Hsp90 could function as a chaperone of IGF-IRβ in neurons, wild-type neurons were treated with 17-AGG and then proteasomal degradation was inhibited using bortezomib (BTZ). As expected, Hsp90 inhibition led to reduced IGF-IRβ levels, and this reduction was prevented with BTZ. Interestingly, the reduction in S6 phosphorylation was also prevented by BTZ in these neurons (FIGS. 3K-L). Without wishing to be bound by theory, it is likely that Hsp90 protects IGF-IRβ against proteasomal degradation. To test whether IGF-IRβ protein stability and signaling may represent a potential mechanism by which Hsp90 inhibition reduces mTORC1 activity, IGF-IRβ activation was assessed by autophosphorylation at Tyr1135/1136. Consistent with mTOR-dependent negative feedback on the RTK/Akt pathway (Zhang et al., S6K1 regulates GSK3 under conditions of mTOR-dependent feedback inhibition of Akt; Molecular cell 24, 185-197 (2006)), IGF-IRβ phosphorylation was reduced in the vehicle-treated Tsc2-sh neurons compared to controls (FIGS. 3G, J). Notably, 17-AGG further reduced phosphorylation of IGF-IRβ receptor, and IGF-IRβ phosphorylation was essentially absent in Tsc2-knockdown neurons at doses that reduced IGF-IRβ levels and inhibited mTORC1, demonstrating that 17-AGG inhibits IGF-IRβ activity in neurons. (FIGS. 3G, J). Downstream components of IGF-IRβ signaling were examined, which indicated that phosphorylation of Akt and of its substrate the proline-rich Akt substrate 40 (PRAS40) were also reduced by 17-AGG (FIGS. 9B-C, E). Interestingly, PRAS40 is an inhibitor of mTORC1, and its phosphorylation reduces PRAS40 binding to mTORC1 (Sancak et al., PRAS40 is an insulin-regulated inhibitor of the mTORC1 protein kinase; Mol Cell 25, 903-915 (2007)). Taken together, these data indicate that IGF-IRβ and Akt activity may be permissive to ongoing activation of mTORC1 and that Hsp90 inhibition completely suppresses components of the PI3K/Akt signaling pathway, further contributing to mTORC1 inhibition.

TABLE 3
List of compounds and Z scores of the mTORCI screen.

| | | T2 Zscore | T2 Zscore |
|---|---|---|---|
| Compound Name | Compound description | (Replica 1) | (Replica 2) |
| Rapamycin | Inhibitors: FRAP inhibitor | −3.605714807 | −3.324484711 |
| Geldanamycin | Inhibitors: HSP-90 inhibitor | −3.035804295 | −2.855719006 |
| 17-Allylamino- | Inhibitors: HSP-90 inhibitor | −1.923988377 | −4.360690318 |
| geldanamycin | | | |
| MCI-186 | Inhibitors: antioxidant, | −2.965206994 | −3.128197545 |
| | cytoprotectant | | |
| Nicardipine HQ | Ion channel ligands: Calcium | −3.417906703 | −3.073795294 |
| | channels | | |
| K252A | Kinase inhibitors: Kinase | −2.514559807 | −2.779086739 |
| | inhibitor (Broad spectrum) | | |
| Tyrphostin 9 | Kinase inhibitors: PDGF-R | −1.94482648 | −2.09736229 |
| | tyrosine kinase inhibitor | | |
| Etoposide | Inhibitors: topoisomerase II | −4.201814383 | 0.844178636 |
| | inhibitor | | |
| Minoxidil sulfate | Ion channel ligands: Potassium | −4.113025678 | 1.003628691 |
| | channels | | |
| Acetyl (N)-s-farnesyl-1- | Inhibitors: farnesylation | −3.910951731 | −0.263482361 |
| cysteine | inhibitor | | |
| AG-370 | Kinase inhibitors: PDGF | −3.069206112 | −1.458750054 |
| | receptor kinase inhibitor | | |
| LY-294002 | Lipid biosynthesis: PI-3- | −2.066495193 | −2.444479285 |
| | Kinase inhibitor | | |
| Tetrandine | Ion channel ligands: Calcium | −3.008612626 | −1.087945552 |
| | channels | | |
| Thapsigargin | Ion channel ligands: | −1.102903078 | −2.819443895 |
| | Intracellular calcium | | |
| Cytochalasin B | Inhibitors: F actin capper | −0.425880459 | −3.902035405 |
| Hinokitiol | Inhibitors: Iron chelator | −1.553127065 | −2.36312517 |
| 5,8,11,14-Eicosatetraynoic | Bioactive lipids: | −3.233474597 | 1.309121426 |
| acid | Cyclooxygenase & | | |
| | lipoxygenase inhibitor | | |
| D-erythro-MAPP | Lipid biosynthesis: | −3.004942103 | 0.518642606 |
| | Ceramidase inhibitor | | |
| Camptothecin | Inhibitors: Topoisomerase 1 | −1.729505926 | −2.009309467 |
| | inhibitor | | |
| Calphostin C | Kinase inhibitors: PKC | −2.289412803 | −1.268854598 |
| | inhibitor | | |
| N-Acetyl-leukotriene E4 | Bioactive lipids: Bioactive | −2.974886294 | 1.160843492 |
| | arachidonic acid metabolite | | |
| Aristolochic acid | Lipid biosynthesis: | −2.220042659 | −1.171866835 |
| | phospholipase A2 inhibitor | | |
| H-89 | Kinase inhibitors: PKA | −1.534424407 | −1.943146138 |
| | inhibitor | | |
| 4-Amino-1,8- | Inhibitors: PARP inhibitor | −1.72993979 | −1.728502835 |
| naphthalimide | | | |
| NSC-95397 | Inhibitors: CDC25 | −1.927502223 | −1.510361112 |
| | phosphatase inhibitor | | |
| Doxorubicin | Inhibitors: topoisomerase II | −1.880563881 | −1.520715689 |
| | inhibitor, induces apoptosis | | |
| MG-132 | Protease inhibitors: | −1.116941831 | −2.17860697 |
| | Proteasome inhibitor | | |
| Valinomycin | on channel ligands: K + | −1.656383415 | −1.687728978 |
| | ionophore | | |
| Tyrphostin AG-126 | Kinase inhibitors: tyrosine | −2.110552614 | −1.006953048 |
| | kinase inhibitor | | |
| Lycorine | Inhibitors: inhibits TNFalpha | −1.444107571 | −1.777689685 |
| | production | | |
| Actinomycin D | Inhibitors: transcription | −1.646900132 | −1.522624888 |
| | inhibitor | | |
| TMB-8 | Ion channel ligands: | −2.49968736 | 0.0562997 |
| | Intracellular calcium | | |
| 5-Iodotubercidin | Kinase inhibitors: ERK-2 | −1.537358326 | −1.555286427 |
| | inhibitor | | |
| Nafamostat mesylate | Inhibitors: Serine protease | −1.201986214 | −1.808878032 |
| | inhibitor | | |
| Mead ethanolamide | Endocannabinoids: | 0.239625364 | −2.454881279 |
| | Cannabinoid receptor agonist | | |
| MY-5445 | Inhibitors: phosphodiesterase | −1.029331424 | −1.797809409 |
| | (PDE5) inhibitor | | |
| C8 Dihydroceramide | Bioactive lipids: Negative | −2.012806756 | −0.465956537 |
| | control for C8 ceramide | | |
| S-farnesyl-L-cysteine | Bioactive lipids: MDR ATPase | −1.254283022 | −1.474876553 |
| | activator | | |
| L-744,832 | Inhibitors: Ras | −0.876186231 | −1.704921868 |
| | farnesyltransferase inhibitor | | |
| 10-hydroxycamptothecin | Inhibitors: topoisomerase 1 | −0.717233558 | −1.672584409 |
| | inhibitor | | |
| Prostaglandin A2 | Bioactive lipids: Bioactive | −1.618264072 | −0.78094607 |
| | prostaglandin | | |
| DL-PDMP | Lipid biosynthesis: | −0.759807652 | −1.57819199 |
| | Glucosylceramide synthase | | |
| | inhibitor | | |
| Dimethyloxaloylglycine | Inhibitor: Prolyl-4-hydroxylase | −1.336719715 | −1.039458876 |
| | inhibitor | | |
| FCCP | Inhibitors: mitochondrial | −0.755412351 | −1.489806322 |
| | uncoupler | | |
| Niflumic acid | Ion channel ligands: Mise, | −1.610993034 | −0.391567297 |
| | channels | | |
| Eicosadienoic acid (20:2 n- | Bioactive lipids: | −0.205933991 | −1.688205222 |
| 6) | Polyunsaturated fatty acid | | |
| Siguazodan | Inhibitors: phosphodiesterase | −0.745628329 | −1.35200196 |
| | (PDE3) inhibitor | | |
| N-acetyl-S-geranylgeranyl- | Bioactive lipids: ICMT | −1.347536849 | −0.739547924 |
| L-Cysteine | inhibitor | | |
| Wortmannin | Lipid biosynthesis: PI- | −1.168045228 | −0.783866025 |
| | 3Kinase, other kinases | | |
| | inhibitor | | |
| Nimodipine | Ion channel ligands: Calcium | −1.826647282 | 1.108951742 |
| | channels | | |
| Diazoxide | Ion channel ligands: Potassium | −0.383286527 | −1.364906416 |
| | channels | | |
| BAY 11-7082 | Inhibitors: Inhibits IKK kinase | −1.184700615 | −0.629484674 |
| | activation | | |
| N-linoleoylglycine | Endocannabinoids: FAAH | −1.373393343 | −0.305265913 |
| | inhibitor | | |
| Verapimil | Ion channel ligands: Calcium | −0.414497295 | −1.255322187 |
| | channels | | |
| Estradiol | Nuclear receptor ligands: | 1.198690022 | −1.745172051 |
| | estrogen | | |
| Dipalmitoylphosphatidic | Bioactive lipids: Activates | −1.633139036 | 0.530649647 |
| acid | MAP kinase cascade | | |
| E-Capsaicin | Inhibitors: Topoisomerase 1 | −1.628292711 | 0.597101354 |
| | inhibitor | | |
| 13(S)-HPODE | Bioactive lipids: Fatty acid | 0.288188519 | −1.527059609 |
| | hydroperoxide | | |
| Sphingosine | Bioactive lipids: PKC inhibitor | −1.14423344 | −0.501448098 |
| Leukotoxin A (9,10- | Bioactive lipids: Bioactive | −1.35583535 | −0.111625859 |
| EODE) | linoleic acid metabolite | | |
| Amiodarone•HCl | Ion channel ligands: Calcium | −0.997905239 | −0.667441038 |
| | channels | | |
| Phenytoin | Inhibitors: MAP kinase | −0.944457072 | −0.705089003 |
| | activator | | |
| WY-14643 | Nuclear receptor ligands: | −0.067447056 | −1.340235576 |
| | PPAR alpha agonist | | |
| Dantrolene | Ion channel ligands: | 0.279774968 | −1.466267304 |
| | Intracellular calcium | | |
| Enantio-PAF C16 | Bioactive lipids: Negative | −1.476444135 | 0.325752822 |
| | control for PAF | | |
| Zaprinast | Inhibitors: phosphodiesterase | 0.006307903 | −1.348568456 |
| | (PDE1) inhibitor | | |
| 4-Oxatetradecanoic acid | Bioactive lipids: Myristic acid | 0.002896416 | −1.330557654 |
| | analog | | |
| D12-Prostaglandin J2 | Bioactive lipids: Bioactive | 0.714933708 | −1.544643298 |
| | prostaglandin | | |
| Prostaglandin F1a | Bioactive lipids: Prostaglandin | −0.879930871 | −0.680351819 |
| | FP receptor agonist | | |
| Linoleic acid | Bioactive lipids: | 0.211232669 | −1.388741132 |
| | Polyunsaturated fatty acid | | |
| 1,2-Dioctanoyl-SN- | Bioactive lipids: Activates | −1.617869339 | 1.405234686 |
| glycerol | PKC | | |
| 3-aminobenzamide (3- | Inhibitors: ADP ribose | −1.512827152 | 0.67785977 |
| ABA) | polymerase, apoptosis inhibitor | | |
| 9b, 11a-Prostaglandin F2 | Bioactive lipids: Bioactive | −0.208869231 | −1.160348899 |
| | prostaglandin | | |
| Cyclosporin A | Inhibitors: calcineurin inhibitor | −1.12451491 | −0.182627612 |
| Trequinsin | Inhibitors: phosphodiesterase | −0.950624031 | −0.445076628 |
| | (PDE3) inhibitor | | |
| Cycloheximide | Inhibitors: protein synthesis | −0.503940482 | −0.904618184 |
| | inhibitor | | |
| Docosapentaenoic acid | Bioactive lipids: | −0.954203701 | −0.4014532 |
| | Polyunsaturated fatty acid | | |
| C2 Dihydroceramide | Bioactive lipids: Negative | −0.107912336 | −1.130748358 |
| | control for C2 ceramide | | |
| 6-Keto-prostaglandin F1a | Bioactive lipids: Bioactive | −1.08330032 | −0.152720168 |
| | prostaglandin | | |
| Loperamide•HCl | Ion channel ligands: Calcium | 0.817125002 | −1.394324803 |
| | channels | | |
| Nigericin | nhibitors: induces intracellular | −1.366489651 | 0.734531333 |
| | acidification | | |
| ZM226600 | Ion channel ligands: Potassium | −0.491198164 | −0.790544264 |
| | channels | | |
| Minoxidil | Ion channel ligands: Potassium | 0.074550053 | −1.133115998 |
| | channels | | |
| Rottierin | Kinase inhibitors: PKC delta | −0.338085997 | −0.871565405 |
| | inhibitor | | |
| QX-314 | Ion channel ligands: Sodium | 1.300696113 | −1.406428341 |
| | channels | | |
| Tunicamycin | Inhibitors: glycosylation | −0.910279501 | −0.248054378 |
| | inhibitor | | |
| Leukotriene B4 | Bioactive lipids: Leukotriene | 0.680824686 | −1.288310997 |
| | B4 receptor agonist | | |
| AG1478 | Kinase inhibitors: Tyrosine | 0.15655814 | −1.11393544 |
| | kinase inhibitor. Broad | | |
| | spectrum | | |
| IBMX | Inhibitors: PDE inhibitor | −0.676425666 | −0.511600335 |
| | (broad spec), adenosineR | | |
| | agonist | | |
| EHNA | nhibitors: Phosphodiesterase | 0.191867317 | −1.11021477 |
| | (PDE2) inhibitor/adenosine | | |
| | deaminase inhibitor | | |
| 1-Hexadecyl-2-O-acetyl- | Bioactive lipids: Blocks DAG | −0.987330289 | −0.056285327 |
| glycerol | activation of PKC | | |
| SKF-96365 | Ion channel ligands: Calcium | −1.160598648 | 0.35426311 |
| | channels | | |
| Tolazamide | Ion channel ligands: Potassium | 0.821656963 | −1.286178246 |
| | channels | | |
| 15(S)-HETE | Bioactive lipids: Bioactive | −0.876888153 | −0.192152162 |
| | arachidonic acid metabolite | | |
| Curcumin | Inhibitors: NFkappaB inhibitor | −1.018182696 | 0.078082411 |
| H7 | Kinase inhibitors: kinase | −0.315124333 | −0.773204911 |
| | inhibitor | | |
| Mitomycin C | Inhibitors: cross links DNA | −0.367411436 | −0.722515624 |
| PAF C16 | Bioactive lipids: PAF receptor | −0.605477676 | −0.501411131 |
| | agonist | | |
| Flecainide acetate | Ion channel ligands: Sodium | −0.814358555 | −0.227694141 |
| | channels | | |
| Quercetin•2H2O | Kinase inhibitors: kinase | −0.284430889 | −0.736375459 |
| | inhibitor (plus other) | | |
| Pepstatin | Protease inhibitors: protease | −0.455550629 | −0.590793979 |
| | inhibitor | | |
| Eicosapentaenoic acid | Bioactive lipids: | 0.151411406 | −0.976926639 |
| (20:5 n-3) | Polyunsaturated fatty acid | | |
| 15-Ketoicosatetraenoic | Bioactive lipids: Bioactive | 0.695516113 | −1.149059016 |
| acid | arachidonic acid metabolite | | |
| 1α,25-Dihydroxyvitamin | Nuclear receptor ligands: | 0.338589402 | −1.035058464 |
| D3 | Vitamin D receptor agonist | | |
| Nifedipine | Ion channel ligands: Calcium | −1.265041154 | 1.472015548 |
| | channels | | |
| NapSul-Ile-Trp-CHO | Protease inhibitors: Cathepsin | 0.2641671 | −0.993142746 |
| | L inhibitor | | |
| Benzamil•HCl | Ion channel ligands: Calcium | 0.15056397 | −0.941626351 |
| | channels | | |
| Farnesylthioacetic acid | Bioactive lipids: | −0.285092776 | −0.651108747 |
| | Carboxymethylation inhibitor | | |
| E-4031 | Ion channel ligands: Potassium | 0.234083465 | −0.951776544 |
| | channels | | |
| 1,2-Dioleoyl-glycerol | Bioactive lipids: Activates | −0.363563422 | −0.568468127 |
| (18:1) | PKC | | |
| KT-5720 | Kinase inhibitors: PKA | −0.210518282 | −0.686007444 |
| | inhibitor | | |
| Indirubin | Kinase inhibitors: GSK-3beta | −0.258102608 | −0.6434732 |
| | inhibitor | | |
| N9-Isopropylolomoucine | Kinase inhibitors: CDC-2 | −0.759364439 | −0.080608904 |
| | kinase inhibitor | | |
| 17-Octadecynoic acid | Lipid biosynthesis: Inhibits | 1.553454056 | −1.207339011 |
| | fatty acid omega oxidation | | |
| Olomoucine | Kinase inhibitors: CDK | −0.172365778 | −0.681079964 |
| | inhibitor | | |
| 1400W•2HCl | Inhibitors: iNOS inhibitor | −0.559282551 | −0.317548331 |
| BAPTA-AM | Inhibitors: cell permeable | −0.165133551 | −0.67805263 |
| | Ca++ chelator | | |
| Manumycin A | Inhibitors: ras farnesylation | −0.433923 | −0.421343016 |
| | inhibitor | | |
| LFM-A13 | Kinase inhibitors: BTK | −0.843740984 | 0.166768235 |
| | inhibitor | | |
| PD 98059 | Kinase inhibitors: MEK | −0.478695551 | −0.338156745 |
| | inhibitor | | |
| U-75302 | Bioactive lipids: Leukotriene | −0.777650601 | 0.108822147 |
| | B4 receptor antagonist | | |
| Prostaglandin J2 | Bioactive lipids: Bioactive | 1.33952185 | −1.118719839 |
| | prostaglandin | | |
| Splitomycin | Inhibitors: sir2p inhibitor | −0.031199617 | −0.687259932 |
| Pimozide | Ion channel ligands: Calcium | 0.022184681 | −0.716709345 |
| | channels | | |
| AG-879 | Kinase inhibitors: NGF | 0.731600149 | −0.981513753 |
| | receptor inhibitor | | |
| HA-1004 | Kinase inhibitors: kinase | −0.419291298 | −0.300145529 |
| | inhibitor | | |
| 5,6-Epoxy eicosatrienoic | Bioactive lipids: Bioactive | −0.609230807 | −0.049748106 |
| acid | arachidonic acid metabolite | | |
| bezafibrate | Nuclear receptor ligands: | −0.220306726 | −0.467158525 |
| | PPAR alpha agonist | | |
| 1-Acyl-PAF | Bioactive lipids: PAF agonist | −0.117714664 | −0.546399026 |
| FPL-64176 | Ion channel ligands: Calcium | 0.251261064 | −0.760898201 |
| | channels | | |
| Dexamethasone | Nuclear receptor ligands: | −0.077763289 | −0.543178778 |
| | corticosteroid | | |
| RG-14620 | Kinase inhibitors: EGF-R | −0.946285626 | 0.844986876 |
| | tyrosine kinase inhibitor | | |
| Deprenyl | Inhibitors: inhibits | −0.41096313 | −0.23795297 |
| | glyceraldehyde-3-phosphate | | |
| | dehydrogenase | | |
| U-74389G | Inhibitors: superoxide/free- | −0.728529209 | 0.242064676 |
| | radical inhibitor | | |
| CITCO | Nuclear receptor ligands: | −0.641333514 | 0.0834122 |
| | Const, androstane receptor | | |
| | (CAR) agonist | | |
| 1-stearoyl-2-arachidonyl- | Bioactive lipids: PKC activator | −0.389851475 | −0.249495098 |
| glycerol | | | |
| 1-Hexadecyl-2-O-methyl- | Bioactive lipids: Blocks DAG | −0.727795084 | 0.253835012 |
| glycerol | activation of PKC | | |
| 24(S)-hydroxycholesterol | Nuclear receptor ligands: LXR | −0.482244968 | −0.112472108 |
| | agonist | | |
| U-46619 | Bioactive lipids: Thromboxane | 0.889164521 | −0.924598612 |
| | TP receptor agonist | | |
| Adrenic acid (22:4, n-6) | Bioactive lipids: | −0.259045661 | −0.347037926 |
| | Polyunsaturated fatty acid | | |
| Z-VAD(OMe)-FMK | Protease inhibitors: Caspase | −0.529400534 | −0.035239829 |
| | inhibitor (broad spectrum) | | |
| L-NASPA | Bioactive lipids: LPA agonist/ | −0.22672302 | −0.373581763 |
| | antagonist | | |
| Cyclopiazonic acid | Ion channel ligands: | 0.116447463 | −0.593075498 |
| | Intracellular calcium | | |
| 8-epi-Prostaglandin F2a | Bioactive lipids: Thromboxane | −0.375877227 | −0.180806139 |
| | TP receptor agonist | | |
| Lidocaine•HCl•H2O | Ion channel ligands: Sodium | −0.527458778 | 0.022717262 |
| | channels | | |
| Propafenone | Ion channel ligands: Potassium | 0.239389658 | −0.653157397 |
| | channels | | |
| Arachidonoyl-PAF | Bioactive lipids: PAF | 0.363402154 | −0.709196042 |
| | precursor | | |
| PCA 4248 | Bioactive lipids: PAF | −0.367755495 | −0.166126462 |
| | antagonist | | |
| AM-580 | Nuclear receptor ligands: | −0.281270846 | −0.256071395 |
| | Retinoid RAR agonist | | |
| 5′-N- | CNS receptor ligands: | 0.447893185 | −0.730671847 |
| Ethylcarboxamidoadenosine | adenosine receptor agonist | | |
| (NECA) | | | |
| N-arachidonoylglycine | Endocannabinoids: FAAH | −0.132915665 | −0.376076849 |
| | inhibitor | | |
| MnTBAP chloride | Inhibitors: SOD mimetic | −0.147831289 | −0.357387591 |
| Arachidonic acid | Bioactive lipids: | −0.597625325 | 0.198606166 |
| (20:4, n-6) | Polyunsaturated fatty acid | | |
| Capsazepine | Ion channel ligands: vanilloid | 0.478516261 | −0.720581413 |
| | receptor antagonist | | |
| SB-431542 | Kinase inhibitors: ALK4, | 1.005786821 | −0.869566234 |
| | ALK5, ALK7 inhibitor | | |
| 2-Arachidonoylglycerol | Endocannabinoids: | 1.604133536 | −0.946476261 |
| | Cannabinoid CB1 receptor | | |
| | agonist | | |
| 9(S)-HPODE | Bioactive lipids: Fatty acid | −0.146480895 | −0.336132322 |
| | hydroperoxide | | |
| Prostaglandin B2 | Bioactive lipids: Bioactive | −0.565742101 | 0.198701267 |
| | prostaglandin | | |
| Lyso-PAF C16 | Bioactive lipids: Inactive PAF | −0.367521264 | −0.076098065 |
| | metabolite | | |
| BADGE | Nuclear receptor ligands: | 0.786243792 | −0.784001508 |
| | PPAR gamma antagonist | | |
| Fluprostenol | Bioactive lipids: Prostaglandin | −0.623206339 | 0.358677407 |
| | FP receptor agonist | | |
| L-cis-Diltiazem-HCl | Ion channel ligands: Calcium | −0.827206167 | 1.040754253 |
| | channels | | |
| Cirazoline | CNS receptor ligands: | −0.237575551 | −0.180654008 |
| | adrenoreceptor agonist (alpha) | | |
| CDC | Lipid biosynthesis: 12- | 0.233806707 | −0.537840805 |
| | Lipoxygenase inhibitor | | |
| Amantadine • HCl | Ion channel ligands: Mise, | −0.311482694 | −0.091330759 |
| | channels | | |
| Prostaglandin B1 | Bioactive lipids: Bioactive | 0.192544947 | −0.498546513 |
| | prostaglandin | | |
| Leukotriene D4 | Bioactive lipids: CysLT | 0.263474136 | −0.536328765 |
| | receptor agonist | | |
| CAPE | Inhibitors: Antioxidant/ | 0.379084543 | −0.592030338 |
| | NFkappa B inhibitor | | |
| Diphenyleneiodonium | Inhibitors: flavoprotein | −0.199215742 | −0.181116979 |
| | inhibitor | | |
| Dibutyrylcyclic GMP | Activators: PKA activator | −0.056311495 | −0.310907896 |
| Gamma-linolenic acid | Bioactive lipids: | 0.596186137 | −0.675838556 |
| (18:3 n-6) | Polyunsaturated fatty acid | | |
| N-Acetyl- S-geranyl-L- | Lipid biosynthesis: 5- | 0.2499217 | −0.513972796 |
| cysteine | Lipoxygenase inhibitor | | |
| Mevinolin (Lovastatin) | Lipid biosynthesis: Inhibitor | −0.734422638 | 0.847395653 |
| | HMG-CoA reductase | | |
| C-PAF | Bioactive lipids: PAF receptor | −0.105845265 | −0.227537795 |
| | agonist | | |
| PAF C18:1 | Bioactive lipids: PAF receptor | 0.377565375 | −0.551854512 |
| | agonist | | |
| Prostaglandin A1 | Bioactive lipids: Bioactive | −0.68108565 | 0.731857768 |
| | prostaglandin | | |
| 1-Hexadecyl-2- | Bioactive lipids: DAG analog | 0.584500067 | −0.61158625 |
| arachidonoyl-glycerol | | | |
| Dihomo-gamma-linolenic | Bioactive lipids: | −0.0687076 | −0.212958821 |
| acid | Polyunsaturated fatty acid | | |
| YC-1 | Activators: GC stimulator/ | −0.242854485 | −0.034027653 |
| | Hif-1 alpha inhibitor | | |
| 15-deoxy-Prostaglandin J2 | Bioactive lipids: Bioactive | 0.218942353 | −0.420235453 |
| | prostaglandin | | |
| Penitrem A | Ion channel ligands: Potassium | −0.845357806 | 2.555766093 |
| | channels | | |
| Damnacanthal | Kinase inhibitors: p561ck | 0.184425154 | −0.384341112 |
| | inhibitor | | |
| GW-9662 | Nuclear receptor ligands: | −0.024218317 | −0.227286139 |
| | PPAR gamma antagonist | | |
| Linolenic acid (18:3 n-3) | Bioactive lipids: | 0.377355787 | −0.48867246 |
| | Polyunsaturated fatty acid | | |
| 1-Oleoyl 2-acetyl-glycerol | Bioactive lipids: PKC activator | 0.225279743 | −0.397872337 |
| Linoleamide | Endocannabinoids: Bioactive | 0.067169328 | −0.288717877 |
| | linoleic acid metabolite | | |
| Mastoparan | Activators: activates | 0.10644236 | −0.31533421 |
| | heterotrimeriuc GTPases | | |
| Tyrphostin AG-825 | Kinase inhibitors: HER-1,2 | −0.5509699 | 0.547341202 |
| | tyrosine kinase inhibitor | | |
| AM-251 | CNS receptor ligands: | −0.309956892 | 0.113171598 |
| | Cannabinoid CB1 receptor | | |
| | antagonist | | |
| 8,9-Epoxyeicosatrienoic | Bioactive lipids: Bioactive | 0.318058603 | −0.42411077 |
| acid | arachidonic acid metabolite | | |
| Mycophenolic acid | Inhibitors: Inosine-5′- | 1.097982176 | −0.691322524 |
| | monophosphate dehydrogenase | | |
| | inhibitor | | |
| SDZ-201106 | Ion channel ligands: Sodium | 1.321592947 | −0.721075805 |
| | channels | | |
| Methoprene acid | Nuclear receptor ligands: | 0.295652604 | −0.39261324 |
| | Retinoid RXR agonist | | |
| Resveratrol | Activators: SIRTI activator | −0.114323538 | −0.070917639 |
| Thalidomide | Inhibitors: TNFalpha synthesis | −0.097756993 | −0.076250014 |
| | inhibitor | | |
| Cloprostenol | Bioactive lipids: Prostaglandin | −0.491853735 | 0.514980743 |
| | FP receptor agonist | | |
| Leukotriene C4 | Bioactive lipids: CysLT | 0.019719072 | −0.169053412 |
| | receptor agonist | | |
| LY-171883 | Bioactive lipids: Leukotriene | 0.051186158 | −0.177392066 |
| | D4 receptor antagonist | | |
| Tolbutamide | Ion channel ligands: Potassium | −0.22164075 | 0.121251722 |
| | channels | | |
| ndirubin-3′-monoxime | Kinase inhibitors: GSK-3beta | −0.096339621 | −0.021183928 |
| | inhibitor | | |
| CGP-37157 | Inhibitors: inhibitor of | −0.399747096 | 0.412765362 |
| | mitochondrial Na + Ca + 2 | | |
| | exchange | | |
| 4- | Nuclear receptor ligands: | −0.29683937 | 0.237915763 |
| hydroxyphenylretinamide | Retinoid receptor agonist/ | | |
| | apoptosis inducer | | |
| Y-27632 | Kinase inhibitors: ROCK | 0.432130169 | −0.392475905 |
| | inhibitor | | |
| Pinacidil | Ion channel ligands: Potassium | −0.572723271 | 0.914690544 |
| | channels | | |
| Clonidine | CNS receptor ligands: | 1.179390416 | −0.631379908 |
| | adrenoreceptor agonist (alpha) | | |
| N-Phenylanthranilic acid | Ion channel ligands: Mise, | 0.138806672 | −0.202166325 |
| | channels | | |
| Anandamide (18:2,n-6) | Endocannabinoids: | −0.013011222 | −0.051248919 |
| | Cannabinoid receptor agonist | | |
| Manoalide | Lipid biosynthesis: | −0.207652206 | 0.185427867 |
| | Phospholipase A2 inhibitor | | |
| SB-415286 | Kinase inhibitors: GSK3 beta | −0.305260427 | 0.342932748 |
| | inhibitor | | |
| Tyrphostin-8 | Inhibitors: Calcineurin | −0.529697342 | 0.884124732 |
| | inhibitor | | |
| 5(S)-HETE | Bioactive lipids: Bioactive | 0.234245609 | −0.225840509 |
| | arachidonic acid metabolite | | |
| DRB (Benzimidazole) | Kinase inhibitors: CKII | −0.115395393 | 0.112156496 |
| | inhibitor | | |
| Nitrendipine | Ion channel ligands: Calcium | 0.159494968 | −0.150770509 |
| | channels | | |
| Ryanodine | Ion channel ligands: | 0.328148987 | −0.255974565 |
| | Intracellular calcium | | |
| Thiorphan | Protease inhibitors: Neutral | −0.504081805 | 0.939681654 |
| | endopeptidase inhibitor | | |
| 13(S)-HODE | Bioactive lipids: Bioactive | 1.518126958 | −0.598532595 |
| | linoleic acid metabolite | | |
| Phenamil | Ion channel ligands: Sodium | 0.646635498 | −0.381028586 |
| | channels | | |
| Propidium iodide | Inhibitors: DNA intercalator | −0.102279987 | 0.190916327 |
| Prostaglandin E1 | Bioactive lipids: Prostaglandin | 1.252443482 | −0.524830182 |
| | EP receptor agonist | | |
| 9a, 11b-Prostaglandin F2 | Bioactive lipids: Bioactive | 0.356036168 | −0.19864377 |
| | prostaglandin | | |
| 5-Ketoeicosatetraenoic | Bioactive lipids: 5-KETE | 0.159453777 | −0.054725967 |
| acid | receptor (R527) agonist | | |
| Paxilline | Ion channel ligands: Potassium | 0.600283782 | −0.328267306 |
| | channels | | |
| Genistein | Kinase inhibitors: Tyrosine | 0.256068478 | −0.119259834 |
| | kinase inhibitor | | |
| 6,7-ADTN | CNS receptor ligands: | 0.488368484 | −0.263207643 |
| | Dopamine agonist | | |
| NS-398 | Lipid biosynthesis: Cox-2 | 1.289114831 | −0.514085855 |
| | inhibitor | | |
| Z-prolyl-prolinal | Protease inhibitors: Prolyl | 0.242990548 | −0.10145265 |
| | endopeptidase inhibitor | | |
| AM 92016 • HCl | Ion channel ligands: Potassium | 0.468590599 | −0.240653175 |
| | channels | | |
| 9-cis Retinoic acid | Nuclear receptor ligands: | 1.134856761 | −0.47609896 |
| | Retinoid RXR agonist | | |
| 1,2-Didecanoyl-glycerol | Bioactive lipids: Activates | 1.059069216 | −0.453365054 |
| (10:0) | PKC | | |
| Anandamide (20:4, n-6) | Endocannabinoids: | 0.085985731 | 0.056581574 |
| | Cannabinoid receptor agonist | | |
| 17-Phenyl-trinor- | Bioactive lipids: Prostaglandin | 0.239354849 | −0.074459176 |
| prostaglandin E2 | EPl receptor agonist | | |
| Fluspirilene | Inhibitors: bNOS/iNOS | −0.196611059 | 0.425869953 |
| | inhibitor | | |
| Blebbistatin | Inhibitors: Myosin II inhibitor | −0.001023465 | 0.161120657 |
| Bepridil • HCl | Ion channel ligands: Calcium | 0.600784887 | −0.283049741 |
| | channels | | |
| Kavain (+/−) | Ion channel ligands: voltage- | −0.009243649 | 0.174932974 |
| | dependent Na channel inhibitor | | |
| T etrahy drocannabinol-7- | Nuclear receptor ligands: | −0.043616513 | 0.22948894 |
| oic acid | PPAR gamma agonist | | |
| 2-APB | Inhibitors: IP3 receptor blocker | 0.141747381 | 0.033096654 |
| Pifithrin-α | Inhibitors: p53 inhibitor | 0.559886177 | −0.24881667 |
| 13-Keto-octadeca-9Z,11E− | Bioactive lipids: Bioactive | 0.265397202 | −0.062695452 |
| dienoic acid | linoleic acid metabolite | | |
| PAF C18 | Bioactive lipids: PAF receptor | 0.123756549 | 0.068648642 |
| | agonist | | |
| ICRF-193 | Inhibitors: topo II inhibitor that | 0.028870945 | 0.179379167 |
| | does not cause DNA breaks | | |
| RHC-80267 | Lipid biosynthesis: DAG | 0.35078499 | −0.100173002 |
| | lipase inhibitor | | |
| Dipyridamole | Inhibitors: cGMP | 0.459346378 | −0.167866208 |
| | phosphodiesterase inhibitor | | |
| Eicosatrienoic acid (20:3 | Bioactive lipids: | −0.362627963 | 0.925695961 |
| n-3) | Polyunsaturated fatty acid | | |
| 6(5H)-Phenanthridinone | Inhibitors: PARP inhibitor | −0.141614018 | 0.43603046 |
| GM6001 | Kinase inhibitors: PKC | −0.341932826 | 0.885975453 |
| | inhibitor | | |
| 1-Deoxymannojirimycin | Inhibitors: mannosidase | −0.458222137 | 1.454420221 |
| hydrochloride | inhibitornhibitors: | | |
| | mannosidase inhibitor | | |
| Fumonisin B1 | Lipid biosynthesis: inhibits | −0.239393311 | 0.64946654 |
| | ceramide synthase | | |
| 1-Deoxynojirimycin | Inhibitors: glucosidase | 0.401165699 | −0.100969578 |
| | inhibitor | | |
| Yohimbine | CNS receptor ligands: | 0.663783668 | −0.240521271 |
| | Adrenoreceptor antagonist | | |
| | (alpha) | | |
| 13-cis retinoic acid | Nuclear receptor ligands: | 0.296006144 | −0.016932652 |
| | Retinoid receptor ligand | | |
| Clozapine | CNS receptor ligands: | 0.420008735 | −0.101970406 |
| | Dopamine antagonist | | |
| Rolipram | Inhibitors: phosphodiesterase | 0.255299282 | 0.023869723 |
| | (PDE4 ) inhibitor | | |
| Diehlorobenzamil • HCl | Ion channel ligands: Calcium | 0.374438145 | −0.060059689 |
| | channels | | |
| 9,10-Octadecenoamide | Endocannabinoids: | −0.188106889 | 0.607880575 |
| | Endogenous sleep inducing | | |
| | lipid | | |
| All trans retinoic acid | Nuclear receptor ligands: | −0.251103427 | 0.750121911 |
| | Retinoid RAR agonist | | |
| Phorbol 12-myristate 13- | Activators: PKC activator | −0.144150552 | 0.525420365 |
| acetate | | | |
| Pregnenolone-16α- | Nuclear receptor ligands: | 0.898959495 | −0.304615083 |
| carbonitrile | PXR/SXR agonist | | |
| SB 203580 | Kinase inhibitors: Suppressor | 0.274445191 | 0.031016706 |
| | of MAPKAP kinase-2 | | |
| Veratridine | Ion channel ligands: Sodium | 0.736324334 | −0.230995331 |
| | channels | | |
| SQ22536 | Inhibitors: adenylate cyclase | −0.087971233 | 0.458346003 |
| | inhibitor | | |
| Lavendustin A | Kinase inhibitors: Tyrosine | 0.530057633 | −0.114463947 |
| | kinase inhibitor EGF-R) | | |
| Lysophosphatidic acid • Na | Bioactive lipids: LPA receptor | 0.45179505 | −0.067296214 |
| | antagonist | | |
| SB 202190 | Kinase inhibitors: MAP kinase | −0.014293354 | 0.380886338 |
| | inhibitor | | |
| Latrunculin B | Inhibitors: Actin inhibitor | −0.250643678 | 0.841947105 |
| NS-1619 | Ion channel ligands: Potassium | 0.05511162 | 0.291918828 |
| | channels | | |
| L-NAME | Inhibitors: NO synthesis | 0.016155546 | 0.343252653 |
| | inhibitor | | |
| Z-Leu3-VS | Protease inhibitors: | −0.262561549 | 0.883226061 |
| | Proteasome inhibitor | | |
| B581 | Inhibitors: farnesyltransferase | −0.121374004 | 0.587982557 |
| | inhibitor | | |
| TTNPB | Nuclear receptor ligands: | 0.478762416 | −0.04759953 |
| | Retinoid RAR agonist | | |
| Diltiazem • HCl | Ion channel ligands: Calcium | −0.38758112 | 1.628816567 |
| | channels | | |
| Eicosa-5,8-dienoic acid | Bioactive lipids: | 0.878044931 | −0.230227957 |
| (20:2 n-12) | Polyunsaturated fatty acid | | |
| U-37883A | Ion channel ligands: Potassium | 0.695693366 | −0.153288253 |
| | channels | | |
| 16,16-Dimethyl- | Bioactive lipids: Prostaglandin | 0.60496754 | −0.103455014 |
| prostaglandin E2 | EP receptor agonist | | |
| L-erythro-MAPP | Lipid biosynthesis: Negative | 0.697958005 | −0.145719784 |
| | control for D-erythro-MAPP | | |
| IB-MECA | CNS receptor ligands: | 1.326739754 | −0.32782005 |
| | Adenosine receptor agonist | | |
| 6-Formylindolo [3,2-B] | Bioactive lipids: AHR agonist | 0.721529446 | −0.137291252 |
| carbazole | | | |
| AG-1296 | Kinase inhibitors: c-kit, FGF | 0.124751639 | 0.312273157 |
| | and PDGF kinase inhibitor | | |
| Arvanil | Ion channel ligands: Vaniloid | 0.687656918 | −0.115434611 |
| | receptor agonist | | |
| SP-600125 | Kinase inhibitors: JNK | 0.406850293 | 0.052145104 |
| | inhibitor | | |
| Piroxicam | Lipid biosynthesis: COX1 | 0.573755863 | −0.048338018 |
| | inhibitor | | |
| 4-Aminopyridine | Ion channel ligands: Potassium | 0.146245017 | 0.309840426 |
| | channels | | |
| 5-Hydroxydecanoate | Ion channel ligands: Potassium | 0.218098512 | 0.241377267 |
| | channels | | |
| Bongkrekic acid | Inhibitors: ANT inhibitor | −0.232385218 | 1.063464104 |
| Furoxan | Activators: NO donor | 0.702151659 | −0.08245164 |
| BML-190 | Bioactive lipids: Cannabinoid | 0.325510801 | 0.172527007 |
| | CB1 inverse agonist | | |
| 12(S)-HPETE | Bioactive lipids: Fatty acid | 0.552163672 | 0.007363475 |
| | hydroperoxide | | |
| C8 Ceramine | Bioactive lipids: Ceramide | 0.494775378 | 0.058381852 |
| | analog. Apoptosis inducer | | |
| 15(S)-HPETE | Bioactive lipids: Fatty acid | −0.017282827 | 0.644598218 |
| | hydroperoxide | | |
| U-0126 | Kinase inhibitors: MEK | 0.033780443 | 0.57251652 |
| | inhibitor | | |
| lonomycin | Ion channel ligands: Ca++ | 0.126121785 | 0.439268115 |
| | ionophore | | |
| 24,25-Dihydroxyvitamin | Nuclear receptor ligands: | 0.530685086 | 0.06636352 |
| D3 | Vitamin D receptor ligand | | |
| Fluspirilene | Ion channel ligands: Potassium | 1.009548104 | −0.153395973 |
| | channels | | |
| Glyburide | Ion channel ligands: Potassium | −0.002265688 | 0.67389511 |
| | channels | | |
| GF-109203X | Kinase inhibitors: PKC | 0.842396801 | −0.075210459 |
| | inhibitor | | |
| 1-Hexadecyl-2- | Bioactive lipids: PAF receptor | 0.682868361 | 0.001074866 |
| methylglycero-3 PC | agonist | | |
| 8-Bromo-cGMP | Activators: PKG activator | 0.141419123 | 0.46110096 |
| Vinpocetin | Inhibitors: phosphodiesterase | −0.263057932 | 1.69153184 |
| | (PDE1) inhibitor | | |
| Brefeldin A | Inhibitors: ARF GEF inhibitor | 1.16810521 | −0.174719662 |
| FK-506 | Inhibitors: FKBP ligand | −0.023014149 | 0.752326934 |
| 2-Fluoropalmitic acid | Bioactive lipids: Protein | 0.805227569 | −0.046161422 |
| | palmitoylation inhibitor | | |
| HNMPA-(AM)3 | Kinase inhibitors: Insulin | 1.369653253 | −0.210316924 |
| | receptor TK inhibitor | | |
| H9 | Kinase inhibitors: kinase | 0.443028595 | 0.181665431 |
| | inhibitor | | |
| 7,7-Dimethyleicosadienoic | Bioactive lipids: PLA2 | −0.115387578 | 1.084791505 |
| acid | inhibitor | | |
| Phenoxybenzamine | Inhibitors: Calmodulin | 0.320457905 | 0.319158713 |
| | antagonist | | |
| Cytochalasin D | Inhibitors: F actin capper | 0.731907297 | 0.024581727 |
| Arachidonamide | Endocannabinoids: Bioactive | 0.020149882 | 0.792705881 |
| | arachidonic acid metabolite | | |
| Decoyinine | Inhibitors: lowers GTP levels | 0.342526631 | 0.350022262 |
| Phentolamine | Ion channel ligands: Potassium | 1.59055954 | −0.178949161 |
| | channels | | |
| N-Acetyl-S-geranyl-L- | Bioactive lipids: Negative | 0.004110044 | 0.874788752 |
| cysteine | control for AGGC and AFC | | |
| RK-682 | Inhibitors: VHR phosphatase | 1.329356816 | −0.132749392 |
| | inhibitor | | |
| ML7 | Kinase inhibitors: kinase | 0.810968203 | 0.038254229 |
| | inhibitor | | |
| A-3 | Kinase inhibitors: kinase | −0.050323034 | 1.065766254 |
| | inhibitor | | |
| Diazoxide | Ion channel ligands: Potassium | −0.099472187 | 1.244121914 |
| | channels | | |
| Grayanotoxin III | Ion channel ligands: Sodium | 0.97674696 | −0.017019657 |
| | channels | | |
| 9(S)-HODE | Bioactive lipids: Bioactive | 0.288065238 | 0.447478303 |
| | linoleic acid metabolite | | |
| Mead acid (20:3 n-9) | Bioactive lipids: | 1.221308279 | −0.086436882 |
| | Polyunsaturated fatty acid | | |
| (R)-(+)-Methandamide | CNS receptor ligands: | 0.113882241 | 0.718478377 |
| | Cannabinoid CB1 receptor | | |
| | agonis | | |
| Wiskostatin | Inhibitors: N-WASP inhibitor | −0.055664318 | 1.137050228 |
| Ro 20-1724 | Inhibitors: phosphodiesterase | 0.713112403 | 0.128156306 |
| | (PDE4 ) inhibitor | | |
| Diidolylmethane | Nuclear receptor ligands: AHR | 0.610453607 | 0.208326001 |
| | agonist | | |
| Cerulenin | Lipid biosynthesis: Fatty acid | 0.061695288 | 0.883132484 |
| | biosynthesis inhibitor | | |
| HBDDE | Kinase inhibitors: PKC | 1.213596337 | −0.042071031 |
| | inhibitor | | |
| Quinine HCl • 2H2O | Ion channel ligands: Potassium | 0.422484039 | 0.390749466 |
| | channels | | |
| swainsonine | nhibitors: protein glycosylation | 0.330258336 | 0.502315761 |
| | inhibitor | | |
| Tyrphostin 1 | Inhibitors: Calcineurin | 0.531665168 | 0.311857386 |
| | inhibitor | | |
| W7 | Inhibitors: calmodulin | 0.205579646 | 0.697926055 |
| | antagonist | | |
| Betulinic acid | Inhibitors: induces | 0.632557531 | 0.248630995 |
| | mitochondrial permeability | | |
| | pore opening | | |
| C2 Ceramide | Bioactive lipids: Apoptosis | 0.143702205 | 0.818539349 |
| | inducer | | |
| Methoxyverapimil • HCl | Ion channel ligands: Calcium | 0.572116091 | 0.299057976 |
| | channels | | |
| Flufenamic acid | Ion channel ligands: Potassium | 0.865473851 | 0.124111466 |
| | channels | | |
| Procainamide | Ion channel ligands: Sodium | 0.360169627 | 0.530424295 |
| | channels | | |
| Anandamide (22:4,n-6) | Endocannabinoids: | 0.756202763 | 0.205066195 |
| | Cannabinoid receptor agonist | | |
| OBAA | Inhibitors: phospholipase A2 | 0.17261678 | 0.821219271 |
| | inhibitor | | |
| C8 Ceramide | Bioactive lipids: Stimulates | 0.093600109 | 1.008494108 |
| | Cer-activated PK | | |
| 2,5- | Ion channel ligands: ER Ca++ | 0.897749195 | 0.144284654 |
| Ditertbutylhydroquinone | ATPase inhibitor | | |
| DL-PPMP | Lipid biosynthesis: | 0.479465854 | 0.428930389 |
| | Glucosylceramide synthase | | |
| | inhibitor | | |
| Dihydrosphingosine | Bioactive lipids: Apoptosis | 0.04571841 | 1.202287388 |
| | inducer | | |
| HAI 077 | Kinase inhibitors: inhibitor of | 0.548086085 | 0.414506741 |
| | Rho-dependent kinases | | |
| Palmitylethanolamide | Endocannabinoids: | −0.036033065 | 1.796165228 |
| | Cannabinoid CB2 receptor | | |
| | agonist | | |
| AG-490 | Kinase inhibitors: JAK2 | 0.368300767 | 0.613930446 |
| | inhibitor | | |
| PPI | Protease inhibitors: src family | 0.678320134 | 0.321502113 |
| | trosine kinase inhibitor | | |
| Calpeptin | Protease inhibitors: Calpain | 1.453486753 | 0.018644576 |
| | inhibitor | | |
| Cimaterol | CNS receptor ligands: | 0.338343906 | 0.659238733 |
| | adrenoceptor agonist (beta) | | |
| Cycloheximide-N- | Inhibitors: FKBP12 inhibitor | 0.081882681 | 1.20832405 |
| ethylethanoate | | | |
| PP2 | Kinase inhibitors: Src family | 0.742681791 | 0.29427215 |
| | tyrosine kinase inhibitor | | |
| GW-5074 | Kinase inhibitors: cRAF1 | 0.410236344 | 0.595679693 |
| | kinase inhibitor | | |
| Dibutyrylcyclic AMP | Activators: PKA activator | 0.187845893 | 1.03621908 |
| Glipizide | Ion channel ligands: Potassium | 0.381052549 | 0.6916373 |
| | channels | | |
| Ciglitazone | Bioactive lipids: PPAR delta | 0.98382165 | 0.22944822 |
| | agonist | | |
| Fipronil | Ion channel ligands: Mise, | 1.475052128 | 0.082613154 |
| | channels | | |
| Docosatrienoic acid (22:3 | Bioactive lipids: | 0.441017423 | 0.644039876 |
| n-3) | Polyunsaturated fatty acid | | |
| Decylubiquinone | Inhibitors: inhibits | 0.642268405 | 0.444211674 |
| | mitochondrial permeability | | |
| | pore opening | | |
| D609 | Lipid biosynthesis: PC-PLC | 0.355060251 | 0.768767767 |
| | inhibitor | | |
| YS035 | Ion channel ligands: Calcium | 0.709565967 | 0.407185249 |
| | channels | | |
| Quinidine • HCl • H2O | Ion channel ligands: Sodium | 1.363929705 | 0.12906881 |
| | channels | | |
| Juglone | Inhibitors: PIN1 inhibitor | 0.843026273 | 0.33083877 |
| KN-62 | Kinase inhibitors: CaM kinase | 0.283082214 | 0.936219415 |
| | II inhibitor | | |
| Ascomycin (FK-520) | Inhibitors: binds to FKBP | 1.137290473 | 0.215890497 |
| | inhibits calcineurin | | |
| Flunarizine • 2HCl | Ion channel ligands: Calcium | 0.42182209 | 0.744761844 |
| | channels | | |
| ML9 | Kinase inhibitors: kinase | 0.951183016 | 0.299571365 |
| | inhibitor | | |
| R)-(+)-BAY K-8644 | Ion channel ligands: Calcium | 0.940193089 | 0.309974846 |
| | channels | | |
| AA-861 | Lipid biosynthesis: 5- | 0.592385427 | 0.560256234 |
| | lipoxygenase inhibitor | | |
| LeukotoxinB (12,13- | Bioactive lipids: Bioactive | 0.178076963 | 1.32151963 |
| EODE) | linoleic acid metabolite | | |
| C16 Ceramide | Bioactive lipids: Activates | 0.986196963 | 0.300403628 |
| | PKC zeta | | |
| RWJ-60475-(AM)3 | Inhibitors: CD45 phosphatase | 0.18677847 | 1.380779076 |
| | inhibitor | | |
| ABC294640 | SK2 inhib. | 0.731119845 | 0.483576294 |
| 1-Octadecyl-2- | Bioactive lipids: Inhibits PI- | 0.617763099 | 0.595988803 |
| methylglycero-3 PC | specific PLC | | |
| R(+)-IAA-94 | Ion channel ligands: Mise, | 0.640162788 | 0.578908808 |
| | channels | | |
| SQ-29548 | Bioactive lipids: Thromboxane | 0.356660767 | 0.963984894 |
| | A2 antagonist | | |
| 1-Stearoyl-2-lineoyl- | Bioactive lipids: PKC activator | 0.170141729 | 1.5606247 |
| glycerol | | | |
| SU-4312 | Kinase inhibitors: VEGF-R | 0.894401344 | 0.401555037 |
| | (Flk-1) tyrosine kinase | | |
| 12-Methoxydodecanoic | Bioactive lipids: Myristic acid | 0.637937839 | 0.594614542 |
| acid | analog | | |
| Castanospermine | Inhibitors: glucosidase | 0.143488904 | 1.757869642 |
| | inhibitor | | |
| U-50488 | Ion channel ligands: Calcium | 0.323584517 | 1.108152437 |
| | channels | | |
| Ala-Ala-Phe-CMK | Protease inhibitors: Tripeptidyl | 1.365685146 | 0.25659919 |
| | peptidase II inhibitor | | |
| Tosyl-Phe-CMK (TPCK) | Protease inhibitors: Serine | 0.829239411 | 0.499871643 |
| | protease inhibitor | | |
| Tanshinone IIA | nhibitors: AP-1 inhibitor | 0.371351498 | 1.071587276 |
| Go6976 | Kinase inhibitors: PKC | 0.280063755 | 1.337845515 |
| | inhibitor | | |
| Lipoxin A4 | Bioactive lipids: Bioactive | 1.261639872 | 0.304411714 |
| | arachidonic acid metabolite | | |
| Shikonin | Inhibitors: Apoptosis inducer, | 0.228089572 | 1.574331422 |
| | p53 dependent | | |
| (S)-(−)-propranolol • HCl | CNS receptor ligands: | 0.899783847 | 0.473784189 |
| | adrenoceptor antagonist (beta) | | |
| Trifluoperazine | Inhibitors: calmodulin | 0.727494712 | 0.617743312 |
| | inhibitor-possibly only at high | | |
| | concentrations! | | |
| Misoprostol, free acid | Bioactive lipids: Prostaglandin | 0.73251658 | 0.625015982 |
| | EP receptor agonist | | |
| Monastrol | Inhibitors: Eg5 inhibitor | 0.529455325 | 0.862077937 |
| Monensin sodium | Ion channel ligands: Na+ | 0.557036024 | 0.833464141 |
| | ionophore | | |
| Prazocin | CNS receptor ligands: | 0.480929385 | 0.959144846 |
| | adrenoreceptor agonist | | |
| 8-Bromo-cAMP | Activators: PKA activator | 0.199482408 | 2.180715558 |
| Cyclopamine | Inhibitors: Hedgehog pathway | 0.522441705 | 0.942098476 |
| | inhibitor | | |
| 8-methoxymethyl-IBMX | Inhibitors: phosphodiesterase | 0.628093268 | 0.796887226 |
| | (PDE1) inhibitor | | |
| BW-B 70C | Lipid biosynthesis: 5 | 0.819066844 | 0.614747878 |
| | lipoxygenase inhibitor | | |
| A-23187 | Nuclear receptor ligands: | 1.013582567 | 0.503906761 |
| | PPAR gamma agonist | | |
| AG213 (Tyrphostin 47) | Kinase inhibitors: EGF-R | 0.583064627 | 0.920301211 |
| | tyrosine kinase inhibitor | | |
| Thiocitrulline [L- | Inhibitors: bNOS inhibitor | 0.532849437 | 1.002278889 |
| Thiocitrulline] | | | |
| 13,14-Dihydro-PGE1 | Bioactive lipids: Bioactive | 0.611667856 | 0.889676169 |
| | prostaglandin | | |
| 3,4-dichloroisocoumarin | Protease inhibitors: Granzyme | 0.345330654 | 1.548291881 |
| | B inhibitor | | |
| 2-methoxyantimycin A3 | Inhibitors: Bcl-2/Bcl-XL | 0.378591998 | 1.462164585 |
| | ligand induces apoptosis | | |
| CinnGEL 2Me | Inhibitors: PTP1B inhibitor | 0.603302283 | 0.946263688 |
| PRIMA-1 | Inhibitors: p53 reactivator | 0.631506774 | 0.921866952 |
| Parthenolide | Inhibitors: IkappaB kinase | 0.496333602 | 1.195407326 |
| | inhibitor | | |
| Bromo-7-nitroindazole | Inhibitors: NO synthase | 0.542540337 | 1.132112407 |
| | inhibitor | | |
| Bafilomycin A1 | Inhibitors: vaculolar ATPase | 0.34414636 | 1.894344631 |
| | inhibitor | | |
| Piceatannol | Kinase inhibitors: Syk | 0.413255655 | 1.537841143 |
| | inhibitor | | |
| U73122 | Lipid biosynthesis: PLC | 0.404489429 | 1.599661972 |
| | inhibitor | | |
| Bestatin | Protease inhibitors: | 0.957414033 | 0.672687601 |
| | Aminopeptidase inhibitor | | |
| (−)-Huperzine A | Inhibitors: acetylcholinesterase | 1.175656028 | 0.580883075 |
| | inhibitor | | |
| Histamine | CNS receptor ligands: | 1.284948591 | 0.534784124 |
| | Histamine receptor agonist | | |
| Flunarizine • 2HCl | Inhibitors: glutathione | 0.491144281 | 1.41078689 |
| | peroxidase mimetic | | |
| Nocodazole | Inhibitors: tubulin inhibitor | 1.308435715 | 0.531800759 |
| Cypermethrin | Inhibitors: calcineurin inhibitor | 0.768888171 | 0.930452865 |
| MDL-28170 | Protease inhibitors: Calpain | 0.598219729 | 1.214290536 |
| | inhibitor | | |
| ZM336372 | Protease inhibitors: Calpain | 0.6465571 | 1.124948408 |
| | inhibitor | | |
| (±)11(12)- | Bioactive lipids: Bioactive | 0.877557864 | 0.82851602 |
| Epoxy eicosatrienoic acid | arachidonic acid metabolite | | |
| Tamoxifen | Nuclear receptor ligands: | 0.750686051 | 0.977059527 |
| | estrogen antagonist | | |
| Methotrexate | CNS receptor ligands: DHFR | 1.222359082 | 0.611169777 |
| | inhibitor | | |
| 5,8,11-Eicosatriynoic acid | Bioactive lipids: Lipoxygenase | 1.07520212 | 0.707588935 |
| | inhibitor | | |
| Nimesulide | Lipid biosynthesis: Cox 2 | 0.743134155 | 1.024105253 |
| | inhibitor | | |
| Serotonin | CNS receptor ligands: | 0.836714967 | 0.926373517 |
| | serotonin receptor agonist | | |
| E6 berbamine | Inhibitors: calmodulin | 0.679846659 | 1.1789043 |
| | inhibitor | | |
| Leupeptin | Protease inhibitors: protease | 0.913893945 | 0.88734649 |
| | inhibitor | | |
| Prostaglandin F2a | Bioactive lipids: Prostaglandin | 0.954419924 | 0.852667163 |
| | FP receptor agonist | | |
| Docosahexaenoic | Bioactive lipids: | 1.066713184 | 0.781250842 |
| acid(22:6 n-3) | Polyunsaturated fatty acid | | |
| Aphidicolin | Inhibitors: DNA polymerase | 1.019136865 | 0.818533159 |
| | inhibitor | | |
| Cyclo [Arg-Gly-Asp-D- | Inhibitors: integrin inhibitor | 0.674795375 | 1.272716228 |
| Phe-Val] | | | |
| Leukotriene E4 | Bioactive lipids: CysLT1 | 1.060679099 | 0.833929516 |
| | receptor agonist | | |
| Prostaglandin E2 | Bioactive lipids: Prostaglandin | 0.693837262 | 1.301217809 |
| | EP receptor agonist | | |
| Indomethacin | Lipid biosynthesis: | 0.716892476 | 1.356958734 |
| | cyclooxygenase inhibitor | | |
| (±)-Epibatidine | CNS receptor ligands: | 1.862556821 | 0.584137558 |
| | nicotinic cholinergic agonist | | |
| Ouabain | Inhibitors: Na + K + ATPase | 1.300328161 | 0.76197975 |
| | inhibitor | | |
| Anandamide (20:3,n-6) | Endocannabinoids: | 0.609153582 | 2.0823477 |
| | Cannabinoid receptor agonist | | |
| Bumetanide | Ion channel ligands: Potassium | 1.388372276 | 0.783253392 |
| | channels | | |
| PCO-400 | Ion channel ligands: Potassium | 0.769881854 | 1.481393595 |
| | channels | | |
| Clofibrate | Nuclear receptor ligands: | 0.718430673 | 1.714865674 |
| | PPAR alpha agonist | | |
| Forskolin | Activators: Adenylate cyclase | 1.605749861 | 0.750451581 |
| | activator | | |
| 12(S)-HETE | Bioactive lipids: Bioactive | 0.966280623 | 1.194112095 |
| | arachidonic acid metabolite | | |
| Zardaverine | Inhibitors: phosphodiesterase | 0.732851173 | 1.770717348 |
| | (PDE1/2) inhibitor | | |
| Prostaglandin D2 | Bioactive lipids: Prostaglandin | 0.773483268 | 1.618494293 |
| | DP receptor agonist | | |
| NPPB | Ion channel ligands: Mise, | 0.882059822 | 1.477904919 |
| | channels | | |
| Aconitine | Ion channel ligands: Sodium | 1.129223092 | 1.169761206 |
| | channels | | |
| 25-Dihydroxyvitamin D3 | Nuclear receptor ligands: | 0.799793868 | 2.010291274 |
| | Vitamin D receptor ligand | | |
| Alrestatin | Inhibitors: aldose reductase | 0.830730845 | 2.015960074 |
| | inhibitor | | |
| E-64-d | Protease inhibitors: | 1.178262081 | 1.202555669 |
| | calpain/cathepsin inhibitor | | |
| CA-074-Me | Protease inhibitors: Cathepsin | 1.643869743 | 0.92587054 |
| | B inhibitor | | |
| Taxol = Paclitaxel | Inhibitors: microtubule | 1.487847478 | 1.061129071 |
| | stabilizer | | |
| (±)14,15-Epoxyeicosa- | Bioactive lipids: Bioactive | 1.340922848 | 1.193458842 |
| 5Z,8Z,l1Z- trienoic acid | arachidonic acid metabolite | | |
| 6-Gingerol | Ion channel ligands: | 1.124726981 | 1.459691953 |
| | Intracellular calcium | | |
| 5(S)-HPETE | Bioactive lipids: Fatty acid | 1.034353347 | 1.732189248 |
| | hydroperoxide | | |
| Vinblastine | Inhibitors: tubulin inhibitor | 1.075975466 | 1.712645652 |
| Roscovitine | Kinase inhibitors: CDK | 1.55274612 | 1.224456671 |
| | inhibitor | | |
| DL-Dihydrosphingosine | Lipid biosynthesis: | 1.975844602 | 1.252807234 |
| | Sphingosine kinase inhibitor | | |
| WIN 55,212-2 mesylate | CNS receptor ligands: | 1.287094225 | 1.960137321 |
| | Cannabinoid CB1/CB2 | | |
| | receptor agonist | | |
| Boc-GVV-CHO | Protease inhibitors: Gamma | 2.100909292 | 1.714851841 |
| | secretase inhibitor | | |

### Example 5: Reduced Ciliation in the Tsc2-Knockdown Neurons is Prevented by mTORC1 and Hsp90 Inhibition During an Age- and Time-Sensitive Window

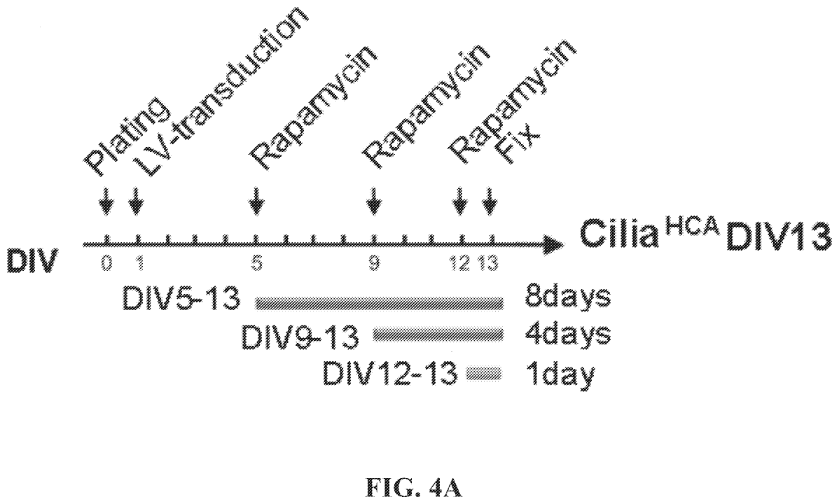

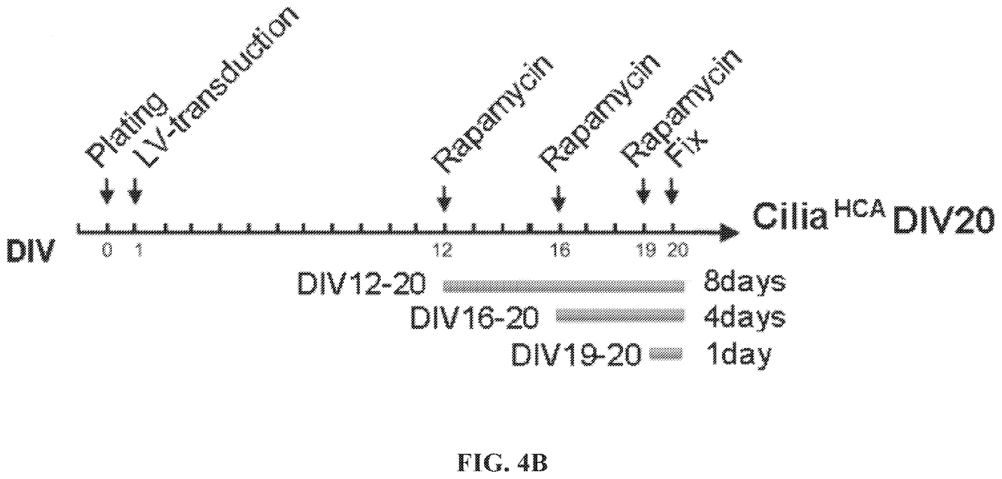

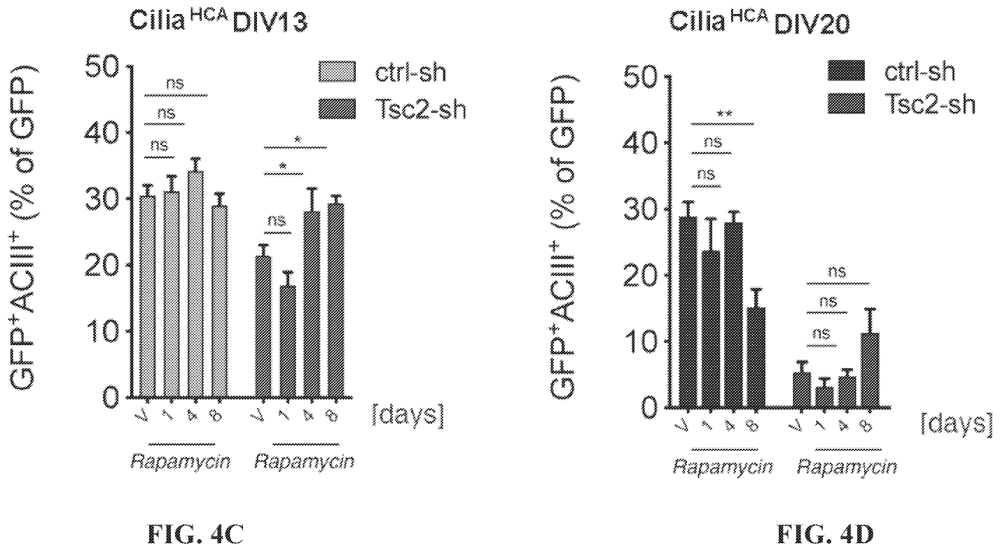

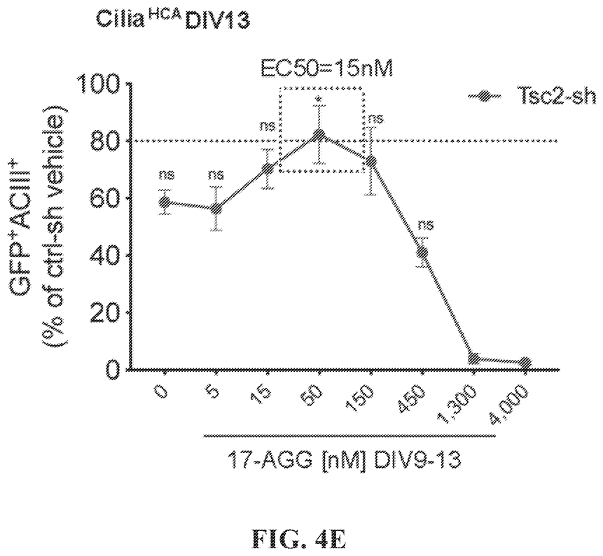

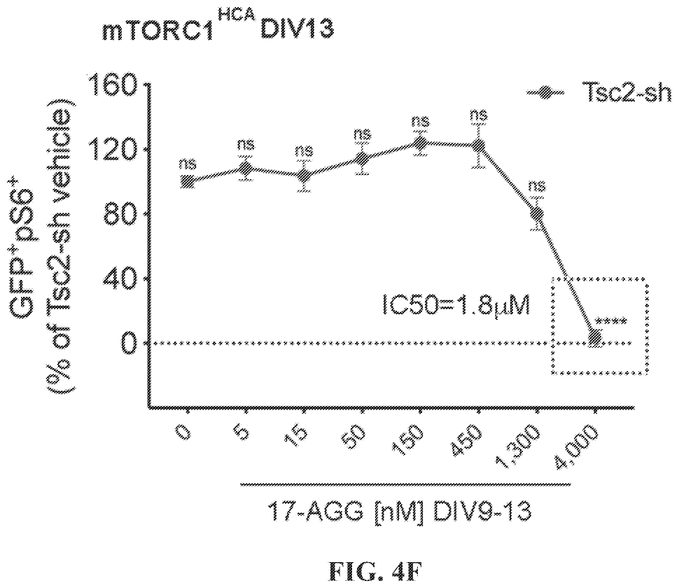

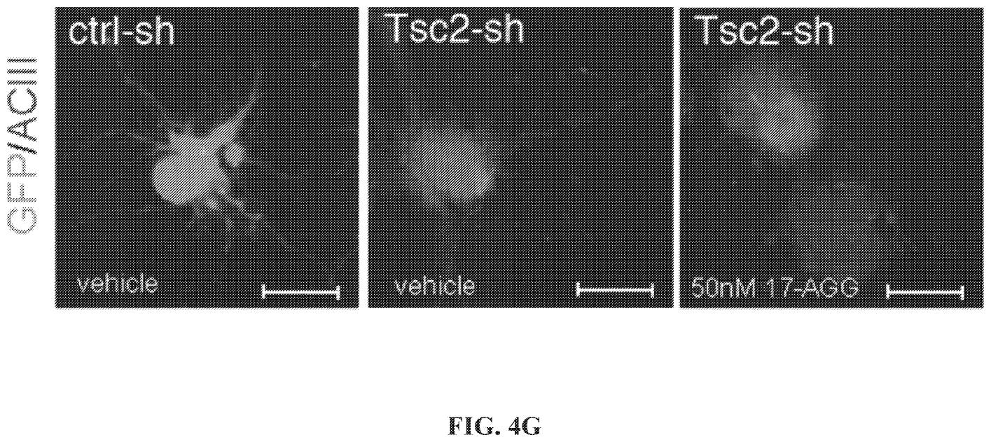

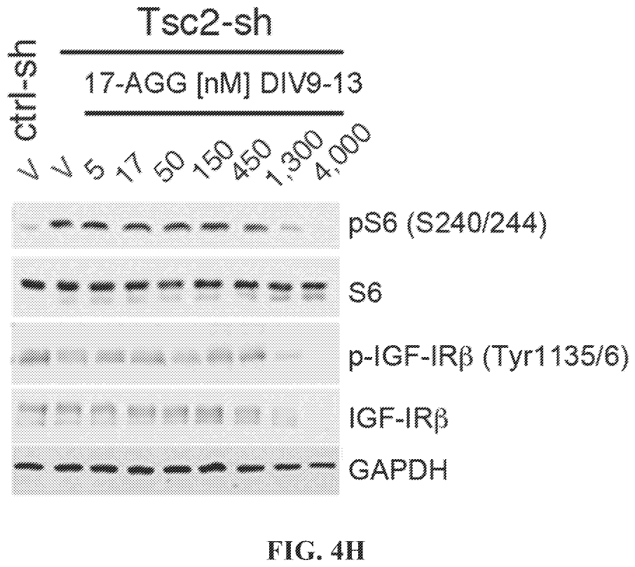

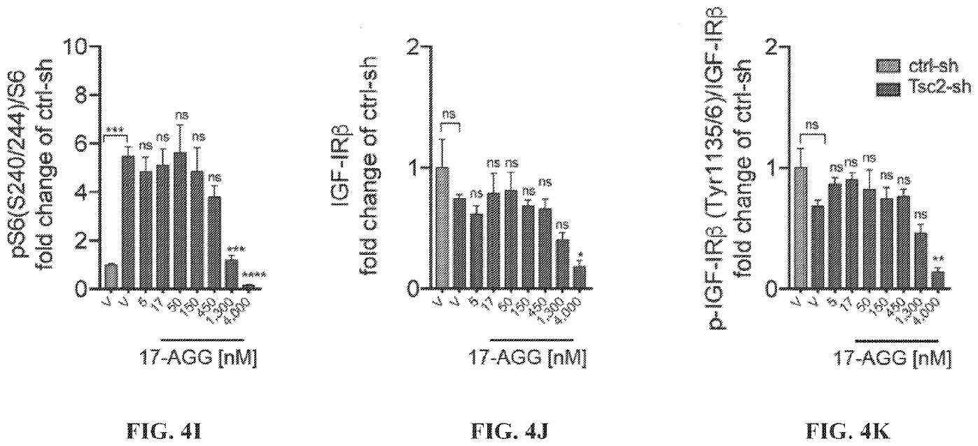

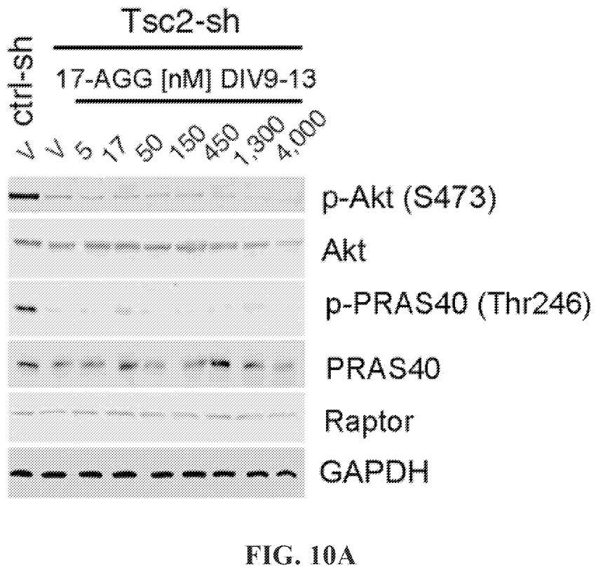

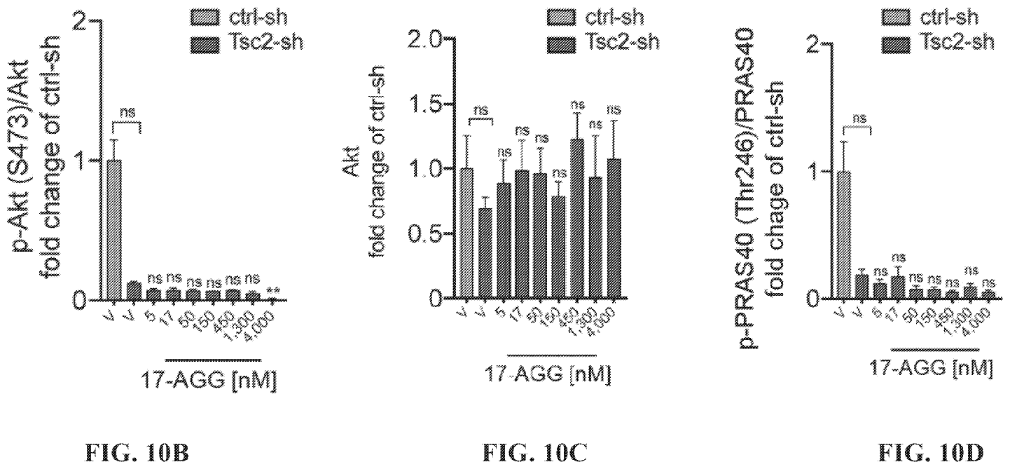

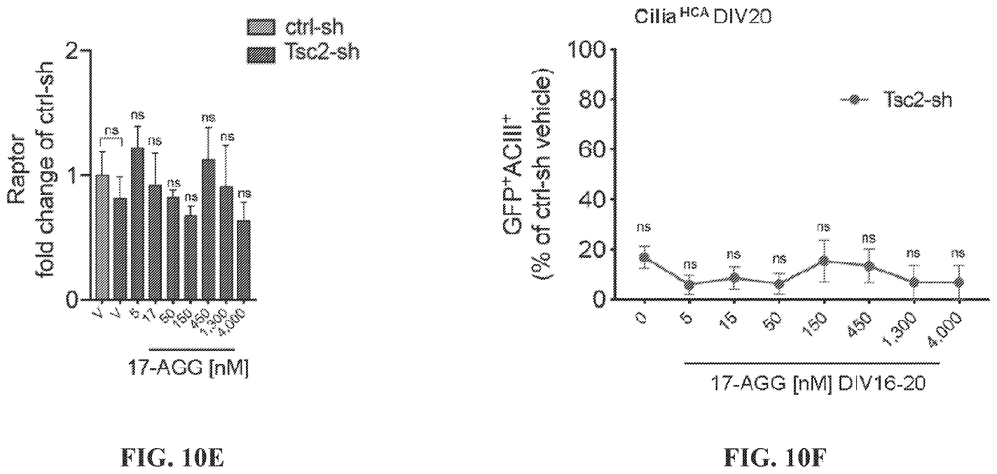

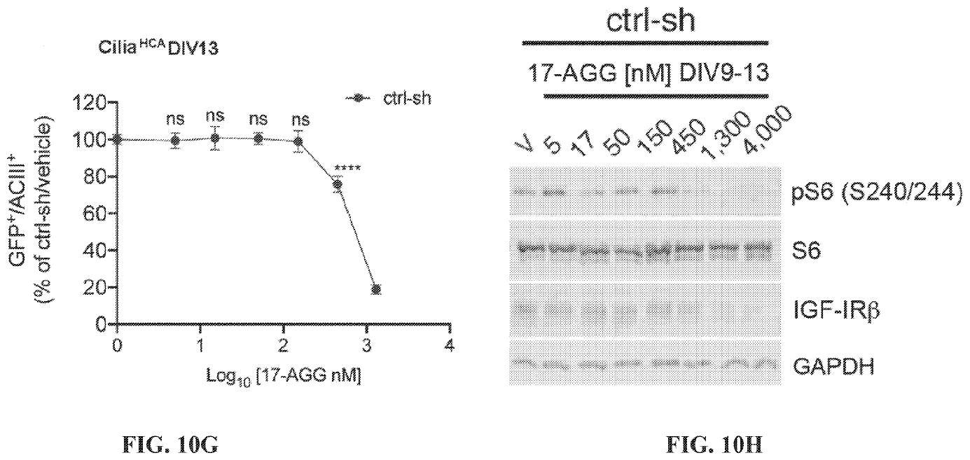

The acute effect of GA, 17-AGG and rapamycin on ciliation at concentrations that inhibited mTORC1-inhibition in Tsc2-knockdown neurons was investigated. Neither of the Hsp90 inhibitors nor rapamycin rescued ciliation under these conditions (FIG. 9M). Given that reduced ciliation was improved in the brain of Tsc1 and Tsc2 mouse models by fourteen and forty-nine days of rapamycin treatment, respectively (FIGS. 7A-C, FIGS. 1D-E), it was tested whether changing the timing or increasing the duration of rapamycin treatment in Tsc2-knockdown neurons might show an effect. Ciliation at DIV13 and DIV20 was assayed using both acute (1 day) and prolonged (4-8 days) rapamycin treatment (FIGS. 4A-D). A significant increase in ciliation of Tsc2-sh neurons treated with rapamycin for four or eight days (between DIV9-13 and DIV5-13) was observed, while no effect was seen after acute treatment or at later ages (DIV20) in culture (FIGS. 4C-D), suggesting that defective ciliation due to TSC1/2 loss can be prevented by early mTORC1 inhibition but is irreversible at later times. Based on these results, the potency of 17-AGG on ciliation and S6 phosphorylation between DIV9-13 was examined. Strikingly, 17-AGG was −100 times more potent at restoring ciliation than it was at inhibiting mTORC1 and IGF-IRβ/Akt signaling (Cilia rescue: EC50^{cilia}=15 nM; mTORC1 inhibition: IC50^{pS6}=1.80 μM) (FIGS. 4E-K, FIGS. 10A-E). Interestingly, 17-AGG did not increase ciliation in ctrl-sh neurons, suggesting that the mechanism of 17-AGG specifically prevents loss of cilia in Tsc2 knockdown neurons (FIGS. 10G-J). Neither rapamycin nor a four-day 17-AGG treatment regimen was able to rescue the cilia in Tsc2-sh neurons when tested at later ages in culture (FIG. 10F). These data show that mTORC1 hyper-activation can be suppressed by Hsp90 inhibition regardless of the age of the culture (assay endpoint respectively DIV13 and DIV20) while amelioration of the cilia phenotype only occurs during an early critical period (DIV9-13). In addition, these data show that 17-AGG likely prevents cilia loss via a distinct mechanism that is independent of inhibition of mTORC1.

### Example 6: Hsp27 Upregulation Due to Neuronal Tsc1/2 Loss is Reduced by 17-AGG and by Rapamycin in the Dose Range and within the Critical Window that Restores Ciliation

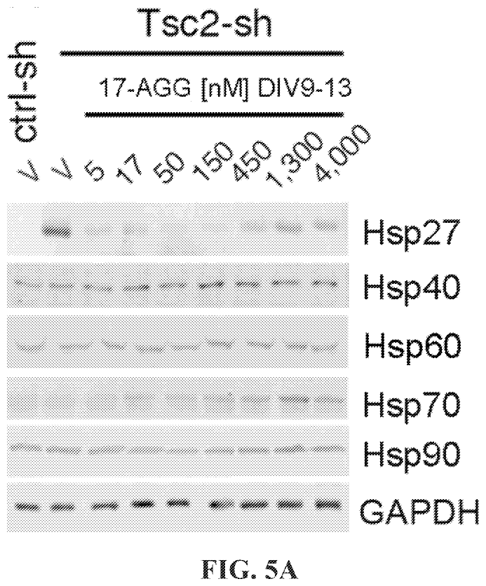

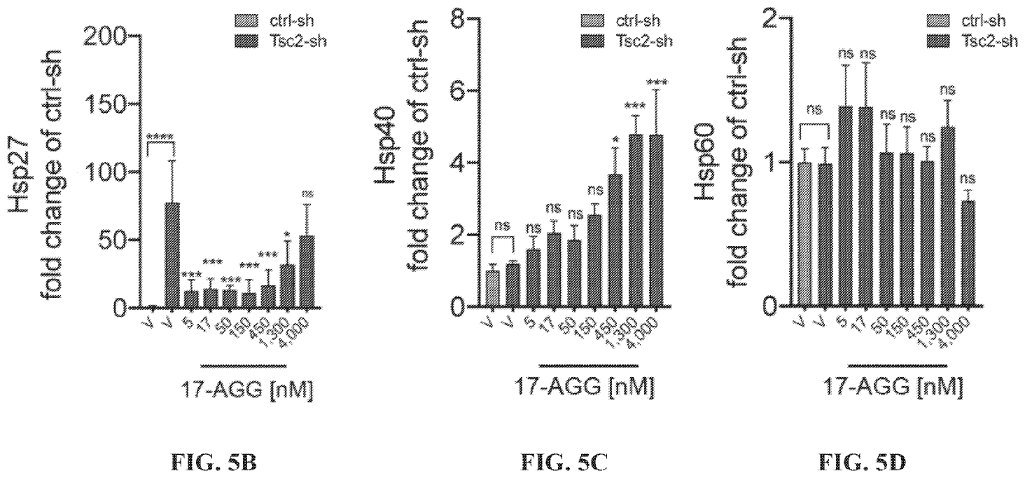

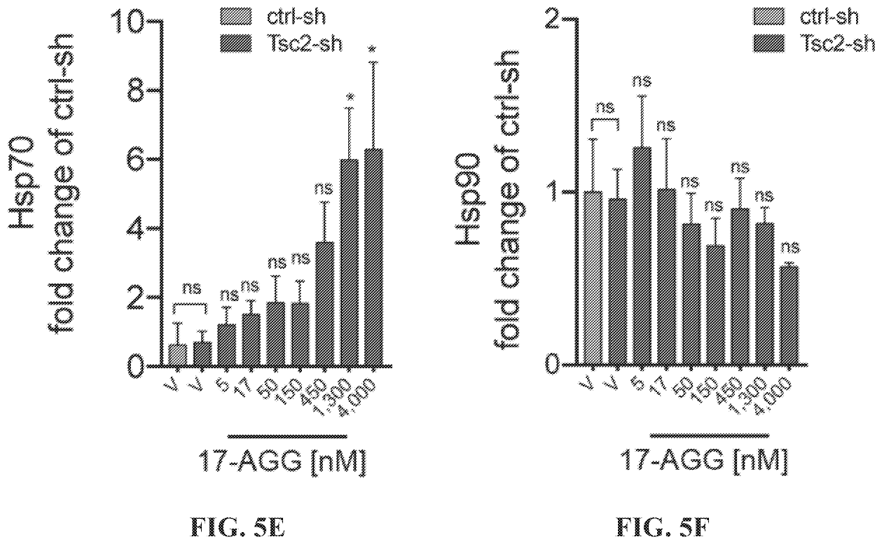

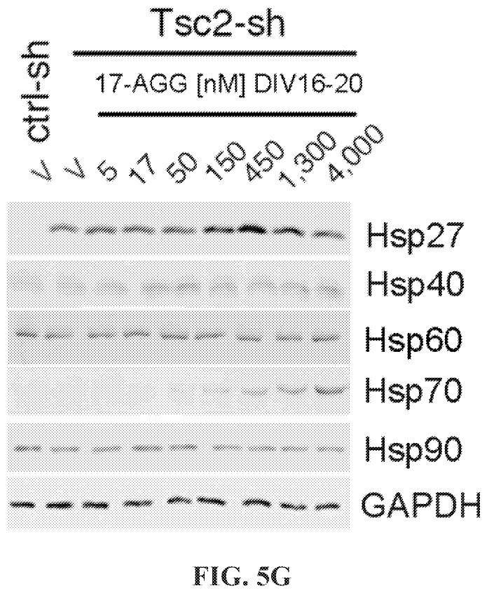

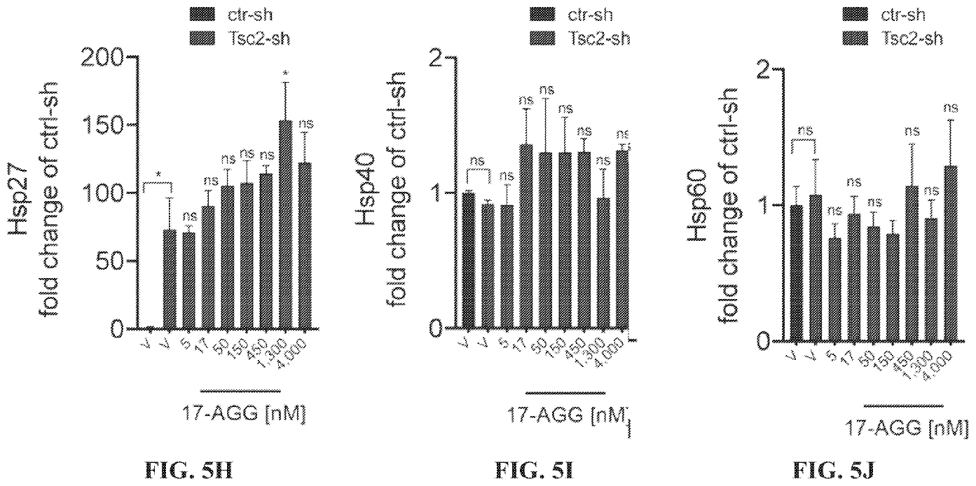

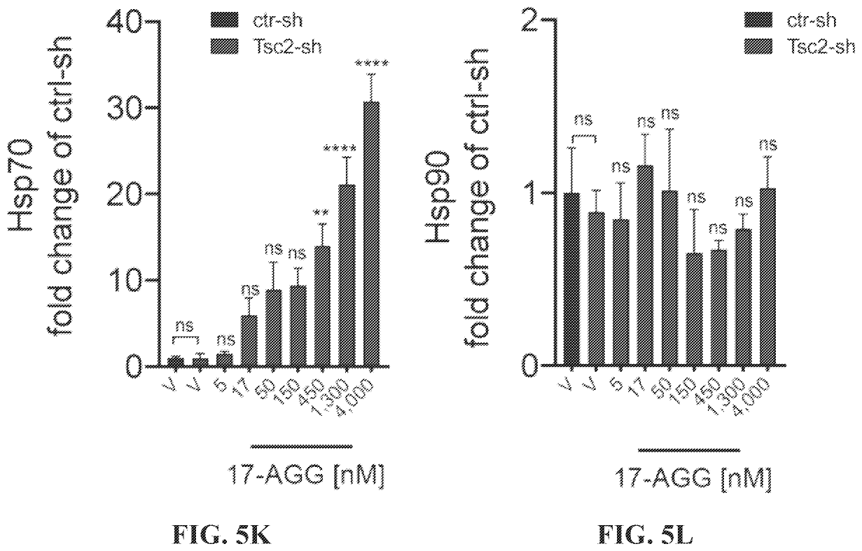

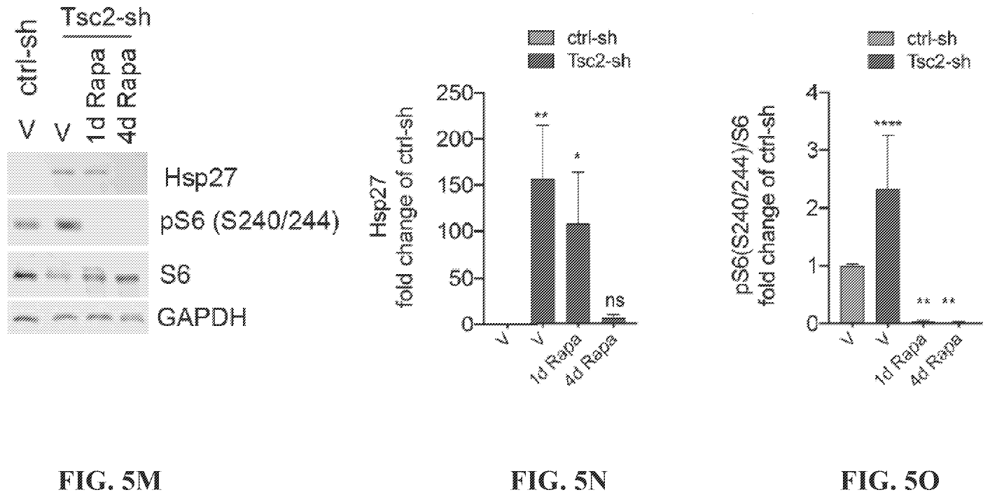

To investigate the mechanism and explore the pharmacological window by which 17-AGG increases ciliation in Tsc2-knockdown neurons, the heat shock response in these neurons was examined. The heat shock proteins (Hsps) are divided into six families based on their size, and these proteins function in multi-component complexes that are closely inter-related (Chatterjee and Burns, Targeting Heat Shock Proteins in Cancer: A Promising Therapeutic Approach; Int J Mol Sci 18 (2017)). In addition, while most of them are constitutively expressed, some are expressed only under stress (Garrido et al., Heat shock proteins 27 and 70: anti-apoptotic proteins with tumorigenic properties; Cell Cycle 5, 2592-2601 (2006)). Expression levels of Hsp90 and other HSP family members at DIV13 and DIV20 was assessed following four days of treatment with different concentrations of the Hsp90 inhibitor 17-AGG. In the absence of any compound, Tsc2-knockdown neurons had increased levels of the small heat shock protein 27 (Hsp27) with no change in Hsp90, Hsp70, Hsp60 and Hsp40 (FIGS. 5A-L). Remarkably, the aberrant Hsp27 expression was reduced in the Tsc2 knockdown cultures at DIV13 following four days of treatment with 17-AGG (ranging from 5-450 nM) at concentrations that were similar to that required to prevent cilia loss without any effect on mTORC1 activation (FIGS. 5A-B, FIGS. 4H-I). In addition, there was no change observed in Hsp27 expression due to treatment with 17-AGG in Tsc2 knockdown neurons at DIV20, consistent with the lack of effect on cilia at that age (FIGS. 5G-H, FIG. 10F). Inhibition of the Hsp90 pathway by 17-AGG was confirmed by induction of Hsp40 and Hsp70, consistent with the reported release of Hsp90-dependent inhibition on HSF1 (Zou et al., Repression of heat shock transcription factor HSF1 activation by HSP90 (HSP90 complex) that forms a stress-sensitive complex with HSF1; Cell 94, 471-480 (1998)) (FIGS. 5A, C, E, G, K). It was then evaluated whether Hsp27 upregulation was linked to mTORC1 hyper-activation. Acute (one day) and prolonged (four days) rapamycin treatment was performed in Tsc2-sh neurons and Hsp27 levels were measured at DIV13. Remarkably, Hsp27 upregulation was reduced by mTORC1 inhibition with rapamycin in the same pharmacological window (between DIV9-13) and dosing regimen that restored the cilia (FIGS. 5M-O). Together, these data indicate that increased expression Hsp27 is mTORC1-dependent and it can be down-regulated through either Hsp90 or mTORC1 inhibition using dosing regimen and concentrations that restores ciliation in Tsc2-deficient neurons. These findings indicate that Hsp27 contributes to the mTORC1-dependent mechanism implicated in defective ciliation under loss of neuronal TSC1/2 activity.

### Example 7: 17-AGG Improved Ciliation Downstream of mTORC1 Activation Through the Downregulation of HspB1 Gene Expression in the Tsc2 Knockdown Neurons

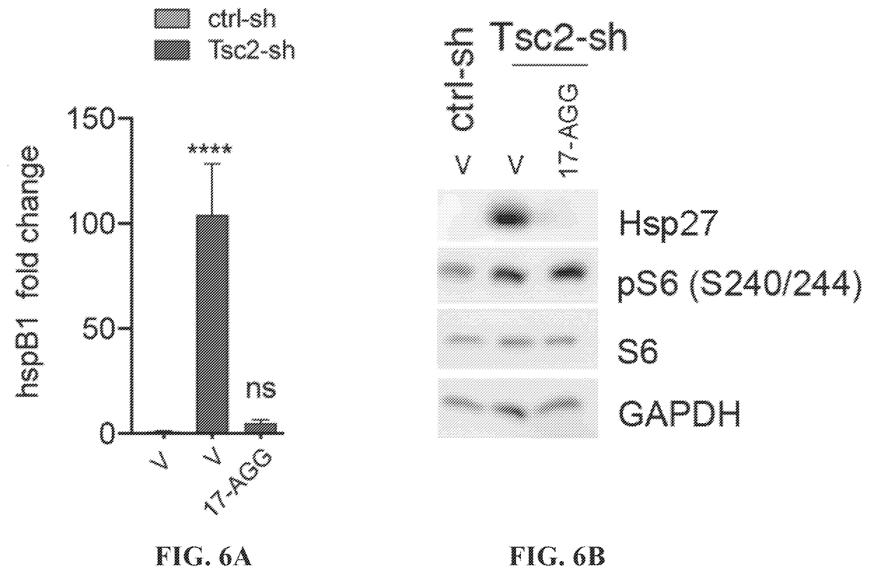

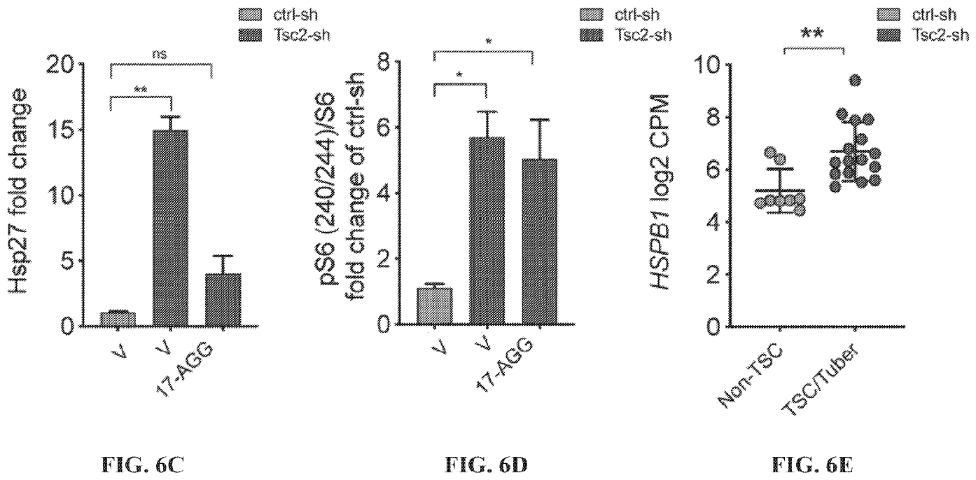

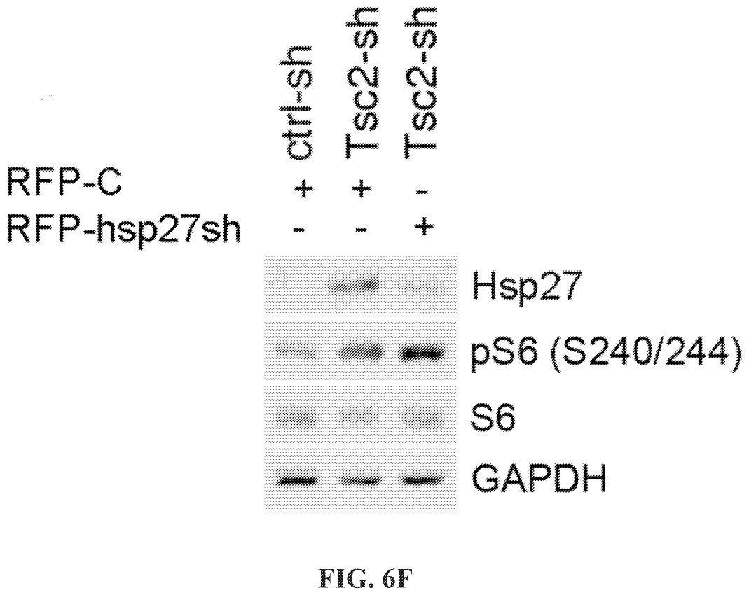

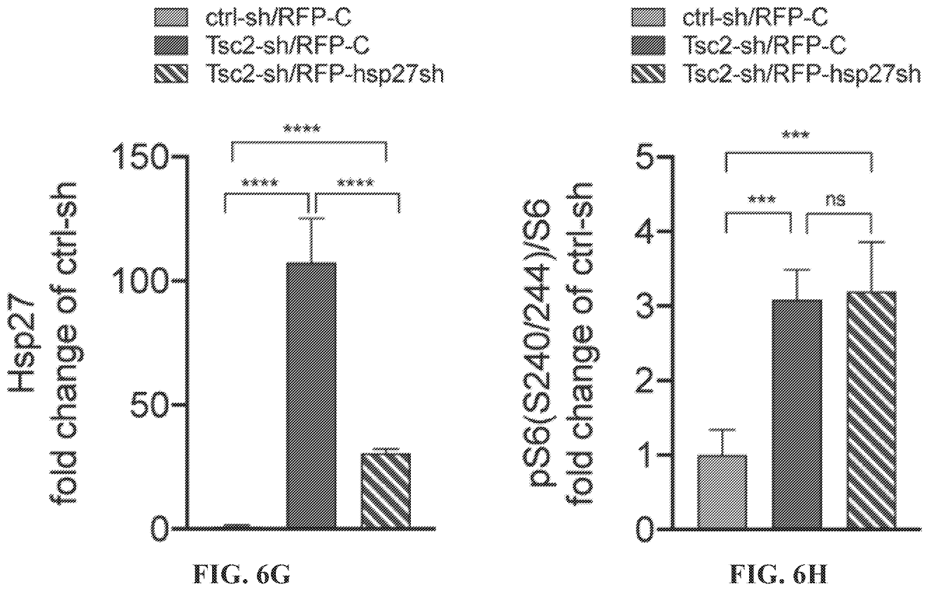

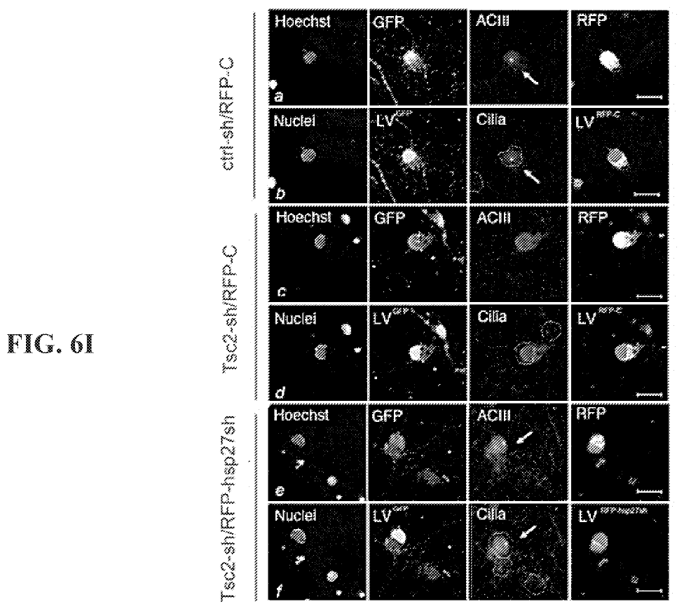

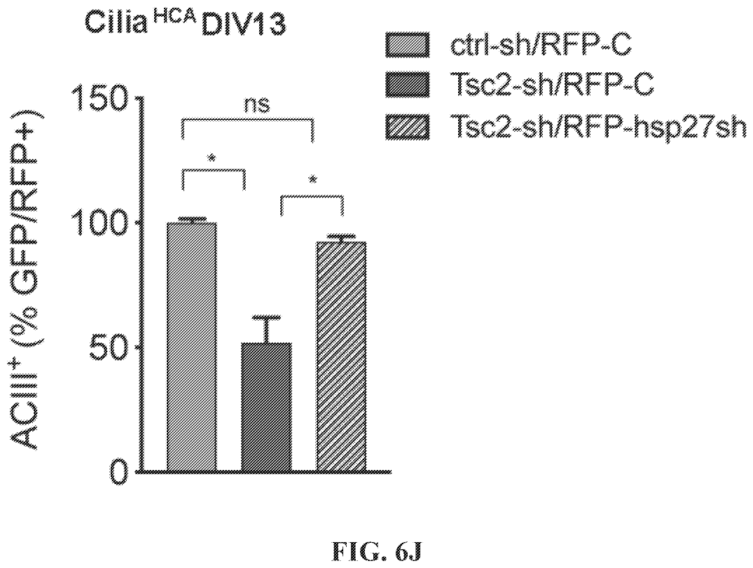

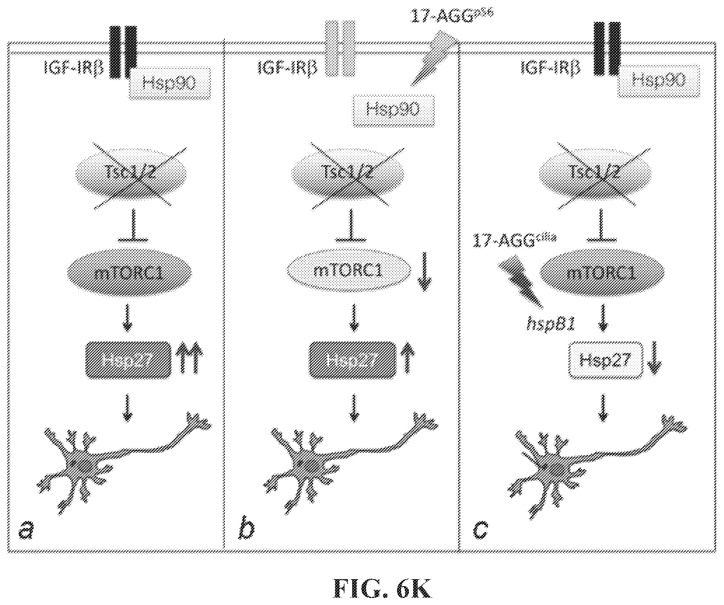

Tsc2 knockdown in neurons was found to increase the expression of hspB1, which encodes Hsp27 (Nie et al., The Stress-Induced Atf3-Gelsolin Cascade Underlies Dendritic Spine Deficits in Neuronal Models of Tuberous Sclerosis Complex; J Neurosci 35, 10762-10772 (2015)). Therefore, it was examined whether 17-AGG downregulated transcript levels of hspB1, leading to the decrease in Hsp27. Tsc2-sh neurons showed increased hspB1 expression (FIG. 6A). Tsc2-sh neurons were treated with 17-AGG and it was observed that increased hspB1 expression was reversed by the same dosing scheme that prevented cilia loss (50 nM between DIV9-13) (FIGS. 6A-D). To determine whether a similar dysregulation is present in the brains of TSC patients, the gene expression data from cortical tubers and unaffected brain tissue (Martin et al., The genomic landscape of tuberous sclerosis complex; Nat Commun 8, 15816 (2017)) was examined. Strikingly, HSPB1 expression was also significantly increased in cortical tubers (FIG. 6E).

To test whether Hsp27 down-regulation may contribute to restoring ciliation in Tsc2-deficient neurons, Hsp27 was knocked-down by hspB1 gene silencing using lentiviral expression of RFP-tagged Hsp27shRNA (RFP-Hsp27sh) or scrambled shRNA as a control (RFP-C). The levels of phosphorylated ribosomal protein S6 (pS6) were similar to Tsc2-deficient neurons transduced with a scrambled shRNA confirming that knockdown of Hsp27 in the Tsc2-knockdown cultures significantly reduced Hsp27 expression without affecting mTORC1 activation (FIGS. 6F-61I).

Ciliation in Tsc2-deficient neurons was examined with concomitant Hsp27 knockdown using the cilia^{HCA}. Tsc2-deficient neurons were identified based on the expression of GFP from the same vector as Tsc2 shRNA, and Hsp27 knockdown cells were identified based on the expression of RFP from the same vector as the Hsp27 shRNA. Remarkably, hspB1 knockdown resulted in a significant increase in ciliation in the Tsc2-deficient neurons (FIGS. 6I-6J). Neurons that had both Tsc2 and Hsp27 knockdown displayed similar levels of ciliation to control neurons that had been transduced with two non-targeting shRNA (FIGS. 6I-6J). These data identify Hsp27 as part of the signaling cascade downstream mTORC1 affecting cilia.

Taken together, multiple pharmacological effects of 17-AGG were identified that act at distinct nodes in TSC1/2-deficient neurons, blocking mTORC1 through the disinhibition of Hsp90-regulated degradation of PI3K/Akt signaling components and improving the cilia deficits with a 100-fold greater potency through the transcriptional downregulation of Hsp27 (model in FIG. 6K).

In this study, mTORC1 hyperactivation was shown to be caused by neuronal loss of Tsc1/2 leads to disruption of cilia, and a potential molecular mechanism was identified by which Hsp90 inhibition can reverse these pathological processes. Functional links observed between cilia and mTORC1 signaling appear to be critically dependent upon cellular context. TSC loss in kidney epithelial cells of zebrafish and mice results in longer cilia without affecting the number of ciliated cells (Armour et al., Cystogenesis and elongated primary cilia in Tsc1-deficient distal convoluted tubules; Am J Physiol Renal Physiol 303, F584-592 (2012); DiBella et al., Zebrafish Tsc1 reveals functional interactions between the cilium and the TOR pathway; Hum Mol Genet 18, 595-606 (2009)). In contrast, studies with Tsc1 or Tsc2 deficient MEFs showed a rapamycin-insensitive enhancement of cilia formation (Hartman et al., The tuberous sclerosis proteins regulate formation of the primary cilium via a rapamycin-insensitive and polycystin 1-independent pathway; Hum Mol Genet 18, 151-163 (2009)) or a different cilium phenotype with either longer or shorter cilia depending on the TSC gene affected (Rosengren et al., TSC1 and TSC2 regulate cilia length and canonical Hedgehog signaling via different mechanisms; Cell Mol Life Sci 75, 2663-2680 (2018)). One possible explanation for these divergent outcomes could be that ciliation is differentially regulated under specific cellular metabolic conditions. For instance, mTORC1-inducing stimuli can promote cilia disassembly through progression into mitotic phase in cycling cells (Yeh et al., IGF-1 activates a cilium-localized noncanonical Gbetagamma signaling pathway that regulates cell-cycle progression. Dev Cell 26, 358-368 (2013)). By contrast, mTORC1-inhibitory stimuli can promote ciliation through autophagy-dependent (Pampliega et al., Functional interaction between autophagy and ciliogenesis; Nature 502, 194-200 (2013); Tang et al., Autophagy promotes primary ciliogenesis by removing OFD1 from centriolar satellites; Nature 502, 254-257 (2013)) or autophagy-independent (Takahashi et al., Glucose deprivation induces primary cilium formation through mTORC1 inactivation; J Cell Sci 131 (2018)) mechanisms in dividing cells. Ciliary signaling has an established role in many developmental settings including cell cycle progression, proliferation, and differentiation (Kirschen and Xiong, Primary cilia as a novel horizon between neuron and environment; Neural Regen Res 12, 1225-1230 (2017)). Critical roles for cilia in the development of the CNS include roles in neuronal migration, neurogenesis, plasticity, and maturation. Interestingly, appearance of neuronal cilia coincides with onset of functional glutamatergic synaptic activity, which suggests that the protein machinery that functions in ciliogenesis might also be involved in synaptogenesis and that cilia may signal to the synapse (Kumamoto et al., A role for primary cilia in glutamatergic synaptic integration of adult-born neurons; Nature neuroscience 15, 399-405, S391 (2012)).

An inverse relationship was observed between ciliation and mTORC1 activity in hippocampal cultures at the time when neurons begin to polarize. This represents a time-sensitive regulatory role for the Tsc1/2 complex that may act as a brake on mTORC1 signaling to promote neuronal maturation through cilia assembly. Rapamycin has been shown to improve several neurological phenotypes in animal models of TSC, including epilepsy, cognition, and social behavior (Meikle et al., Response of a neuronal model of tuberous sclerosis to mammalian target of rapamycin (mTOR) inhibitors: effects on mTORC1 and Akt signaling lead to improved survival and function; J Neurosci 28, 5422-5432 (2008); Tsai et al., Autistic-like behaviour and cerebellar dysfunction in Purkinje cell Tsc1 mutant mice; Nature 488, 647-651 (2012); Tsai et al., Sensitive Periods for Cerebellar-Mediated Autistic-like Behaviors; Cell Rep 25, 357-367 e354 (2018)). The fact that rapamycin treatment also reversed the ciliary phenotype suggests that defective ciliary signaling might be contributing to these neurological symptoms.

Cortical tubers are a pathological hallmark of TSC characterized by the presence of immature giant cells and dysplastic neurons and are associated with disorganized connectivity and astrogliosis (Curatolo et al., Neurological and neuropsychiatric aspects of tuberous sclerosis complex; Lancet Neurol 14, 733-745 (2015)). Given the role of cilia in cell fate choice (Kim et al., Ndel-mediated inhibition of ciliogenesis affects cell cycle re-entry. Nature cell biology 13, 351-360 (2011); Li et al., Ciliary transition zone activation of phosphorylated Tctex-1 controls ciliary resorption, S-phase entry and fate of neural progenitors; Nat Cell Biol 13, 402-411 (2011); Yeh et al., IGF-1 activates a cilium-localized noncanonical Gbetagamma signaling pathway that regulates cell-cycle progression; Dev Cell 26, 358-368 (2013)), lack of ciliation could impact neuronal maturation and contribute to the development of the undifferentiated giant cells present in cortical tubers. Disorganized connectivity could also be a TSC-associated manifestation arising from alterations in cilia-dependent functions such as defective neuronal migration, polarization or cortical lamination (Park et al., Brain Somatic Mutations in MTOR Disrupt Neuronal Ciliogenesis, Leading to Focal Cortical Dyslamination; Neuron 99, 83-97 e87 (2018); Pruski and Lang, Primary Cilia—An Underexplored Topic in Major Mental Illness; Front Psychiatry 10, 104 (2019); Sarkisian and Guadiana, Influences of primary cilia on cortical morphogenesis and neuronal subtype maturation; Neuroscientist 21, 136-151 (2015)). Finally, given the role of cilia in the control of cell cycle and cell proliferation, altered cilia signaling might also have an impact in the transformation of the subependymal nodules into the low-grade subependymal giant cell astrocytoma (SEGAs) seen in the CNS of TSC patients (Alvarez-Satta and Matheu, Primary cilium and glioblastoma; Ther Adv Med Oncol 10, 1758835918801169 (2018); Chan et al., Pathogenesis of tuberous sclerosis subependymal giant cell astrocytomas: biallelic inactivation of TSC1 or TSC2 leads to mTOR activation; J Neuropathol Exp Neurol 63, 1236-1242 (2004); Ess et al., Expression profiling in tuberous sclerosis complex (TSC) knockout mouse astrocytes to characterize human TSC brain pathology; Glia 46, 28-40 (2004); Sarkisian and Semple-Rowland, Emerging Roles of Primary Cilia in Glioma; Front Cell Neurosci 13, 55 (2019)). Interestingly, studies have found that altered expression of genes associated with cilia is a risk factor for several neuropsychiatric disorders (Marley and von Zastrow, A simple cell-based assay reveals that diverse neuropsychiatric risk genes converge on primary cilia. PLoS One 7, e46647 (2012); Migliavacca et al., A Potential Contributory Role for Ciliary Dysfunction in the 16p11.2 600 kb BP4-BP5 Pathology; American journal of human genetics 96, 784-796 (2015)). Therefore, the observation that the expression of cilia genes is more likely to be altered in cortical tubers indicates that the ciliary dysfunction present in patients with TSC may contribute to the neuropsychiatric symptoms associated with TSC.

The phenotypic screen identified compounds that revealed that Hsp90 inhibition is capable of normalizing overactive mTORC1 and was associated with degradation of the RTK, mTORC1 controls RTKs such as IGF-IRf3 through negative-feedback, limiting their activation (Zhang et al., S6K1 regulates GSK3 under conditions of mTOR-dependent feedback inhibition of Akt; Molecular cell 24, 185-197 (2006)). However, RTK signaling can also be limited through ubiquitination, endocytosis, and degradation, and Hsp90 can regulate stability of IGF-IRβ as well as other oncogenic RTKs (Zsebik et al., Hsp90 inhibitor 17-AAG reduces ErbB2 levels and inhibits proliferation of the trastuzumab resistant breast tumor cell line JIMT-1; Immunol Lett 104, 146-155 (2006)). Hsp90 inhibition led to increased IGF-IRβ degradation in Tsc2-knockdown neurons. This reduction in IGF-IRβ combined with reduced activity due to mTOR hyperactivation leads to full suppression of Akt activity. Together these data indicate that further inhibition of upstream components of the PI3/Akt signaling pathway, such as the IGF-IRβ, contributes to reduce mTORC1 activity. Tsc2-knockdown neurons had increased Hsp27 expression at the transcriptional level and that knockdown of the hspB1 gene prevented the decrease in cilia due to loss of Tsc2, directly implicating Hsp27 as a downstream effector of mTORC1 hyperactivation in the disruption of cilia. Hsp27 is an ATP-independent chaperone expressed at low levels under physiological conditions that is induced by stress (Garrido et al., Heat shock proteins 27 and 70: anti-apoptotic proteins with tumorigenic properties; Cell Cycle 5, 2592-2601 (2006)). Misfolded proteins bind to small oligomers of Hsp27 that shift to large oligomers under stress. Therefore, elevated Hsp27 chaperone activity under pathological conditions has proliferative and anti-apoptotic functions. Disinhibition of mTORC1 signaling due to loss of neuronal Tsc1/2 causes oxidative and endoplasmic reticulum stress (Di Nardo et al., Tuberous sclerosis complex activity is required to control neuronal stress responses in an mTOR-dependent manner; J Neurosci 29, 5926-5937 (2009)). Thus, increased baseline hspB1 gene expression in the Tsc2-knockdown neurons might represent a compensatory stress-induced response to mTORC1-dependent accumulation of misfolded proteins and reactive oxygen species. In line with this, higher doses of 17-AGG may exacerbate the stress response, leading to persistent Hsp27 expression and lack of efficacy in preventing cilia loss. Interestingly, under stress conditions dynamic structural changes in Hsp27 oligomerization stabilize actin filament formation, which is a known inhibitor of ciliogenesis (Drummond et al., Actin polymerization controls cilia-mediated signaling; J Cell Biol 217, 3255-3266 (2018)). Therefore, Hsp27-dependent modulation of actin cytoskeleton could be part of the mechanism by which exaggerated Hsp27 expression inhibits cilia in the Tsc2-knockdown cultures. In addition, the fact that altered ciliation was rescued within a critical developmental period by pharmacological inhibition with 17-AGG through suppression of hspB1 suggests the existence of a distinct mechanism involving regulation of ciliogenesis at the transcriptional level (Choksi et al., Switching on cilia: transcriptional networks regulating ciliogenesis. Development 141, 1427-1441 (2014)) that can be prevented by early intervention but is irreversible at later times.

Several manifestations of TSC can be alleviated by mTOR inhibitors including rapamycin and its analog everolimus (Winden et al., Abnormal mTOR Activation in Autism; Annu Rev Neurosci 41, 1-23 (2018)); however, the beneficial effects are lost when the therapy is discontinued. Furthermore, many aspects of TSC, in particular neurocognitive deficits, are not reversed by mTOR inhibitors (Krueger et al., Everolimus for treatment of tuberous sclerosis complex-associated neuropsychiatric disorders; Ann Clin Transl Neurol 4, 877-887 (2017)), highlighting the need to identify alternative therapies. The mTORC1^{HCA} provided a valuable screening platform as it identified inhibition of Hsp90 with GA and 17-AGG as an alternative strategy to reverse disrupted mTORC1 signaling in TSC. In addition, the fact that the cilia^{HCA} uncovered 17-AGG as a compound capable of restoring defective ciliation in a time-sensitive window, independent of disrupted mTORC1 inhibition underscores the translational implication of the study. Together, the HCAs developed and optimized for high-throughput quantitation of mTORC1 and cilia with primary neurons represent broadly applicable platforms for compound screening and/or therapeutic testing of drug candidates.

### Example 8: Effect of HSP90 Inhibitors on Ciliation in TSC2-Deficient Neurons

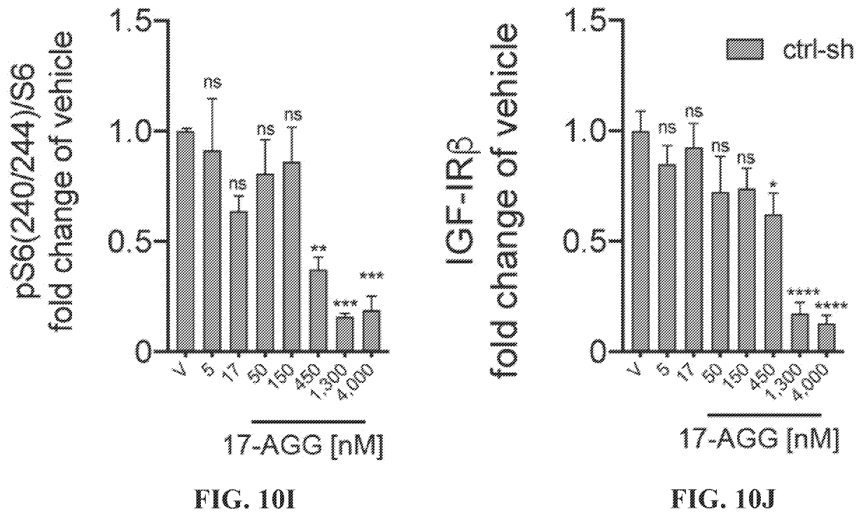

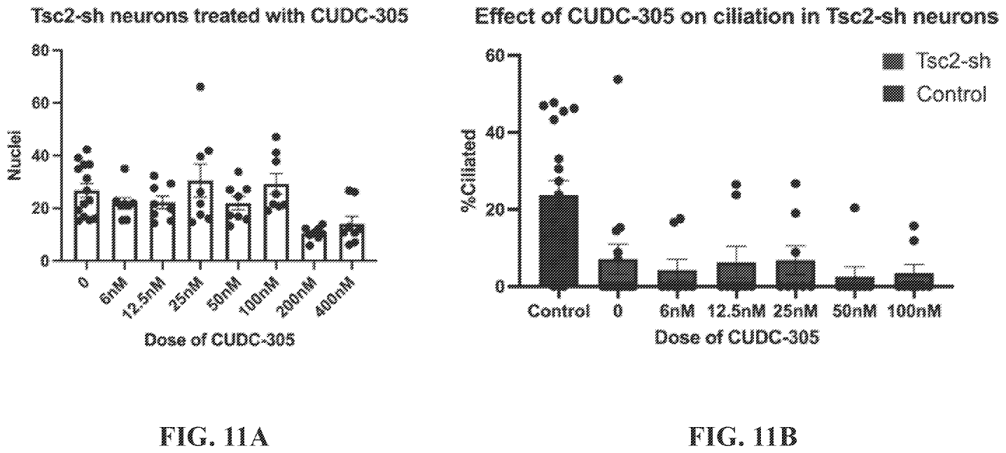

HSP90 inhibitors were tested to determine their effect on ciliation in TSC2-deficient neurons. Tsc2 was knocked down using an shRNA in primary rat neurons. The neurons were then treated with different doses of either CUDC-305 or NPV-HSP-990. To assess toxicity for CUDC-305, Tsc2-sh neurons were treated with increasing concentrations (6 nM, 12.5 nM, 25 nM, 50 nM, 100 nM, 200 nM, 400 nM) of CUDC-305 for four days (DIV9-13) and then stained for nuclei at DIV13 to determine the number of nuclei per field. At doses above 100 nM of CUDC-305, the number of nuclei began to reduce, which was suggestive of toxicity of the compound at those doses (FIG. 11A). The effect of ciliation of Tsc2-sh neurons after treatment with CUDC-305 was then examined. No increase in ciliation in Tsc2-sh neurons was observed at any dose of CUDC-305, suggesting that this compound was not capable of preventing reduced ciliation in Tsc2-sh neurons (FIG. 11B).

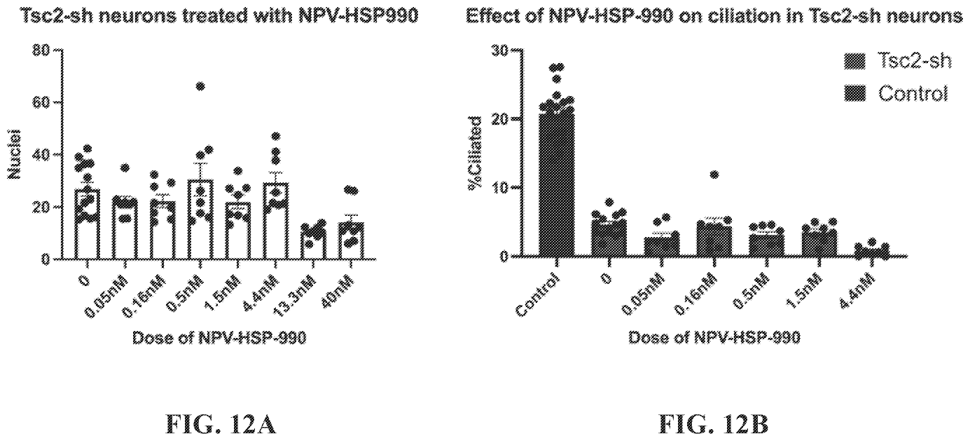

Another HSP90 inhibitor, NPV-HSP-990, was examined for its effect on ciliation in TSC2-deficient neurons. To assess toxicity for NPV-HSP-990, Tsc2-sh neurons were treated with increasing concentrations (0.05 nM, 0.16 nM, 0.5 nM, 1.5 nM, 4.4 nM, 13.3 nM, 40 nM) of NPV-HSP-990 for four days (DIV9-13) and then stained for nuclei at DIV 13 to determine the number of nuclei per field. At doses above 4.4 nM of NPV-HSP-990, the number of nuclei began to reduce, which was suggestive of toxicity of the compound at those doses (FIG. 12A), but a rescue in ciliation of Tsc2-sh neurons was not observed at any dose (FIG. 12B).

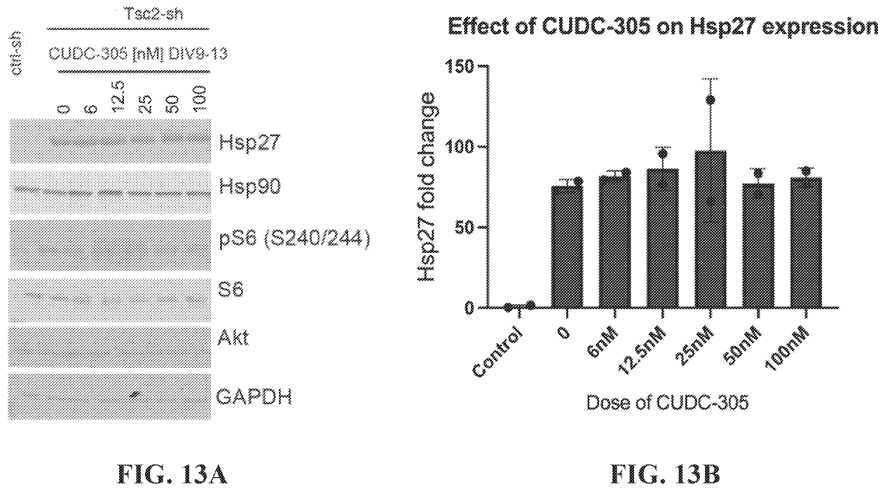

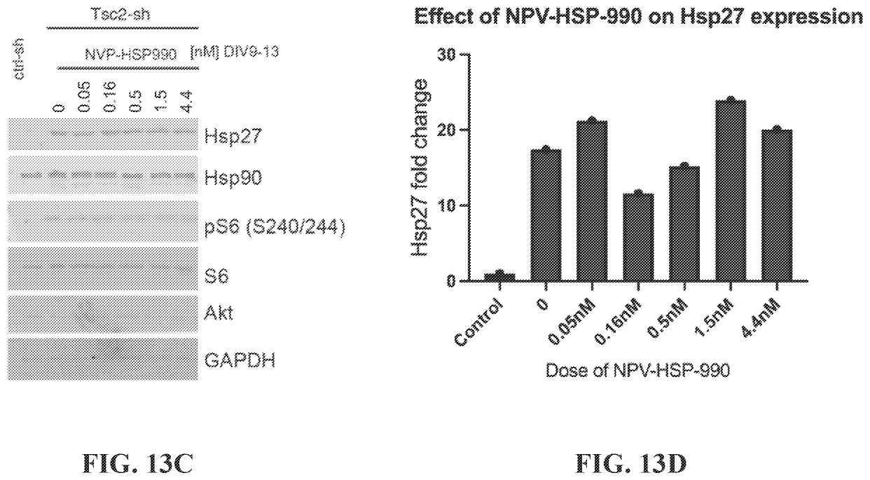

The effect of CUDC-305 and NPV-HSP-990 on Hsp27 expression was then tested. Neither compound had a significant effect on down-regulating Hsp27 expression in Tsc2-sh neurons (FIGS. 13A-13D). These data demonstrate that other HSP90 inhibitors are not capable of preventing cilia loss and reducing Hsp27 expression.

### Example 9: Characterizing Ciliation on TSC2-Deficient Human Neurons

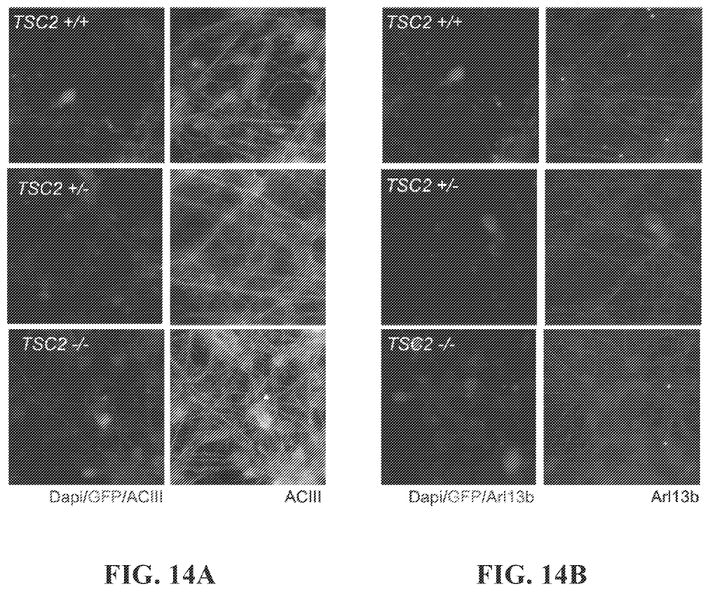

In order to determine whether reduced cilia are a phenotype of TSC2 deficient human neurons, iPSC-derived neurons were characterized from patients with mutations in TSC2. Three isogenic iPSC lines were used. The initial line was derived from a patient with TSC due to a heterozygous mutation in TSC2 (TSC2+/−). This line was further engineered to either have a mutation in the second TSC2 allele (TSC2−/−) or correct the original TSC2 mutation (TSC2+/+). Each of these lines have been previously demonstrated to be pluripotent and have a normal karyotype. To differentiate these cells into neurons, stem cells were transduced with a vector that expressed the transcription factor NGN2 under a doxycycline inducible promoter, which has been previously demonstrated to yield robust differentiation into excitatory cortical neurons. These neurons were fixed and immunostained for different cilia markers, including ACIII and Arl13b. No reliable staining of ACIII in any of the iPSC-derived neurons was observed (FIG. 14A). Arl13b staining was observed in iPSC-derived neurons of all three genotypes, which is depicted by bright punctae near several cell bodies consistent with labeling of cilia FIG. 14B). Together, these data suggest that the effect of 17-AGG on ciliation may be independent of its effects on HSP90.

The results obtained above were obtained using the following methods and materials.

### Animal Models

All experimental procedures were done in agreement with animal protocols approved by the Institutional Animal Care and Use Committee at Boston Children's Hospital. Both female and male mice were used in the experiments. Mice were maintained on a 12-h light/dark cycle with free access to food and water according to the Animal Research Committee at Boston Children's Hospital.

Tsc1 mutant mice: The Tsc1 control (Tsc1 w/w Syn^{Cre}) and Tsc1 mutant (Tsc1 c/c Syn^{Cre}) mice were described previously (Ercan et al., Neuronal CTGF/CCN2 negatively regulates myelination in a mouse model of tuberous sclerosis complex; The Journal of experimental medicine 214, 681-697 (2017)). All mice were in mixed background, derived from C57BL/6, CBA, and 129S4/SvJae, strains. The use of c and w was used to denote the conditional (foxed) and wild-type alleles of Tsc1, respectively; the formal name of the c allele is Tsc1^{tm1Djk} (Meikle et al., A mouse model of tuberous sclerosis: neuronal loss of Tsc1 causes dysplastic and ectopic neurons, reduced myelination, seizure activity, and limited survival; J Neurosci 27, 5546-5558 (2007)). To generate Tsc1 c/c Syn Cre⁺ mice, first Tsc1 c/w Syn Cre⁺ females were crossed with Tsc1 c/c Syn Cre⁻ male mice. Rapamycin treatment was performed by injecting 6 mg/kg intra-peritoneally every other day beginning at P7 until sacrifice (P21). These rapamycin treatment timing and dosing were chosen based on pharmacokinetics and pharmacodynamics of rapamycin in the brain (Meikle et al., Response of a neuronal model of tuberous sclerosis to mammalian target of rapamycin (mTOR) inhibitors: effects on mTORC1 and Akt signaling lead to improved survival and function; J Neurosci 28, 5422-5432 (2008)). Brain levels were above the level required to inhibit mTORC1 and effective in reversing the hypomyelination phenotype (Meikle et al., Response of a neuronal model of tuberous sclerosis to mammalian target of rapamycin (mTOR) inhibitors: effects on mTORC1 and Akt signaling lead to improved survival and function; J Neurosci 28, 5422-5432 (2008)).

Tsc2 mutant mice: the formal name of the c allele is Tsc1^{Tm2.1Djk} (Pollizzi et al., A hypomorphic allele of Tsc2 highlights the role of TSC1/TSC2 in signaling to AKT and models mild human TSC2 alleles; Hum Mol Genet 18, 2378-2387 (2009)). Litters were generated from crosses between mixed background animals containing Tsc2 k/c Syn Cre⁻ and Tsc2 c/c Syn Cre⁺ in which the k allele was a full knockout of the Tsc2 gene and the c allele was a conditional mutation of the Tsc2 gene that results in −7% expression of TSC2. All pups in a litter were treated with either vehicle or rapamycin and animals of the genotype Tsc2 c/c Syn Cre⁻ were used as control mice and Tsc2 k/c Syn Cre+ were used as mutant mice. For rapamycin treatment, animals were dosed every other day beginning at P7 with 3 mg/kg rapamycin in vehicle at a volume of 30 μl until P21 and then at a total volume of 100 μl from P21 to P56. Vehicle consisted of 5% PEG 400 and 5% Tween 80; 4% ethanol was added to the vehicle for control treated animals.

### Human Subjects

Cortical tubers were collected from patients clinically and neuropathologically diagnosed with TSC, at the time of surgery. Tissues were fixed in 4% phosphate-buffered paraformaldehyde pH 7.4 (PFA), subjected to sucrose gradient and stored frozen before further processing. The control samples were prepared in a similar fashion and were processed together. All patients suffered from chronic epilepsy, with a seizure history. See Table 2A and Table 2B for details. The subjects enrolled in this study were recruited through Boston Children's Hospital, and the protocol was approved by Boston Children's Hospital IRB (P0008224). IRB protocol number for the Repository Core was CHERP 09-02-0043. Informed consents were obtained from all participants and/or their parents as appropriate.

### Neuronal Cultures

Hippocampi and cortexes from 18-day-old rat embryos (Charles River CD1) were isolated under the microscope and collected in Hank's Balanced Salt Solution containing 10 mM MgCl₂, 1 mM kynurenic acid, 10 mM HEPES and penicillin/streptomycin. After 5 min dissociation at 37° C. in 30 U/ml of papain, neurons were mechanically triturated and plated in Neurobasal (NB) medium containing B27 supplement, 2 mM L-glutamine, penicillin/streptomycin and primocin (NB/B27). Biochemical analysis was performed on cortical cultures plated at 1×10⁶ cells/well onto six-well plates. Immunofluorescent analysis was performed on hippocampal cultures plated at 20×10³ cells/well onto 96-well plates. All plates were coated with 20 μg/ml poly-D-lysine (PDL).

### Lentivirus Production and Transduction

Viral stocks for lentiviral infection were prepared by co-transfection of the two packaging plasmids psPAX2 and pMD2.G into HEK293T cells with the plasmid to be co-expressed using PEI. Viral particles were collected 48 hrs and 72 hrs after transfection and filtered through a 0.45 μm membrane. Hippocampal neurons were infected at 1 day in vitro (1 DIV) in the presence of polybrene at 0.6 μg/ml. Six hours after infection, the virus-containing medium was replaced by fresh NB/B27 medium. GFP-tagged control shRNA construct (here referred as ctrl-sh) against the luciferase gene was use as previously described (Flavell et al., Activity-dependent regulation of MEF2 transcription factors suppresses excitatory synapse number; Science 311, 1008-1012 (2006)). The lentiviral Tsc2 shRNA construct was used as previously described (Di Nardo et al., Tuberous sclerosis complex activity is required to control neuronal stress responses in an mTOR-dependent manner; J Neurosci 29, 5926-5937 (2009)). The target sequence for the Tsc2 gene was the following: 5′-GGTGAAGAGAGCCGTATCACA-3′. RFP-tagged Hsp27-shRNA (referred here as RFP-Hsp27sh) was purchased from Sigma; pLKO-RFP-shControl (referred here as RFP-C) was purchased from Addgene.

RNA Preparation and Quantitative Real Time PCR (qPCR)

Total RNA was prepared with the RNA kit (Zimo Research) following the instructions of the manufacturer and quantified by a spectrophotometer. A total of 1 μg of poly(A) mRNA was used for reverse transcription using the Reverse Transcriptase (BIO-RAD). Real-time PCRs were performed using Power SYBG Green PCR Master Mix (Applied Biosystems). All quantitative PCR (qPCR) reactions were performed in triplicate and normalized against glyceraldehyde-3-phosphate dehydrogenase (GAPDH). Analysis was performed using QuantStudio™ 3 Real-Time PCR System (Thermo Fisher Scientific). The qPCR cycle was: 95° C. for 10 min followed by 40 cycles at 95° C. for 15 sec and 60° C. for 1 min.

### Immunohistochemistry of Brain Tissue and Manual Cilia Counting

For histological analysis, animals were anesthetized (1 ml of 2.5% Avertin) and perfused transcardially with 4% PFA. Brains were dissected and fixed in 4% PFA for another 24 hours. Brains were slowly frozen by cooling down in dry-ice cold isopentane and then prepared by cryostat sectioning at a thickness of 25 μm. Sections were washed 4 times with Tris Buffered Saline pH 7.4 (TBS) and blocked for 2 hours at room temperature in blocking buffer (5% BSA, 0.1% Triton X-100, 10% goat serum). The incubation with the primary antibody was done in 1% BSA, 0.1% Triton X-100, at 4° C. for overnight. The day after, sections were washed three times in TBS buffer before being incubated with the appropriate fluorochrome-coupled secondary antibody. Stained sections were air-dried, dehydrated and mounted. Imaging of the Tsc1 control and mutant brains was performed by dividing the cortex into 6 layers and by imaging 5 random regions per layer. Imaging of the Tsc2 control and mutant brains was performed by imaging 6-9 random regions in the CA1 of the hippocampus. Finally, the average percentage of neurons with cilia (NeuN⁺/ACIII⁺) was calculated in each of the images. For the human tissue, an average of 40-60 images were acquired in random fields of the specimen. The average percentage of SMI311⁺ or SMI311⁻ with cilia was calculated. The measurement of the cilia length was performed by tracing the ACIII stained cilia using the ImageJ Software freehand tool. A threshold for cilia count was set such that only ACIII positive objects that measured longer than 1 μm were counted as cilia. All the imaging and the quantification were done in a blinded way. Confocal images were acquired with a Nikon Ultraview Vox Spinning Disk Confocal microscope using 63×oil-immersion objective equipped with Hamamatsu camera. All quantifications were performed in a blinded fashion.

### Drug Treatments

Stocks of drugs were freshly diluted in NB media before performing each experiment. The same amount of vehicle was used as vehicle-only control. For dose response curves serial dilutions were freshly made in NB media by a manual multichannel pipette using compound dilution plates. Proteasomal inhibition experiments were performed by pretreatment with 100 nM bortezomib (BTZ) for 6 hrs before incubation with 17-AGG at 404 for 24 hrs. The BIOMOL Collection Library used for the screen was obtained by the ICCB-Longwood Screening Facility. According to Biomol, all of the compounds in this collection have known and well-characterized bioactivities and have undergone safety and bioavailability testing. The compounds were carefully selected to maximize chemical and pharmacological diversity.

### Western Blot

Protein extracts were lysed in 1×SDS (22 mM Tris-HCl pH 6.8, 4% Glycerol, 0.8% SDS, 1.6% β-mercaptoethanol, bromphenol blue) sample buffer, heated to 95° C. for 5 min and stored frozen. Before being subjected to discontinuous gel electrophoresis, equal amounts of all protein lysates were verified by Coomassie gel staining. For immunoblotting, equal amounts of protein lysates were subjected to SDS-PAGE, transferred to Immobilon-P Millipore and incubated in LI-COR blocking solution at RT for 2 hrs. Primary and secondary antibodies were diluted in LI-COR blocking solution to the appropriate concentrations. LI-COR IRDye secondary antibodies were used. All images were acquired using the LI-COR Odyssey Classic imager and associated Image Studio Lite analysis software (version 5.2.5). Quantification of protein expression was performed by densitometry scans of immunoblots using LI-COR Odyssey imaging system.

### Neuronal Culturing and Processing for High-Content Assays

Rat hippocampal neurons were plated at a density of 20,000/well in 96-well plates (Greiner #655090) coated with Poly-D-Lysine (PDL) at 20 μg/ml. Neurons were transduced with lentivirus and processed for immunofluorescent staining at endpoint assay by fixation with 4% PFA followed by permeabilization in ice cold 100% Methanol. Neurons were then washed 3 times in PBS/0.05% Tween (PBT) containing 50 mM glycine and blocked in 2% bovine serum albumin (Sigma) in PBT (PBT/Block) at RT. For consistent and robust identification of the LV-infected neurons, cultures of both the cilia^{HCA} and mTORC1^{HCA} were stained with GFP antibody and co-labeled with ACIII or pS6 for the identification of respectively cilia and mTORC1 activity. Primary antibodies were incubated in PBT/Block overnight. The following day, neurons were washed in PBT and incubated with secondary antibodies followed by Hoechst staining. After washing with PBT, neurons were kept in PBS buffer. Aside from primary and secondary antibody administration, all the washing for immunofluorescent staining was done using the Agilent bravo automated liquid handler.

### Neuronal Culturing and Processing for Manual Cilia Counting

For manual cilia counting rat hippocampal neurons were plated at a density of 150,000/well onto coverslips coated with Poly-D-Lysine (PDL) at 20 μg/ml. Neuronal culturing and processed was performed. Manual imaging and cilia counting were performed in ctrl-sh and Tsc2-sh cultures stained with GFP antibodies to identify the LV-infected neurons, co-labeled with ACIII and centrin antibodies to identify cilia and basal bodies, respectively. Secondary antibodies goat anti-chicken alexa Fluor 488, goat anti-rabbit alexa Fluor 595 and goat anti-mouse alexa Fluor 647 were used. Coverslips were mounted on glass slides in Vectashield with DAPI (Vector Laboratories). Random images of GFP⁺ cells were obtained using an Eclipse Ti-E (Nikon) microscope with a Plan Apo 100×1.49 NA oil objective, an Evolve electron-multiplying charge-coupled device camera (Photometrics), and MetaMorph software (Molecular Devices). Images were acquired as a z series (0.2-μm z interval) and are presented as maximum-intensity projections. Cilia length was measured in maximum-intensity projections using ImageJ Software (Feret measurement of ROI identifying cilia). The same ROI was used to determine the total intensity of ACIII from the sum projection of the z-stacks. ACIII total intensity was then normalized to the corresponding cilium length. All quantifications were performed in a blinded fashion.

High-Throughput Imaging and Quantification of Cilia and of mTORC1

Neurons were imaged using the high content analysis platform Cellomics Arrayscan XTI available at the Human Neuron Core of the Translational Neuroscience Center (Boston Children's Hospital). The Arrayscan XTI was equipped with Zeiss optics, a 7-color solid state LED light engine, and a large format X1 CCD camera (2208×2208). The DAPI channel was used for focal plane acquisition by the software with an intra-well autofocus interval of one (refocus in each subsequent well). When needed, focal planes were adjusted to the best optimal resolution for each channel. Once optimized, Z offsets were kept the same throughout the scans. Imaging of the cilia^{HCA} was done with a 40× objective on eighty fields of view per well (20% of the well). The nuclei were detected by Hoechst staining at 386 nm emission for 30 ms, the LV-transduced neurons were detected using GFP staining at 485 nm emission for 20 ms at a 1.9 μm Z offset above the focal plane of the nuclei, and cilia were detected using ACIII staining at 647 emission for 130 ms using the “image projection tool” which allowed the acquisition of a stack of images at three different focal planes for optimal cilia imaging with a step size of 1.9 μm/step for a total of 5.7 μm (FIGS. 8C-D). Object selection for GFP and ACIII spots identification were filtered using area and shape measurements.

Imaging of mTORC1^{HCA} was performed with a 10× objective on the whole well. The nuclei were detected by Hoechst staining at 386 nm emission for 70 ms, the LV-transduced neurons were detected using GFP staining at 485 nm emission for 50 ms at a 11.7 μm Z offset above the nuclei focal plane, and mTORC1 activity was detected using pS6 staining at 546 emission for 40 ms (FIGS. 8E-F).

Data analysis was performed using an optimized version of Spot Detector algorithm available with the HCS Studio™ Cell Analysis Software which allowed the detection of subcellular structures and their co-localization. Nuclei identification was done using a circular mask and the identified objects functioned as a region of interest (ROI) for the subsequent channels. GFP⁺ spots were identified using a circular mask of the size of the nuclei, pS6+ spots were identified using a ring mask with a width of 8 μm outside the nuclei ROI, ACIII⁻ spots were identified using a circular mask with a diameter of 32 μm which included the nuclei ROI. Object selection for GFP and pS6 spots were filtered using area, and intensity measurements. Objects at the border of the well were always excluded.

mTORC1 High-Content Screen

A screen was performed using the mTORC1^{HCA}. Assay robustness and screening window between positive (ctrl-sh neurons) and negative controls (Tsc2-sh neurons) were assessed by Z-score and Z′-factor calculation (Zhang, X. D.; Illustration of SSMD, z score, SSMD*, z* score, and t statistic for hit selection in RNAi high-throughput screens. Journal of biomolecular screening 16, 775-785 (2011)). The following formula was used for Z-score calculation: X−P^{ave}/SDₚ (X=% of GFP⁺ pS6⁺ neurons in the well; P^{ave}=mean of % GFP⁺ pS6⁺ neurons in Positive control wells, SD=Standard Deviation of the values measured in P). The following formula was used for Z-prime calculation: 1−[3*sum (SDₙ+SDₚ)/Yₙ−Y_{p]}]; Yₙ=average negative controls, Yₚ=average positive controls (FIG. 9A). For the screen, the Biomol collection library was used, which includes 480 bioactive compounds with known MOA (ICCB-Longwood screening facility and Human Neuron Core of the Translational Neuroscience Center at BCH). Every compound of the Biomol library was at a concentration optimized to reflect their potency for known target. The library was used a dilution of roughly 30× the potency for each compound for its target. The screen was performed in 96-well plates in duplicate and edge wells were excluded to avoid variability. Neurons were transduced with lentivirus and treated either with vehicle (0.1% v/v DMSO) or with the compounds at DIV19 for 24 hrs. Each plate included three to four vehicle-treated control-sh neurons and three to four vehicle-treated Tsc2-sh neurons. Compounds were administered using the Agilent bravo automated liquid. A Z-score was calculated for each compound in terms of standard deviations from the mean distribution of all the compound-treated Tsc2-knockdown wells assuming that most compounds would not have an effect on S6 phosphorylation (Zhang, X. D.; Illustration of SSMD, z score, SSMD*, z* score, and t statistic for hit selection in RNAi high-throughput screens. Journal of biomolecular screening 16, 775-785 (2011)). The following formula was used for Z-score calculation of the screen compounds: X−C^{ave}/SD of C^{ave} (X=% GFP⁺ pS6⁺ neurons in Tsc2-sh compound-treated well, C^{ave}=average % of GFP⁺ pS6⁺ neurons in all the Compound-treated Tsc2-sh wells, SD of the values measured C). Potential hits were considered compounds that had a z-score less than −1.8 (p<0.0) in both replicas. Total DAPI⁺ counts were used as a criterion for toxic compounds exclusion, toxic compound excluded were 5.8%.

### Differentially Expressed Genes

RNA sequencing data from normal brain and cortical tubers were obtained, and they were normalized using trimmed mean of M values summarized using counts per million (Martin et al., The genomic landscape of tuberous sclerosis complex; Nat. Commun. 8, 15816 (2017)). A list of cilia genes was obtained from Syscilia (syscilia.org/goldstandard.shtml), and cilia gene expression was quantified within normal brain and cortical tubers. To determine whether cilia genes were more likely to be dysregulated in the cortical tubers versus normal brain, LIMMA was used to determine the number of genes expressed at different levels in cortical tubers versus normal brain using an uncorrected p-value of 0.05. As controls, random groups of genes of the same size as the group of cilia genes were selected and utilized to identify the number of genes dysregulated within these random groups. A Z-score was then calculated by comparing the number of dysregulated genes in the cilia group versus the random groups and a p-value based on this Z-score.

### Quantifications and Statistical Analysis

Western blot quantifications were performed by protein normalization using GAPDH as loading control. Level of phosphorylated proteins was expressed as the ratio of phosphorylated/total level after GAPDH normalization. Low sample size datasets were tested for normality and when appropriate they were analyzed with non-parametric tests. IC50 values were calculated using the nonlinear regression equation “dose-response curves—Inhibition” of GraphPad PRISM. Data were expressed as percent of vehicle-treated Tsc2-sh neurons which were considered 100% and drug's concentrations were transformed to logarithmic 10 scale. EC50 values were calculated using the nonlinear regression equation “dose-response curves—Stimulation” of Prism. Data were expressed as percent of vehicle-treated ctrl-sh neurons which were considered 100% and drug's concentrations were transformed to logarithmic 10 scale.

### Other Embodiments

From the foregoing description, it will be apparent that variations and modifications may be made to the disclosure described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of some embodiments herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method for increasing or normalizing ciliation or reducing a ciliation defect in a cell, the method comprising contacting the cell with one or more mTOR inhibitors selected from the group consisting of MCI-186, Nicardipine-HCl, K252A, Tyrphostin 9, LY-294002, rapamycin and everolimus; and/or one or more heat shock protein (Hsp) inhibitors selected from the group consisting of inhibitory nucleic acids, 17-Allylamino-geldanamycin (17-AGG), Geldanamycin (GA), CUDC-305, and NVP-HSP990, thereby increasing or normalizing ciliation or reducing a ciliation defect.

2-8. (canceled)

9. The method of claim 1, wherein ciliation is increased or normalized relative to a reference.

10-14. (canceled)

15. The method of claim 1, wherein the cell is a neuron.

16. The method of claim 1, wherein the cell is in vivo or in vitro.

17-19. (canceled)

20. The method of claim 19, wherein the shRNA is at least 85%, at least 90%, at least 95%, at least 98%, at least 99%, or 100% identical to the following nucleic acid sequence:
5′-CAC TGG CAA GCA CGA AGA AAG-3′

21. The method of claim 19, wherein the shRNA is at least 85%, at least 90%, at least 95%, at least 98%, at least 99%, or 100% identical to the following nucleic acid sequence:
5′-CAC CGG CAA GCA CGA GGA GCG-3′

22. A pharmaceutical composition comprising
one or more Hsp inhibitors is selected from the group consisting of inhibitory nucleic acids, 17-Allylamino-geldanamycin (17-AGG), Geldanamycin (GA), CUDC-305, and NVP-HSP990, and/or
one or more mTOR inhibitors is selected from the group consisting of MCI-186, Nicardipine-HCl, K252A, Tyrphostin 9, LY-294002, rapamycin, and everolimus.

23-35. (canceled)

36. A method of treating a subject with a neurodevelopmental disorder, the method comprising administering to the subject an effective amount of the pharmaceutical composition of claim 22, thereby treating the neurodevelopmental disorder.

37. A method of treating a subject with a neurodevelopmental disorder or a mTORopathy, the method comprising administering to the subject an effective amount of a pharmaceutical composition comprising one or more mTOR inhibitors selected from the group consisting of MCI-186, Nicardipine-HCl, K252A, Tyrphostin 9, LY-294002, rapamycin, and everolimus, and/or one or more heat shock protein (Hsp) inhibitors selected from the group consisting of inhibitory nucleic acids, 17-Allylamino-geldanamycin (17-AGG), Geldanamycin (GA), CUDC-305, and NVP-HSP990, thereby treating the neurodevelopmental disorder or mTORopathy.

38. (canceled)

39. The method of claim 3, wherein the neurodevelopmental disorder is caused by a mutation in a mechanistic target of rapamycin (mTOR) regulatory gene.

40. The method of claim 39, wherein the mTOR regulatory gene is selected from the group consisting of TSC1, TSC2, AKT3, and DEPDC5.

41. (canceled)

42. The method of claim 36, wherein the neurodevelopmental disorder is associated with dysfunctional mechanistic target of rapamycin complex 1 (mTORC1) activity.

43-44. (canceled)

45. The method of claim 36, wherein the neurodevelopmental disorder is associated with a decrease in neuronal cilia.

46. The method of claim 36, wherein the neurodevelopmental disorder is Tuberous Sclerosis Complex (TSC), intellectual disability, brain malformations, cortical tubers, neural ciliopathy, epilepsy, neuropathy, autism, hemimegalencephaly, cortical dysplasia, focal cortical dysplasia, traumatic brain injury, brain tumours, and/or dementia, or a combination thereof.

47-50. (canceled)

51. A method of treating a subject with Tuberous Sclerosis Complex (TSC) or neuronal ciliopathy, the method comprising administering to a subject an effective amount of a pharmaceutical composition comprising one or more mTOR inhibitors selected from the group consisting of MCI-186, Nicardipine-HCl, K252A, Tyrphostin 9, LY-294002, rapamycin, and everolimus; and/or
contacting the cell with one or more heat shock protein (Hsp) inhibitors selected from the group consisting of inhibitory nucleic acids, 17-Allylamino-geldanamycin (17-AGG), Geldanamycin (GA), CUDC-305, and NVP-HSP990, thereby treating TSC or neuronal ciliopathy.

52-65. (canceled)

66. The method of claim 60, wherein the pharmaceutical composition administered to the subject comprises a vector encoding the inhibitory nucleic acid.

67. The method of claim 66, wherein the vector is a viral vector.

68. The method of claim 66, wherein the vector is an adeno-associated virus (AAV) vector.

69. The method of claim 66, wherein the vector comprises a promoter that drives expression of the inhibitory nucleic acid.

70. The method of claim 36, wherein the method is in vivo or in vitro.

71-76. (canceled)

77. A kit for the treatment of a subject with a neurodevelopmental disorder, the kit comprising one or more mTOR inhibitors selected from the group consisting of MCI-186, Nicardipine-HCl, K252A, Tyrphostin 9, LY-294002, rapamycin, and everolimus; and/or
contacting the cell with one or more heat shock protein (Hsp) inhibitors selected from the group consisting of inhibitory nucleic acids, 17-Allylamino-geldanamycin (17-AGG), Geldanamycin (GA), CUDC-305, and NVP-HSP990 for administration to the subject.

78-80. (canceled)

81. The kit of claim 76, further comprising instructions for treating the subject.
